(19) United States
(12) Patent Application Publication
(10) Pub. No.: US 2015/0168830 A1
(43) Pub. Date: Jun. 18, 2015

(54) ACTINIC RAY-SENSITIVE OR RADIATION-SENSITIVE RESIN COMPOSITION, RESIST FILM USING THE SAME, PATTERN FORMING METHOD, AND METHOD FOR MANUFACTURING ELECTRONIC DEVICE AND ELECTRONIC DEVICE, AND COMPOUND

(71) Applicant: FUJIFILM CORPORATION, Tokyo (JP)

(72) Inventors: Akiyoshi GOTO, Haibara-gun (JP); Akinori SHIBUYA, Haibara-gun (JP); Shohei KATAOKA, Haibara-gun (JP); Shuhei YAMAGUCHI, Haibara-gun (JP); Tomoki MATSUDA, Haibara-gun (JP); Keita KATO, Haibara-gun (JP)

(73) Assignee: FUJIFILM CORPORATION, Tokyo (JP)

(21) Appl. No.: 14/633,721

(22) Filed: Feb. 27, 2015

Related U.S. Application Data
(63) Continuation of application No. PCT/JP2013/072484, filed on Aug. 16, 2013.

(30) Foreign Application Priority Data
Aug. 31, 2012 (JP) 2012-191849

(51) Int. Cl. G03F 7/027; G03F 7/20; C07D 327/06; C07D 279/12; G03F 7/038; C07D 217/08; C07D 335/02; C07D 333/46; C07C 323/21; G03F 7/30; C07D 411/04

(52) U.S. Cl. CPC G03F 7/027; G03F 7/30; G03F 7/2041; C07D 327/06; C07D 279/12; C07D 411/04; C07D 217/08; C07D 335/02; C07D 333/46; C07C 323/21; G03F 7/038

(57) ABSTRACT

There is provided an actinic ray-sensitive or radiation-sensitive resin composition containing a compound represented by Formula (1):

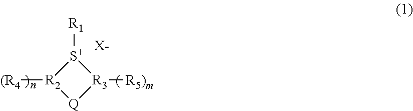

wherein R₁ represents a polycyclic aromatic group or a polycyclic heterocyclic aromatic group,
R₂ represents a (n+2)-valent saturated hydrocarbon group.
R₃ represents a (m+2)-valent saturated hydrocarbon group,
R₄ and R₅ each independently represent a substituent,
Q represents a linking group containing a heteroatom,
m and n each independently represent an integer of 0 to 12, when n is 2 or more, R₄'s may be the same or different, R₄'s may be linked to each other to form a non-aromatic ring together with R₂, when m is 2 or more, R₅'s may be the same or different, and R₅'s may be linked to each other to form a non-aromatic ring together with R₃, and
X⁻ represents a non-nucleophilic anion.

### CROSS REFERENCE TO RELATED APPLICATION

This is a continuation of International Application No. PCT/JP2013/072484 filed on Aug. 16, 2013, and claims priority from Japanese Patent Application No. 2012-191849 filed on Aug. 31, 2012, the entire disclosures of which are incorporated herein by reference.

### BACKGROUND

1. Technical Field

The present invention relates to an actinic ray-sensitive or radiation-sensitive resin composition whose properties are changed by subjecting to reaction upon irradiation with an acitinic ray or radiation, a resist film formed by using the composition, a pattern forming method using the composition, a method for manufacturing an electronic device, an electronic device, and a compound. More specifically, the present invention relates to an actinic ray-sensitive or radiation-sensitive resin composition used for a manufacturing process of a semiconductor such as an IC, a manufacturing process of a circuit board of a liquid crystal, a thermal head or the like, other photofabrication processes, lithographic printing plate and an acid-curable composition, a resist film formed by using the composition, a pattern forming method using the composition, a method for manufacturing an electronic device, an electronic device, and a compound.

2. Background Art

A chemical amplification resist composition is a pattern forming material which generates an acid hi an exposed portion upon irradiation with radiation such as far-ultraviolet rays, and changes the solubility of an actinic radiation-irradiated portion and a non-irradiated portion in a developer by a reaction using the acid as a catalyst to form a pattern on a substrate.

In the case of using a KrF excimer laser as an exposure light source, since a resin containing poly(hydroxystyrene) as a basic structure, which has a small absorption at a region of 248 nm, is used as a main component, a good pattern having a high sensitivity and high resolution may be formed. Accordingly, the resin is regarded as a better system as compared with a typical naphthoquinone diazide/novolak resin system.

Meanwhile, in the case of using a light source having a shorter wavelength, for example, in the case of using an ArF excimer laser (193 nm) as a light source, since a compound having an aromatic group intrinsically has a large absorption at a resion of 193 nm, even the aforementioned chemical amplification system has not been sufficient. On this account, there has been developed a resist for an ArF eximer laser containing an alicyclic hydrocarbon structure.

However, from the viewpoint of the general performance as a resist, it is difficult to find out an appropriate combination of resins, photo-acid generators, basic compounds, additives, solvents and the like to be used, and thus, there still has been no sufficient resist. For example, there is a demand to develop a resist having small pattern collapse, excellent pattern roughness characterises such as exposure latitude and line width roughness (LWR), and small change in performance over time.

In such a situation, various compounds have been developed for a photo-acid generator, which is a main component of a chemical amplification resist compound. For example, Japanese Patent Application Laid-Open No. 2004-59882 describes a photo-acid generator of a naphthylsullonium salt, and Japanese Patent Application Laid-Open No. 2012-31145 and Japanese Patent Application Laid-Open No. 2012-97074 discloses a photo-acid generator of a phenylsulfonium salt in order to improve exposure latitude or depth of focus.

Further, a photo-acid generator is generally required to have high acid generating efficiency, but the high acid generating efficiency means that the photo-acid generator is easily decomposed, and is in a trade-off relationship between the storage stability in many cases. Accordingly, it is required to combine the high acid generating efficiency and the storage stability.

Further, since the photo-acid generator is contained in a large amount in the chemical amplification resist composition, the photo-acid generator is affected considerably on basic performances such as pattern collapse, exposure latitude and LWR.

However, the photo-acid generator as described in Patent Document 1 does not have sufficient performances for acid generating efficiency and storage stability, and thus, further performance enhancement has been required. The photo-acid generators as described in Japanese Patent Application Laid-Open No. 2012-31145 and Japanese Patent Application Laid-Open No. 2012-97074 are not sufficient for basic performances such as exposure latitude. It is required to realize the establishment of all the performances including the storage stability in the development of the photo-acid generator for a photoresist.

In consideration of the background arts, an object of the present invention is to provide an actinic ray-sensitive or radiation-sensitive resin composition satisfying reduction in pattern collapse, enhancement of pattern roughness characteristics such as exposure latitude and LWR, and excellent aging stability at the same time, a resist film and a pattern forming method using the same, a method for manufacturing an electronic device and an electronic device.

### SUMMARY OF INVENTION

The present inventors have studied intensively in order to solve the aforementioned problems, and as a result, the present invention has been achieved.

That is, the present invention has the following constitution.

[1] An actinic ray-sensitive or radiation-sensitive resin composition containing, a compound represented by Formula (1):

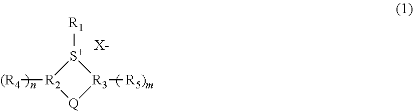

wherein R₁ represents a polycyclic aromatic group or a polycyclic heterocyclic aromatic group.

R₂ represents a (n+2)-valent saturated hydrocarbon group.

R₃ represents a (m+2)-valent saturated hydrocarbon group,

R₄ and R₅ each independently represent a substituent,

Q represents a linking group containing a heteroatom.

m and n each independently represent an integer of 0 to 12, when n is 2 or more, R₄'s may be the same or different, R₄'s may be linked to each other to form a non-aromatic ring together with R₂, when in is 2 or more, R₅'s may be the same or different, and R₅'s may be linked to each other to form a non-aromatic ring together with R₃, and

X′ represents a non-nucleophilic anion.

[2] The actinic ray-sensitive or radiation-sensitive resin composition according to [1],

wherein in Formula (1), Q is any one linking group selected from the group (G) consisting of the following linking groups:

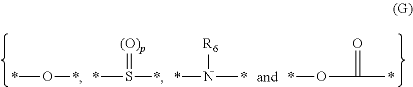

wherein R₆ represents a hydrogen atom or a substituent.

p represents an integer of 0 to 2, and

* represents a bonding hand linking to R₂ or R₃ in Formula (1).

[3] The actinic ray-sensitive or radiation-sensitive resin composition according to [1] or [2],

wherein in Formula (1), X′ is a non-nucleophilic anion represented by Formula (2):

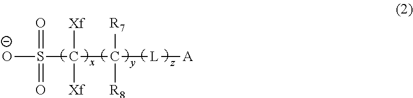

wherein in Formula (2). Xf's each independently represent a fluorine atom or an alkyl group substituted with at least one fluorine atom,

R₇ and R₈ each independently represent a hydrogen atom, a florine atom, an alkyl group or an alkyl group substituted with at least one fluorine atom, when a plurality of R₇'s is present, R₇'s may be the same or different, and when a plurality of R₈'s is present, R₈'S may be the same or different,

L represents a divalent linking group, and when a plurality of L's is present, L's may be the same as or different,

A represents an organic group containing a cyclic structure,

x represents an integer of 1 to 20,

y represents an integer of 0 to 10, and

z represents an integer of 0 to 10.

[4] The actinic ray-sensitive or radiation-sensitive resin composition according to any one of [1] to [3],

wherein a fluorine content of the compound represented by Formula (1) is 0.25 or less, as calculated by (sum of mass of total fluorine atoms contained in the compound represented by Formula (1))/(sum of mass of total atoms contained in the compound represented by Formula (1)).

[5] The actinic ray-sensitive or radiation-sensitive resin composition according to any one of [1] to [4],

wherein in Formula (1). R₁ represents a naphthyl group.

[6] The actinic ray-sensitive or radiation-sensitive resin composition according to [5],

wherein the compound represented by Formula (1) is a compound represented by

Formal (1a):

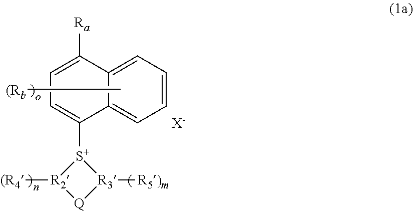

wherein in Formula (1a), Ra represents a hydrogen atom or a substituent,

Rb represents a substituent,

R₂′ and R₃′ each independently represent an alkylene group, and R₄′ and R₅′ each independently represent a substituent,

Q represents a linking group containing a heteroatom,

o represents an integer of it to 6. When o is 2 or more, Rb's may be the same or different,

n and m each independently represent an integer of 0 to 12, when n is 2 or more, R₄′'s may be the same or different, and R₄′'s may be linked to each other to form a non-aromatic ring together with R₂′, and when to is 2 or more, R₅′'s may be the same or different, and R₅′'s may be linked to each other to form a non-aromatic ring together with R₃′, and

X′ represents a non-nucleophilic anion.

[7] the actinic ray-sensitive or radiation-sensitive resin composition according to [1],

wherein in Formula (1a), Ra represents a group represented by Formula (1a′)

*-A-R₆)ₛ (1a′)

wherein in Formula (1a′). A represents a divalent or trivalent heteroatom.

R₆ represents a hydrogen atom or a substituent,

s represents 1 when A is a divalent heteroatom, and s represents 2 when A is a trivalent heteroatom, and when s is 2, two R₆'s may be the same or different, and

* represents a bonding hand connecting to the benzene ring in Formula (1a)

[8] The actinic ray-sensitive or radiation-sensitive resin composition according to any one of [1] to [7], to further containing a resin which is decomposed by the action of an acid to change solubility in a developer.

[9] The actinic ray-sensitive or radiation-sensitive resin composition according to any one of [1] to [8], further containing a low molecular weight compound having a nitrogen atom and a group capable of leaving by the action of an acid, or a basic compound.

[10] A resist film formed by the actinic ray-sensitive or radiation-sensitive resin composition according to any one of [1] to [9].

[11] A pattern forming method including:
exposing the resist film according to [10]; and

developing the exposed resist film.

[12] The pattern forming method according to [11],

wherein the exposing is liquid immersion exposure.

[13] A method for manufacturing an electronic device, containing the pattern forming method according to [11] or [12].

[14] An electronic, device manufactured by the method for manufacturing an electronic device according to [13].

[15] A compound represented by Formula (4):

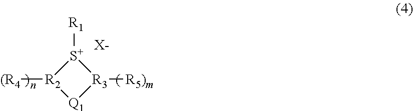

wherein in Formula (4), R₁ represents a polycyclic aromatic group ro a polycyclic heterocyclic aromatic group,

R₂ and R₃ each independently represent a (m+2)-valent saturated hydrocarbon group,

R₄ and R₅ each independently represent a substituent,

n and m each independently represent an integer of 0 to 12, when n is 2 or more. R₄'s may be the same or different, R₄'s may be linked to each other to form a non-aromatic ring together with R₂, and when m is 2 or more, R₅'s may be the same or different, and R's may be linked to each other to form a non-aromatic ring together with R₃,

X⁻ represents a non-nucleophilic anion, and

Q₁ represents any one linking group selected from the group consisting of the linking groups shown below:

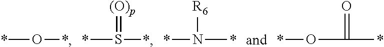

wherein R₆ represents a hydrogen atom or a substituent,
p represents an integer of 0 to 2, and

* represents a bonding hand linking to R₂ or R₁ in Formula (4).

### DETAILED DESCRIPTION OF EXEMPLARY EMBODIMENTS

Hereinafter, embodiments of the present invention will be described in detail.

In representing a group (atomic group) in the present specification, the representation which does not describe the substitution and unsubstitution also includes having substituents along with having no substituent. For example, “an alkyl group” includes not only an alkyl group having no substituent (an unsubstituted alkyl group) but also an alkyl group having a substituent (a substituted alkyl group).

The term “actinic ray” or “radiation” in the present specification refers to, for example, a bright line spectrum of a mercury lamp, far-ultraviolet rays represented by an excimer laser, extreme ultraviolet (FAN) rays, X-rays, an electron beam (EB) and the like. Further, the term “light” in the present invention refers to the actinic rays or the radiations.

In addition, unless otherwise specifically indicated, the term “exposure” in the present specification includes not only the exposure performed using a mercury lamp, far-ultraviolet rays represented by an excimer laser, extreme ultraviolet rays, X-rays, EUV rays and the like, but also drawing performed by a particle beam such as an electron beam and an ion beam.

The actinic ray-sensitive or radiation-sensitive resin composition of the present invention contains (A) a compound capable of generating an acid upon irradiation with an actinic ray or radiation (hereinafter, also referred to as a “compound (A)” or “acid generator (A)”).

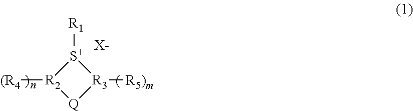

In Formula (1),

R₁ represents a polycyclic aromatic group or a polycyclic heterocyclic aromatic group.

R₂ represents a (n+2)-valent saturated hydrocarbon group.

R₃ represents a (m+2)-valent saturated hydrocarbon group.

R₄ and R₅ each independently represent a substituent.

Q represents a linking group containing a heteroatom.

n and m each independently represent an integer of 0 to 12. When n is 2 or more, R₄'s may be the same or different, and R₄'s may be linked to each other to form a non-aromatic ring together with R₂. When m is 2 or more, R₅'s may be the same or different, and R₅'s may be linked to each other to form a non-aromatic ring together with R₃.

X⁻ represents a non-nucleophilic anion.

By the actinic ray-sensitive or radiation-sensitive resin composition of the present invention, it is possible to achieve enhancement of pattern roughness characteristics such as exposure latitude and LWR, and excellent, aging stability at the same time. The reason is unclear, but it is assumed as follows.

First, the actinic ray-sensitive or radiation-sensitive resin composition according to the present invention contains the compound (A). Since the compound (A) has a ring structure containing a heteroatom and also, has a polycyclic structure at the position of R₁ in Formula (1) it is considered that the compound (A) interacts with a substrate to enhance the adhesion between the resist film and the substrate, and consequently contributes to the improvement in pattern collapse in the formed pattern.

Further, since the compound (A) undergoes light absorption and C—S⁺ bond cleavage after excitation with high efficiency, acid is generated in a large amount after exposure, and the acid is uniformly distributed in the photosensitive resist film. It is considered that this fact contributes to the improvement in MR.

Further, since the compound (A) has a bulky polycyclic aromatic group, it is considered that diffusion of the compound (A) in the resist film is suppressed, and as a result, the exposure latitude is improved. In addition, since the compound (A) has a ring structure containing a heteroatom, the compound (A) interacts with a resin component in the resist film at a high level. It is also assumed that this becomes a main factor that reduces the diffusivity of the compound (A).

Furthermore, since a heteroatom having high polarity is present in Q around the sulfur atom of the sulfonium cation in the compound (A), the affinity with a hydrophobic component in the resist is suppressed. Accordingly, it is difficult to undergo nucleophilic addition decomposition. Further, since the compound (A) has a polycyclic aromatic group, it is considered that the steric hindrance of the compound (A) becomes larger to suppress the nucleophilic attack by a component in the resist to the sulfur atom in the sulfonium cation, and consequently, change in performances over time may be suppressed.

[1] Compound (A) Represented by Formula (1)

The actinic ray-sensitive or radiation-sensitive resin composition of the present invention contains a compound (A) represented by the following Formula (1) as described above. The compound (A) is a compound capable of generating an acid upon irradiation with an actinic ray or radiation.

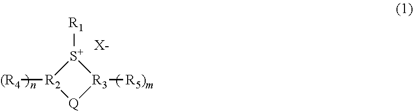

In Formula (1),

R₁ represents a polycyclic aromatic group or a polycyclic heterocyclic aromatic group.

R₂ represents a (n+2)-valent saturated hydrocarbon group.

R₃ represents a (m+2)-valent saturated hydrocarbon group.

R₄ and R₅ each independently represent a substituent.

Q represents a linking group containing a heteroatom,

n and m each independently represent an integer of 0 to 12. When n is 2 or more. R₄'s may be the same or different, and R₄'s may be linked to each other to form a non-aromatic ring together with R₂. When m is 2 or more. R₅'s may be the same or different, and R₅'s may be linked to each other to form a non-aromatic ring together with R₃.

X′ represents a non-nucleophilic anion.

Hereinafter, the compound (A) will be described in detail.

The polycyclic aromatic group as R₁ is preferably a polycyclic aromatic hydrocarbon group having 10 to 20 carbon atoms or a polycyclic heterocyclic aromatic group having 8 to 20 carbon atoms. Specific examples of the polycyclic aromatic hydrocarbon group may include a naphthyl group, an azulenyl group, an acenaphthylenyl group, a phenanthrenyl group, a phenalenyl group, a phenanthracenyl group, a fluorenyl group, an anthracenyl group, a pyrenyl group, a benzopyrenyl group, a biphenyl group and the like. Specific examples of the polycyclic heterocyclic aromatic group may include an acridine group, a xanthene group, a carbazole group, an indole group, a benzopyran group, a dihydronaphthopyran group, a benzothiazole group, a benzoxazole group, a benzofuran group, a dibenzofuran group, a dihydrobenzofuran group, a benzothiophene group, a dihydrobenzothiophene group, a chroman group, a thiochroman group, a benzodioxin group, a dibenzodidoxane group, a phenoxathiin group, a dibenzo-1,4-dithiane group, a phenothiazine group, a dibenzothiophene group and the like.

R₁ is preferably a naphthyl group, an anthracenyl group, a fluorenyl group, a carbazole group, an indole group, a benzopyran group, a dihydronaphthopyran group, a benzoxazole group, a benzofuran group, a dibenzofuran group, a dihydrobenzofuran group, a dihydrobenzothiophene group, a chroman group, a dibenzodioxin group, a phenoxathiin group, dibenzo-1,4-dithiane group, a phenothiazine group or a dibenzothiophene group, more preferably a naphthyl group, an anthracenyl group, a carbazole group, a dibenzofuran group, a dihydrobenzofuran group, a chroman group or a dibenzodioxin group, and, from the viewpoint of establishment of exposure latitude, aging stability and transparency, still more preferably a naphthyl group.

R₁ may have a substituent, and the position and the number of substituents which may be introduced are not particularly limited. The substituent which may be introduced is, for example, a halogen atom (for example, fluorine, chlorine and bromine), an alkyl group (preferably a straight or branched alkyl group having 1 to 20 carbon atoms, and may have an oxygen atom, a sulfur atom or a nitrogen atom in its alkyl chain. Specific examples thereof may include a straight alkyl group such as a methyl group, an ethyl group, a n-propyl group, a n-butyl group, a n-pentyl group, a n-hexyl group, a n-octyl group, a n-dodecyl group, a n-tetradecyl group and a n-octadecyl group, and a branched alkyl group such as an isopropyl group, an isobutyl group, a t-butyl group, a neopentyl group and a 2-ethylhexyl group), a cycloalkyl group (preferably a cycloalkyl group having 3 to 20 carbon atoms, and may have an oxygen atom or a sulfur atom in its ring. Specific examples thereof may include a cyclopropyl group, a cyclopentyl group, a cyclohexyl group, a norbornyl group, an adamantyl group and the like), an alkenyl group (preferably an alkenyl group having 2 to 48 carbon atoms, more preferably 2 to 18 carbon atoms, and examples thereof include vinyl, allyl and 3-buten-1-yl), an aryl group (preferably an aryl group having 6 to 48 carbon atoms, more preferably 6 to 24 carbon atoms, and examples thereof include phenyl or naphthyl), a heterocyclic group (a heterocyclic group having 1 to 32 carbon atoms, more preferably 1 to 18 carbon atoms, and examples thereof include 2-thienyl, 4-pyridyl, 2-furyl, 2-pyrimidyl, 1-pyridyl, 2-benzothiazolyl, 1-imidazolyl, 1-pyrazolyl and benzotriazol-1-yl), a silyl group (preferably a silyl group having 3 to 38 carbon atoms, more preferably 3 to 18 carbon atoms, and examples thereof include trimethylsilyl, triethylsilyl, tributylsilyl, t-butyldimethylsilyl and t-hexyldimethylsilyl), a hydroxyl group, a cyano group, a nitro group, an alkoxy group (preferably an alkoxy group having 1 to 48 carbon atoms, more preferably 1 to 24 carbon atoms; and examples thereof include an alkyloxy group such as methoxy, ethoxy, 1-butoxy, 2-butoxy, isopropoxy, t-butoxy and dodecyloxy; and a cycloalkyloxy group such as cyclopentyloxy and cyclohexyloxy), an aryloxy group (preferably an aryloxy group having 6 to 48 carbon atoms, more preferably 6 to 24 carbon atoms, and examples thereof include phenoxy and 1-naphthoxy), a heterocyclic oxy group (preferably a heterocyclic oxy group having 1 to 32 carbon atoms, more preferably 1 to 18 carbon atoms, and examples thereof include 1-phenyltetrazol-5-oxy and 2-tetrahydropyranyloxy),

a silyloxy group (preferably a silyloxy group having 1 to 32 carbon atoms, more preferably 1 to 18 carbon atoms, and examples thereof include trimethylsilyloxy, t-butyldimethylsilyloxy and diphenylmethylsilyloxy), an acyloxy group (preferably an acyloxy group having 2 to 48 carbon atoms, more preferably 2 to 24 carbon atoms, and examples thereof include acetoxy, pyvaloyloxy, benzoyloxy and dodecanoyloxy), an alkoxycarbonyloxy group (preferably an alkoxycarbonyloxy group having 2 to 48 carbon atoms, more preferably 2 to 24 carbon atoms, and examples thereof include an alkyloxycarbonyloxy group such as ethoxycarbonyloxy and t-butoxycarbonyloxy; and a cycloalkyloxycarbonyloxy group such as a cyclohexyloxycarbonyloxy group), an aryloxycarbonyloxy group (preferably an aryloxycarbonyloxy group having 7 to 32 carbon atoms, more preferably 7 to 24 carbon atoms, and examples thereof include phenoxycarbonyloxy), a carbamoyloxy group (preferably a carbamoyloxy group having 1 to 48 carbon atoms, more preferably 1 to 24 carbon atoms, and examples thereof include N,N-dimethylcarbamoyloxy, n-butylcarbamyloxy, n-phenylcarbamoyloxy or n-ethyl-N-phenylcarbamoyloxy), a sulfamoyloxy group (preferably a sulfamoyloxy group having 1 to 32 carbon atoms, more preferably 1 to 24 carbon atoms, and examples thereof include N,N-dimethylsulfamoyloxy and n-propylsulfamoyloxy), an alkylsulfonyloxy group (preferably an alkylsulfonyloxy group having 1 to 38 carbon atoms, more preferably 1 to 24 carbon atoms, and examples thereof may include methylsulfonyloxy, hexadecylsulfonyloxy and cyclohexylsulfonyloxy),

an arylsulfonyloxy group (preferably an arylsulfonyloxy group having 6 to 32 carbon atoms, more preferably 6 to 24 carbon atoms, and examples thereof include phenylsulfonyloxy), an acyl group (preferably an acyl group having 1 to 48 carbon atoms, more preferably 1 to 24 carbon atoms, and examples thereof include formyl, acetyl, pyvaloyl, benzoyl, tetradecanoyl and cyclohexanoyl), an alkoxycarbonyl group (preferably an alkoxycarbonyl group having 2 to 48 carbon atoms, more preferably 2 to 24 carbon atoms, and examples thereof include methoxycarbonyl, ethoxycarbonyl, octadecyloxycarbonyl, cyclohexyloxycarbonyl and 2,6-di-tert-butyl-4-methylcyclohexyloxycarbonyl), an aryloxycarbonyl group (preferably an aryloxycarbonyl group having 7 to 32 carbon atoms, more preferably 7 to 24 carbon atoms, and examples thereof include phenoxycarbonyl), a carbamoyl group (preferably a carbamoyl group having 1 to 48 carbon atoms, more preferably 1 to 24 carbon atoms, and examples thereof include carbamoyl. N,N-diethylcarbamoyl, n-ethyl-N-octylcarbamoyl, N,N-dibutylcarbamoyl, n-propylcarbamoyl, n-phenylcarbamoyl, n-methyl-N-phenylcarbamoyl and N,N-dicyclohexylcarbamoyl), an amino group (preferably an amino group having 32 or less carbon atoms, more preferably 24 or less carbon atoms, and examples thereof include amino, methylamino, N,N-dibutylamino, tetradecylamino, 2-ethylhexylamino and cyclohexylamino), an anilino group (preferably an anilino group having 6 to 32 carbon atoms, more preferably 6 to 24 carbon atoms, and examples thereof include aniline and n-methylanilino), a heterocyclic amino group (preferably a heterocyclic amino group having 1 to 32 carbon atoms, more preferably 1 to 18 carbon atoms, and examples thereof include 4-pyridylamino), a carbonamide group (preferably a carbonamide group having 2 to 48 carbon atoms, more preferably 2 to 24 carbon atoms, and examples thereof include acetamide, benzamide, tetradecanamide, pyvaloylamide and cyclohexanamide), a ureido group (preferably a ureido group having 1 to 32 carbon atoms, more preferably 1 to 24 carbon atoms, and examples thereof include ureido, N,N-dimethylureido and n-phenylureido), an imide group (preferably an imide group having 36 or less carbon atoms, more preferably 24 or less carbon atoms, and examples thereof include n-succinimide and n-phthalimide), an alkoxycarbonylamino group (preferably an alkoxycarbonylamino group having 2 to 48 carbon atoms, more preferably 2 to 24 carbon atoms, and examples thereof include methoxycarbonylamino, ethoxycarbonylamino, t-butoxycarbonylamino, octadecyloxycarbonylamino and cyclohexyloxycarbonylamino), an aryloxycarbonylamino group (preferably an aryloxycarbonylamino group having 7 to 32 carbon atoms, more preferably 7 to 24 carbon atoms, and examples thereof include phenoxycarbonylamino), a sulfonamide group (preferably a sulfonamide group having 1 to 48 carbon atoms, more preferably 1 to 24 carbon atoms, and examples thereof include methanesulfonamide, butanesulfonamide, benzenesulfonamide, hexadecanesulfonamide and cyclohexanesulfonamide), a sulfamoylamino group (preferably a sulfamoylamino group having 1 to 48 carbon atoms, more preferably 1 to 24 carbon atoms, and examples thereof include N,N-dipropylsulfamoylamino, a n-ethyl-N-dodecylsulfamoylamino), an azo group (preferably an azo group having 1 to 32 carbon atoms, more preferably 1 to 24 carbon atoms, and examples thereof include phenylazo and 3-pyrazolylazo), an alkylthio group (preferably an alkylthio group having 1 to 48 carbon atoms, more preferably 1 to 24 carbon atoms, and examples thereof include methylthio, ethylthio, octylthio and cyclohexylthio), an arylthio group (preferably an arylthio group having 6 to 48 carbon atoms, more preferably 6 to 24 carbon atoms, and examples thereof include phenylthio), a heterocyclic thio group (preferably a heterocyclic thio group having 1 to 32 carbon atoms, more preferably 1 to 18 carbon atoms and examples thereof include 2-benzothiazolylthio, 2-pyridylthio, l-phenyltetrazolylthio), an alkylsulfinyl group (preferably an alkylsulfinyl group having 1 to 32 carbon atoms, more preferably 1 to 24 carbon atoms, and examples thereof include dodecanesulfinyl), an arylsulfinyl group (preferably an arylsulfinyl group having 6 to 32 carbon atoms, more preferably 6 to 24 carbon atoms, and examples thereof include phenylsulfinyl), an alkylsulfonyl group (preferably an alkylsulfonyl group having 1 to 48 carbon atoms, more preferably 1 to 24 carbon atoms, and examples thereof include methylsulfonyl, ethylsulfonyl, propylsulfonyl, butylsulfonyl, isopropylsulfonyl, 2-ethylhexylsulfonyl, hexadecylsulfonyl, octylsulfonyl and cyclohexylsulfonyl), an arylsulfonyl group (preferably an arylsulfonyl group having 6 to 48 carbon atoms, more preferably 6 to 24 carbon atoms, and examples thereof include phenylsulfonyl, 1-naphthylsulfonyl), a sulfamoyl group (preferably a sulfamoyl group having 32 or less carbon atoms, more preferably 24 or less carbon atoms, and examples thereof include sulfamoyl, N,N-dipropylsulfamoyl, n-ethyl-N-dodecylsulfamoyl, n-ethyl-N-phenylsulfamoyl and n-cyclohexylsulfamoyl), a sulfo group, a phosphonyl group (preferably a phosphonyl group having 1 to 32 carbon atoms, more preferably 1 to 24 carbon atoms, and examples thereof include phenoxyphosphonyl, octyloxyphosphonyl and phenylphosphonyl) or a phosphinoylamino group (preferably a phosphinoylamino group having 1 to 32 carbon atoms, more preferably 1 to 24 carbon atoms, and examples thereof include diethoxyphosphinoylamino and dioctyloxyphosphinoylamino).

The substituent which may be possessed by R₁ may be preferably a straight or branched alkyl group (preferably having 1 to 10 carbon atoms), a cycloalkyl group (preferably having 3 to 20 carbon atoms), a hydroxyl group, an alkoxy group (preferably having 1 to 10 carbon atoms), an amino group or an alkoxycarbonylamino group (preferably having 1 to 10).

The substituent which may be possessed by R₁ may be more preferably a straight or branched alkyl group (preferably having 1 to 10 carbon atoms), a cycloalkyl group (preferably having 3 to 20 carbon atoms), a hydroxyl group, an alkoxy group (preferably having 1 to 10 carbon atoms) or an alkoxycarbonylamino group (preferably having 1 to 10 carbon atoms).

The substituent which may be possessed by R₁ may be still more preferably a straight or branched alkyl group (preferably having 1 to 10 carbon atoms), a cycloalkyl group (preferably having 3 to 20 carbon atoms) or an alkoxy group (preferably having 1 to 10 carbon atoms).

R₂ represents a (n+2)-valent saturated hydrocarbon group, and is preferably a straight or branched saturated hydrocarbon group having preferably 1 to 6 carbon atoms, more preferably 1 to 4 carbon atoms, and still more preferably 2 or 3 carbon atoms.

R₂ is preferably a (n+2)-valent straight saturated hydrocarbon group having 1 to 4 carbon atoms, more preferably a (n+2)-valent straight saturated hydrocarbon group having 1 to 3 carbon atoms, and particularly preferably (n+2)-valent straight saturated hydrocarbon group having 2 carbon atoms.

R₃ represents a (m+2)-valent saturated hydrocarbon group, and is preferably a straight or branched saturated hydrocarbon group having preferably 1 to 6 carbon atoms, more preferably 1 to 4 carbon atoms, and still more preferably 2 or 3.

R₃ is preferably a (m+2)-valent straight saturated hydrocarbon group having 1 to 4 carbon atoms, more preferably a (m+2)-valent straight saturated hydrocarbon group having 1 to 3 carbon atoms, and particularly preferably a (m+2)-valent straight saturated hydrocarbon group having 2 carbon atoms.

R₄ and R₅ may be exemplified by the aforementioned substituents as a substituent which may be possessed by R₁. R₄ and R₅ may be preferably an alkyl group which may have a substituent, a cycloalkyl group which may have a substituent, an alkenyl group which may have a substituent or an aryl group which may have a substituent, more preferably an alkyl group which may have a substituent, and still more preferably an unsubstituted alkyl group.

The alkyl group as R₄ and R₅ is preferably a straight or branched alkyl group having 1 to 20 carbon atoms, and may have an oxygen atom, a sulfur atom or a nitrogen atom in its alkyl chain. Specific examples thereof may include a straight alkyl group such as a methyl group, an ethyl group, a n-propyl group, a n-butyl group, a n-pentyl group, a n-hexyl group, a n-octyl group, a n-dodecyl group, a n-tetradecyl group and a n-octadecyl group, and a branched alkyl group such as an isopropyl group, an isobutyl group, a t-butyl group, a neopentyl group and a 2-ethylhexyl group. Examples of the alkyl group having a substituent may include a cyanomethyl group, a 2,2,2-trifluoroethyl group, a methoxycarbonylmethyl group, an ethoxycarbonylmethyl group and the like.

The cycloalkyl group as R₄ and R₅ may be preferably a cycloalkyl group having 3 to 20 carbon atoms, and may have an oxygen atom in its ring. Specific examples thereof may include a cyclopropyl group, a cyclopentyl group, a cyclohexyl group, a norbornyl group, an adamantyl group and the like.

The aryl group as R₄ and R₅ may be preferably an aryl group having 6 to 14 carbon atoms, and examples thereof may include a phenyl group, a naphthyl group and the like.

The alkenyl group as R₄ and R₅ may be preferably an alkenyl group having 2 to 20 carbon atoms, and specific examples thereof may include a group having a double bond at any position in the alkyl group as R₄ and R₅ as described above.

Examples of the substituent which may be possessed by these groups may include the same specific examples as the specific examples as described above as a substituent which may be possessed by R₁.

n and m each independently represent an integer of 0 to 12, as described above. When n is 2 or more. R₄'s may be the same or different, and when m is 2 or more, R₅'s may be the same or different. n and m are preferably an integer of 0 to 3, and more preferably 0.

As described above, when n is 2 or more. R₄'s may be linked to each other to form a non-aromatic ring together with R₂, and the non-aromatic ring is preferably a 5- or 6-membered ring, and particularly preferably a 6-membered ring.

Further, as described above, when m is 2 or more, R₅'s may be linked to each other to form a non-aromatic ring together with R₃, and the non-aromatic ring is preferably a 5- or 6-membered ring, and particularly preferably a 6-membered ring.

Q represents a linking group containing a heteroatom, as described above, and is preferably any one linking group selected from the group (G) consisting of the following linking groups.

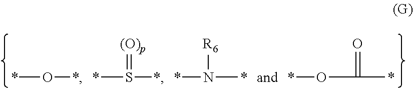

In the formulas. R₆ represents a hydrogen atom or a substituent. p represents an integer of 0 to 2. * represents a bonding hand linking to R₂ or R in Formula (1).

Examples of R₁₀ as a substituent may include the same groups as R₁ to R₄ as described above.

In an aspect of the present invention, the linking group represented by Q is preferably —O—.

The substituent represented by R₁₀ is preferably a group capable of decreasing the basicity of the nitrogen atom. Specific examples thereof may include a group having an electron-withdrawing group such as an acyl group and a sulfonate group. Examples of the acyl group may include a formyl group, an acetyl group, a pyvaloyl group, a benzoyl group, a tetradecanoyl group and a cyclohexanoyl group. Examples of the sulfonate group may include a methanesulfonate group, an ethanesulfonate group, a propanesulfonate group, a butanesulfonate group, a para-toluenesulfonate group and a trifluoromethanesulfonate group.

In an aspect of the present invention, it is preferred that the compound (A) is a compound represented by the following Formula (1a).

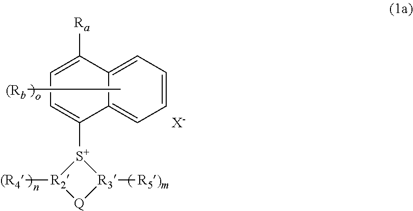

In Formula (1a),

Ra represents a hydrogen atom or a substituent.

Rb represents a substituent.

R₂′ and R₃′ each independently represent an alkylene group, and R₄′ and R₅′ each independently represent a substituent.

Q represents a linking group having a heteroatom.
o represents an integer of 0 to 6. When o is 2 or more, Rb's may be the same or different.

n and m each independently represent an integer of 0 to 12. When n is 2 or more, R₄′'s may be the same or different, and R₄′'s may be linked to each other to form a non-aromatic ring together with R₂′. When m is 2 or more, R₅′'s may be the same or different, and R₅′'s may be linked to each other to form a non-aromatic ring together with R₃′.

X′ represents a non-nucleophilic anion.

R₂′, R₃, R₄′, R₅′, Q, m, n and X′ in the formula have the same meaning as R₂, R₃, R₄, R₅, Q, m, n and X′ in Formula (1) as described above.

The substituent represented by Rb may include the same specific examples as the substituent which may be possessed by R₁ in Formula (1) as described above, and preferred ranges are also the same.

Further, the substituent represented by Ra may include the same specific examples as the substituent which may be possessed by R₁ in Formula (1).

It is preferred that Ra is a group represented by the following Formula (1a′) from the viewpoint of improving the exposure latitude by increasing the interaction between the compound (A) and the resin component.

*-A-R₆)ₛ (1a)

In Formula (1a′),

A represents a divalent or trivalent heteroatom.

R₆ represents a hydrogen atom or a substituent.

s represents 1 when A is a divalent heteroatom, or s represents 2 when A is a trivalent heteroatom. When s is 2, two R₆'s may be the same or different.

* represents a bonding hand connecting to the benzene ring in Formula (1a).

A represents a divalent or trivalent heteroatom, and is preferably an oxygen atom, a sulfur atom or a nitrogen atom, more preferably an oxygen atom or a nitrogen atom, and still more preferably an oxygen atom.

R₆ represents a hydrogen atom or a substituent. Examples of the substituent may include the same specific examples as the substituent which may be possessed by R₁ in Formula (1) as described above. Preferred examples of the substituent R₆ may include the same specific examples and preferred examples exemplified as the alkyl group, the cycloalkyl group, the alkenyl group, the aryl group and the acyl group in the examples of the substituent which may be possessed by R₁. The substituent represented by R₆ may further have a substituent, and examples of the substituent may include a halogen atom such as a fluorine atom, a hydroxyl group, a nitro group, a cyano group, a carboxyl group, a carbonyl group, a alkyl group (preferably having 1 to 6 carbon atoms), a cycloalkyl group (preferably having 3 to 10 carbon atoms), an aryl group (preferably having 6 to 14 carbon atoms), an alkoxy group (preferably having 1 to 10 carbon atoms), an acyl group (preferably having 2 to 20 carbon atoms), an acyloxy group (preferably having 2 to 10 carbon atoms), an alkoxycarbonyl group (preferably having 2 to 20 carbon atoms), an aminoacyl group (preferably having 2 to 10 carbon atoms) and the like. The cycloalkyl group, aryl group, alkoxy group and alkoxycarbonyl group as the substituent may be substituted with a halogen atom such as a fluorine atom. The ring structure such as an aryl group and a cycloalkyl group may further substituted with an alkyl group (preferably having 1 to 10 carbon atoms). The aminoacyl group may be further substituted with an alkyl group (preferably having 1 to 10 carbon atoms).

Examples of the non-nucleophilic anion represented by X′ in Formula (1) may include a sulfonate anion, a carboxylate anion, a sulfonylimide anion, a bis(alkylsulfonyl)imide anion, a tris(alkylsulfonyl)methyl anion and the like. The non-nucleophilic anion is an anion having an extremely low ability of causing a nucleophilic reaction and capable of suppressing the decomposition with time due to an intramolecular nucleophilic reaction. Accordingly, the aging stability of the resist liquid is improved.

Examples of the sulfonate anion may include an alkylsulfonate anion, an arylsulfonate anion, a camphorsulfonate anion and the like.

Examples of the carboxylate anion may include an alkylcarboxylate anion, an arylcarboxylate anion, an aralkylcarboxylate anion and the like.

The alkyl group in the alkylsulfonate anion is preferably an alkyl group having 1 to 30 carbon atoms, and examples thereof may include a methyl group, an ethyl group, a propyl group, an isopropyl group, a n-butyl group, an isobutyl group, a sec-butyl group, a pentyl group, a neopentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a cyclopropyl group, a cyclopentyl group, a cyclohexyl group, an adamantyl group, a norbornyl group, a bornyl group and the like.

The aryl group in the arylsulfonate anion is preferably an aryl group having 6 to 14 carbon atoms, and examples thereof may include a phenyl group, a tolyl group, a naphthyl group and the like.

The alkyl group and the aryl group in the alkylsulfonate anion and the arylsulfonate anion may have a substituent.

Examples of the substituent may include a halogen atom, an alkyl group, an alkoxy group, an alkylthio group and the like.

Examples of the halogen atom may include a chlorine atom, a bromine atom, a fluorine atom, an iodine atom and the like.

The alkyl group is, for example, preferably an alkyl group having 1 to 15 carbon atoms, and examples thereof may include a methyl group, an ethyl group, a propyl group, an isopropyl group, a n-butyl group, an isobutyl group, a sec-butyl group, a pentyl group, a neopentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group and the like.

The alkoxy group is, for example, preferably an alkoxy group having 1 to 5 carbon atoms, and examples thereof may include a methoxy group, an ethoxy group, a propoxy group, a butoxy group and the like.

The alkylthio group is, for example, preferably an alkylthio group having 1 to 15 carbon atoms, and examples thereof may include a methylthio group, an ethylthio group, a propylthio group, an isopropylthio group, a n-butylthio group, an isobutylthio group, a sec-butylthio group, a pentylthio group, a neopentylthio group, a hexylthio group, a heptylthio group, an octylthio group, a nonylthio group, a decylthio group, an undecylthio group, a dodecylthio group, a tridecylthio group, a tetradecylthio group, a pentadecylthio group, a hexadecylthio group, a heptadecylthio group, an octadecylthio group, a nonadecylthio group, an eicosylthio group and the like. Meanwhile, the alkyl group, the alkoxy group and the alkylthio group may be further substituted with a halogen atom (preferably a fluorine atom).

Examples of the alkyl group in the alkylcarboxylate anion may include the same alkyl group at that in the alkylsulfonate anion.

Examples of the aryl group in the arylcarboxylate anion may include the same aryl group as that in the arylsulfonate anion.

The aralkyl group in the aralkylcarboxylate anion is preferably an aralkyl group having 7 to 12 carbon atoms, and examples thereof may include a benzyl group, a phenethyl group, a naphthylmethyl group, a naphthylethyl group, a naphthylmethyl group and the like.

The alkyl group, the aryl group and the aralkyl group in the alkylcarboxylate anion, the arylcarboxylate anion and the aralkylcarboxylate anion may have a substituent, and examples of the substituent may include the same halogen atom, alkyl group, alkoxy group, alkylthio group and the like as those in the arylsulfonate anion.

Examples of the sulfonylimide anion may include a saccharin anion.

The alkyl group in the bis(alkylsulfonyl)imide anion and the tris(alkylsulfonyl)methyl anion is preferably an alkyl group having 1 to 5 carbon atoms, and examples thereof may include a methyl group, an ethyl group, a propyl group, an isopropyl group, a n-butyl group, an isobutyl group, a sec-butyl group, a pentyl group, a neopentyl group and the like. The alkyl group may have a substituent, and examples of the substituent may include a halogen atom, an alkyl group substituted with a halogen atom, an alkoxy group, alkylthio group and the like, and preferably an alkyl group substituted with a fluorine atom.

Examples of other non-nucleophilic anions may include a fluorinated phosphorus, a fluorinated boron, a fluorinated antimony and the like.

The non-nucleophilic anion of X′ is preferably an alkanesulfonate anion which is substituted with a fluorine atom at the α-position of the sulfonic acid, an arylsulfonate anion substituted with a group having a fluorine atom or a fluorine atom, a bis(alkylsulfonyl)imide anion in which the alkyl group is substituted with a fluorine atom, or a tris(alkylsulfonyl)methide anion in which the alkyl group is substituted with a fluorine atom. The non-nucleophilic anion of X is particularly preferably a perfluoroalkanesulfonate anion having 1 to 8 carbon atoms, a nonafluorobutanesulfonate anion or a perfluorooctanesulfonate anion.

In an aspect of the present invention, it is preferred that the non-nucleophilic anion of X′ is represented by Formula (2). In this case, since the generated acid is bulky and the diffusion of the acid is suppressed, it is assumed that the improvement in exposure latituted is further promoted.

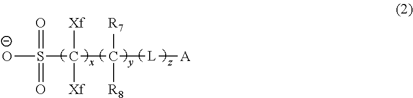

In Formula (2),

Xf's each independently represent a fluorine atom or an alkyl group substituted with at least one fluorine atom.

R₇ and R₈ each independently represent a hydrogen atom, a fluorine atom, an alkyl group or an alkyl group substituted with at least one fluorine atom. When a plurality of R₇'s is present. R₇'s may be the same or different, and when a plurality of R₈'s is present, R₈'s may be the same or different.

L represents a divalent linking group, and when a plurality of L's is present, L's may be the same or different.

A represents an organic group containing a cyclic structure.

x represents an integer of 1 to 20. y represents an integer of 0 to 10. z represents an integer of 0 to 10.

x represents an integer of 1 to 20, preferably 1 to 10, more preferably 1 to 6, and particularly preferably 1 to 3. y represents an integer of 0 to 10. z represents an integer of 0 to 10.

The anion of Formula (2) will be described in detail.

Xf is a fluorine atom or an alkyl group substituted with at least one fluorine atom, and the alkyl group in the alkyl group substituted with a fluorine atom is preferably an alkyl group having 1 to 10 carbon atoms, and more preferably an alkyl group having 1 to 4 carbon atoms. Further, the alkyl group substituted with a fluorine atom of Xf is preferably a perfluoroalkyl group.

Xf is preferably a fluorine atom or a perfluoroalkyl group having 1 to 4 carbon atoms. Specific examples thereof may include a fluorine atom, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉, and CH₂CH₂C₄F₉, and among them, preferably a fluorine atom and CF₃. Particularly, it is preferred that both Xf's are a fluorine atom.

R₆ and R₇ represent a hydrogen atom, a fluorine atom, alkyl group or an alkyl group substituted with at least one fluorine atom as described above, and the alkyl group preferably has 1 to 4 carbon atoms. More preferred is a perfluoroalkyl group having 1 to 4 carbon atoms. Specific examples of the alkyl group substituted with at least one fluorine atom of R₆ and R₇ may include CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉ and CH₂CH₂C₄F₉, and among them, preferably CF₃.

L represents a divalent linking group, and examples thereof may include —COO—, —OCO—, —CO—, —O—. —S—, —SO—, —SO₂—, —N(Ri)- (wherein Ri represents a hydrogen atom or an alkyl), an alkylene group (preferably an alkyl group having 1 to 6, more preferably an alkyl group having 1 to 4 carbon atoms, particularly preferably a methyl group or an ethyl group, and most preferably a methyl group), a cycloalkylene group (preferably having 3 to 10 carbon atoms), an alkenylene group (preferably having 2 to 6 carbon atoms) or a divalent linking group formed by combining two or more thereof, preferably —COO—, —OCO—, —CO—, —SO₂—, —CON(Ri)-, —SO₂N(Ri)-, —CON(Ri)-alkylene group-, —N(Ri)CO-alkylene group-. —COO-alkylene group- or —OCO-alkylene group-, and more preferably —SO₂—, —COO—, —OCO—, —COO-alkylene group- or —OCO-alkylene group-. The alkylene group in —CON(Ri)-alkylene group-, —N(Ri)CO-alkylene group-, —COO-alkylene group- and —OCO-alkylene group- is preferably an alkylene group having 1 to 20 carbon atoms, and more preferably an alkylene group having 1 to 10 carbon atoms. When a plurality of L's is present, L's may be the same or different.

Specific examples and preferred examples of the alkyl group for R₁ may be the same as the specific examples and preferred examples described above as R₁ to R₄ in Formula (1).

The organic group containing a ring structure of A is not particularly limited as long as the group has a ring structure, and examples thereof may include an alicyclic group, an aryl group, a heterocyclic group (including a group having no aromaticity, as well as a group having aromaticity, for example, a tetrahydropyran ring, a lactone ring structure and a sultone ring structure as well).

The alicyclic group may be monocyclic or polycyclic, and is preferably a monocyclic cycloalkyl group such as a cyclopentyl group, a cyclohexyl group and cyclooctyl group, or a polycyclic cycloalkyl group such as a norbornyl group, a norbornenyl group, a tricyclodecanyl group (for example, a tricyclo[5.2.1.0(2,6)]decanyl group), a tetracyclodecanyl group, a tetracyclododecanyl group and an adamantyl group, and particularly preferably an adamantyl group. Further, also preferred is a nitrogen atom-containing alicyclic group such as a piperidine group, a decahydroquinoline group and a decahydroisoquinoline group. Among them, an alicyclic group having a bulky structure with 7 or more carbon atoms, such as a norbornyl group, a tricyclodecanyl group, a tetracyclodecanyl group, a tetracyclododecanyl group, an adamantyl group, a decahydroquinoline group or a decahydroisoquinoline group, is preferred from the viewpoint of suppressing diffusion in a film during a PEB (post-exposure baking) process and improving the exposure latitude. Among them, particularly preferred is an adamantyl group or a decahydroisoquinoline group.

Examples of the aryl group may include a benzene ring, a naphthalene ring, a phenanthrene ring and an anthracene ring. Among them, a naphthyl group having low light absorbance is preferred from the viewpoint of light absorbance at at 193 nm.

Examples of the heterocyclic ring may include a furan ring, a thiophene ring, a benzofuran ring, a benzothiophene ring, a dibenzofuran ring, a dibenzothiophene ring and a pyridine ring. Among them, preferred is a furan ring, a thiophene ring or a pyridine ring. Examples of other preferred heterocyclic group may include structures shown below (in the formulas, X represents a methylene group or an oxygen atom, and R represents a monovalent organic group).

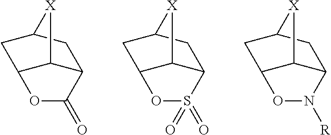

The cyclic organic group may have a substituent, and examples of the substituent may include an alkyl group (which may be either straight, branched or cyclic, and preferably has 1 to 12 carbon atoms), an aryl group (preferably having 6 to 14 carbon atoms), a hydroxyl group, an alkoxy group, an ester group, an amide group, a urethane group, a ureido group, a thioether group, a sulfonamide group, a sulfonate ester group and the like.

Meanwhile, the carbon constituting the organic group having a ring structure (carbon contributing to ring formation) may be carbonyl carbon.

x is preferably 1 to 8, more preferably 1 to 4, and particularly 1. y is preferably 0 to 4, more preferably 0 or 1, and still more preferably 1. z is preferably 0 to 8, more preferably 0 to 4, and still more preferably 1.

Further, in another aspect of the present invention, the non-nucleophilic anion of X′ may be a disulfonylimidate anion.

The disulfonylimidate anion is preferably a bis(alkylsulfonyl)imide anion.

The alkyl group in the bis(alkylsulfonyl)imide anion is preferably an alkyl group having 1 to 5 carbon atoms.

Two alkyl groups in the bis(alkylsulfonyl)imide anion may be linked to each other ot form an alkylene group (preferably having 2 to 4 carbon atoms), which may form a ring together with an imide group and two sulfonyl groups. The ring structure which may be formed by the bis(alkylsulfonyl)imide anion is preferably 5- to 7-membered ring, and more preferably 6-membered ring.

The substituent which may be possessed by the alkyl group and the alkylene group formed by linking two alkyl groups may be a halogen atom, an alkyl group substituted with a halogen atom, an alkoxy group, an alkylthio group, an alkyloxysulfonyl group, an aryloxysulfonyl group, a cycloalkylaryloxysulfonyl group or the like, and preferably a fluorine atom or an alkyl group substituted with a fluorine atom.

From the viewpoint of acid strength, it is preferred that pKa of the acid generated is −1 or less in order to enhance the sensitivity.

Meanwhile, the compound (A) may be a compound having a plurality of structures represented by Formula (1). For example, the compound (A) may be a compound having a structure in which R₅ in Formula (1) is bonded via a single bond or a linking group to another R₅ in Formula (1).

The compound represented by Formula (1) or Formula (1a) has a fluorine content of preferably 0.25 or less, more preferably 0.20 or less, still more preferably 0.15 or less, and particularly preferably 0.10 or less, as calculated by (the sum of mass of the total fluorine atoms contained in the compound)/(the sum of mass of the total atoms contained in the compound), of the compound represented by Formula (1) is 0.25 or less.

Particularly, in Formula (1) or Formula (1a), in the case where X′ is a non-nucleophilic anion represented by Formula (2), the number of fluorine atoms possessed by the organic group A is preferably 0 to 3, more preferably 0 to 2, and still more preferably 0.

Preferred specific examples of the compound (A) represented by Formula (1) are shown below, but the present invention is not limited thereto.

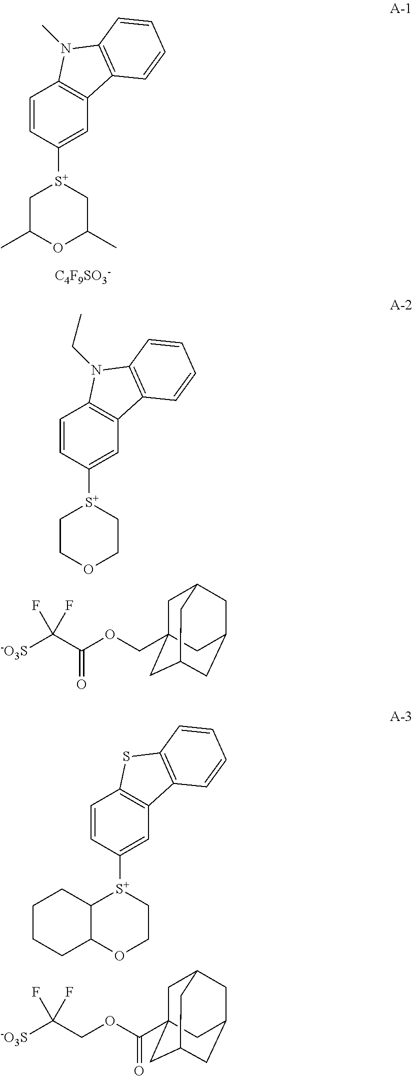

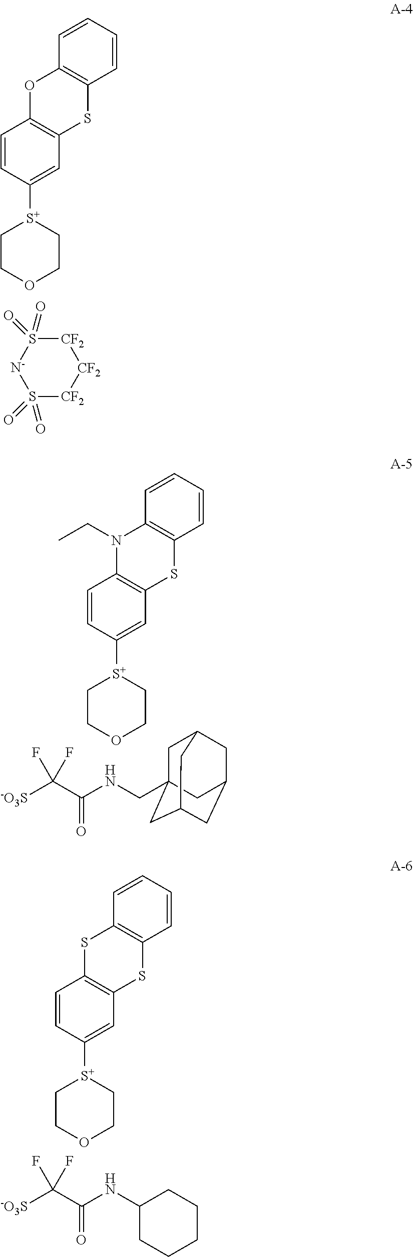

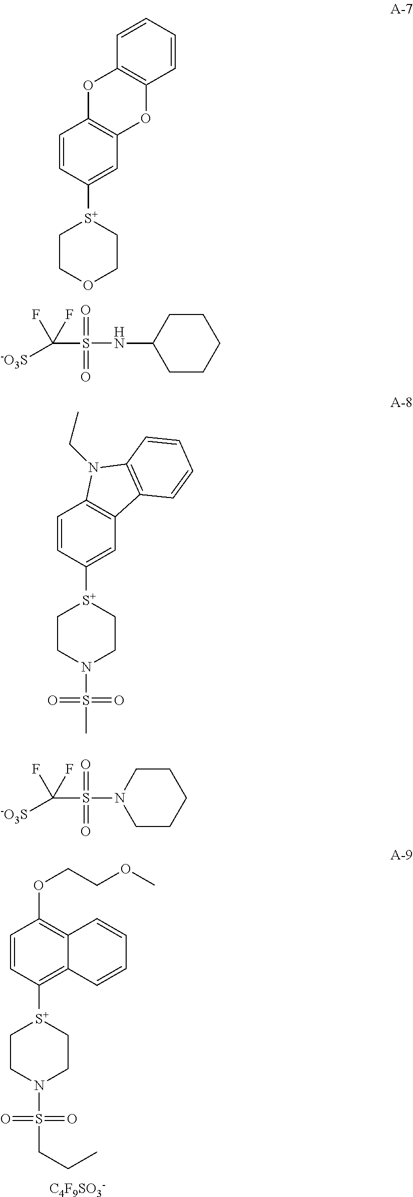

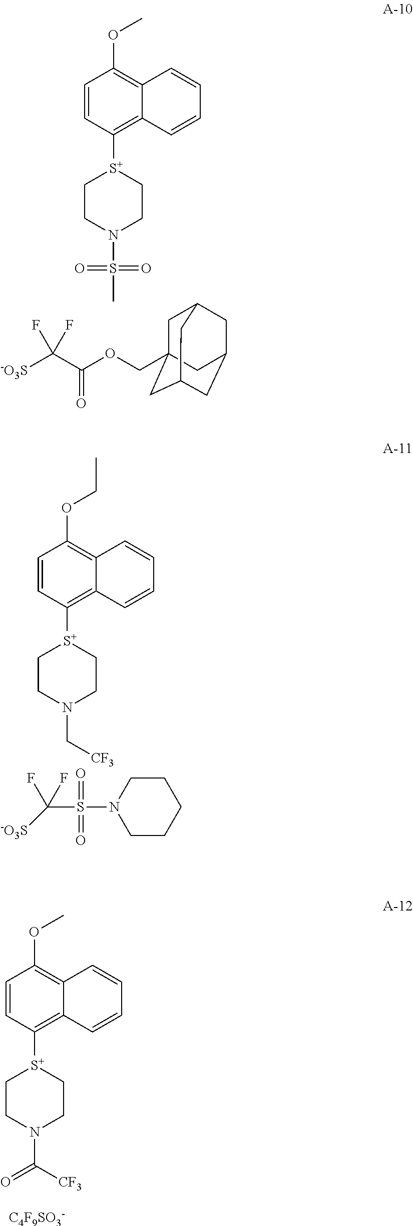

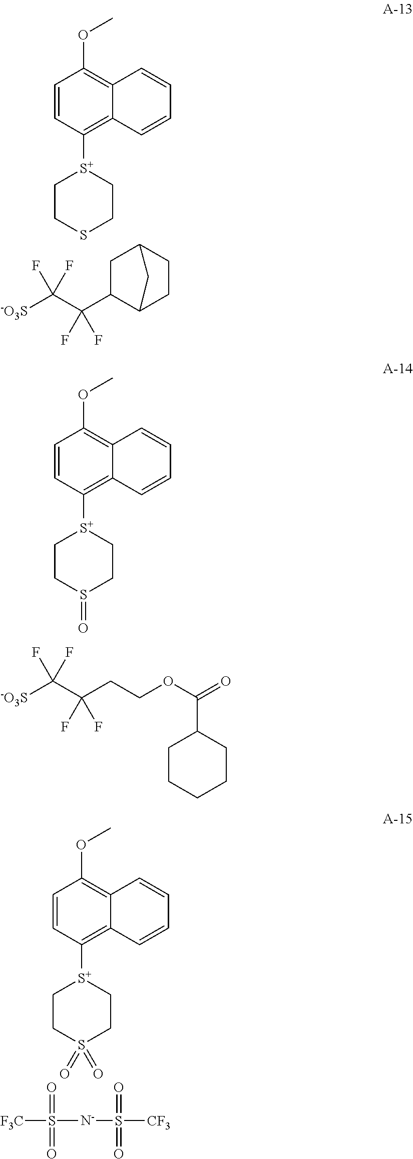

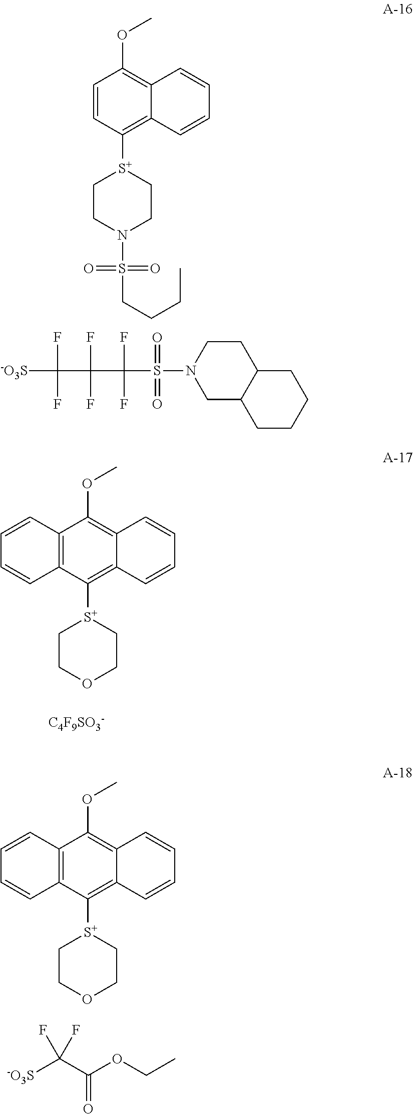

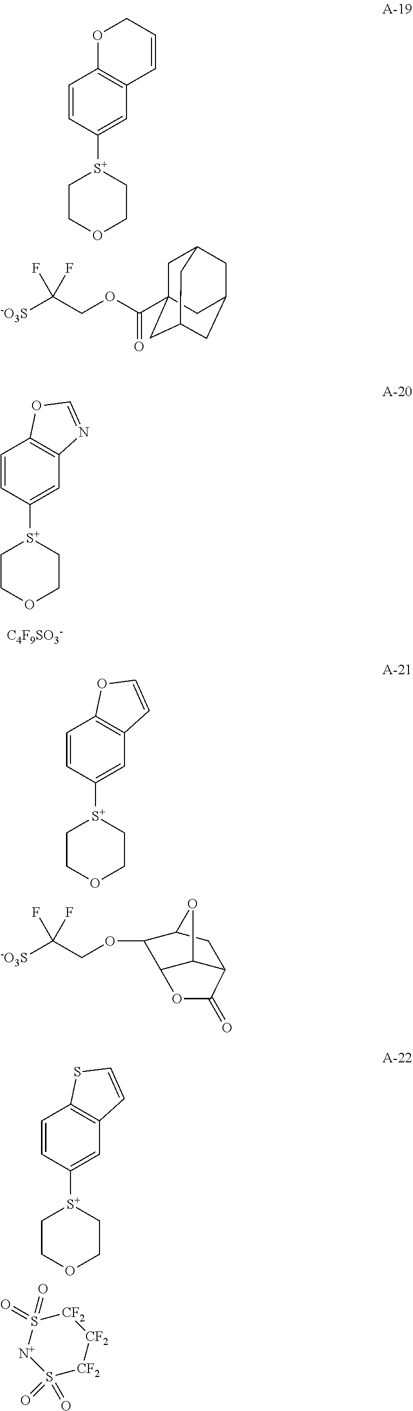

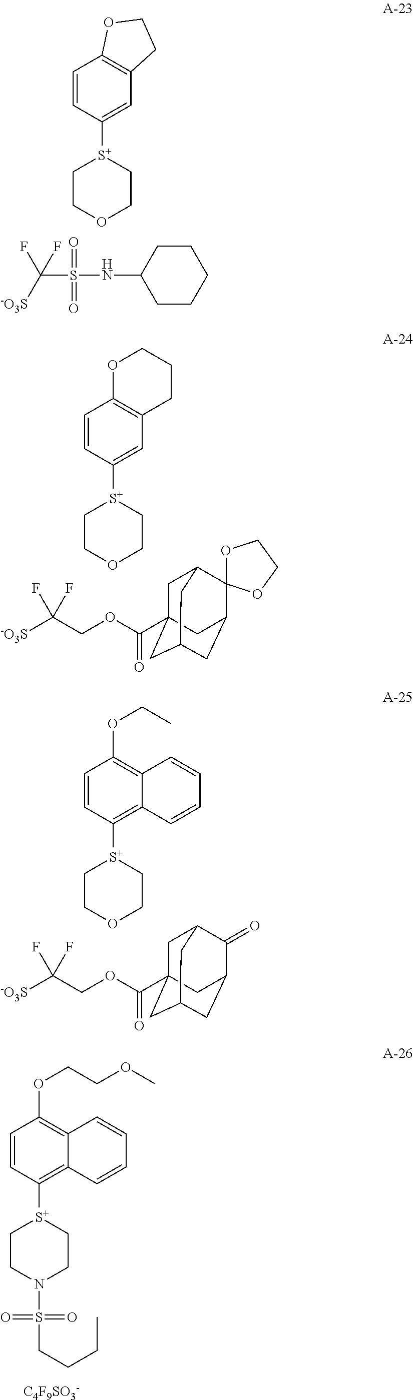

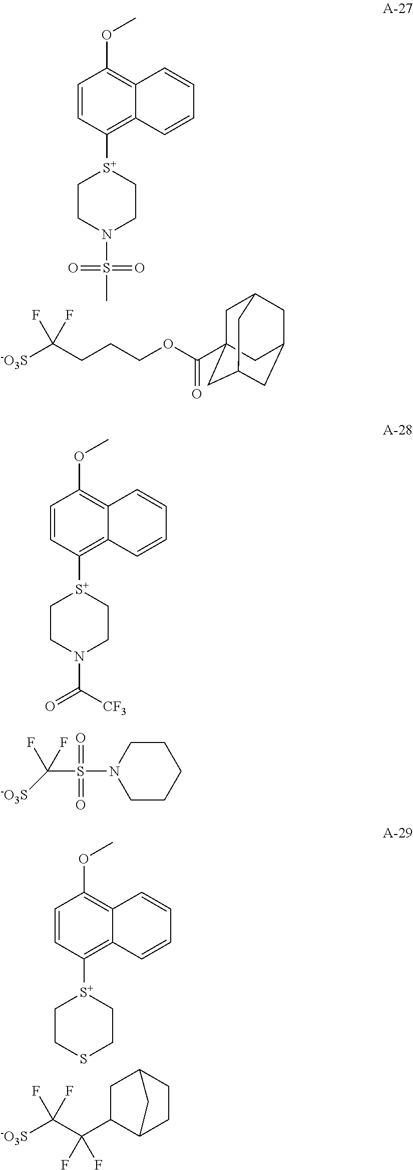

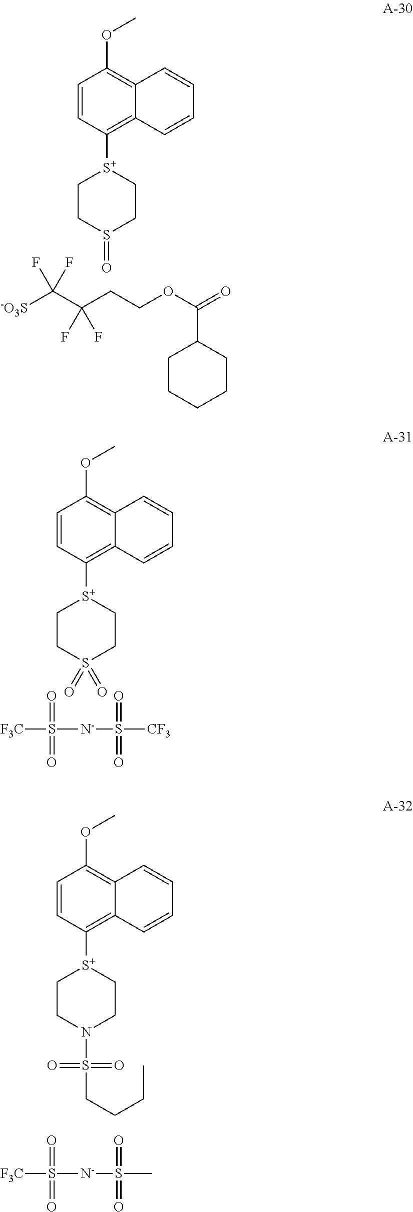

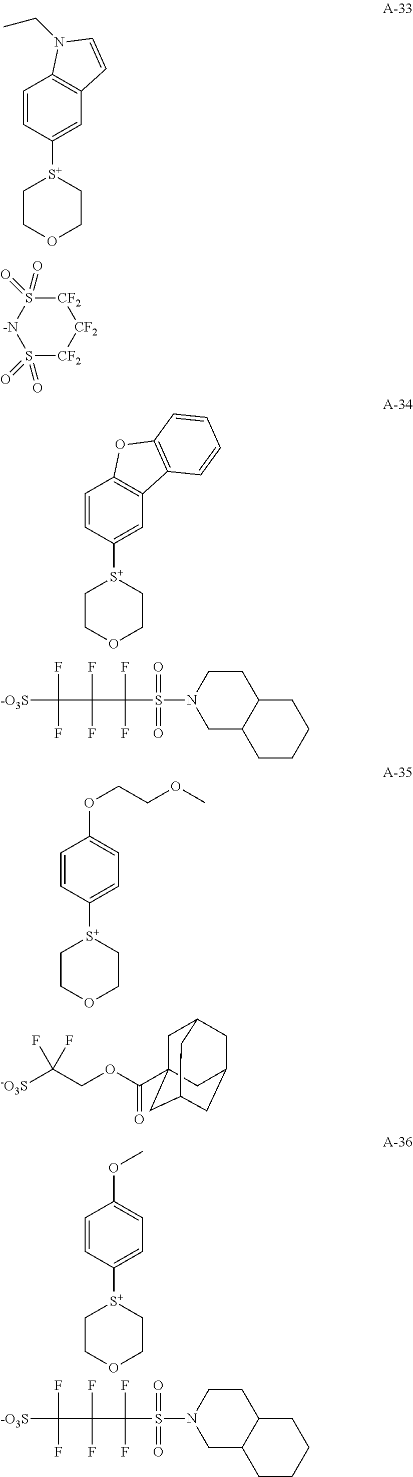

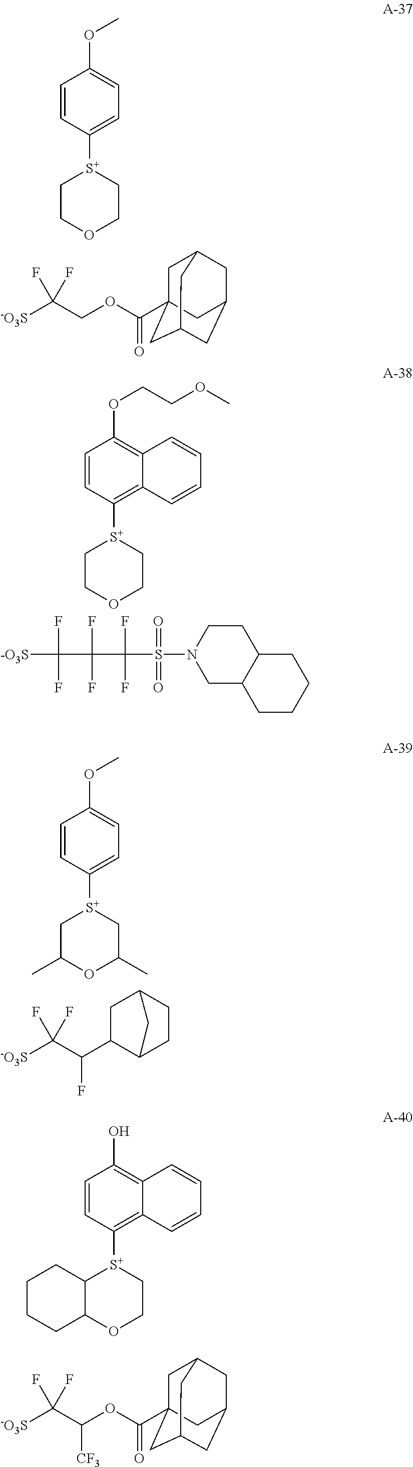

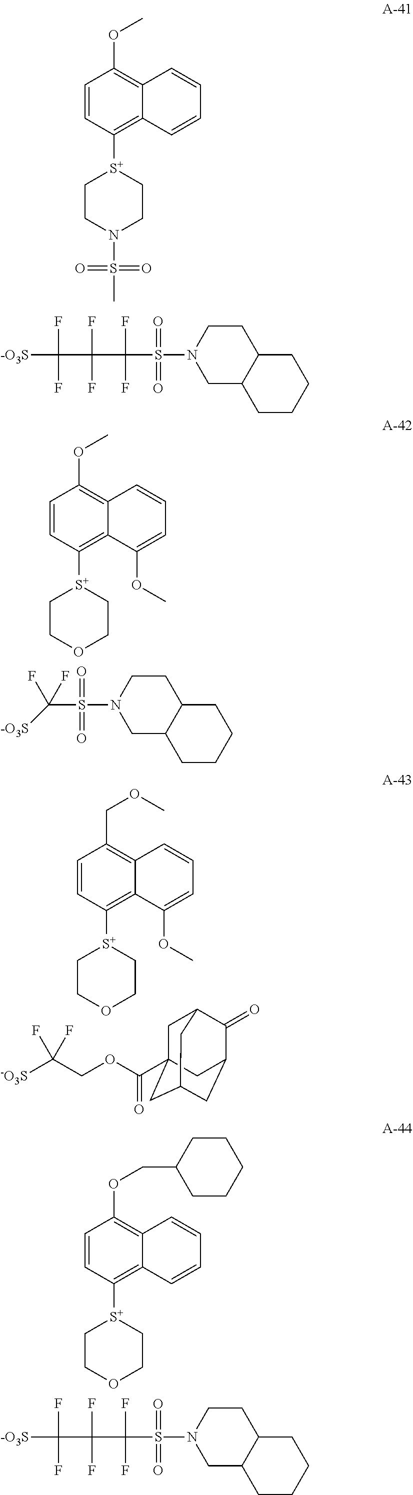

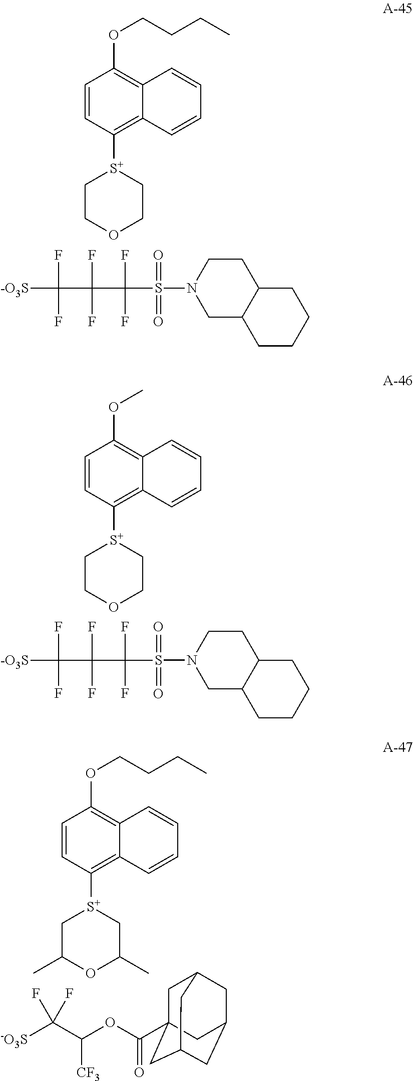

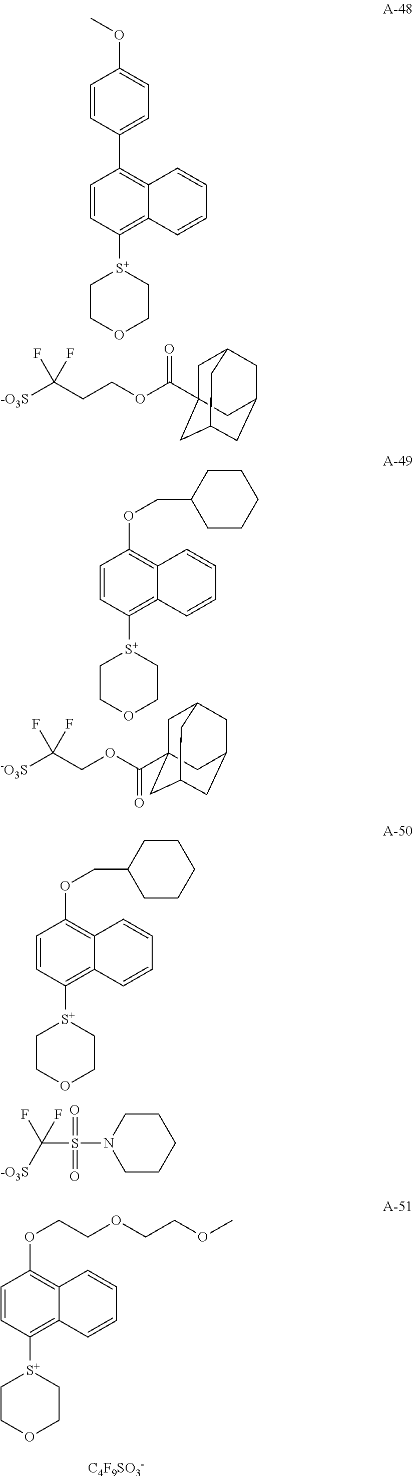

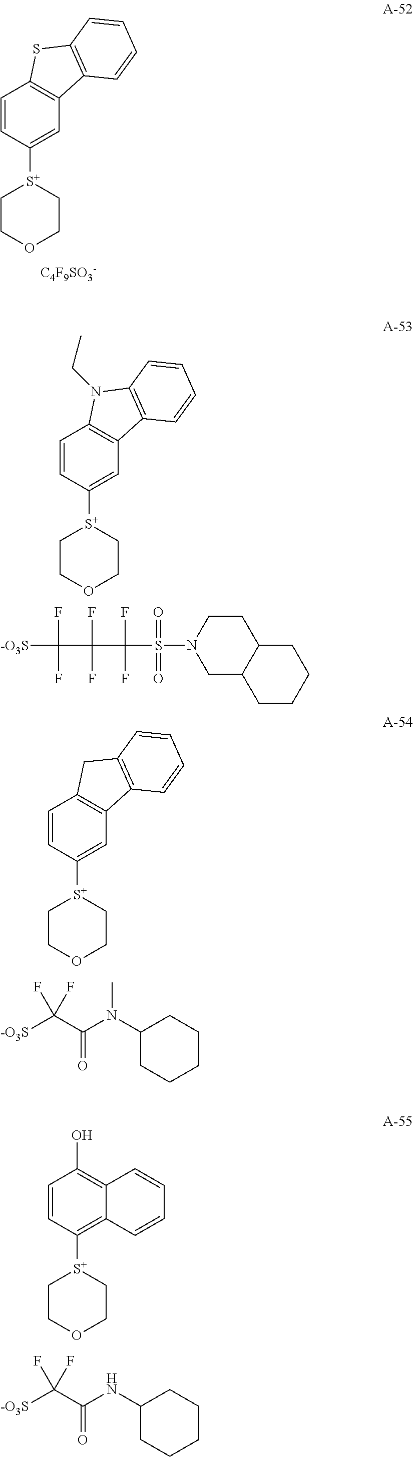

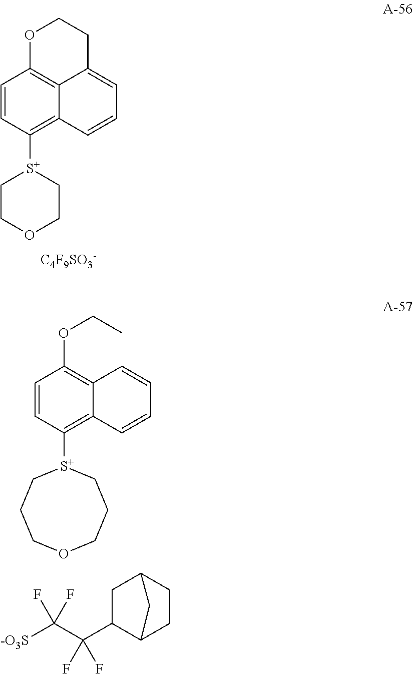

Synthesis of the compound (A) will be described.

The sulfonate anion in Formula (1) or a salt thereof may be used for the synthesis of the compound (A) represented by Formula (1). The sulfonate anion in Formula (1) or a salt thereof (for example, an onium salt or a metal salt), which may be used for the synthesis of the compound (A), may be synthesized by using a general sulfonate esterification reaction or sulfonamidation reaction. For example, the compound may be obtained by a method of selectively reacting one sulfonyl halide moiety of a bissulfonyl halide compound with an amine, alcohol or amide compound to form a sulfonamide bond, a sulfonate ester bond or a sulfonamide bond, and then hydrolyzing the other sulfonyl halide moiety, or a method of ring-opening a cyclic sulfonic anhydride by an amine, alcohol or amide compound.

Examples of the salt of the sulfonate anion in Formula (1) may include a metal salt of sulfonic acid, a sulfonate onium salt and the like. Examples of the metal in the metal salt of the sulfonic acid may include Na⁺, Li⁺, K⁺ and the like. Examples of the onium cation in the sulfonate onium salt may include an ammonium cation, a sulfonium cation, an iodonium cation, a phosphonium cation, a diazonium cation and the like.

The compound (A) may be synthesized by salt-exchanging the sulfonium anion represented by Formula (1) with a photoactive onium salt such as a sulfonium salt corresponding to the sulfonium cation in Formula (1).

In the actinic ray-sensitive or radiation-sensitive resin composition according to the present invention, the compound (A) may be used either alone or in combination of two or more. The content of the compound (A) in the composition of the present invention is preferably 0.1% by mass to 40% by mass, more preferably 0.5% by mass to 30% by mass, still more preferably 5% by mass to 25% by mass based on the total solid of the composition.

Further, the compound (A) may be used in combination with an acid generator (hereinafter, also referred to as a compound (A′) or an acid generator (A′)) other than the compound (A).

The compound (A′) is not particularly limited, but may include compounds represented by the following Formulas (ZI′), (ZII′) and (ZIII′).

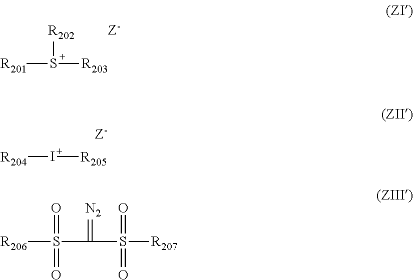

In Formula (ZI′), R₂₀₁, R₂₀₂ and R₂₀₃ each independently represent an organic group.

The organic group as R₂₀₀, R₂₀₂ and R₂₀₃ has generally 1 to 30 carbon atoms, and preferably 1 to 20 carbon atoms.

Further, two of R₂₀₁ to R₂₀₃ may be bound with each other to form a ring structure, which may contain an oxygen atom, a sulfur atom, an ester bond, an amide bond or a carbonyl group in its ring. Examples of the group formed by two of R₂₀₁ to R₂₀₃ being bound with each other may include an alkylene group (for example, a butylene group and a pentylene group).

Examples of the organic group represented by R₂₀₁, R₂₀₂ and R₂₀₃ may include the corresponding groups in the compound (ZI′-1) as described below.

Meanwhile, the compound may be a compound having a plurality of structures represented by Formula (ZI′). For example, the compound may be a compound having a structure in which at least one of R₂₀₁ to R₂₀₃ of a compound represented by Formula (ZI′) is bonded via a single bond or a linking group to at least one of R₂₀₁ to R₂₀₃ of another compound represented by Formula (ZI′).

Z′represents a non-nucleophilic anion (an anion having an extremely low ability of causing a nucleophilic reaction).

Examples of Z′ may include a sulfonate anion (an aliphatic sulfonate anion, an aromatic sulfonate anion, a camphorsulfonate anion and the like), a carboxylate anion (an aliphatic carboxylate anion, an aromatic carboxylate anion, an aralkylcarboxylate anion and the like), a sulfonylimide anion, a bis(alkylsulfonyl)imide anion, tris(alkylsulfonyl)methide and the like.

The aliphatic moiety in the aliphatic sulfonate anion and the aliphatic carboxylate anion may be an alkyl group or a cycloalkyl group, and may be preferably a straight or branched alkyl group having 1 to 30 carbon atoms and a cycloalkyl group having 3 to 30 carbon atoms.

The aromatic group in the aromatic sulfonate anion and an aromatic carboxylate anion may be preferably an aryl group having 6 to 14 carbon atoms, and examples thereof may include a phenyl group, a tolyl group, a naphthyl group and the like.

The alkyl group, the cycloalkyl group and the aryl group as exemplified above may have a substituent. Specific examples thereof may include a nitro group, a halogen atom such as a fluorine atom, a carboxyl group, a hydroxyl group, an amino group, a cyano group, an alkoxy group (preferably having 1 to 15 carbon atoms), a cycloalkyl group (preferably having 3 to 15 carbon atoms), an aryl group (preferably having 6 to 14 carbon atoms), an alkoxycarbonyl group (preferably having 2 to 7 carbon atoms), an acyl group (preferably having 2 to 12 carbon atoms), an alkoxycarbonyloxy group (preferably having 2 to 7 carbon atoms), an alkylthio group (preferably having 1 to 15 carbon atoms), an alkylsulfonyl group (preferably having 1 to 15 carbon atoms), alkyliminosulfonyl group (preferably having 1 to 15 carbon atoms), an aryloxysulfonyl group (preferably having 6 to 20 carbon atoms), an alkylaryloxysulfonyl group (preferably having 7 to 20 carbon atoms), a cycloalkylaryloxysulfonyl group (preferably having 10 to 20 carbon atoms), an alkyloxyalkyloxy group (preferably having 5 to 20), cycloalkylalkyloxyalkyloxy group (preferably having 8 to 20 carbon atoms) and the like. The aryl group and the ring structure possessed by each of the groups may further have an alkyl group (preferably having 1 to 15 carbon atoms) as a substituent.

The aralkyl group in the aralkylcarboxylate anion is preferably an aralkyl group having 7 to 12 carbon atoms, and examples thereof may include a benzyl group, a phenethyl group, a naphthylmethyl group, a naphthylethyl group, a naphthylbutyl group and the like.

Examples of the sulfonylimide anion may include a saccharin anion.

The alkyl group in the bis(alkylsulfonyl)imide anion, tris(alkylsulfonyl)methide anion is preferably an alkyl group having 1 to 5 carbon atoms.

Two alkyl groups in the bis(alkylsulfonyl)imide anion may be linked to each other to form an alkylene group (preferably having 2 to 4 carbon atoms), which may form a ring together with an imide group and two sulfonyl groups.

Examples of the substituent which may be possessed by the alkyl group and the alkylene group formed by two alkyl groups in the bis(alkylsulfonyl)imide anion being linked to each other may include a halogen atom, an alkyl group substituted with a halogen atom, an alkoxy group, an alkylthio group, an alkyloxysulfonyl group, an aryloxysulfonyl group, a cycloalkylaryloxysulfonyl group and the like, and preferably a fluorine atom or an alkyl group substituted with a fluorine atom.

Examples of the other non-nucleophilic anions may include fluorinated phosphorus (for example, PF₆⁻), fluorinated boron (for example, BF₄⁻), fluorinated antimony (for example, SbF₆⁻) and the like.

Z′ is preferably an aliphatic sulfonate anion which is substituted with a fluorine atom at the α-position of the sulfonic acid, an aromatic sulfonate anion substituted with a fluorine atom or a group having a fluorine atom, a bis(alkylsulfonyl)imide anion in which the alkyl group is substituted with a fluorine atom, or a tris(alkylsulfonyl)methide anion in which the alkyl group is substituted with a fluorine atom. The non-nucleophilic anion is more preferably a perfluoroaliphatic sulfonate anion (more preferably having 4 to 8 carbon atoms) and a benzenesulfonate anion having a fluorine atom, and still more preferably a nonafluorobutanesulfonate anion, a perfluorooctanesulfonate anion, a pentafluorobenzenesulfonate anion or a 3,5-bis(trifluoromethyl)benzenesulfonate anion.

From the viewpoint of acid strength, it is preferred that pKa of the acid generated is −1 or less in order to enhance the sensitivity.

a more preferred component (ZI′) may be exemplified by a compound (ZI′−1) as described below.

The compound (ZI′-1) is an arylsulfonium compound in which at least one of R₂₀₁ to R₂₀₃ in Formula (ZI′) is an aryl group, that is, a compound having arylsulfonium as a cation.

In the arylsulfonium compound, all of R₂₀₁ to R₂₀₃ may be an aryl group or a part of R₂₀₁ to R₂₀₃ may be an aryl group, with the remaining being an alkyl group or a cycloalkyl group, but it is preferred that all of R₂₀₁ to R₂₀₃ may be an aryl group.

Examples of the arylsulfonium compound may include a triarylsulfonium compound, a diarylalkylsulfonium compound, an aryldialkylsulfonium compound, a diarylcycloalkylsulfonium compound and an aryldicycloalkylsulfonium compound, and preferably a triarylsulfonium compound.

The aryl group of the arylsulfonium compound is preferably a phenyl group or a naphthyl group, and more preferably a phenyl group. The aryl group may be an aryl group having a heterocylic structure having an oxygen atom, a nitrogen atom, a sulfur atom or the like. Examples of the heterocyclic structure may include include a pyrrole residue, a furan residue, a thiophene residue, an indole residue, a benzofuran residue, a benzothiophene residue and the like. When the arylsulfonium compound has two or more aryl groups, the aryl groups may be the same as or different.

The alkyl group or the cycloalkyl group possessed by the arylsulfonium compound as necessary is preferably a straight or branched alkyl group having 1 to 15 carbon atoms and a cycloalkyl group having 3 to 15 carbon atoms, and examples thereof may include a methyl group, an ethyl group, a propyl group, a n-butyl group, a sec-butyl group, a t-butyl group, a cyclopropyl group, a cyclobutyl group, a cyclohexyl group and the like.

The aryl group, the alkyl group and the cycloalkyl group of R₂₀₁ to R₂₀₃ may have an alkyl group (for example, having 1 to 15 carbon atoms), a cycloalkyl group (for example, having 3 to 15 carbon atoms), an aryl group (for example, having 6 to 14 carbon atoms), an alkoxy group (for example, having 1 to 15 carbon atoms), a halogen atom, a hydroxyl group or a phenylthio group as a substituent. The substituent is preferably a straight or branched alkyl group having 1 to 12 carbon atoms, a cycloalkyl group having 3 to 12 carbon atoms, and a straight, branched or cyclic alkoxy group having 1 to 12 carbon atoms, and more preferably an alkyl group having 1 to 4 carbon atoms and an alkoxy group having 1 to 4 carbon atoms. The substituent may be substituted on any one of three R₂₀₁ to R₂₀₃ or may be substituted on all of the three. Further, when R₂₀₁ to R₂₀₃ are an aryl group, the substituent is preferably substituted at the p-position of the aryl group.

Subsequently, Formulas (ZII′) and (ZIII′) will be described.

In Formulas (ZII′) and (ZIII′),

R₂₀₄ to R₂₀₇ each independently represent an aryl group, alkyl group or a cycloalkyl group.

The aryl group, the alkyl group and the cycloalkyl group of R₂₀₄ to R₂₀₇ may be the same as the aryl group, the alkyl group and the cycloalkyl group of R₂₀₁ to R₂₀₃ in the compound (ZI′-1).

The aryl group, the alkyl group and the cycloalkyl group of R₂₀₄ to R₂₀₇ may have a substituent. Examples of the substituent may also include those which may be possessed by the aryl group, the alkyl group and the cycloalkyl group of R₂₀₁ to R₂₀₃ in the compound (ZI′−1) as described above.

Z′ represents a non-nucleophilic anion, and may be exemplified by the non-nucleophilic anion of Z in Formula (ZI′).

As an acid generator (A′) which may be used in combination with the acid generator of the present invention, a compound represented by the following Formula (ZIV′), (ZV′) or (ZVI′) may also be exemplified.

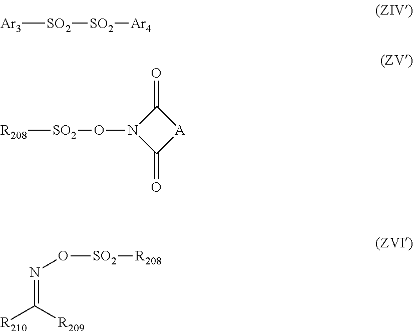

In Formulas (ZIV′) to (ZVI′),

Ar₃ and Ar₄ each independently represent an aryl group.

R₂₀₈, R₂₀₉ and R₂₁₀ each independently represent alkyl group, a cycloalkyl group or an aryl group.

A represents an alkylene group, an alkenylene group or an arylene group.

Specific examples of the aryl group of Ar₃, Ar₄, R₂₀₈, R₂₀₉ and R₂₁₀ may be the same as the specific examples of the aryl group as R₂₀₁, R₂₀₂ and R₂₀₃ in Formula (ZI′−1).

Specific examples of the alkyl group and a cycloalkyl group of R₂₀₈, R₂₀₉ and R₂₁₀ may be the same as the specific examples of the alkyl group and a cycloalkyl group as R₂₀₀, R₂₀₂ and R₂₀₃ in Formula (ZI′−1).

Examples of the alkylene group of A may include an alkylene having 1 to 12 carbon atoms (for example, a methylene group, an ethylene group, a propylene group, an isopropylene group, a butylene group, an isobutylene group and the like), examples of the alkenylene group of A may include an alkenylene group having 2 to 12 carbon atoms (for example, an ethenylene group, a propenylene group, a butenylene group and the like), and examples of the arylene group of A may include an arylene group having 6 to 10 carbon atoms (for example, a phenylene group, a tolylene group, naphthylene group and the like), respectively.

Among acid generators which may be used in combination with the acid generator of the present invention, particularly preferred examples thereof are shown below.

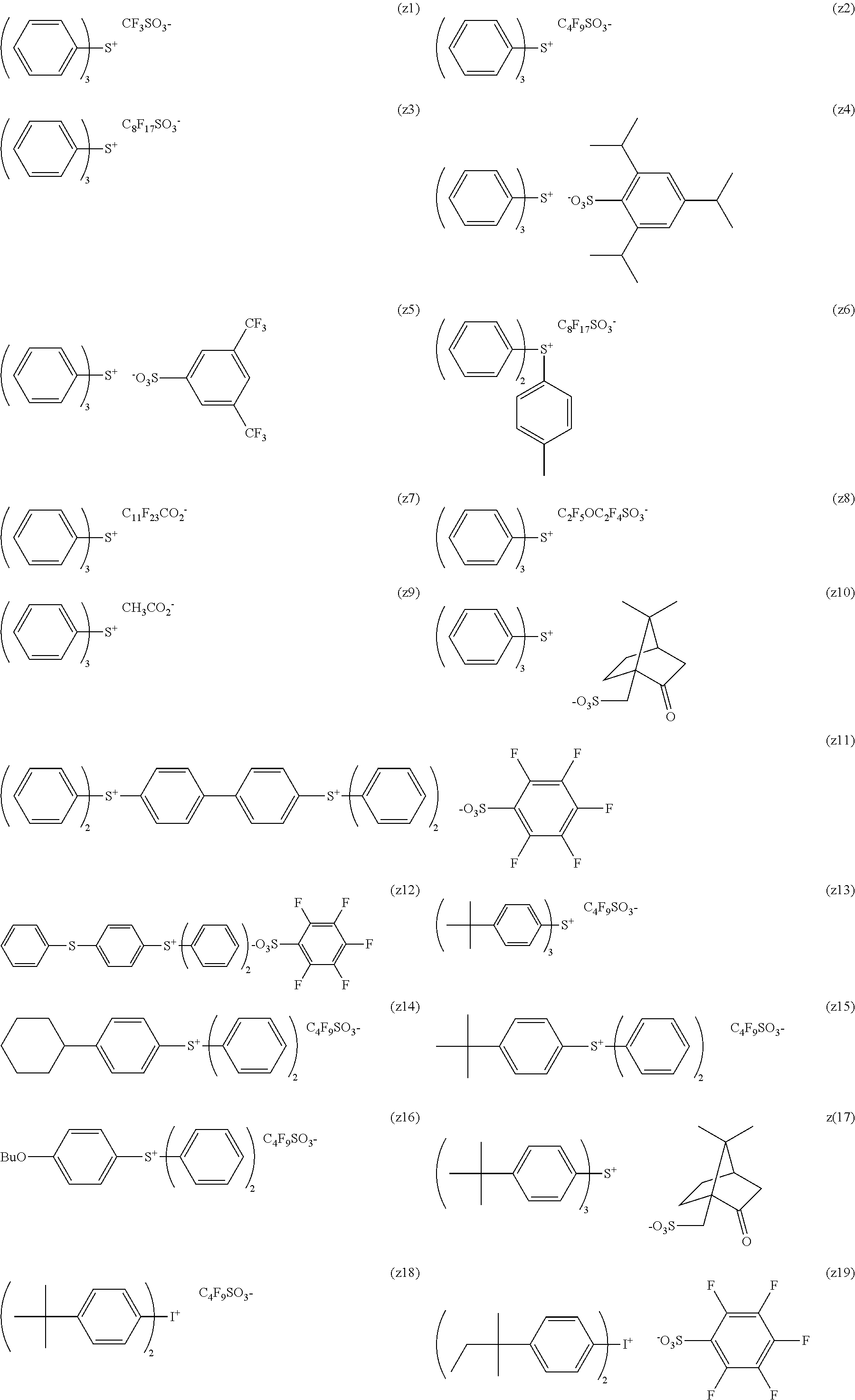

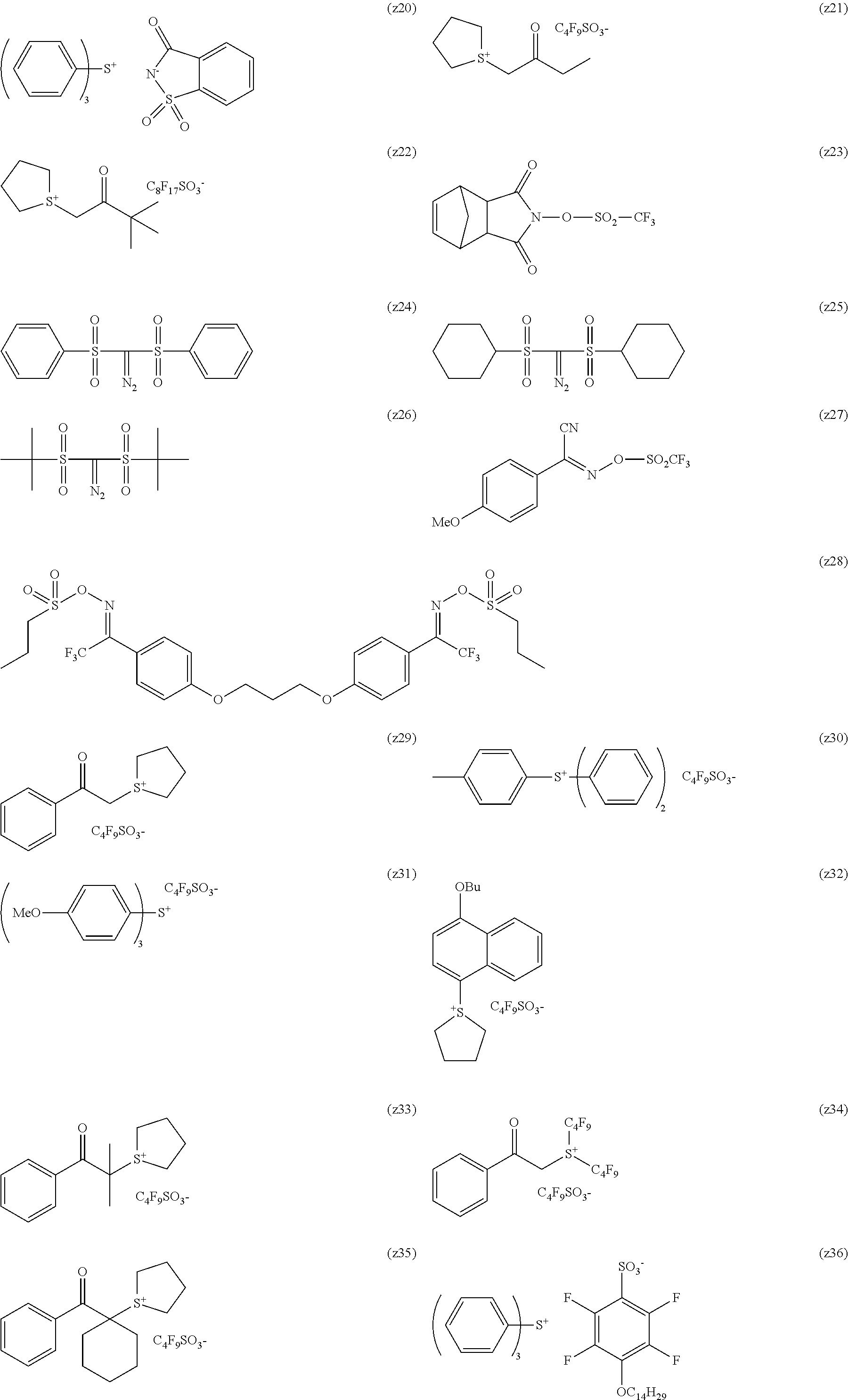

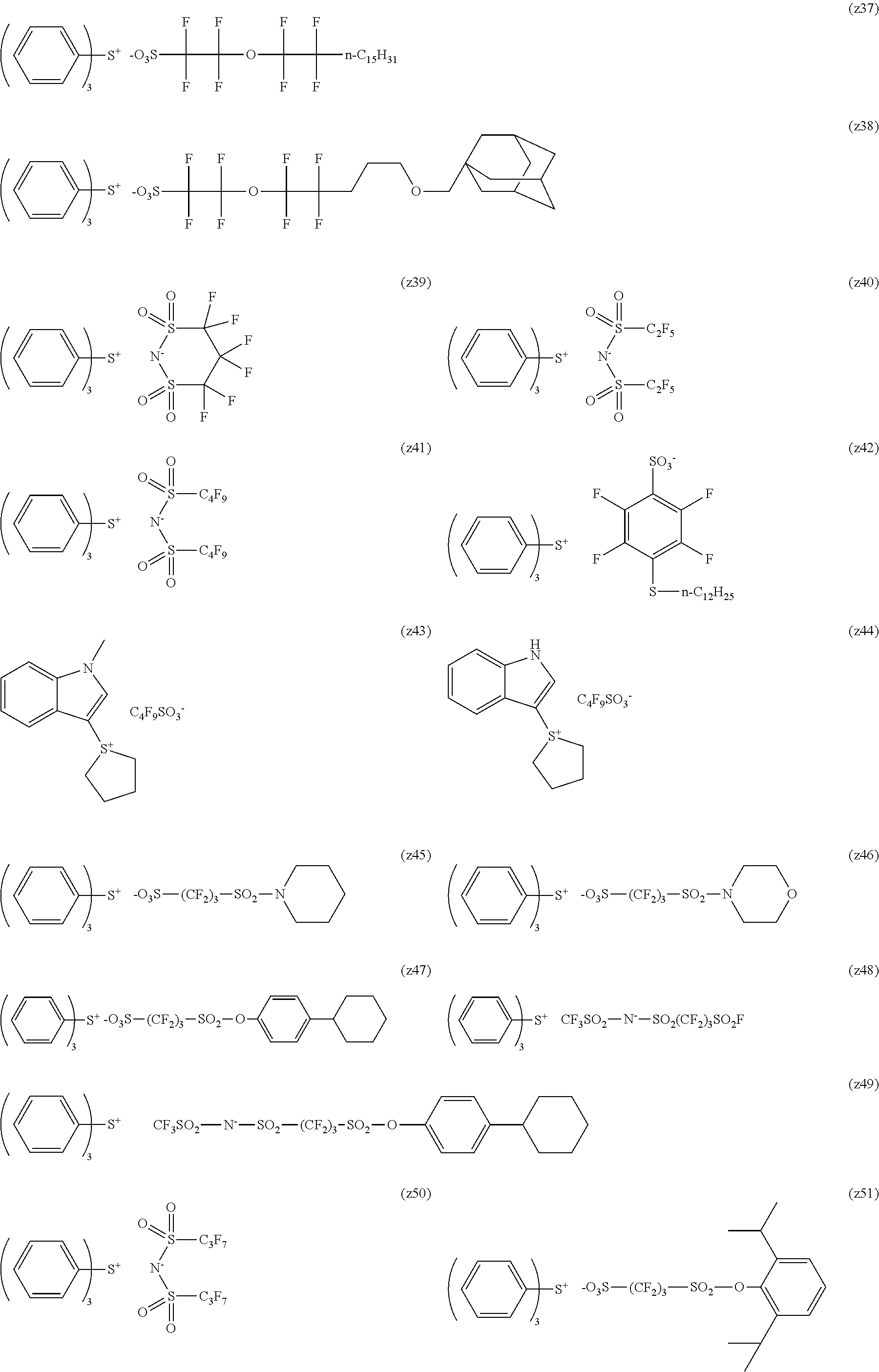

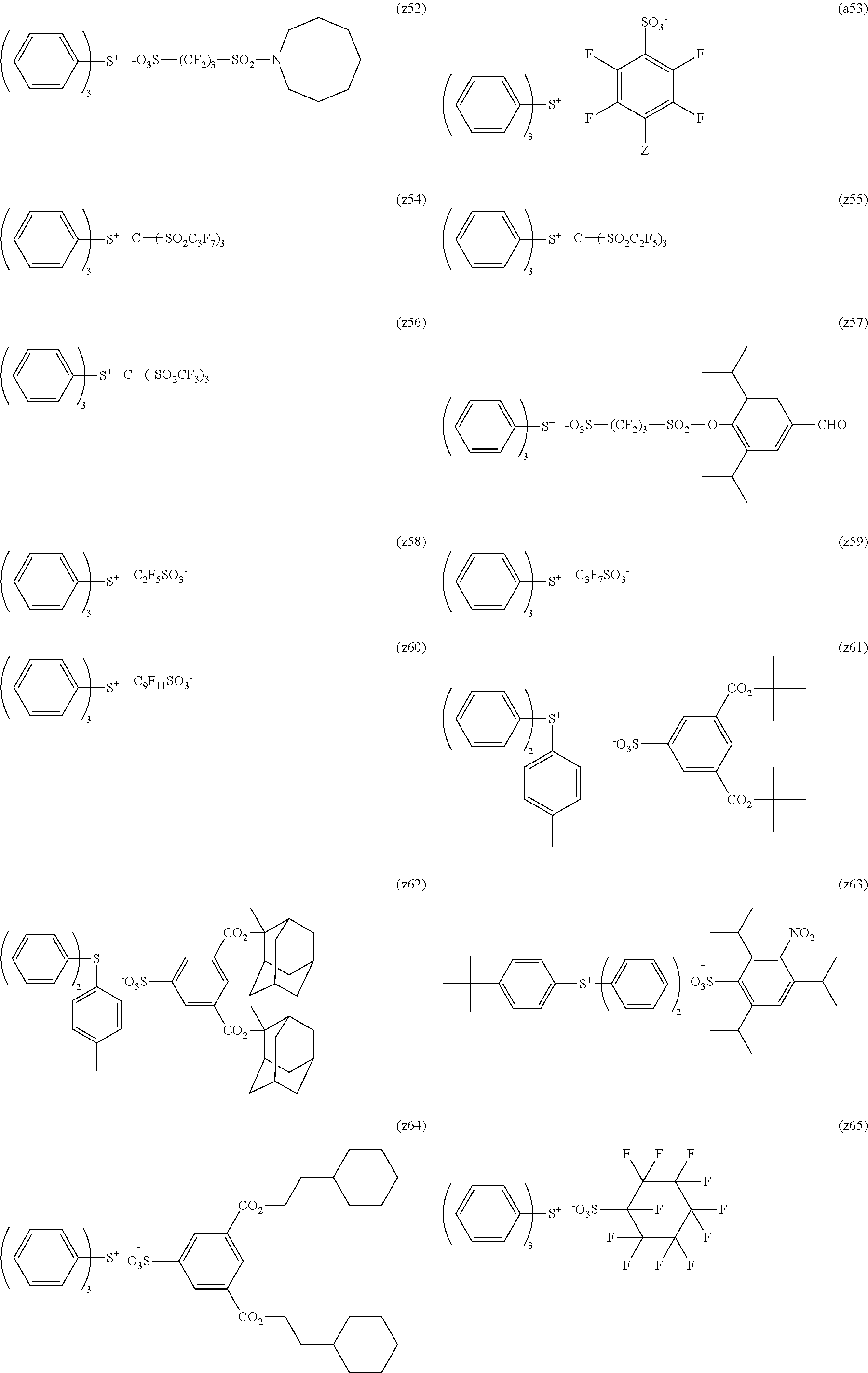

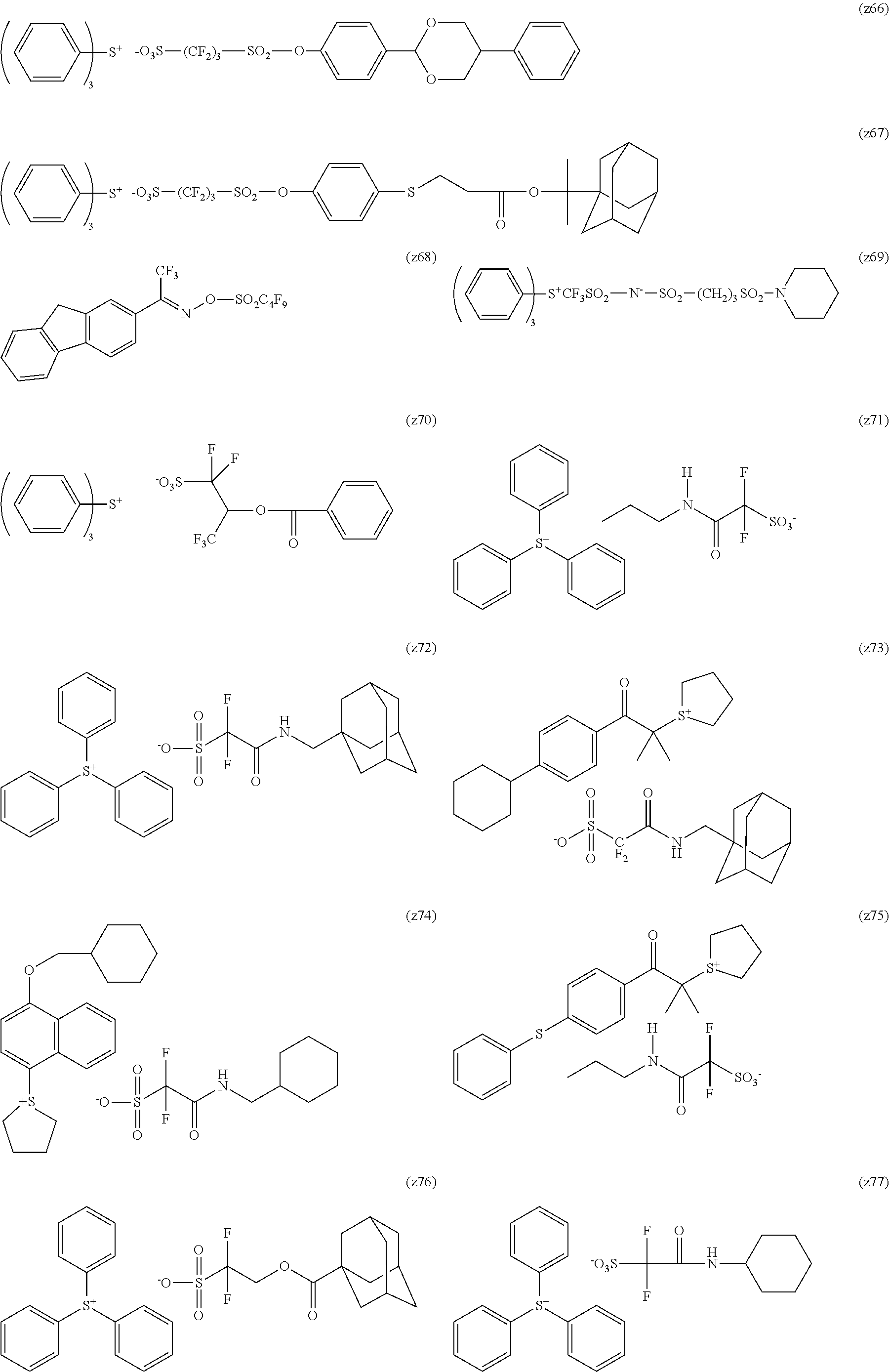

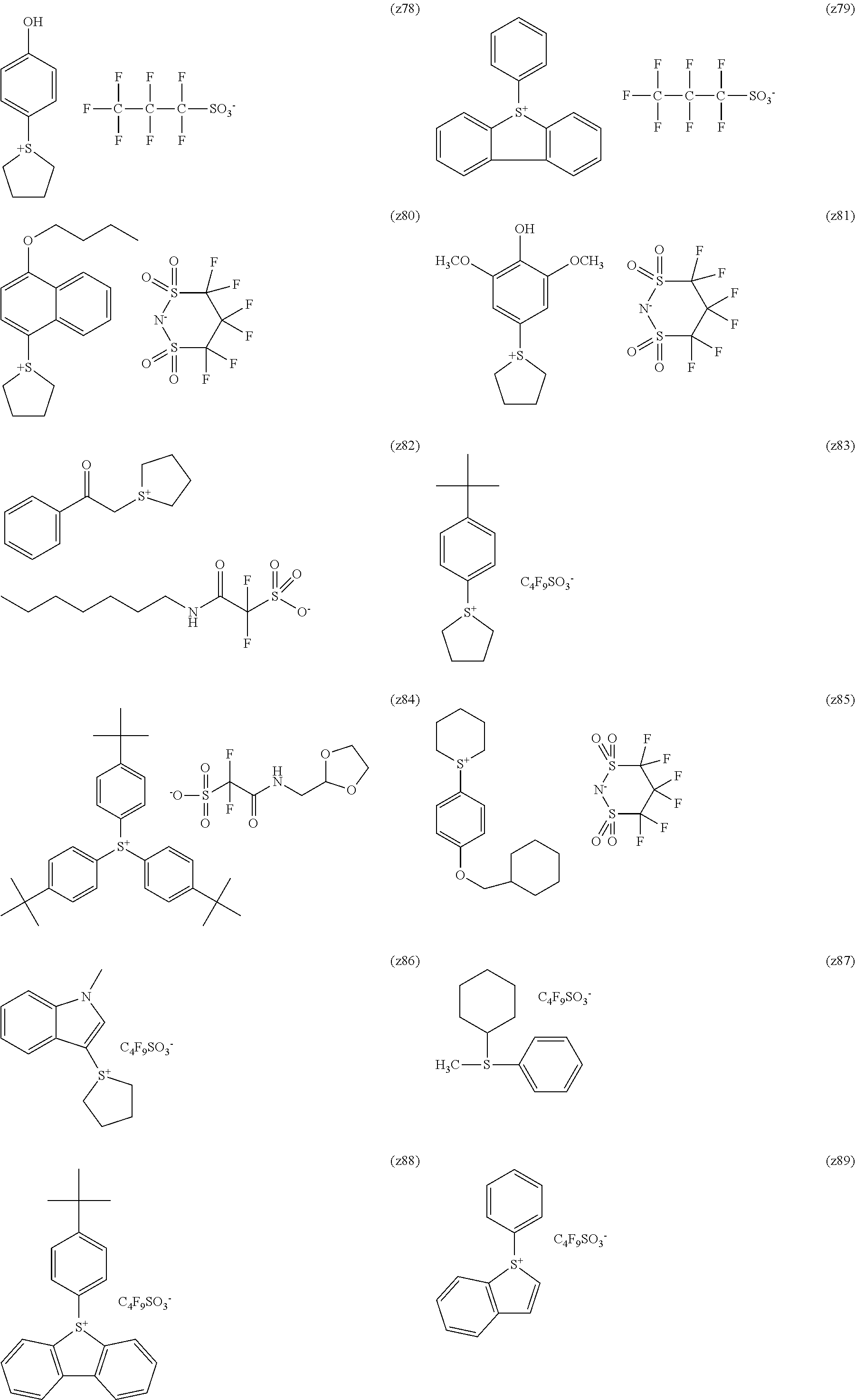

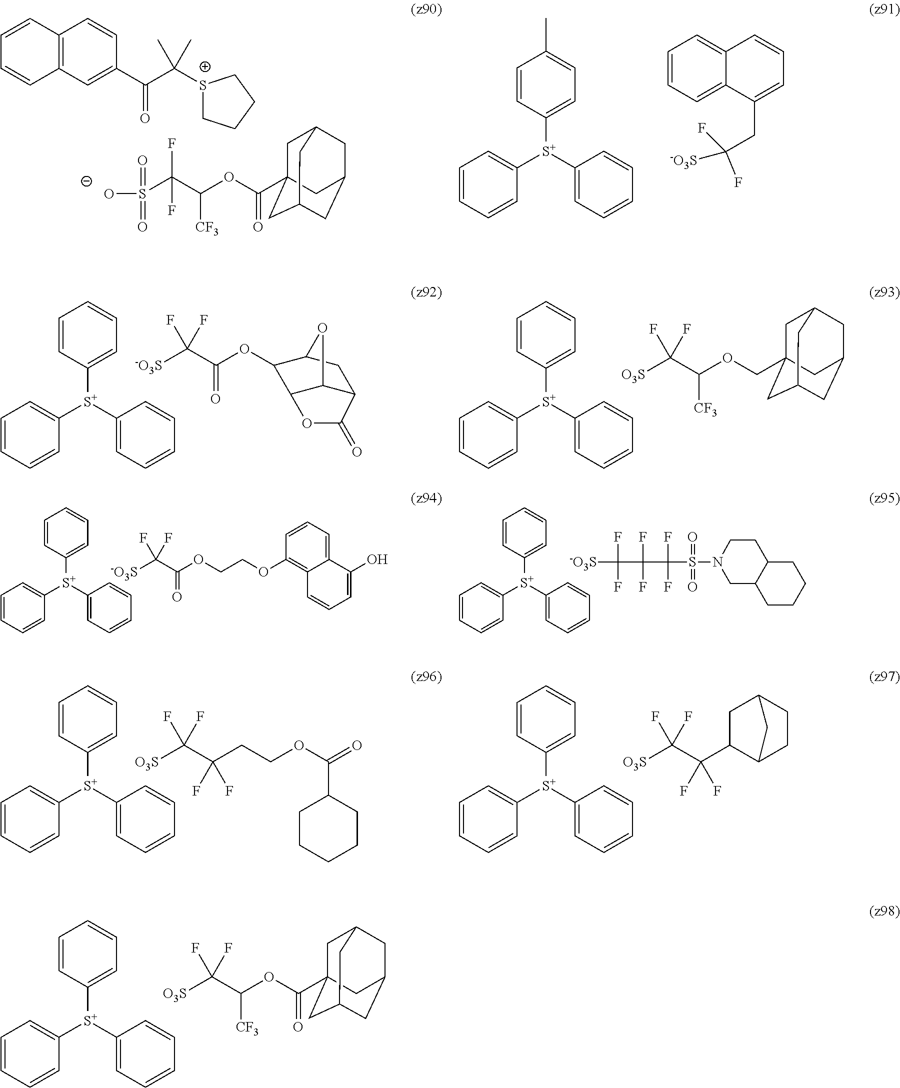

In the case of using the compound (A) in combination with the compound (A′), the amount of the acid generators used is preferably 99/1 to 20/80, more preferably 99/1 to 40/60, and still more preferably 99/1 to 50/50 in a mass ratio (compound (A)/compound (A′)). Further, in the case of using the compound (A) in combination with the compound (A′), preferred is a combination in which the anion moiety of the compound (A) is the same as the anion moiety of the compound (A′).

[2]Resin (B) Capable of Decomposing by the Action of an Acid to Change the Solubility in a Developer

The actinic ray-sensitive or radiation-sensitive resin composition of the present invention preferably contains a resin capable of decomposing by the action of an acid to change the solubility in a developer (hereinafter, also referred to as an “acid-decomposable resin” or a “resin (B)”).

The acid-decomposable resin has a group capable of decomposing by the action of an acid to generate a polar group (hereinafter also referred to as an “acid-decomposable group”), at the main chain or the side chain, or both of the main chain and the side chain.

The resin (B) is preferably insoluble or sparingly soluble in an alkali developer.

The acid-decomposable group preferably has a structure in which the polar group is protected with a group capable of decomposing and leaving by the action of an acid.

Examples of the polar group may include a phenolic hydroxyl group, a carboxyl group, a fluorinated alcohol group, a sulfonate group, a sulfonamide group, a sulfonylimide group, an (alkylsulfonyl)alkylcarbonyl)methylene group, an (alkylsulfonyl)(alkylcarbonyl)imide group, a bis(alkylcarbonyl)methylene group, a bis(alkylcarbonyl)imide group, a bis(alkylsulfonyl)methylene group, a bis(alkylsulfonyl)imide group, a tris(alkylcarbonyl)methylene group, a tris(alkylsulfonyl)methylene group and the like.

Examples of a preferred alkali-soluble group may include a carboxyl group, a fluorinated alcohol group (preferably a hexafluoroisopropanol group) and a sulfonate group.

A preferred group as an acid-decomposable group is a group in which a hydrogen atom of the polar group is substituted by a group capable of leaving by the action of an acid.

Examples of the group capable of leaving by the action of an acid may include —C(R₃₆)(R₃₇)(R₃₈), —C(R₃₆XR₃₇)(OR₃₉), —C(R₀₁)(R₀₂)(OR₃₉) and the like.

In the formulas, R₃₆ to R₃₉ each independently represent an alkyl group, a cycloalkyl group, an aryl group, an aralkyl group or an alkenyl group. R₃₆ and R₃₇ may be bound with each other to form a ring.

R₀₁ and R₀₂ each independently represent a hydrogen atom, an alkyl group, a cycloalkyl group, an aryl group, an aralkyl group or an alkenyl group.

The acid-decomposable group is preferably a cumyl ester group, an enol ester group, an acetal ester group, a tertiary alkyl ester group and the like. The group is more preferably a tertiary alkyl ester group.

In the case of a negative type development using a developer containing an organic solvent, the resin (B) is a resin capable of increasing the polarity by the action of an acid to decrease the solubility in the alkali developer. In addition, in the case of a positive type development using an alkali developer, the resin (B) is also a resin capable of increasing the polarity by the action of an acid to increase the solubility in the alkali developer. Meanwhile, the carboxyl group as a polar group functions as an alkali-soluble group in the case of a positive type development using an alkali developer.

The actinic ray-sensitive or radiation-sensitive resin composition according to the present invention may be used in a negative type development (development in which the exposed portion is remained as a pattern, and the unexposed portion is removed), or in a positive type development (development in which the exposed portion is removed, and the unexposed portion is remained as a pattern). That is, the actinic ray-sensitive or radiation-sensitive resin composition according to the present invention may be an actinic ray-sensitive or radiation-sensitive resin composition for an organic solvent development, which is used in a development using a developer containing an organic solvent, or an actinic ray-sensitive or radiation-sensitive resin composition for an alkai development, which is used in a development using an alkali developer. Here, the term “for an organic solvent development” refers to a use that is used in a process of developing a film using a developer containing at least an organic solvent, and the term “for an alkai development” refers to a use that is used in a process of developing a film using an alkali developer.

The repeating unit having an acid-decomposable group, which may be contained in the resin (B), is preferably a repeating unit represented by the following Formula (AI).

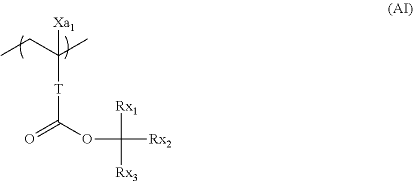

In Formula (AI),

Xa₁ represents a hydrogen atom or an alkyl group which may have a substituent.

T represents a single bond or a divalent linking group.

Rx₁ to Rx₃ each independently represent an alkyl group (straight or branched) or a cycloalkyl group (monocyclic or polycyclic).

Two of Rx₁ to Rx₃ may be bound with each other to form a cycloalkyl group (monocyclic or polycyclic).

Examples of the alky group which may have a substituent, represented by Xa₁, may include a methyl group or a group represented by —CH₂—R₁₁. R₁₁ represents a halogen atom (a fluorine atom and the like), a hydroxyl group or a monovalent organic group, and examples thereof may include an alkyl group having 5 or less carbon atoms and an acyl group having 5 or less carbon atoms, preferably an alkyl group having 3 or less carbon atoms, and more preferably a methyl group. In an aspect, Xa₁ is preferably a hydrogen atom, a methyl group, a trifluoromethyl group or a hydroxymethyl group.

Examples of the divalent linking group of T may include an alkylene group, a —COO-Rt- group, a —O-Rt- group and the like. In the formulas, Rt represents an alkylene group or a cycloalkylene group.

T is preferably a single bond or a —COO-Rt- group. Rt is preferably an alkylene group having 1 to 5 carbon atoms, and more preferably a —CH₂— group, a —(CH₂)₂— group or a —(CH₂)₃— group.

The alkyl group of Rx₁ to Rx₃ is preferably an alkyl group having 1 to 4 carbon atoms such as a methyl group, an ethyl group, a n-propyl group, an isopropyl group, a n-butyl group, an isobutyl group and a t-butyl group.

The cycloalkyl group of Rx₁ to Rx₃ is preferably a monocyclic cycloalkyl group such as a cyclopentyl group and a cyclohexyl group, or a polycyclic cycloalkyl group such as a norbornyl group, a tetracyclodecanyl group, a tetracyclododecanyl group and an adamantyl group.

The cycloalkyl group formed by two of Rx₁ to Rx₃ being bound with each other is preferably a monocyclic cycloalkyl group such as a cyclopentyl group and a cyclohexyl group, or a polycyclic cycloalkyl group such as a norbomyl group, a tetracyclodecanyl group, a tetracyclododecanyl group and an adamantyl group, and particularly preferably a monocyclic cycloalkyl group having 5 to 6 carbon atoms.

In the cycloalkyl group formed by two of Rx₁ to Rx₃ being bound with each other, for example, one of the methylene groups constituting the ring may be substituted by a heteroatom such as an oxygen atom or a group having a heteroatom such as a carbonyl group.

The repeating unit represented by Formula (AI) is preferably, for example, an aspect in which Rx₁ is a methyl group or an ethyl group, and Rx₂ and Rx₃ are bound with each other to form the aforementioned cycloalkyl group.

Each of the groups may have a substituent, and examples of the substituent may include an alkyl group (having 1 to 4 carbon atoms), a halogen atom, a hydroxyl group, an alkoxy group (having 1 to 4 carbon atoms), a carboxyl group, an alkoxycarbonyl group (2 to 6 carbon atoms) and the like, and the substituent preferably has 8 or less carbon atoms.

The content as the sum of the repeating units having an acid-decompable group is preferably 20 mol % to 80 mol %, more preferably 25 mol % to 75 mol %, and still more preferably 30 mol % to 70 mol % based on the whole repeating units in the resin (B).

Specific examples of the preferred repeating units having an acid-decomposable group are shown below, but the present invention is not limited thereto.

In the specific examples, Rx represents a hydrogen atom, CH₃, CF₃ or CH₂OH. Rxa and Rxb each represent an alkyl group having 1 to 4 carbon atoms. Z represents a substituent containing a polar group, and when a plurality thereof is present, Z's are independent from each other. p represents 0 or a positive integer. Examples of the substituent containing a polar group represented by Z may include a polar group itself such as a hydroxyl group, a cyano group, an amino group, an alkylamide group or a sulfonamide group, or a straight or branched alkyl group or a cycloalkyl group having a polar group, and preferably an alkyl group having a hydroxyl group. The branched alkyl group is particularly preferably an isopropyl group.

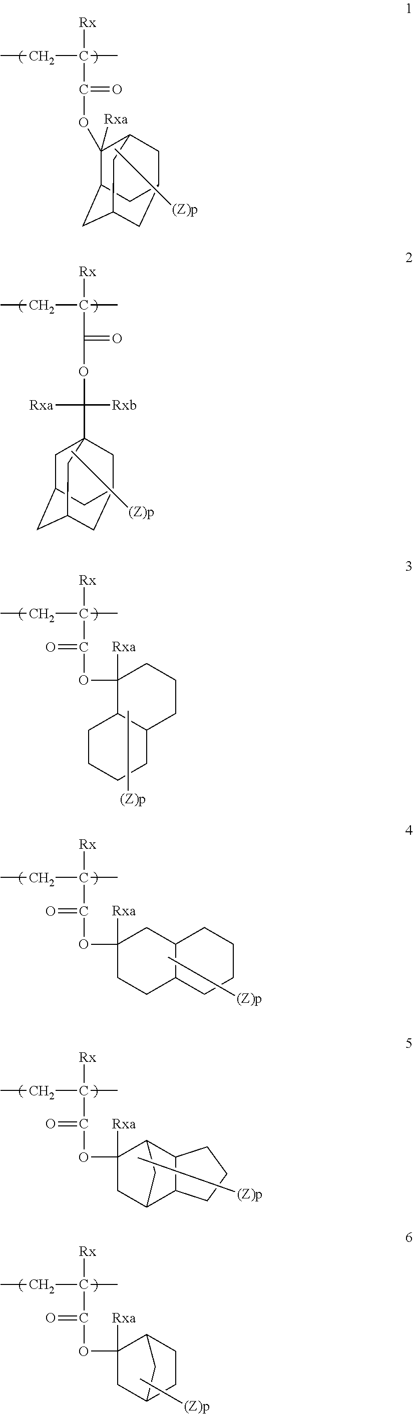

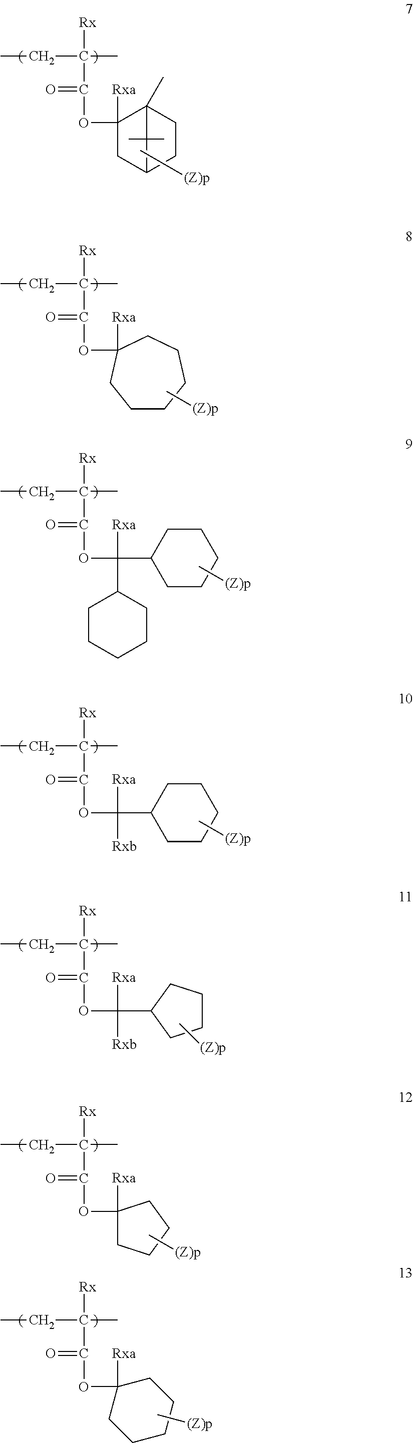

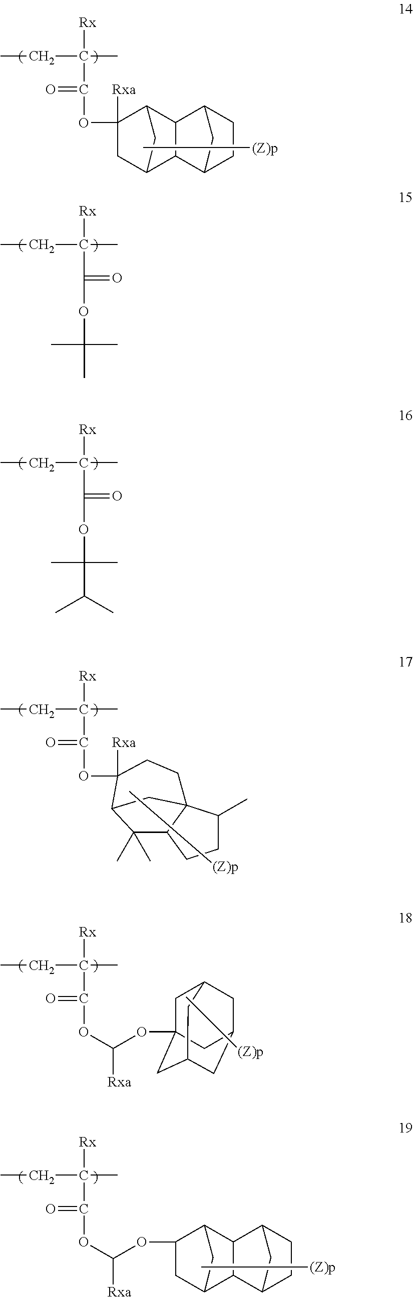

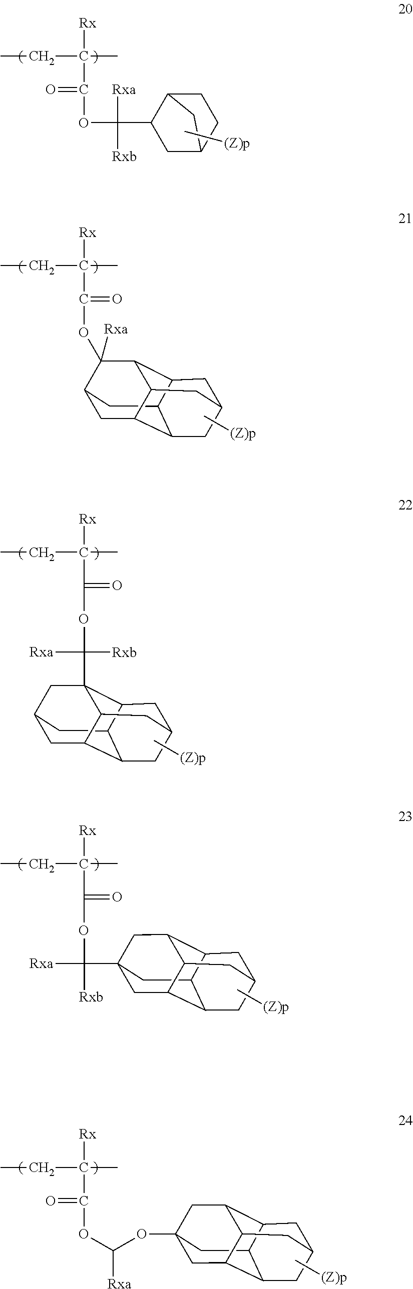

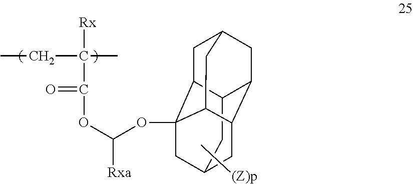

It is preferred that the resin (B) contains, for example, a repeating unit represented by Formula (3), as the repeating unit represented by Formula (AI).

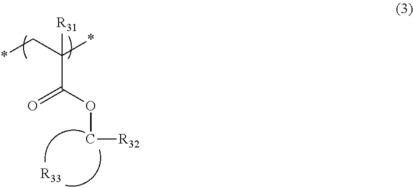

In Formula (3),

R₃₁ represents a hydrogen atom or an alkyl group.

R₃₂ represents a methyl group, an ethyl group, a n-propyl group, an isopropyl group, a n-butyl group, an isobutyl group or a sec-butyl group.

R₃₃ represents an atomic group required to form a monocyclic alicyclic hydrocarbon structure together with a carbon atom to which R₃₂ is bonded. In the alicyclic hydrocarbon structure, a part of carbon atoms constituting the ring may be substituted by a heteroatom or a group having a heteroatom.

The alkyl group of R₃₁ may have a substituent, and examples of the substituent may include a fluorine atom, a hydroxyl group and the like.

R₃₁ represents preferably a hydrogen atom, a methyl group, a trifluoromethyl group or a hydroxymethyl group.

R₃₂ is preferably a methyl group, an ethyl group, a n-propyl group or an isopropyl group, and more preferably a methyl group or an ethyl group.

The monocyclic alicyclic hydrocarbon structure formed by R₃₃ together with a carbon atom is preferably a 3- to 8-membered ring, and more preferably a 5- or 6-membered ring.

In the monocyclic alicyclic hydrocarbon structure formed by R₃₃ together with a carbon atom, examples of the heteroatom which may constitute a ring may include an oxygen atom, a sulfur atom and the like, and examples of the group having a heteroatom may include a carbonyl group and the like. However, it is preferred that the group having a heteroatom is not an ester group (ester bond).

It is preferred that the monocyclic alicyclic hydrocarbon structure formed by R₃₃ together with a carbon atom is formed only of carbon atoms and hydrogen atoms.

It is preferred that the repeating unit represented by Formula (3) is a repeating unit represented by the following Formula (3′).

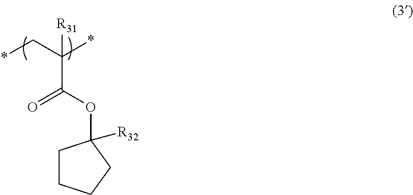

In Formula (3′), R₃₁ and R₃₂ have the same meaning as those in Formula (3), respectively.

Specific examples of the structure represented by Formula (3) are shown below, but not limited thereto.

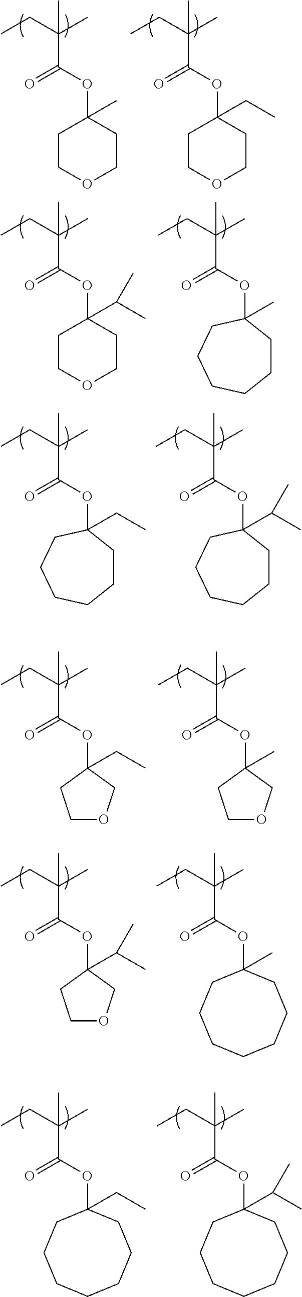

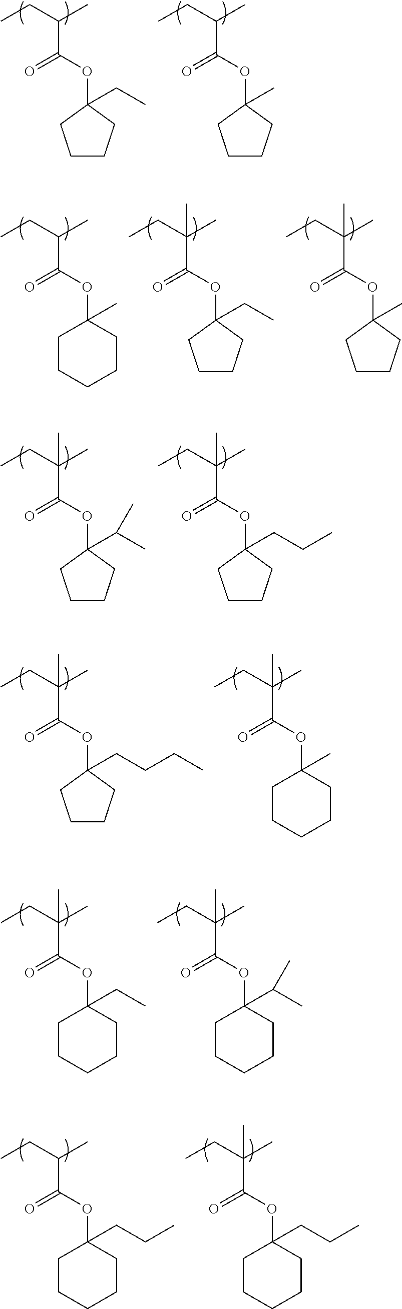

The content of the repeating unit having a structure represented by Formula (3) is preferably 20 mol % to 80 mol %, more preferably 25 mol % to 75 mol %, and still more preferably 30 mol % to 70 mol % based on the whole repeating units in the resin (B).

It is more preferred that the resin (B) contains, for example, at least one of a repeating unit represented by Formula (I) and a repeating unit represented by Formula (II), as the repeating unit represented by Formula (AI).

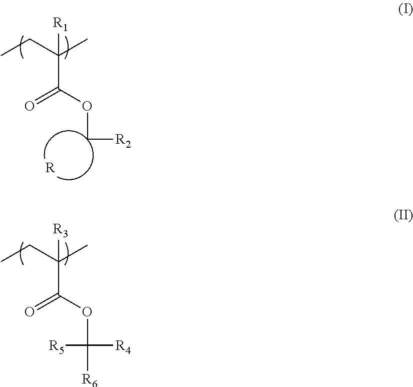

In Formula (I) and Formula (II),

R₁ and R₃ each independently represent a hydrogen atom, a methyl group which may have a substituent or a group represented by —CH₂—R₁₁. R₁₁ represents a monovalent organic group.

R₂, R₄, R₅ and R₆ each independently represent an alkyl group or a cycloalkyl group.

R represents an atomic group required to form an alicyclic ring together with a carbon atoms to which R₂ is bonded.

R₁ and R₃ represent preferably a hydrogen atom, a methyl group, a trifluoromethyl group or a hydroxymethyl group. Specific examples and preferred examples of the monovalent organic group in R₁₁ are the same as those described in R₁₁ in Formula (AI).

The alkyl group in R₂ may be straight or branched, and may have a substituent.

The cycloalkyl group in R₂ may be monocyclic or polycyclic, and may have a substituent.

R₂ is preferably an alkyl group, more preferably an alkyl group having 1 to 10 carbon atoms, and still more preferably 1 to 5 carbon atoms, and examples thereof may include a methyl group, an ethyl group and the like.

R represents an atomic group required to form an alicyclic ring together with a carbon atoms. The alicyclic structure formed by R together with the carbon atoms is preferably a monocyclic alicyclic structure, and has preferably 3 to 7 carbon atoms, and more preferably 5 or 6 carbon atoms.

R₃ is preferably a hydrogen atom or a methyl group, and more preferably a methyl group.

The alkyl group in R₄, R₅ and R₆ may be straight or branched, and may have a substituent. The alkyl group is preferably an alkyl group having 1 to 4 carbon atoms such as a methyl group, an ethyl group, a n-propyl group, an isopropyl group, a n-butyl group, an isobutyl group and a t-butyl group.

The cycloalkyl group in R₄, R₅ and R₆ may be monocyclic or polycyclic, and may have a substituent. The cycloalkyl group is preferably a monocyclic cycloalkyl group such as a cyclopentyl group and a cyclohexyl group, or a polycyclic cycloalkyl group such as a norbomyl group, a tetracyclodecanyl group, a tetracyclododecanyl group and an adamantyl group.

The substituent which may be possessed by each of the groups may include the same group as those described above as the substituent which may be possessed by each of the groups in Formula (AI).

The acid-decomposable resin is more preferably a resin containing the repeating unit represented by Formula (I) and the repeating unit represented by Formula (II), as the repeating unit represented by Formula (AI).

Further, in another aspect, the acid-decomposable resin is more preferably a resin containing at least two or more kinds of the repeating unit represented by Formula (I), as the repeating unit represented by Formula (AI). In the case of containing two or more kinds of the repeating unit of Formula (I), it is preferred to contain a repeating unit in which the alicyclic structure formed by R together with a carbon atom is a monocyclic alicyclic structure, and a repeating unit in which the alicyclic structure formed by R together with a carbon atom is a polycyclic alicyclic structure. The monocyclic alicyclic structure has preferably 5 to 8 carbon atoms, more preferably 5 or 6, and particularly preferably 5 carbon atoms. The polycyclic alicyclic structure is preferably a norbomyl group, a tetracyclodecanyl group, a tetracyclododecanyl group or an adamantyl group.

The repeating unit having an acid-decomposable group contained in the resin (B) may be used alone or in combination of two or more thereof. When used in combination, the combinations exemplified below are preferred. In the following formulas, R's each independently represent a hydrogen atom or a methyl group.

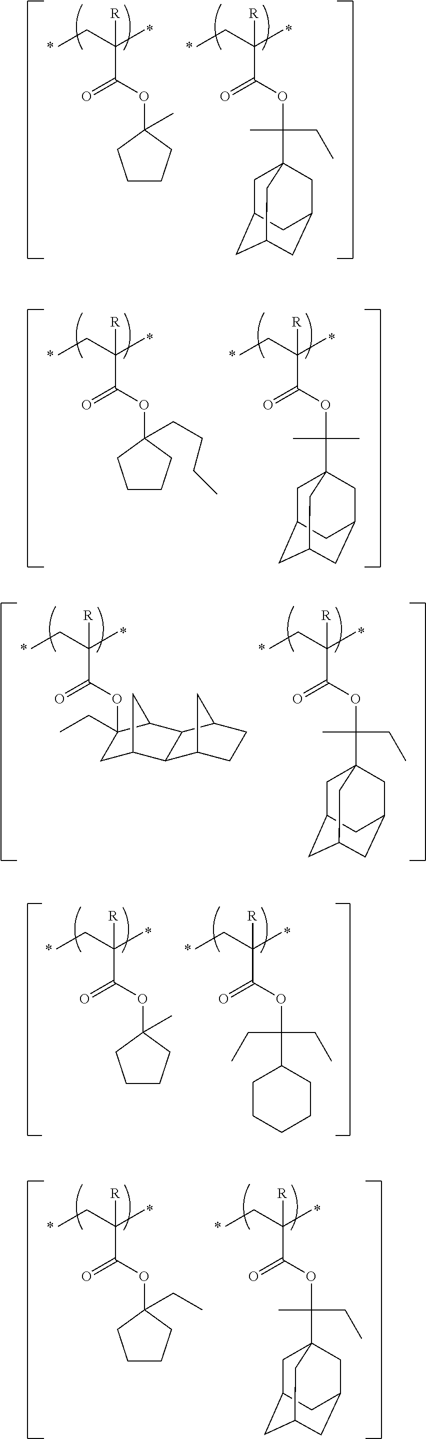

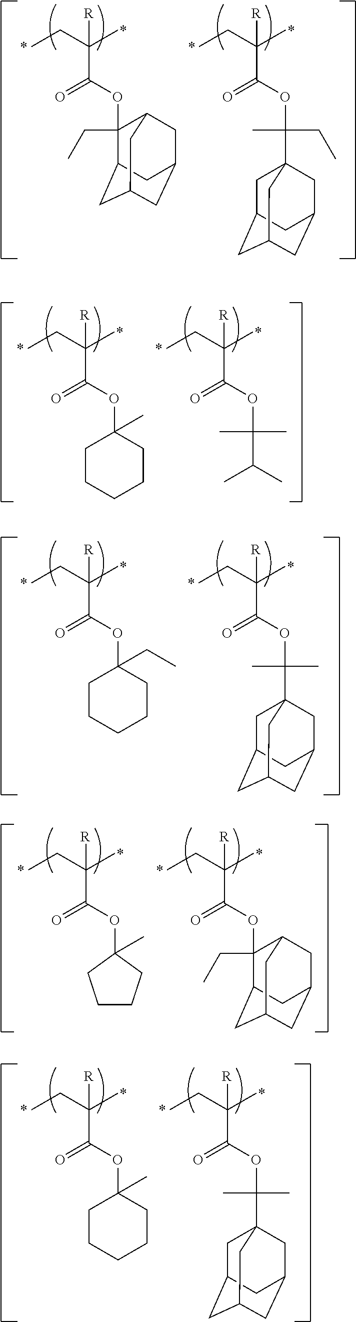

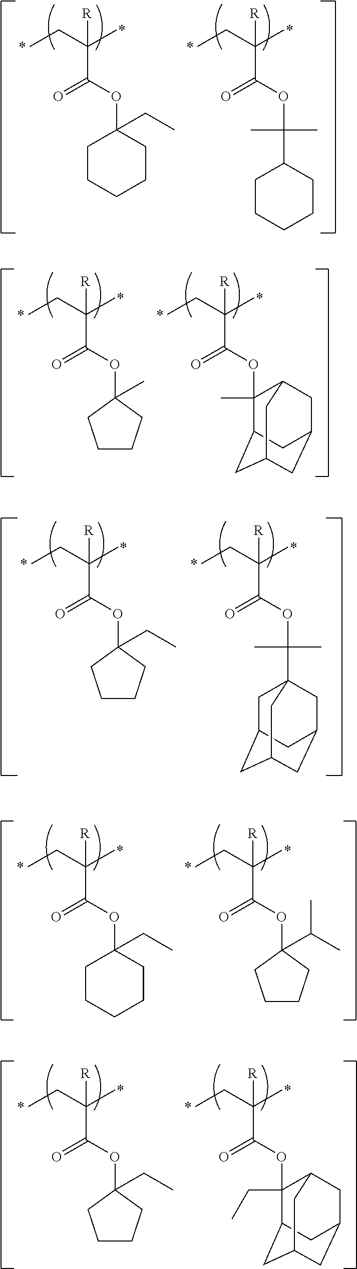

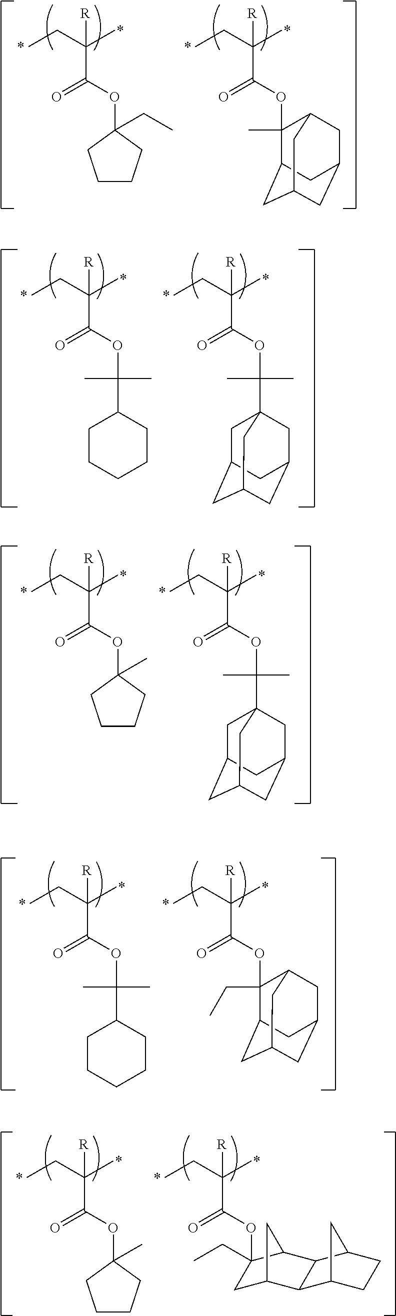

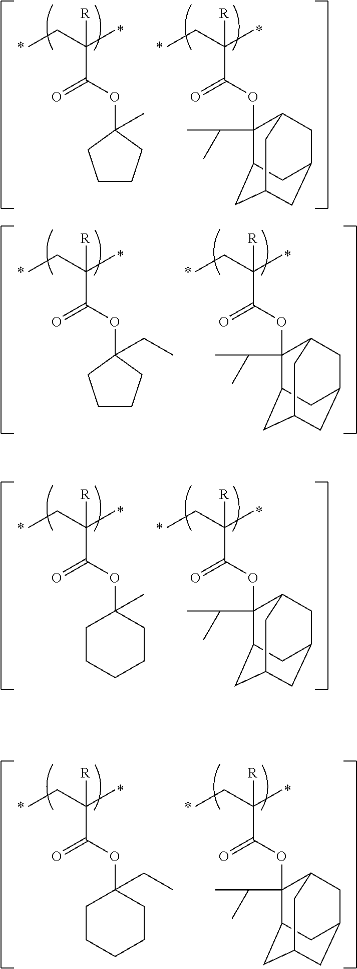

In an aspect, it is preferred that the resin (B) contains a repeating unit having a cyclic carbonate ester structure. The cyclic carbonate ester structure is a structure having a ring containing a bond represented by —O—C(═O)—O— as an atomic group constituting the ring.

The ring containing a bond represented by —O—C(═O)—O— as an atomic group constituting the ring is preferably a 5- to 7-membered ring, and most preferably 5-membered ring. The ring may be condensed with other rings to form a condensed ring.

It is preferred that the resin (B) contains a lactone structure or a sultone (cyclic sulfonate ester) structure.

Although any group may be used as long as a lactone structure or a sultone structure is possessed, the lactone group or the sultone group is preferably a 5- to 7-membered ring lactone structure or sultone structure, and more preferably a 5- to 7-membered ring lactone structure or sultone structure to which another ring structure is condensed to form a bicyclo or spiro structure. It is still more preferred to have a lactone structure or a sultone structure represented by any one of the following Formulas (LC1-1) to (LC1-17), (SL1-1) and (SL1-2). Further, the lactone structure or the sultone structure may be bonded directly to the main chain. A preferred lactone structure or sultone structure is (LC1-1), (LC1-4), (LC1-5), (LC1-6) and (LC1-8), and more preferably (LC-4). By using such a specific lactone structure or sultone structure, LWR and development defects are improved.

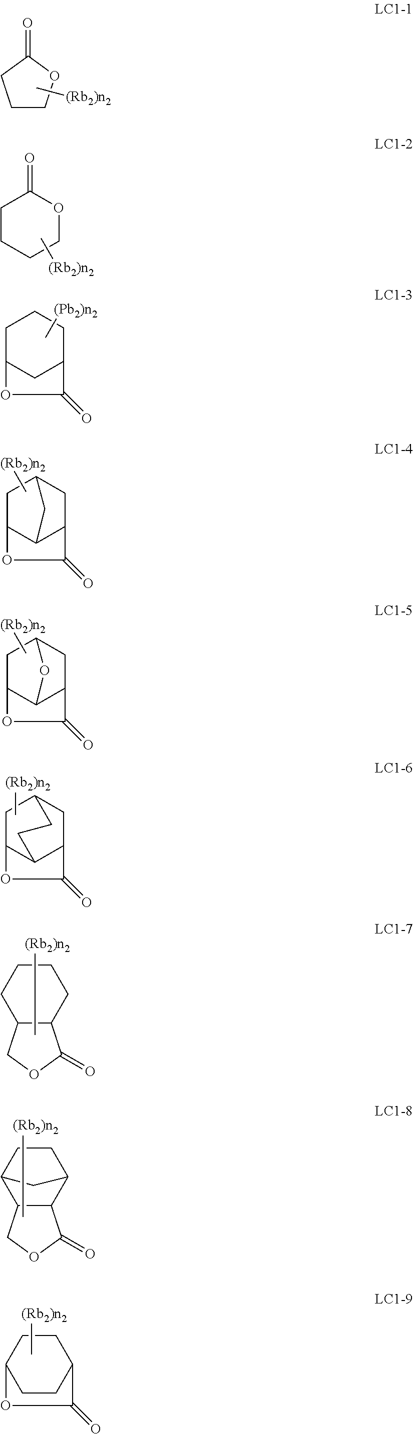

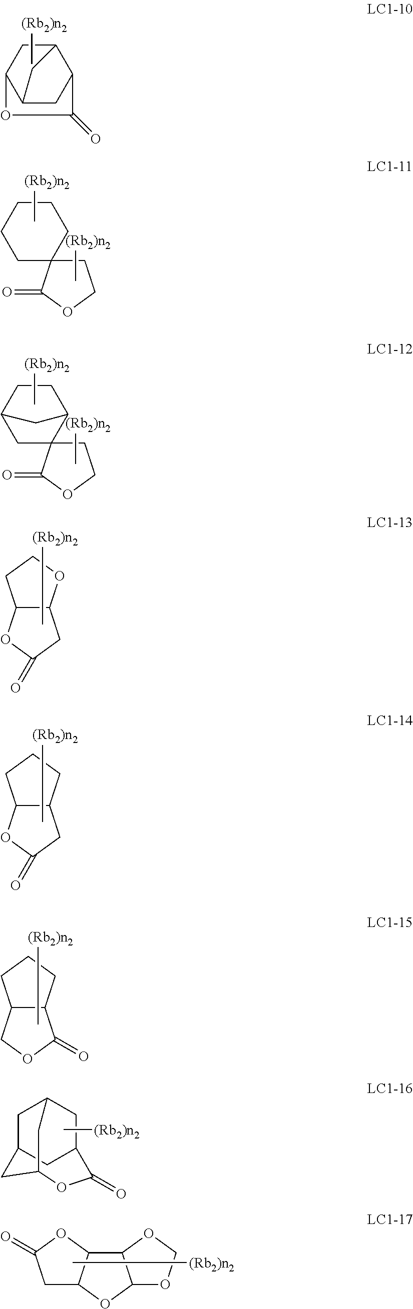

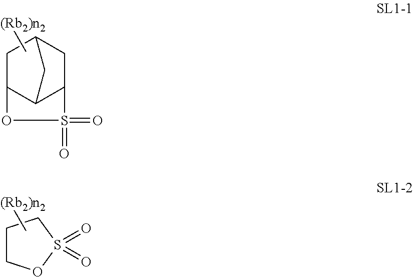

The lactone structure or the sultone structure moiety may or may not have a substituent (Rb₂). Preferred examples of the substituent (Rb₂) may include an alkyl group having 1 to 8 carbon atoms, a cycloalkyl group having 4 to 7 carbon atoms, an alkoxy group having 1 to 8 carbon atoms, an alkoxycarbonyl group having 2 to 8 carbon atoms, a carboxyl group, a halogen atom, a hydroxyl group, a cyano group, an acid-decomposable group and the like. The substituent is more preferably an alkyl group having 1 to 4 carbon atoms, a cyano group and an acid-decomposable group. n₂ represents an integer of 0 to 4. When n₂ is 2 or more, the substituents (Rb₂'s) may be the same as or different. In addition, the substituents (Rb₂'s) may be bound with each other to form a ring.

It is preferred that the resin (B) contains a lactone structure or a sultone structure represented by the following Formula (III).

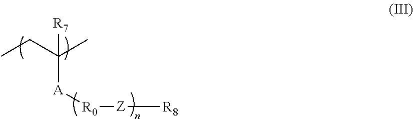

In Formula (III),

A represents an ester bond (a group represented by —COO—), a sulfonyl bond (a group represented by —SO₂—), an amide bond (a group represented by —CONH—) or a group formed by combining the groups.

When a plurality of R₀'s is present, R₀'s each independently represent an alkylene group, a cycloalkylene group or a combination thereof.

When a plurality of Z's is present, Z's each independently represent a single bond, an ether bond, an ester bond, an amide bond, a urethane bond

(a group represented by

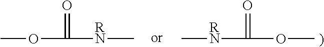

or a urea bond

(a group represented by

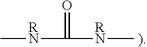

Herein, R's each independently represent a hydrogen atom, an alkyl group, a cycloalkyl group or an aryl group.

R₈ represents a monovalent organic group having a lactone structure or a sultone structure.

n is a repeating number of a structure represented by —R₀—Z—, and represents an integer of 0 to 2.

R₇ represents a hydrogen atom, a halogen atom or an alkyl group.

The alkylene group and the cycloalkylene group of R₀ may have a substituent.

Z is preferably an ether bond or an ester bond, and particularly preferably an ester bond.

The alkyl group of R₇ is preferably an alkyl group having 1 to 4 carbon atoms, more preferably a methyl group and an ethyl group, and particularly preferably a methyl group. Each of the alkylene group and the cycloalkylene group of R₀ and the alkyl group of R₇ may be substituted, and examples of the substituent may include a halogen atom such as a fluorine atom, a chlorine atom and a bromine atom, a mercapto group, a hydroxyl group, an alkoxy group such as a methoxy group, an ethoxy group, an isopropoxy group, a t-butoxy group and a benzyloxy group, and an acyloxy group such as an acetyloxy group and a propionyloxy group. R₇ is preferably a hydrogen atom, a methyl group, a trifluoromethyl group or a hydroxymethyl group.

A preferred chain alkylene group in R₀ is preferably a chain alkylene having 1 to 10 carbon atoms, and more preferably 1 to 5 carbon atoms, and examples thereof may include a methylene group, an ethylene group, a propylene group and the like. A preferred cycloalkylene group is a cycloalkylene group having 3 to 20 carbon atoms, and examples thereof may include a cyclohexylene group, a cyclopentylene group, a norbornylene group, an adamantylene group and the like. In order to exhibit effects of the present invention, a chain alkylene group is more preferred, and a methylene group is particularly preferred.

The monovalent organic group having a lactone structure or a sultone structure represented by R₈ is not limited as long as the organic group has a lactone structure or a sultone structure, specific examples thereof may include a lactone structure or a sultone structure represented by any one of Formulas (LC1-1) to (LC1-17), (SL1-1) and (SL1-2), and among them, a structure represented by (LC1-4) is particularly preferred. Further, n₂ in (LC1-1) to (LC1-17), (SL1-1) and (SL1-2) is more preferably 2 or less.

Further, R₈ is preferably a monovalent organic group having an unsubstituted lactone structure or a sultone structure, or a monovalent organic group having a lactone structure or a sultone structure having a methyl group, a cyano group or an alkoxycarbonyl group as a substituent, and more preferably a monovalent organic group having a lactone structure (cyanolactone) or a sultone structure (cyanosultone) having a cyano group as a substituent.

In Formula (III), n is preferably 1 or 2.

Specific examples of the repeating unit having a group having a lactone structure or a sultone structure represented by Formula (III) will be shown below, but the present is not limited thereto.

In the following specific examples, R represents a hydrogen atom, an alkyl group which may have a substituent or a halogen atom, and preferably a hydrogen atom, a methyl group, a hydroxymethyl group, an acetoxymethyl group.

In the following formulas, Me represents a methyl group.

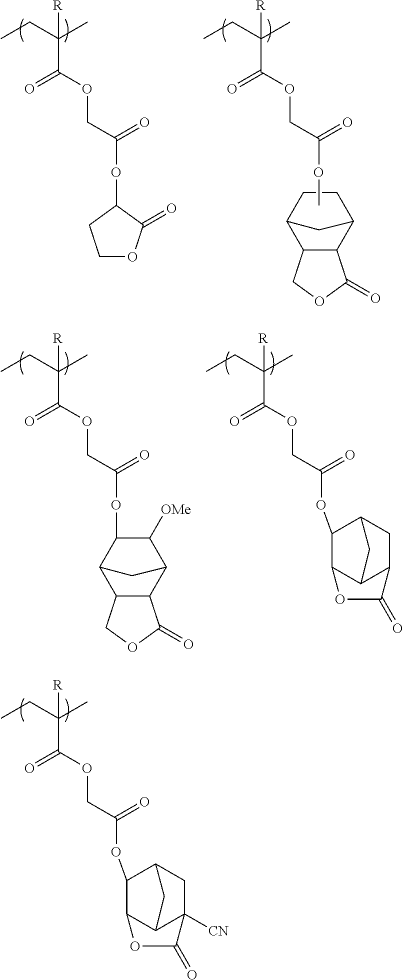

It is more preferred that the repeating unit having a lactone structure or a sultone structure is a repeating unit represented by the following Formula (III-1) or (III-1′).

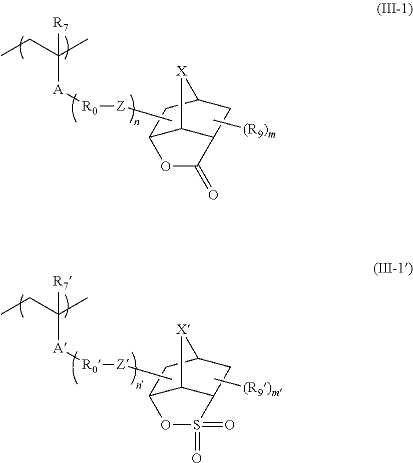

In Formulas (III-1) and (III-1′),

R₇, A, R₀, Z and n have the same meaning as in Formula (III).

R₇′, A′, R₀′, Z′ and n′ have the same meaning as R₇, A, R₀, Z and n in Formula (III), respectively.

When a plurality of R₉'s is present. R₉'s each independently represent an alkyl group, a cycloalkyl group, an alkoxycarbonyl group, a cyano group, a hydroxyl group or an alkoxy group, and when a plurality thereof is present, two R₀'s may be bound with each other to form a ring.

When a plurality of R₉′'s is present, R₉′'s each independently represent an alkyl group, a cycloalkyl group, an alkoxycarbonyl group, a cyano group, a hydroxyl group or an alkoxy group, and when a plurality thereof is present, two R₉′'s may be bound with each other to form a ring.

X and X′each independently represent an alkylene group, an oxygen atom or a sulfur atom.

m and m′ are the number of substituent, and each independently represent an integer of 0 to 5. m and m′ are each independently preferably 0 or 1.

The alkyl group of R₉ and R₉′ is preferably an alkyl group having 1 to 4 carbon atoms, more preferably a methyl group or an ethyl group, and most preferably a methyl group. Examples of the cycloalkyl group may include a cyclopropyl group, a cyclobutyl group, a cyclopentyl group and a cyclohexyl group. Examples of the alkoxycarbonyl group may include a methoxycarbonyl group, an ethoxycarbonyl group, a n-butoxycarbonyl group, a t-butoxycarbonyl group and the like. Examples of the alkoxy group may include a methoxy group, an ethoxy group, a propoxy group, an isopropoxy group, a butoxy group and the like. The group may have a substituent, and examples of the substituent may include a hydroxyl group, an alkoxy group such as a methoxy group and an ethoxy group, a cyano group, and a halogen atom such as a fluorine atom. R₉ and R′ are preferably a methyl group, a cyano group or alkoxycarbonyl group, and more preferably a cyano group.

Examples of the alkylene group of X and X′ may include a methylene group, an ethylene group and the like. X and X′ are preferably an oxygen atom or a methylene group, and more preferably a methylene group.

When m and m′ are 1 or more, it is preferred that at least one of R₉ and R₉′ is substituted at the α-position or β-position of the carbonyl group of the lactone, and particularly preferably at the α-position.

Specific examples of the group having lactone structure or the repeating unit having a sultone structure represented by Formula (III-1) or (III-1′) are shown, but the present invention is not limited thereto. In the following specific examples, R represents a hydrogen atom, an alkyl group which may have a substituent or a halogen atom, and preferably a hydrogen atom, a methyl group, a hydroxymethyl group or an acetoxymethyl group.

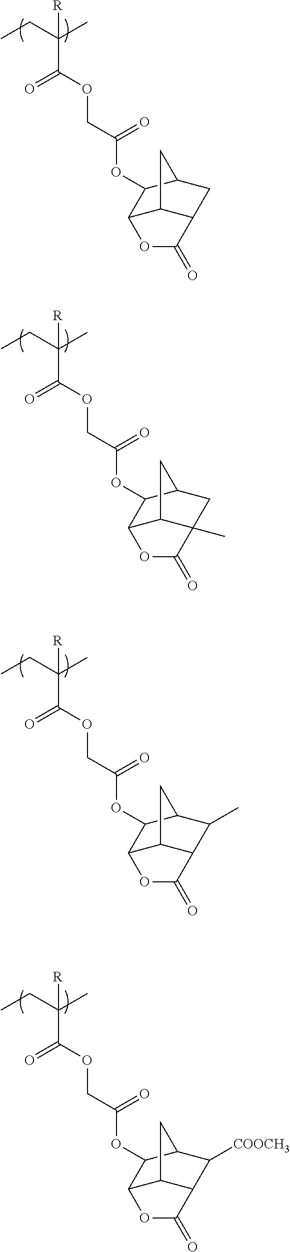

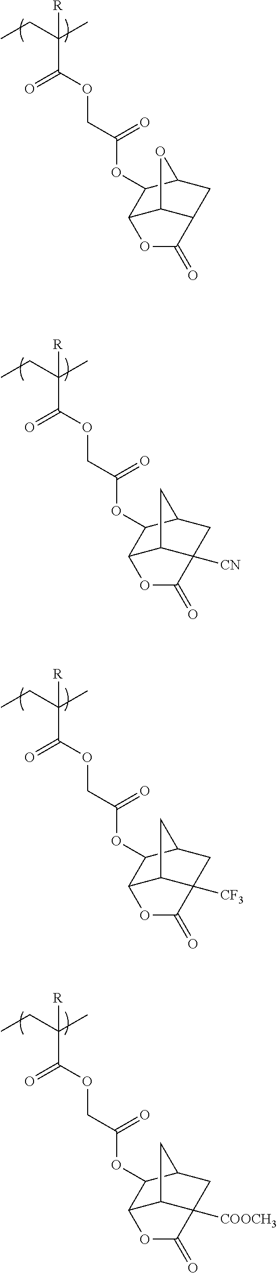

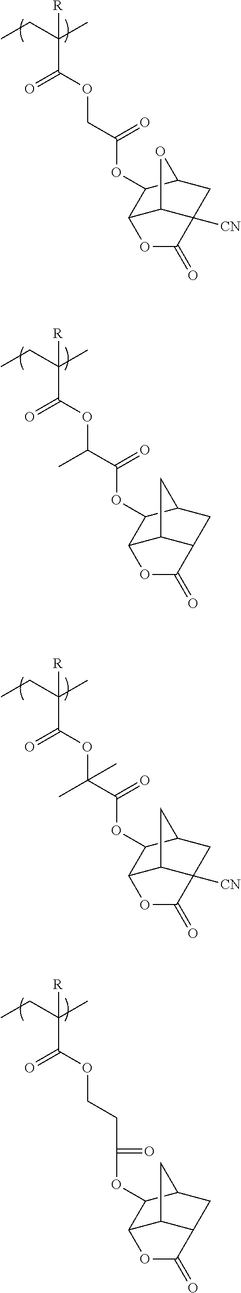

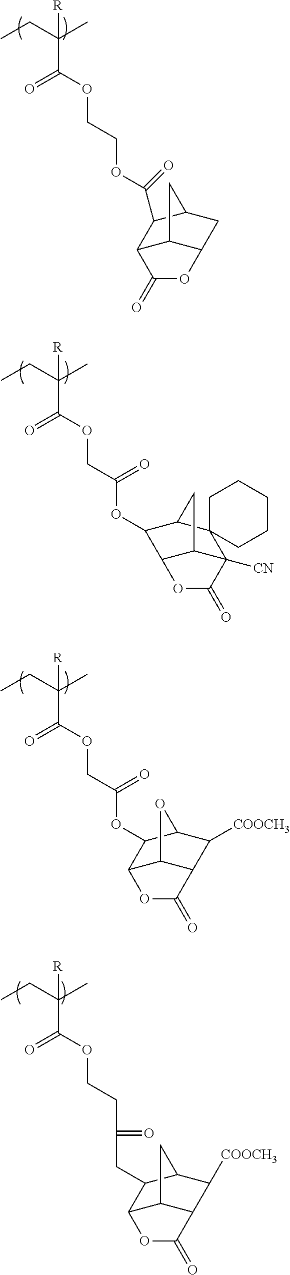

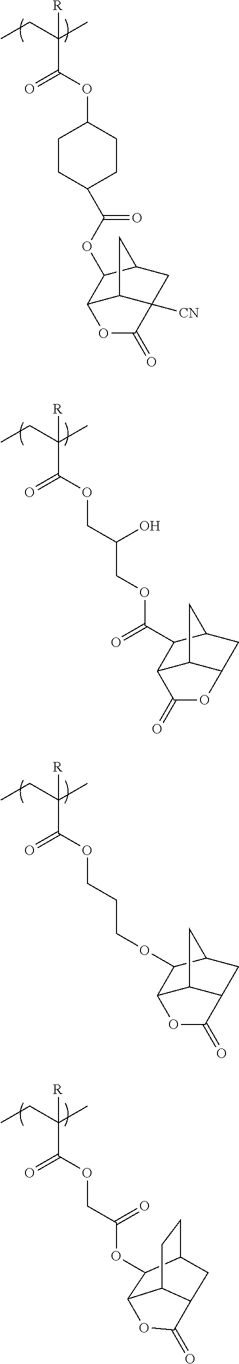

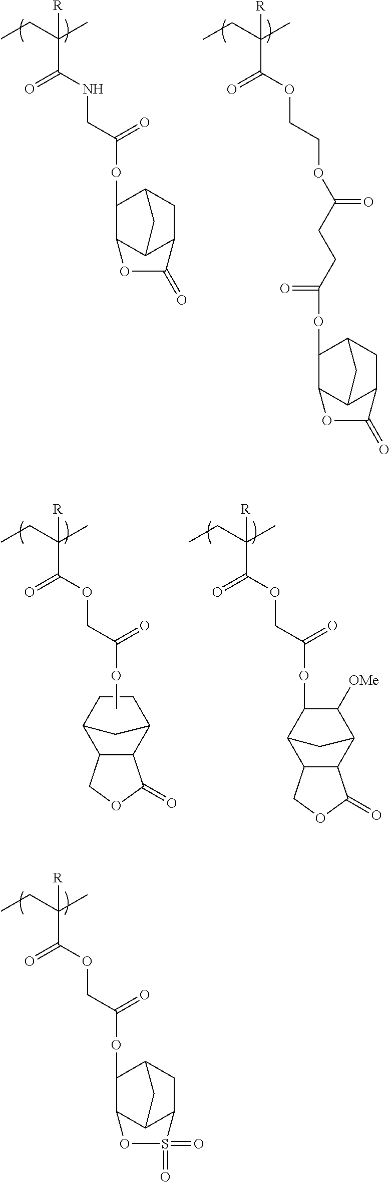

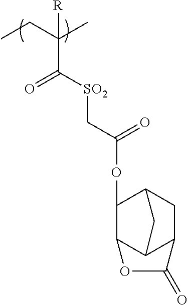

The content of the repeating unit represented by Formula (III), summed if a plurality of kinds thereof is contained, is preferably 15 mol % to 60 mol %, more preferably 20 mol % to 60 mol %, and still more preferably 30 mol % to 50 mol % based on the whole repeating units in the resin (B).

The resin (B) may also contain the repeating unit having a lactone structure or a sultone structure as described above, in addition to the unit represented by Formula (III).

Specific examples of the repeating unit having a lactone group or a sultone group are shown below, in addition to the specific examples as exemplified above, but the present invention is not limited thereto.

(In the formulas, Rx represents H, CH₃, CH₂OH or CF₃.)

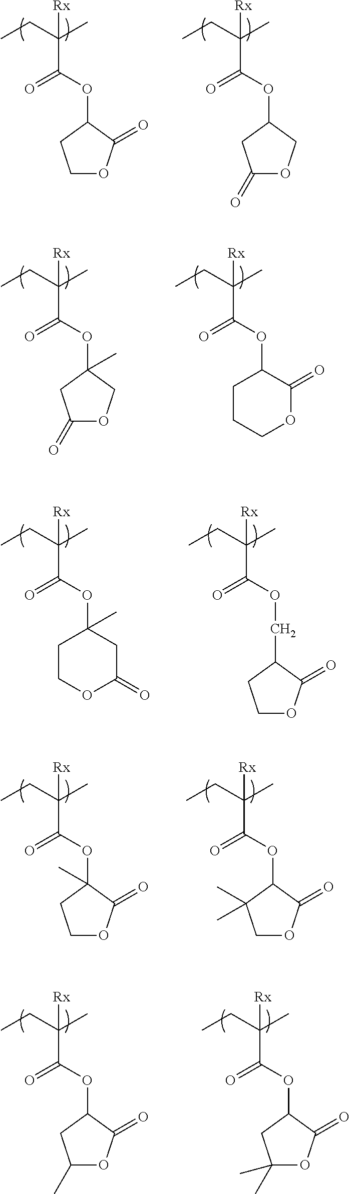

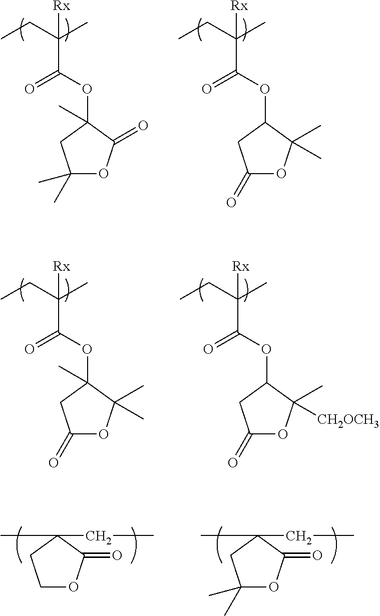

(In the formulas, Rx represents H, CH₃, CH₂OH or CF₃.)

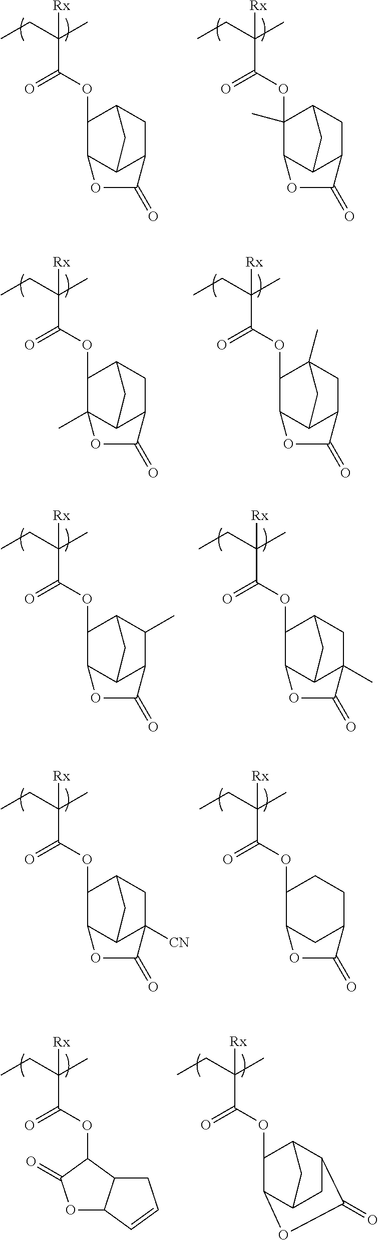

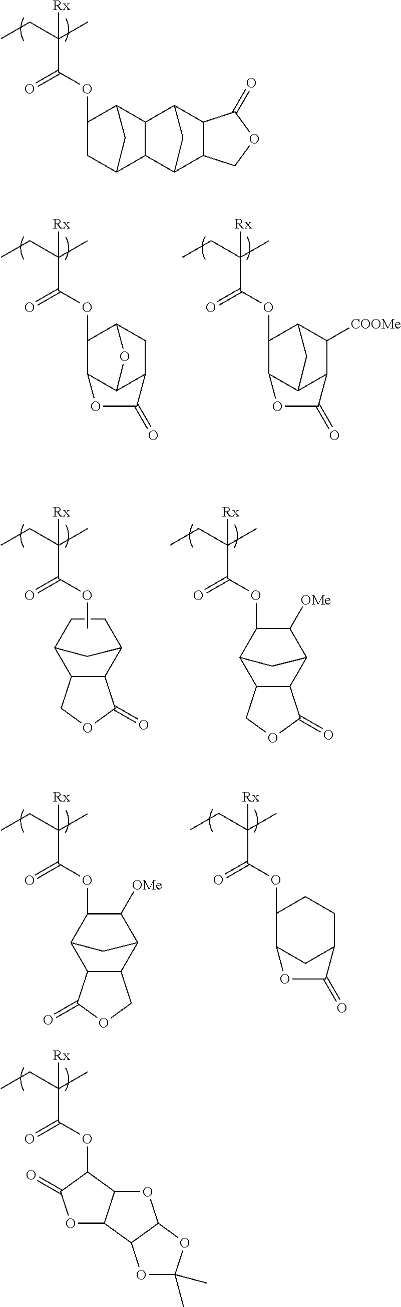

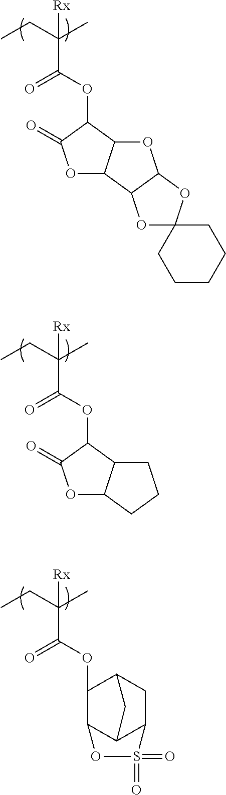

(In the formulas, Rx represents H, CH₃, CH₂OH or CF₃.)

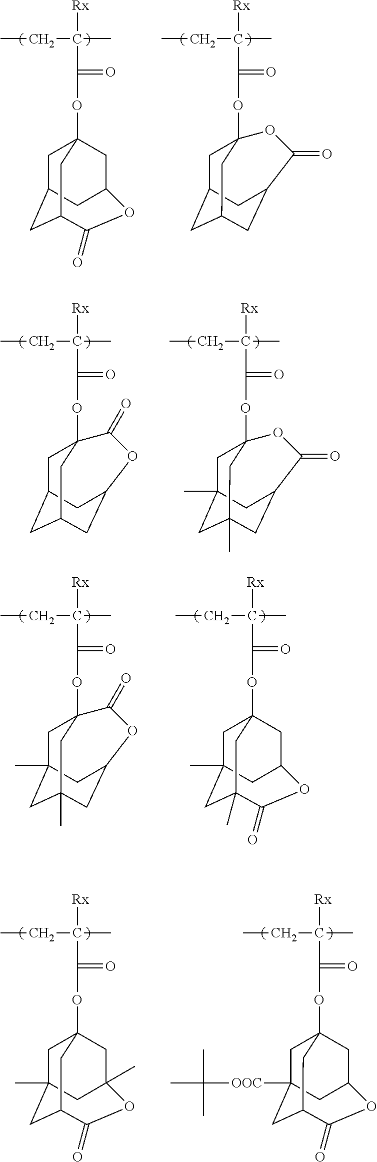

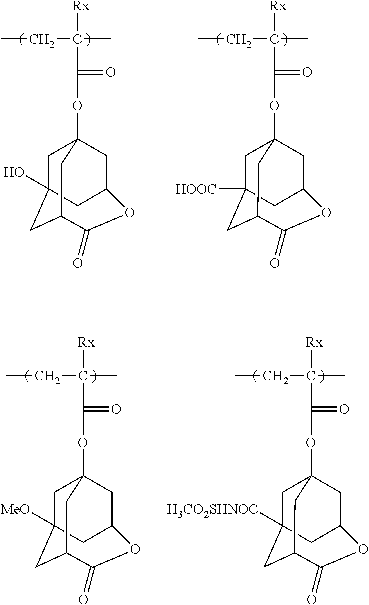

Among the specific examples, particularly preferred repeating units may be the following repeating units. By selecting the optimal lactone group or sultone group, pattern profile and iso/dense bias may be improved.

(In the formulas, Rx represents H, CH₃, CH₂OH or CF₃.)

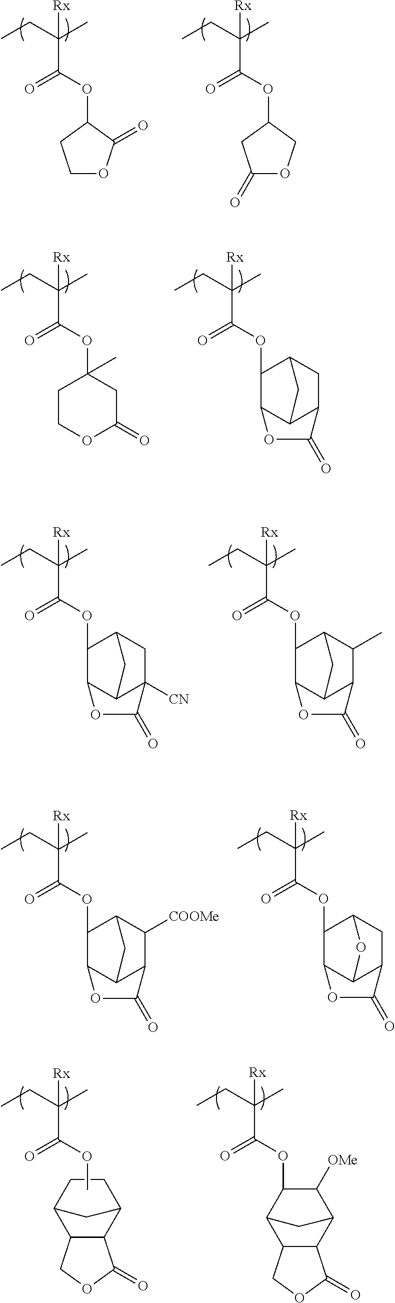

The repeating unit having a lactone group or a sultone structure usually has an optical isomer, but any optical isomer may be used. Further, the optical isomer may be used either alone or as a mixture of two or more thereof. When one kind of the optical isomer is mainly used, the optical purity (ee) thereof is preferably 90% or more, and more preferably 95% or more.

The content of the repeating units having a lactone structure or a sultone structure other than the repeating unit represented by Formula (III), summed if a plurality of kinds thereof is contained, is preferably 15 mol % to 60 mol %, more preferably 20 mol % to 50 mol %, and still more preferably 30 mol % to 50 mol % based on the whole repeating units in the resin.

In order to improve the effects of the present invention, two or more kinds of the lactone or sultone repeating units selected from Formula (III) may be used in combination as well. When used in combination, it is preferred to select two or more kinds from the lactone or sultone repeating units in which n is 1 in Formula (III), and use in combination.

The resin (B) is preferably a repeating unit having a hydroxyl group or a cyano group other than Formulas (AI) and (III). As a result, the adhesion to a substrate and the affinity for a developer are enhanced. The repeating unit having a hydroxyl group or a cyano group is preferably a repeating unit having an alicyclic hydrocarbon structure substituted with a hydroxyl group or a cyano group, and preferably has no acid-decomposable group. The alicyclic hydrocarbon structure in the alicyclic hydrocarbon structure substituted with a hydroxyl group or a cyano group is preferably an adamantyl group, a diadamantyl group or a norbornane group. The preferred alicyclic hydrocarbon structure substituted with a hydroxyl group or a cyano group is preferably a partial structure represented by the following Formulas (VIIa) to (VIId).

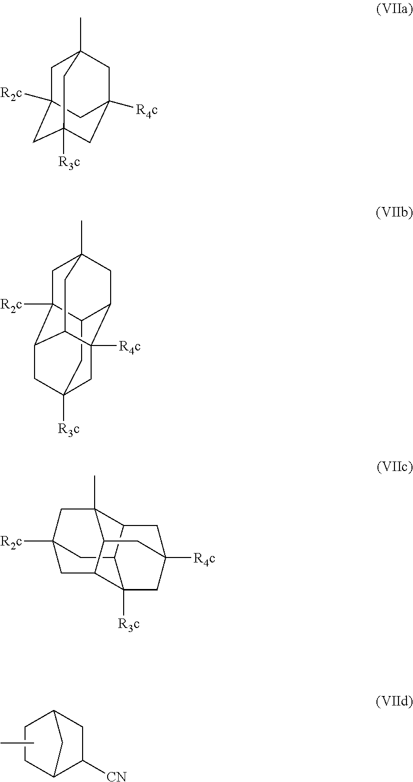

In Formulas (VIIa) to (VIIc),

R_{2c} to R_{4c} each independently represent a hydrogen atom, a hydroxyl group or a cyano group. However, at least one of R_{2c} to R_{4c} represents a hydroxyl group or a cyano group. Preferably, one or two of R_{2c} to R_{4c} are a hydroxyl group, and the rest is a hydrogen atom. In Formula (VIIa), more preferably, two of R_{2c} to R_{4c} are a hydroxyl group, and the rest is a hydrogen atom.

The repeating units having partial structures represented by Formulas (VIIa) to (VIId) may include repeating units represented by the following Formulas (AIIa) to (AIId).

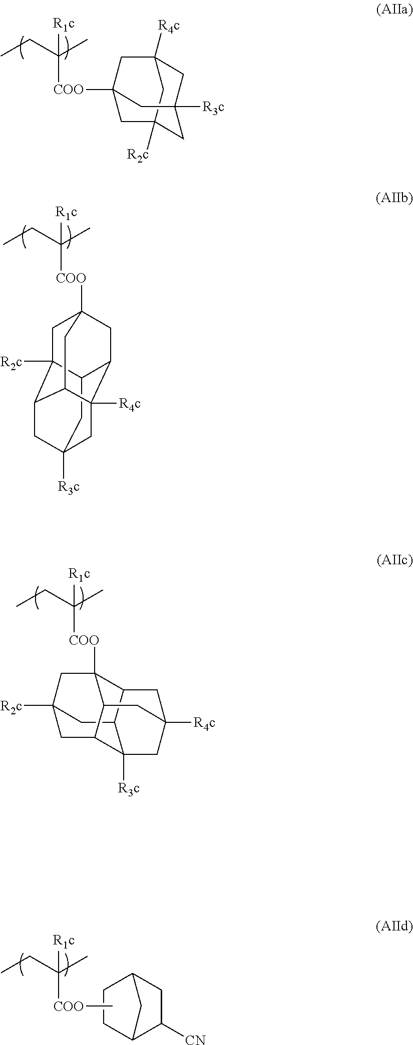

In Formulas (AIIa) to (AIId).

R_{1c} represents a hydrogen atom, a methyl group, a trifluoromethyl group or a hydroxymethyl group.

R_{2c} to R_{4c} have the same meaning as R_{2c} to R_{4c} in Formulas (VIIa) to (VIIc).

The content of the repeating unit having a hydroxyl group or a cyano group is preferably 5 mol % to 40 mol %, more preferably 5 mol % to 30 mol %, and still more preferably 10 mol % to 25 mol % based on the whole repeating units in the resin (B).

Specific examples of the repeating unit having a hydroxyl group or a cyano group are shown below, but the present invention is not limited thereto.

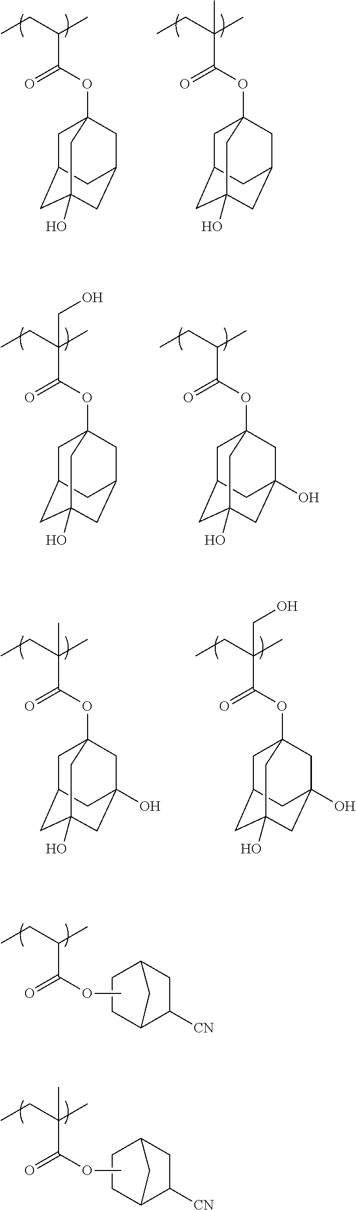

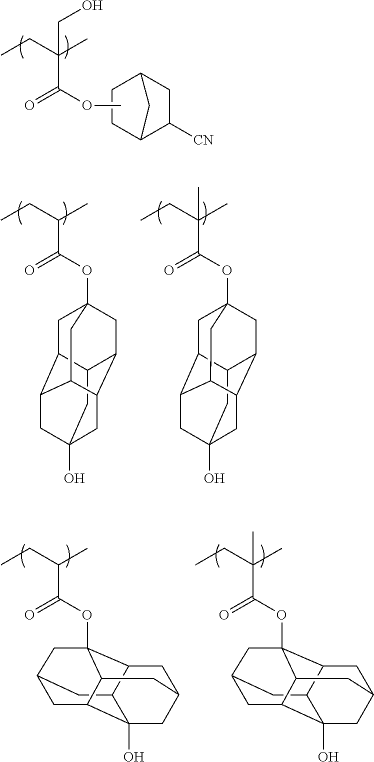

The resin used in the actinic ray-sensitive or radiation-sensitive resin composition of the present invention may have a repeating unit having an alkali-soluble group. Examples of the alkali-soluble group may include a carboxyl group, a sulfonamide group, a sulfonylimide, a bissulfonylimide, a naphthol structure, and an aliphatic alcohol group substituted with an electron-withdrawing group at the α-position (for example, a hexafluoroisopropanol group), and it is more preferred to have a repeating unit having a carboxyl group. By containing the repeating unit having an alkali-soluble group, the resolution increases in the usage of contact holes. As for the repeating unit having an alkali-soluble group, a repeating unit in which the alkali-soluble group is directly bonded to the main chain of the resin, such as repeating unit by an acrylic acid or a methacrylic acid or a repeating unit in which the acid group is bonded to the main chain of the resin through a linking group, and a repeating unit which is introduced into the end of the polymer chain by using a polymerization initiator or a chain transfer agent having an alkali-soluble group at the time of polymerization are all preferred, and the linking group may have a monocyclic or polycyclic cyclic hydrocarbon structure. A repeating unit by an acrylic acid or a methacrylic acid is particularly preferred.

The content of the repeating unit having an alkali-soluble group is preferably 0 mol % to 20 mol %, more preferably 3 mol % to 15 mol %, and still more preferably 5 mol % to 10 mol % based on the whole repeating units in the resin (B).

Specific examples of the repeating unit having an alkali-soluble group are shown below, but the present invention is not limited thereto.

In the specific examples, Rx represents H, CH₃, CH₂OH or CF₃.

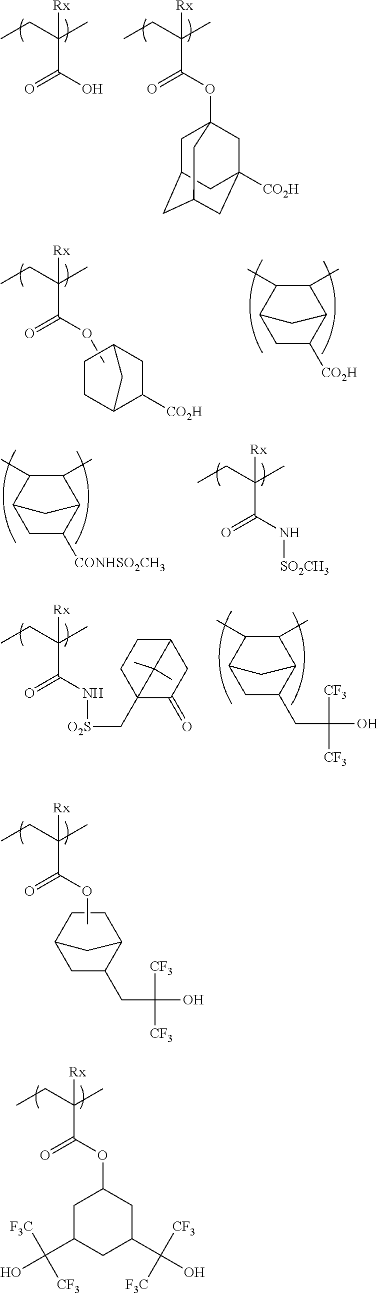

The resin (B) of the present invention may also has a repeating unit having an alicyclic hydrocarbon structure having no polar group (for example, the aforementioned alkali-soluble group, a hydroxyl group, a cyano group and the like), and not exhibiting acid decomposability. The repeating unit may include a repeating unit represented by Formula (IV).

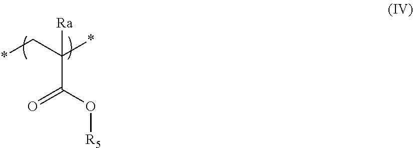

In Formula (IV), R₅ represents a hydrocarbon group having at least one cyclic structure and having no polar group.

Ra represents a hydrogen atom, an alkyl group or a —CH₂—O—Ra₂ group. In the formula, Ra₂ represents a hydrogen atom, an alkyl group or an acyl group. Ra is preferably a hydrogen atom, a methyl group, a hydroxymethyl group or a trifluoromethyl group, and particularly preferably a hydrogen atom or a methyl group.

The cyclic structure possessed by R₅ includes a monocyclic hydrocarbon group and a polycyclic hydrocarbon group. Examples of the monocyclic hydrocarbon group may include a cycloalkyl group having 3 to 12 carbon atoms such as a cyclopentyl group, a cyclohexyl group, a cycloheptyl group and a cyclooctyl group, and a cycloalkenyl group having 3 to 12 carbon atoms such as a cyclohexenyl group. A preferred monocyclic hydrocarbon group is a monocyclic hydrocarbon group having 3 to 7 carbon atoms, and more preferably a cyclopentyl group or a cyclohexyl group.

The polycyclic hydrocarbon group includes a ring-assembled hydrocarbon group and a bridged cyclic hydrocarbon group, and examples of the ring-assembled hydrocarbon group may include a bicyclohexyl group, a perhydronaphthalenyl group and the like. Examples of the bridged cyclic hydrocarbon ring may include a bicyclic hydrocarbon ring such as a pinane ring, a bornane ring, a norpinane ring, a norbornane ring and a bicyclooctane ring (a bicyclo[2.2.2]octane ring, a bicyclo[3.2.1]octane ring and the like), a tricyclic ydrocarbon ring such as a homobledane ring, an adamantine ring, a tricyclo[5.2.1.0^{2,6}]decane ring and a tricyclo[4.3.1.1^{2,5}]undecane ring, a tetracyclic hydrocarbon ring such as a tetracyclol[4.4.0.1^{2,5}.1^{7,10}]dodecane ring and a perhydro-1,4-methano-5,8-methanonaphthalene ring, and the like. Further, the bridged cyclic hydrocarbon ring also includes a condensed cyclic hydrocarbon ring, for example, a condensed ring obtained by condensing a plurality of 5- to 8-membered cycloalkane rings, such as a perhydronaphthalene (decalin) ring, a perhydroanthracene ring, a perhydrophenanthrene ring, a perhydroacenaphthene ring, a perhydrofluorene ring, a perhydroindene ring and a perhydrophenalene ring.

Preferred examples of the bridged cyclic hydrocarbon ring may include a norbornyl group, an adamantyl group, a bicyclooctanyl group, a tricyclo[5,2,1,0^{2,6}]decanyl group and the like. More preferred examples of the bridged cyclic hydrocarbon ring may include a norbornyl group and an adamantyl group.

The alicyclic hydrocarbon groups may have a substituent, and preferred examples of the substituent may include a halogen atom, an alkyl group, a hydroxyl group whose hydrogen atom is substituted, an amino group whose hydrogen atom is substituted and the like. Preferred examples of the halogen atom may include a bromine atom, a chlorine atom and a fluorine atom, and preferred examples of the alkyl group may include a methyl group, an ethyl group, a n-butyl group and a t-butyl group. The aforementioned alkyl group may further have a substituent, and examples of the substituent which may be further possessed by the alkyl group may include a halogen atom, an alkyl group, a hydroxyl group whose hydrogen atom is substituted, and an amino group whose hydrogen atom is substituted.

Examples of the group whose hydrogen atom is substituted may include an alkyl group, a cycloalkyl group, an aralkyl group, a substituted methyl group, a substituted ethyl group, an alkoxycarbonyl group, and an aralkyloxycarbonyl group. Preferred examples of the alkyl group may include an alkyl group having 1 to 4 carbon atoms, preferred examples of the substituted methyl group may include a methoxymethyl group, a methoxythiomethyl group, a benzyloxymethyl group, a t-butoxymethyl group, and a 2-methoxyethoxymethyl group, examples of the substituted ethyl group may include a 1-ethoxy ethyl group and a 1-methyl-1-methoxyethyl group, preferred examples of the acyl group may include an aliphatic acyl group having 1 to 6 carbon atoms, such as a formyl group, an acetyl group, a propionyl group, a butyryl group, an isobutyryl group, a valeryl group and a pivaloyl group, and examples of the alkoxycarbonyl group may include an alkoxycarbonyl group having 1 to 4 carbon atoms and the like.

The resin (B) may or may not contain a repeating unit having an alicyclic hydrocarbon structure having no polar group and not exhibiting acid decomposability, but in the case of containing the repeating unit, the content ratio of the repeating unit is preferably 1 mol % to 40 mol %, and more preferably 2 mol % to 20 mol %, based on the whole repeating units in the resin (B).

Specific examples of the repeating unit having an alicyclic hydrocarbon structure having no polar group and not exhibiting acid decomposability will be shown below, but the present invention is not limited thereto. In the formula, Ra represents H, CH₃, CH₂OH or CF₃.

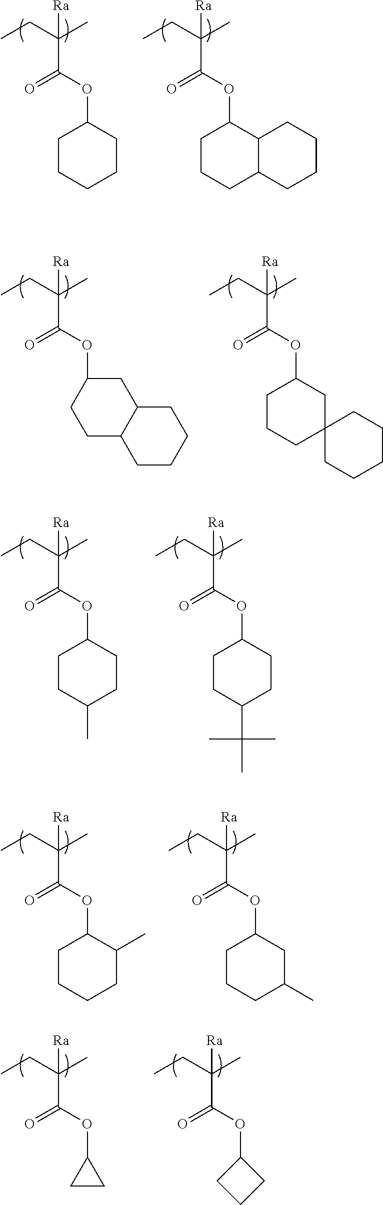

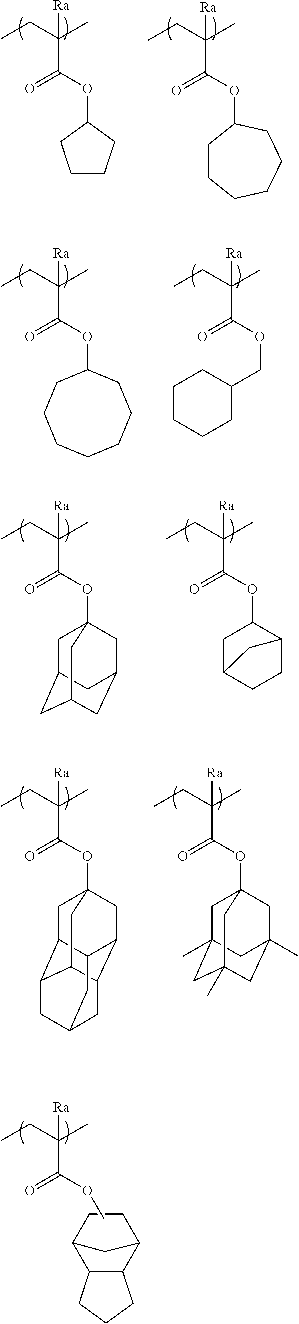

The resin (B) used in the composition of the present invention may have, in addition to the aforementioned repeating structural units, various repeating structural units for the purpose of controlling the dry etching resistance, suitability for a standard developer, adhesion to a substrate and resist profile, and further, resolution, heat resistance, sensitivity and the like, which are properties generally required for a resist.

Examples of the repeating structural units may include repeating structural units corresponding to the monomers described below, but are not limited thereto.

Accordingly, the performance required for the resin used in the composition of the present invention, particularly (1) solubility in a coating solvent, (2) film-forming property (glass transition temperature), (3) alkali developability, (4) film reduction (selection of a hydrophilic, hydrophobic or alkali-soluble group), (5) adhesion of unexposed portion to substrate, and (6) dry etching resistance, and the like may be finely adjusted.

Examples of the monomer may include a compound having one addition-polymerizable unsaturated bond selected from acrylate esters, methacrylate esters, acrylamides, methacrylamides, allyl compounds, vinyl ethers, vinyl esters and the like.

Besides, an addition-polymerizable unsaturated compound that is copolymerizable with the monomers corresponding to the aforementioned various repeating structural units may be copolymerized.

In the resin (B) used in the composition of the present invention, the molar ratio of respective repeating structural units contained is appropriately set in order to control dry etching resistance, suitability for a standard developer, adhesion to a substrate and resist profile of the resist, and further, resolution, heat resistance, sensitivity and the like which are performances generally required for the resist.

When the composition of the present invention is for ArF exposure, from the viewpoint of transparency to ArF light, the resin (B) used in the composition of the present invention preferably has substantially no aromatic group. More specifically, the repeating unit having an aromatic group in the whole repeating unit of the resin (B) is preferably 5 mol % or less, more preferably 3 mol % or less, and ideally 0 mol %, that is, the resin does not have an aromatic group. Further, the resin (B) preferably has a monocyclic or polycyclic alicyclic hydrocarbon structure.

Meanwhile, it is preferred that the resin (A) contains no fluorine atom and no silicon atom from the viewpoint of compatibility with the hydrophobic resin (HR) as describe below.

The resin (B) used in the composition of the present invention is preferably a resin in which all the repeating units are composed of a (meth)acrylate-based repeating unit. In this case, it is possible to use any of a resin in which all the repeating units are methacrylate-based repeating units, a resin in which all the repeating units are an acrylate-based repeating unit, and a resin in which all the repeating units are composed of methacrylate-based repeating units and acrylate-based repeating units, but it is preferred that the acrylate-based repeating unit is present in an amount of 50 mol % or less based on the whole repeating units. Further, also preferred is a copolymer containing 20 mol % to 50 mol % of a (meth)acrylate-based repeating unit having an acid-decomposable group, 20 mol % to 50 mol % of a (meth)acrylate-based repeating unit having a lactone group, 5 mol % to 30 mol % of a (meth)acrylate-based repeating unit having an alicyclic hydrocarbon structure substituted with a hydroxyl group or a cyano group, and 0 mol % to 20 mol % of other (meth)acrylate-based repeating units.

In the case where KrF excimer laser light, electron beam, X-ray or high-energy beam having a wavelength of 50 nm or less (EUV and the like) is irradiated on the composition of the present invention, it is preferred that the resin (B) further has a hydroxystyrene-based repeating unit. The resin (A) has more preferably a hydroxystyrene-based repeating unit, a hydroxystyrene-based repeating unit protected with an acid-decomposable group and an acid-decomposable repeating unit such as tertiary alkyl(meth)acrylate ester.

Preferred examples of the hydroxystyrene-based repeating unit having an acid-decomposable group may include repeating units composed of t-butoxycarbonyloxystyrene, 1-alkoxyethoxystyrene, tertiary alkyl(meth)acrylate ester and the like, and more preferably repeating units composed of 2-alkyl-2-adamantyl(meth)acrylate and dialkyl(l-adamantyl)methyl(meth)acrylate.

The resin (B) in the present invention may be synthesized by a conventional method (for example, radical polymerization). Examples of a general synthesis method may include a batch polymerization method of dissolving monomer species and an initiator in a solvent and heating the solution to perform the polymerization, a dropping polymerization method of adding dropwise a solution containing monomer species and an initiator to a heated solvent over 1 to 10 hours, and the like, and a dropping polymerization method is preferred. Examples of a reaction solvent may include tetrahydrofuran, 1,4-dioxane, ethers such as diisopropyl ether, ketones such as methyl ethyl ketone and methyl isobutyl ketone, an ester solvent such as ethyl acetate, an amide solvent such as dimethylformamide, dimethylacetamide, and a solvent capable of dissolving the composition of the present invention described below, such as propylene glycol monomethyl ether acetate, propylene glycol monomethyl ether and cyclohexanone. The polymerization is more preferably performed by using the same solvent as the solvent used in the photosensitive composition of the present invention. Accordingly, generation of particles during storage may be suppressed.

The polymerization reaction is preferably performed under an inert gas atmosphere such as nitrogen and argon. As for the polymerization initiator, the polymerization is initiated by using a commercially available radical initiator (azo-based initiator, peroxide and the like). The radical initiator is preferably an azo-based initiator, and an azo-based initiator having an ester group, a cyano group or a carboxyl group is preferred. Preferred examples of the initiator may include azobisisobutyronitrile, azobisdimethylvaleronitrile, dimethyl 2,2′-azobis(2-methylpropionate) and the like. The initiator is added additionally or in parts, if desired, and after the completion of reaction, the reaction product is poured in a solvent, and a desired polymer is recovered by a powder or solid recovery method, or the like. The reaction concentration is 5% by mass to 50% by mass, and preferably 10% by mass to 30% by mass. The reaction temperature is usually 10° C. to 150° C., preferably 30° C. to 120° C., and more preferably 60° C. to 100° C.

The weight average molecular weight of the resin (B) of the present invention is preferably 1,000 to 200,000, more preferably 2,000 to 20,000, still more preferably 3,000 to 15,000, and particularly preferably 3,000 to 11,000, in terms of polystyrene by the GPC method. By setting the weight average molecular weight within 1,000 to 200,000, it is possible to prevent deterioration in the heat resistance or dry etching resistance and prevent the film-forming property from deteriorating due to impaired developability or increased viscosity.

The polydispersity (molecular weight distribution) is usually in a range of 1.0 to 3.0, preferably 1.0 to 2.6, and more preferably 1.0 to 2.0. The smaller the molecular weight distribution is, the better the resolution and resist shape are, the smoother the side wall of the resist pattern is, and the better the roughness is.

In the present invention, the content of the resin (B) in the entire composition is preferably 30% by mass to 99% by mass, and more preferably 55% by mass to 95% by mass, based on the total solid.

Further, the resin of the present invention may be used either alone or in combination of two or more thereof.

[3]Basic Compound

The actinic ray-sensitive or radiation-sensitive resin composition of the present invention may contain a basic compound in order to reduce the change in performance over time from exposure to heating.

Preferred examples of the basic compound may include compounds having a structure represented by the following Formulas (A) to (E).

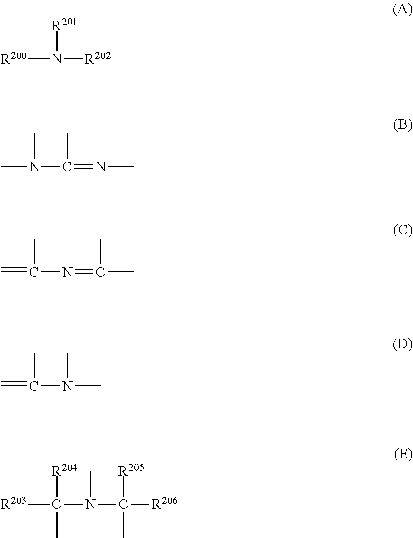

In Formulas (A) and (E),

R²⁰⁰'s, R²⁰¹'s and R²⁰²'s may be the same as or different, and represent a hydrogen atom, an alkyl group (preferably having 1 to 20 carbon atoms), a cycloalkyl group (preferably having 3 to 20 carbon atoms) or an aryl group (having 6 to 20 carbon), and, here, R²⁰¹ and R²⁰² may be bound with each other to form a ring.

R²⁰³'s, R²⁰⁴'s, R²⁰⁵'s and R²⁰⁶'s may be the same as or different, and represent an alkyl group having 1 to 20 carbon atoms.

For the alkyl group, the alkyl group having a substituent is preferably an aminoalkyl group having 1 to 20 carbon atoms, a hydroxyalkyl group having 1 to 20 carbon atoms or a cyanoalkyl group having 1 to 20 carbon atoms.

The alkyl group in Formulas (A) to (E) is more preferably unsubstituted.

Preferred examples of the compound may include guanidine, aminopyrrolidine, pyrazole, pyrazoline, piperazine, aminomorpholine, aminoalkylmorpholine, piperidine and the like, and more preferred specific examples of the compound may include a compound having an imidazole structure, a diazabicyclo structure, an onium hydroxide structure, an onium carboxylate structure, a trialkylamine structure, an aniline structure or a pyridine structure, an alkylamine derivative having a hydroxyl group and/or an ether bond, an aniline derivative having a hydroxyl group and/or an ether bond and the like.

Examples of the compound having an imidazole structure may include imidazole, 2,4,5-triphenylimidazole, benzimidazole, 2-phenylbenzimidazole and the like. Examples of the compound having a diazabicyclo structure may include 1,4-diazabicyclo[2,2,2]octane, 1,5-diazabicyclo[4,3,0]non-5-ene, 1,8-diazabicyclo[5,4,0]undec-7-ene and the like. Examples of the compound having an onium hydroxide structure may include tetrabutylammium hydroxide, triarylsulfonium hydroxide, phenacylsulfonium hydroxide, a sulfonium hydroxide having a 2-oxoalkyl group, specifically, triphenylsulfonium hydroxide, tris(t-butylphenyl)sulfonium hydroxide, bis(t-butylphenyl)iodonium hydroxide, phenacylthiophenium hydroxide, 2-oxopropylthiophenium hydroxide and the like. Examples of the compound having an onium carboxylate structure may include a compound, in which the anion moiety of a compound having an onium hydroxide structure has been converted into carboxylate, such as acetate, adamantane-1-carboxylate and perfluoroalkylcarboxylate. Examples of the compound having a trialkylamine structure may include tri(n-butyl)amine, tri(n-octyl)amine and the like. Examples of the anliline compound may include 2,6-diisopropylaniline, N,N-dimethylaniline, N,N-dibutylaniline, N,N-dihexylaniline and the like. Examples of the alkylamine derivative having a hydroxyl group and/or an ether bond may include ethanolamine, diethanolamine, triethanolamine, n-phenyldiethanolamine, tris(methoxyethoxyethyl)amine and the like. Examples of the aniline derivative having a hydroxyl group and/or an ether bond may include N,N-bis(hydroxyethyl)aniline and the like.

Examples of the preferred basic compound may further include an amine compound having a phenoxy group, an ammonium salt compound having a phenoxy group, an amine compound having a sulfonate ester group, and an ammonium salt compound having a sulfonate ester group.

The amine compound used may include a primary, secondary or tertiary amine compound, and is preferably an amine compound in which at least one alkyl group is bonded to a nitrogen atom. The amine compound is more preferably a tertiary amine compound. In the amine compound, if at least one alkyl group (preferably having 1 to 20 carbon atoms) is bonded to a nitrogen atom, in addition to the alkyl group, a cycloalkyl group (preferably having 3 to 20 carbon atoms) or an aryl group (preferably having 6 to 12 carbon atoms) may be bonded to a nitrogen atom. It is preferred that the amine compound has an oxygen atom in its alkyl chain to form an oxyalkylene group. The number of the oxyalkylene groups is one or more, preferably 3 to 9, and more preferably 4 to 6, in the molecule. Among oxyalkylene groups, an oxyethylene group (—CH₂CH₂O—) or an oxypropylene group (—CH(CH₃)CH₂O— or —CH₂—CH₂CH₂O—) is preferred, and an oxyethylene group is more preferred.

The ammonium salt compound used may include a primary, secondary, tertiary or quaternary compound, and is preferably an ammonium salt compound in which at least one alkyl group is bonded to a nitrogen atom. In the ammonium salt compound, if at least one alkyl group (preferably having 1 to 20 carbon atoms) is bonded to a nitrogen atom, in addition to the alkyl group, a cycloalkyl group (preferably having 3 to 20 carbon atoms) or an aryl group (preferably having 6 to 12 carbon atoms) may be bonded to a nitrogen atom. It is preferred that the ammonium salt compound has an oxygen atom in its alkyl chain to form an oxyalkylene group. The number of the oxyalkylene group is one or more, preferably 3 to 9, and more preferably 4 to 6 in the molecule. Among oxyalkylene groups, an oxyethylene group (—CH₂CH₂O—) or an oxypropylene group (—CH(CH₃)CH₂— or —CH₂CH₂CH₂O—) is preferred, and an oxyethylene group is more preferred.

Examples of the anion of the ammonium salt compound may include a halogen atom, sulfonate, borate, phosphate, but, among them, a halogen atom and sulfonate are preferred. The halogen atom is particularly preferably chloride, bromide or iodide, and the sulfonate is particularly preferably organic sulfonate having 1 to 20 carbon atoms. Examples of the organic sulfonate may include alkylsulfonate having 1 to 20 carbon atoms and arylsulfonate. The alkyl group of the alkylsulfonate may have a substituent, and examples of the substituent may include fluorine, chlorine, bromine, an alkoxy group, an acyl group, an aryl group and the like. Specific examples of the alkylsulfonate may include methanesulfonate, ethanesulfonate, butanesulfonate, hexanesulfonate, octanesulfonate, benzylsulfonate, trifluoromethanesulfonate, pentafluoroethanesulfonate, nonafluorobutanesulfonate and the like. Examples of the aryl group of the arylsulfonate may include a benzene ring, a naphthalene ring and an athracene ring. The benzene ring, the naphthalene ring and the athracene ring may have a substituent, and the substituent is preferably a straight or branched alkyl group having 1 to 6 carbon atoms or a cycloalkyl group having 3 to 6 carbon atoms. Specific examples of the straight or branched alkyl group and the cycloalkyl group may include methyl, ethyl, n-propyl, isopropyl, n-butyl, i-butyl, t-butyl, n-hexyl, cyclohexyl and the like. Examples of other substituents may include an alkoxy group having 1 to 6 carbon atoms, a halogen atom, a cyano group, a nitro group, an acyl group, an acyloxy group and the like.

The amine compound having a phenoxy group or the ammonium salt compound having a phenoxy group means that the compound has a phenoxy group at the opposite end of the alkyl group of the amine compound or the ammonium salt compound to the nitrogen atom.

Examples of the substituent of the phenoxy group may include an alkyl group, an alkoxy group, a halogen atom, a cyano group, a nitro group, a carboxyl group, a carboxylate ester group, a sulfonate ester group, an aryl group, an aralkyl group, an acyloxy group, an aryloxy group and the like. The substitution position of the substituent may be at any of 2- to 6-positions. The number of substituents may be any of 1 to 5.

It is preferred to have at least one oxyalkylene group between the phenoxy group and the nitrogen atom. The number of the oxyalkylene group is one or more, preferably 3 to 9, and more preferably 4 to 6 in the molecule. Among oxyalkylene groups, an oxyethylene group (—CH₂CH₂O—) or an oxypropylene group (—CH(CH₃)CH₂O— or —CH₂CH₂CH₂O—) is preferred, and an oxyethylene group is more preferred.

The sulfonate ester group in the amine compound having a sulfonate ester group and the ammonium salt compound having a sulfonate ester group may be any of alkylsulfonate ester, cycloalkyl group sulfonate ester and arylsulfonate ester, and it is preferred that in the case of alkylsulfonate ester, the alkyl group has 1 to 20 carbon atoms, in the case of cycloalkylsulfonate ester, the cycloalkyl group has 3 to 20 carbon atoms, and in the case of arylsulfonate ester, the aryl group has 6 to 12 carbon atoms. The alkylsulfonate ester, the cycloalkylsulfonate ester and the arylsulfonate ester may have a substituent, and the substituent is preferably a halogen atom, a cyano group, a nitro group, a carboxyl group, a carboxylate ester group or a sulfonate ester group.

It is preferred to have at least one oxyalkylene group between the sulfonate ester group and the nitrogen atom. The number of the oxyalkylene group is one or more, preferably 3 to 9, and more preferably 4 to 6 in the molecule. Among oxyalkylene groups, an oxyethylene group (—CH₂CH₂O—) or an oxypropylene group (—CH(CH₃)CH₂O— or —CH₂CH₂CH₂O—) is preferred, and an oxyethylene group is more preferred.

Further, the following compound is also preferred as a basic compound.

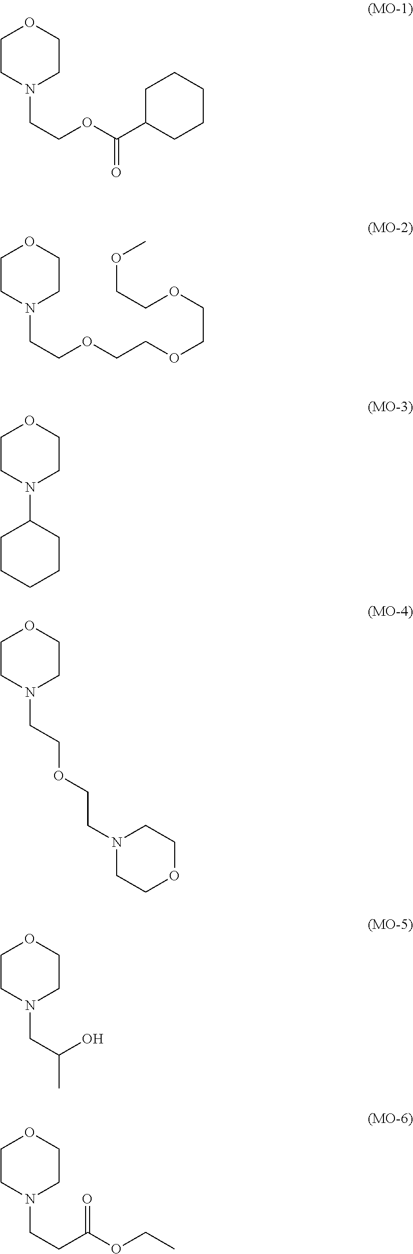

As the basic compound, it is also possible to use, in addition to the aforementioned compound, compounds described in [0259] to [0260] of Japanese Patent Application Laid-Open No. 2011-22560, [0261] to [0262] of Japanese Patent Application Laid-Open No. 2012-137735, and [0263] to [0264] of International Publication WO2011/158687A1. The basic compound may be a basic compound or an ammonium salt compound whose basicity is decreased upon irradiation with an actinic ray or radiation.

The basic compound may be used either alone or in combination of two or more thereof.

The composition of the present invention may or may not contain a basic compound, but in the case of containing a basic compound, the amount of the basic compound used is usually 0.001% by mass to 10% by mass, and preferably 0.01% by mass to 5% by mass, based on the solid of the actinic ray-sensitive or radiation-sensitive resin composition.

The ratio of the acid generator (including the acid generator (A′)) and the basic compound used in the composition is preferably acid generator/basic compound (molar ratio)=2.5 to 300. That is, the molar ratio is preferably 2.5 or more from the viewpoint of sensitivity and resolution, and is preferably 300 or less from the viewpoint of suppressing the reduction in resolution caused by thickness of the resist pattern with time after exposure until heat treatment. The acid generator/basic compound (molar ratio) is more preferably 5.0 to 200, and still more preferably 7.0 to 150.

The basic compound is preferably used in a molar ratio of low molecular weight compound (D)/basic compound=100/0 to 10/90 with respect to the low molecular weight compound (D) described in the following section[4], more preferably in 100/0 to 30/70, and particularly preferably in 100/0 to 50/50.

Meanwhile, the basic compound as described herein does not include (C) a low molecular weight compound containing a nitrogen atom and having a group capable of leaving by the action of an acid as described below.

[4] Low Molecular Weight Compound Containing a Nitrogen Atom and Having a Group Capable of Leaving by the Action of an Acid

It is preferred that the composition of the present invention contains a compound containing a nitrogen atom and having a group capable of leaving by the action of an acid (hereinafter, also referred to as a “compound (C)”).

The group capable of leaving by the action of an acid is, but not particularly limited to, preferably an acetal group, a carbonate group, a carbamate group, a tertiary ester group, a tertiary hydroxyl group or a hemiaminal group, and particularly preferably a carbamate group or a hemiaminal group.

The molecular weight of the compound (C) having a group capable of leaving by the action of an acid is preferably 100 to 1,000, more preferably 100 to 700, and particularly preferably 100 to 500.

The compound (C) is preferably an amine derivative having a group capable of leaving by the action of an acid on its nitrogen atom.

The compound (C) may have a carbamate group having a protecting group on its nitrogen atom. The protecting group constituting the carbamate group may be represented by the following Formula (d-1).

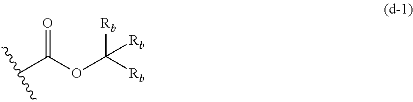

In Formula (d-1),

Rb's each independently represent a hydrogen atom, an alkyl group (preferably having 1 to 10 carbon atoms), a cycloalkyl group (preferably having 3 to 30 carbon atoms), an aryl group (preferably having 3 to 30 carbon atoms), an aralkyl group (preferably having 1 to 10 carbon atoms) or an alkoxyalkyl group (preferably having 1 to 10 carbon atoms). Rb's may be linked to each other to form a ring.

The alkyl group, the cycloalkyl group, the aryl group and the aralkyl group represented by Rb may be substituted with a functional group such as a hydroxyl group, a cyano group, amino group, a pyrrolidino group, a piperidino group, a morpholino group and an oxo group, an alkoxy group or a halogen atom. The same is applied to the alkoxyalkyl group represented by Rb.

Examples of the alkyl group, the cycloalkyl group, the aryl group and the aralkyl group of Rb (the alkyl group, the cycloalkyl group, the aryl group and the aralkyl group may be substituted with the above-mentioned functional group, an alkoxy group or a halogen atom) may include a group derived from a straight or branched alkane such as methane, ethane, propane, butane, pentane, hexane, heptane, octane, nonane, decane, undecane and dodecane, and a group in which the group derived from an alkane is substituted with one or more kinds of or one or more of cycloalkyl groups such as a cyclobutyl group, a cyclopentyl group and a cyclohexyl group, a group derived from a cycloalkane such as a cyclobutane, a cyclopentane, a cyclohexane, cycloheptane, cyclooctane, a norbornane, an adamantane and a noradamantane, and a group in which the group derived from an cycloalkane is substituted with one or more kinds of or one or more of straight or branched alkyl groups such as a methyl group, an ethyl group, a n-propyl group, an i-propyl group, a n-butyl group, a 2-methylpropyl group, a 1-methylpropyl group and a t-butyl group, a group derived from an aromatic compound such as benzene, naphthalene and anthracene, and a group in which the group derived from an aromatic compound is substituted with one or more kinds of or one or more of straight or branched alkyl groups such as a methyl group, an ethyl group, a n-propyl group, an i-propyl group, a n-butyl group, a 2-methylpropyl group, a 1-methylpropyl group and a t-butyl group, a group derived from a heterocyclic compound such as pyrrolidine, piperidine, morpholine, tetrahydrofuran, tetrahydropyran, indole, indoline, quinoline, perhydroquinoline, indazole and benzimidazole, and a group in which the group derived from a heterocyclic compound is substituted with one or more kinds of or one or more of groups derived from a straight or branched alkyl group or an aromatic compound, a group in which the group derived from a straight or branched alkane—the group derived from a cycloalkane are substituted with one or more kinds or one or more of the group derived from an aromatic comound such as a phenyl group, a naphthyl group and an anthracenyl group, or a group in which the abve-mentioned substituent is substituted with a functional group such as a hydroxyl group, a cyano group, an amino group, a pyrrolidino group, a piperidino group, a morpholino group and an oxo group.

Rb is preferably a straight or branched alkyl group, a cycloalkyl group or an aryl group. Rb is more preferably a straight or branched alkyl group or a cycloalkyl group.

Examples of the ring formed by two Rb's being linked to each other may include an alicyclic hydrocarbon group, an aromatic hydrocarbon group, a heterocyclic hydrocarbon group or a derivative thereof.

Specific structures of the group represented by Formula (d-1) are shown below.

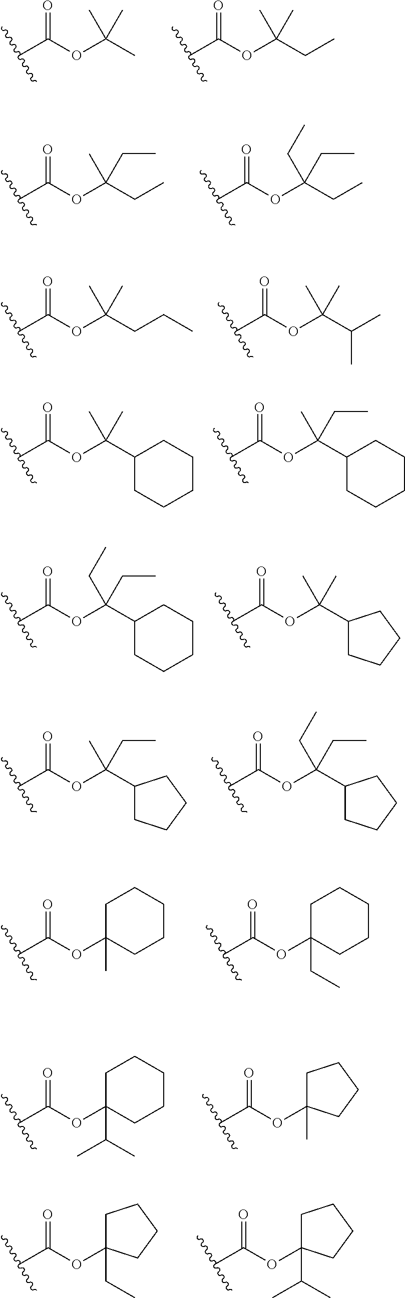

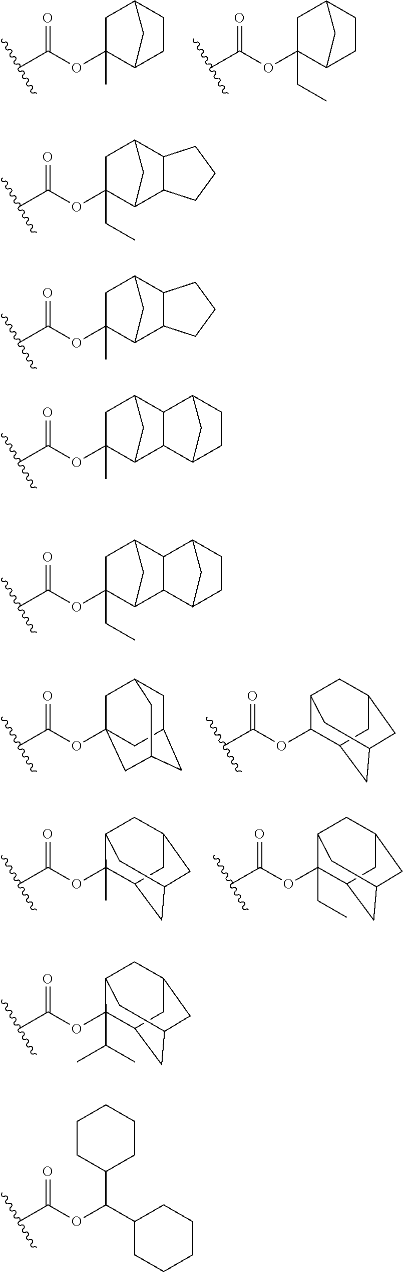

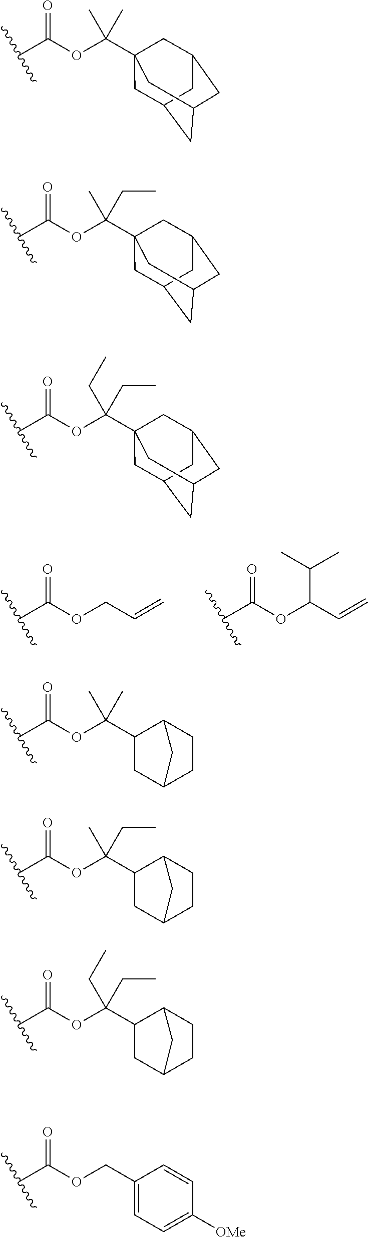

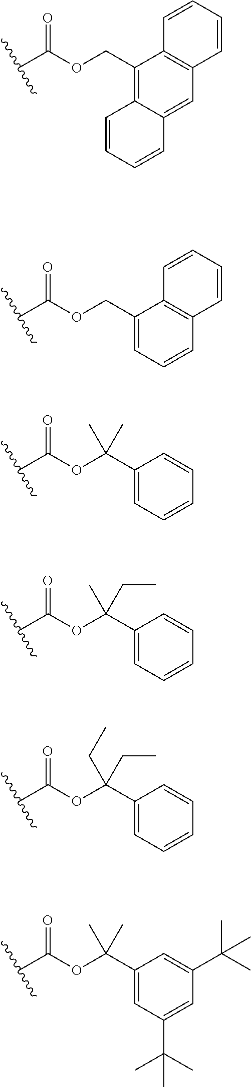 [SMILES: *C(=O)OCc1c2ccccc2cc2ccccc12 ; *C(=O)OCc1cccc2ccccc12 ; *C(=O)OC(C)(C)c1ccccc1 ; *C(=O)OC(C)(CC)c1ccccc1 ; *C(=O)OC(CC)(CC)c1ccccc1 ; *C(=O)OC(C)(C)c1cc(C(C)(C)C)cc(C(C)(C)C)c1]

It is particularly preferred that the compound (C) has a structure represented by the following Formula (6).

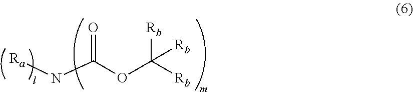

In Formula (6), Ra represents a hydrogen atom, an alkyl group, a cycloalkyl group, an aryl group or an aralkyl group. When 1 is 2, two Ra's may be the same or different, and two Ra's may be linked to each other to form a heterocyclic ring together with the nitrogen atom in the formula. The heterocyclic ring may contain a heteroatom other than the nitrogen atom in the formula.

Rb has the same meaning as Rb in Formula (d-1), and preferred examples thereof are also the same.

1 represents an integer of 0 to 2, and m represents an integer of 1 to 3, satisfying l+m=3.

In Formula (6), the alkyl group, the cycloalkyl group, the aryl group and the aralkyl group as Ra may be substituted with the same group as described above as a group with which the alkyl group, the cycloalkyl group, the aryl group and the aralkyl group as Rb may be substituted.

Specific examples of the alkyl group, the cycloalkyl group, the aryl group and the aralkyl group of Ra (the alkyl group, the cycloalkyl group, the aryl group and the aralkyl group may be substituted with the aforementioned group) may include the specific examples as described above with respect to Rb.

Further, the heterocyclic hydrocarbon group formed by Ra's being linked to each other preferably has 1 to 20 carbon atoms, and examples thereof may include a group derived from a heterocyclic compound such as pyrrolidine, piperidine, morpholine, 1,4,5,6-tetrahydropyrimidine, 1,2,3,4-tetrahydroquinoline, 1,2,3,6-tetrahydropyridine, homopiperazine, 4-azabenzimidazole, benzotriazole, 5-azabenzotriazole, 1H-1,2,3-triazole, 1,4,7-triazacyclononane, tetrazole, 7-azaindole, indazole, benzimidazole, imidazo[1,2-a]pyridine, (1S,4S)-(+)-2,5-diazabicyclo[2.2.1]heptane, 1,5,7-triazabicyclo[4.4.0]dec-5-ene, indole, indoline, 1,2,3,4-tetrahydroquinoxaline, perhydroquinoline and 1,5,9-triazacyclododecane, and a group in which the group derived from a heterocyclic compound is substituted with one or more kinds of or one or more of the groups derived from a straight or branched alkane, the groups derived from a cycloalkane, the groups derived from an aromatic compounds, the groups derived from a heterocyclic compounds or the functional groups such as a hydroxyl group, a cyano group, amino group, pyrrolidino group, a piperidino group, a morpholino group and an oxo group.

Specific examples of the particularly preferred compounds (C) in the present inventions are shown, but the present invention is not limited thereto.

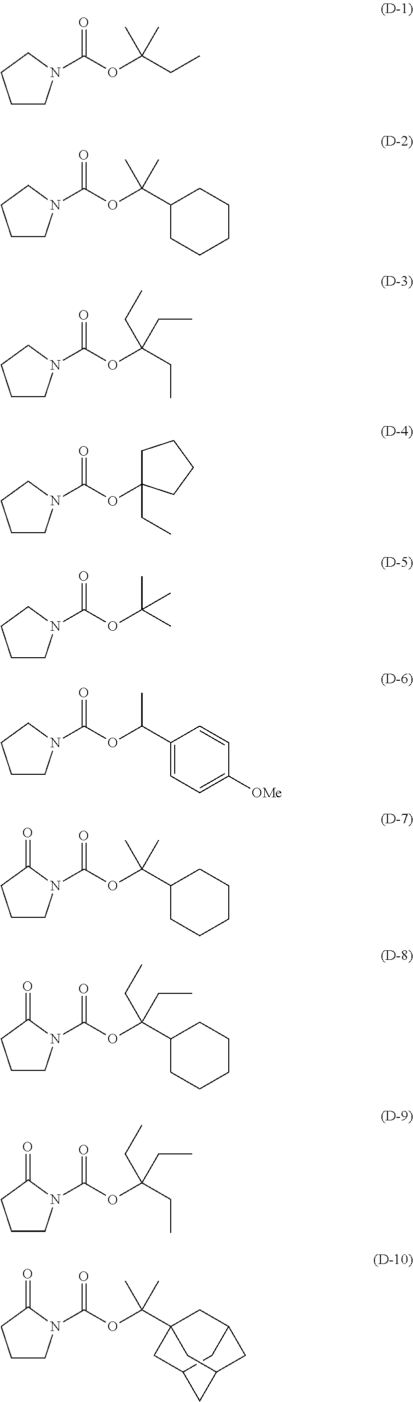

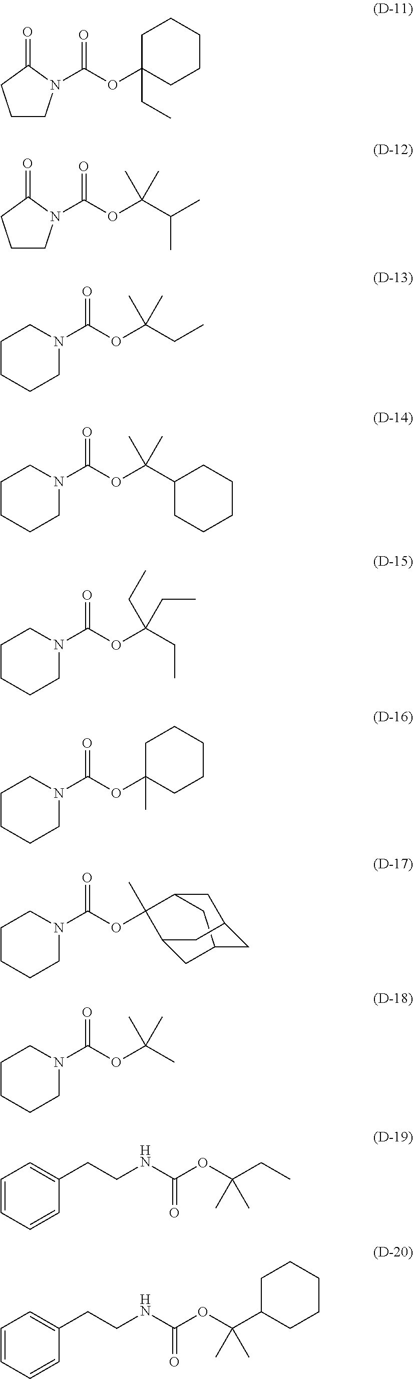

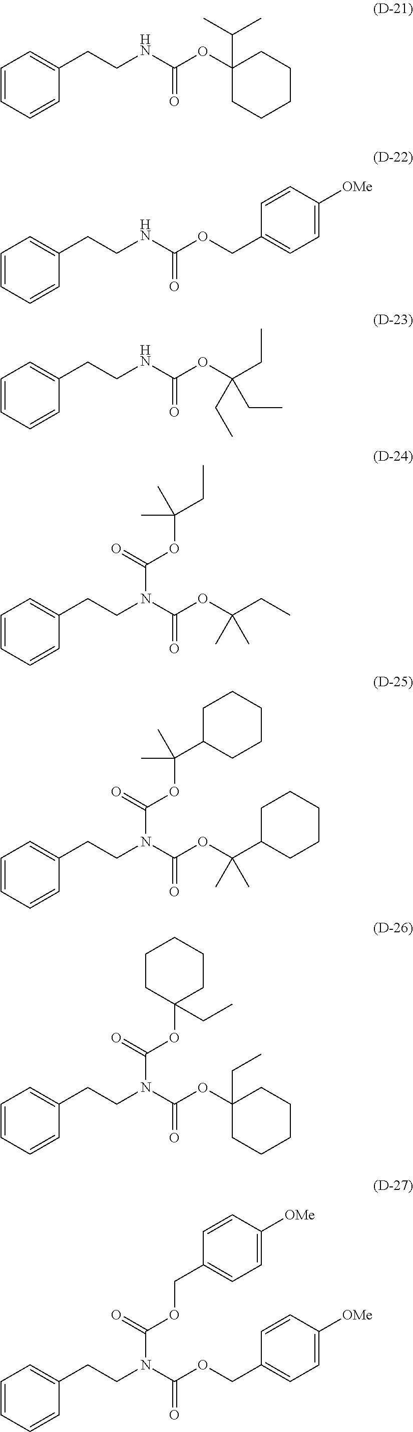

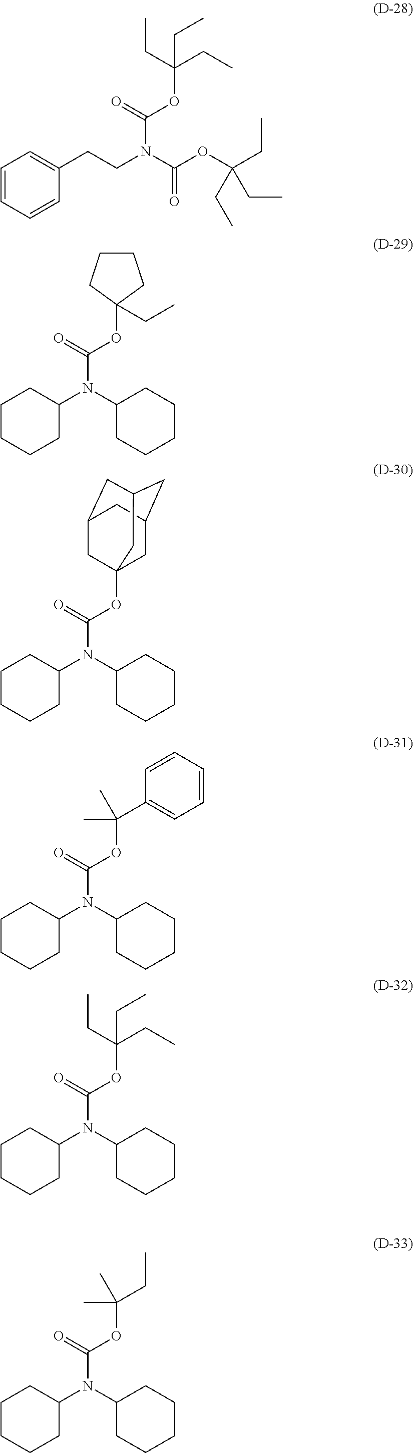

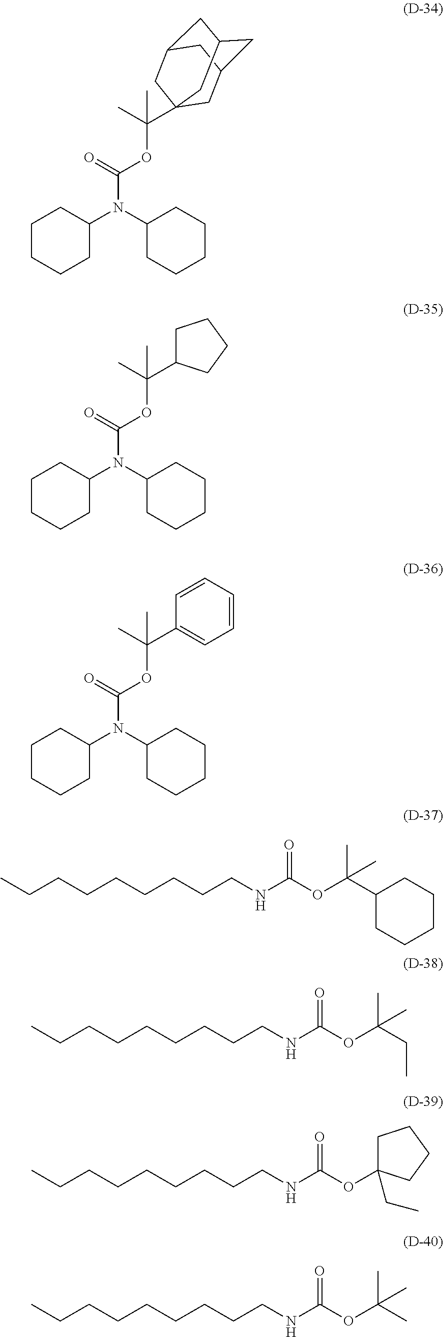

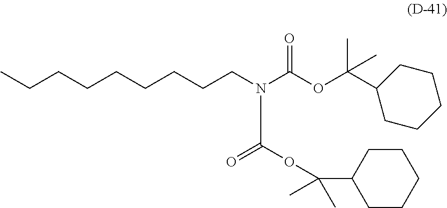 [SMILES: CCCCCCCCCN(C(=O)OC(C)(C)C1CCCCC1)C(=O)OC(C)(C)C1CCCCC1]

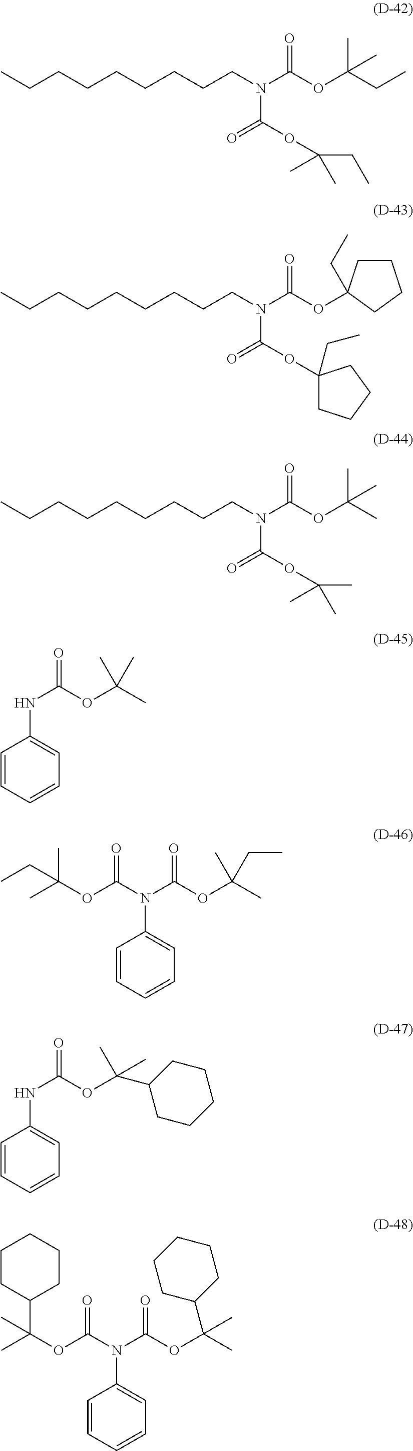

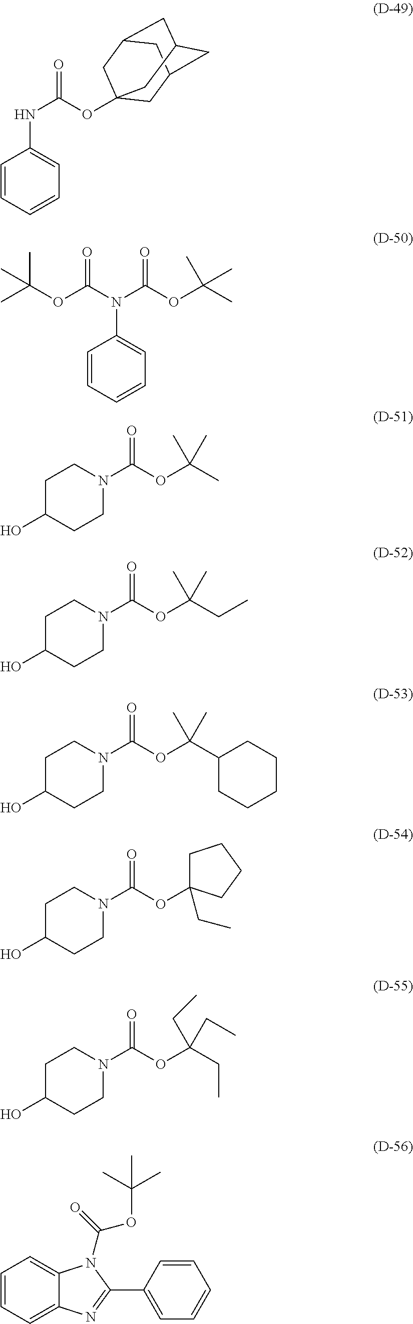

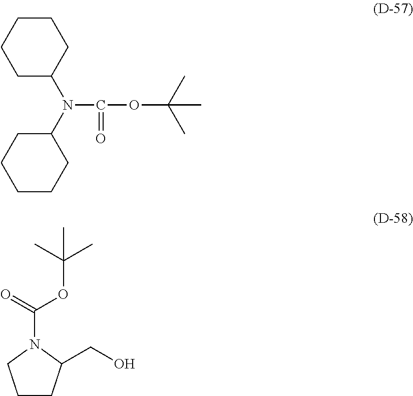

The compound represented by Formula (6) may be synthesized based on Japanese Patent Application Laid-Open No. 2007-298569, Japanese Patent Application Laid-Open No. 2009-199021 and the like.

In the present invention, the low molecular weight compound (C), which has a group capable of leaving by the action of an acid on its nitrogen atom, may be used either alone or in combination of two or more thereof.

The content of the compound (C) in the actinic ray-sensitive or radiation-sensitive resin composition of the present invention is preferably 0.001% by mass to 20% by mass, more preferably 0.001% by mass to 10% by mass, and still more preferably 0.01% by mass to 5% by mass based on the total solid of the composition.

[5] Hydrophobic Resin (HR)

Particularly when applied to liquid immersion exposure, the actinic ray-sensitive or radiation-sensitive resin composition of the present invention may further contain a hydrophobic resin having at least one of a fluorine atom and a silicon atom (hereinafter, also referred to as a “hydrophobic resin (HR)”). Accordingly, when the hydrophobic resin (14R) is localized on the film top layer and the immersion medium is water, the static/dynamic contact angle of the resist film surface against water may be improved, thereby improving an immersion liquid follow-up property.

The hydrophobic resin (HR) is localized at the interface as described above, but unlike a surfactant, the hydrophobic resin (HIR) does not necessarily have a hydrophilic group in the molecule thereof, and may not contribute to the mixing of polar/non-polar materials homogeneously.

A hydrophobic resin typically contains a fluorine atom and/or a silicon atom. The fluorine atom and/or the silicon atom in the hydrophobic resin (HR) may be contained in the main chain of the resin, or may be contained in the side chain thereof.

In the case where the hydrophobic resin contains a fluorine atom, it is preferred that the resin has an alkyl group having a fluorine atom, a cycloalkyl group having a fluorine atom, or an aryl group having a fluorine atom as a partial structure having a fluorine atom.

The alkyl group having a fluorine atom is a straight or branched alkyl group in which at least one hydrogen atom is substituted by a fluorine atom, and preferably has 1 to 10 carbon atoms, and more preferably 1 to 4 carbon atoms, and may further have other substituents.

The cycloalkyl group having a fluorine atom is a monocyclic or polycyclic cycloalkyl group in which at least one hydrogen atom is substituted by a fluorine atom, and may further have other substituents.

The aryl group having a fluorine atom may be an aryl group such as a phenyl group and a naphthyl group, in which at least one hydrogen atom is substituted by a fluorine atom, and may further have other substituents.

Examples of the alkyl group having a fluorine atom, the cycloalkyl group having a fluorine atom or the aryl group having a fluorine atom may include groups represented by any of the following Formulas (F2) to (F4), but the present invention is not limited thereto.

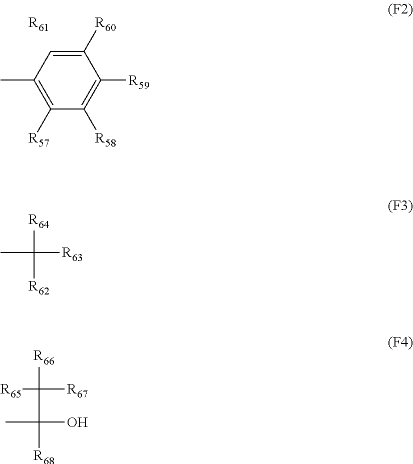

In Formulas (F2) to (F4),

R₅₇ to R₆₈ each independently represent a hydrogen atom, a fluorine atom or an alkyl group (straight or branched). However, at least one of R₅₇ to R₆₁, at least one of R₆₂ to R₆₄ and at least one of R₆₅ to R₆₈ represent a fluorine atom or an alkyl group (preferably having 1 to 4 carbon atoms) in which at least one hydrogen atom is substituted by a fluorine atom.

It is preferred that all of R₅₇ to R₆₁ and R₆₅ to R₆₇ are a fluorine atom. R₆₂, R₆₃ and Rat are preferably a fluoroalkyl group (preferably having 1 to 4 carbon atoms), and more preferably a perfluoroalkyl group having 1 to 4 carbon atoms. When R₆₂ and R₆₃ are a perfluoroalkyl group, R₆₄ is preferably a hydrogen atom. R₆₂ and R₆₃ may be linked to each other to form a ring.

Specific examples of the group represented by Formula (F2) may include a p-fluorophenyl group, a pentafluorophenyl group and a 3,5-di(trifluoromethyl)phenyl group.

Specific examples of the group represented by Formula (F3) may include a trifluoromethyl group, a pentafluoropropyl group, a pentafluoroethyl group, a heptafluorobutyl group, a hexafluoroisopropyl group, a heptafluoroisopropyl group, a hexafluoro(2-methyl)isopropyl group, a nonafluorobutyl group, an octafluoroisobutyl group, a nonafluorohexyl group, a nonafluoro-t-butyl group, perfluoroisopentyl group, a perfluorooctyl group, a perfluoro(trimethyl)hexyl group, a 2,2,3,3-tetrafluorocyclobutyl group and a perfluorocyclohexyl group. The group is more preferably a hexafluoroisopropyl group, a heptafluoroisopropyl group, a hexafluoro(2-methyl)isopropyl group, an octafluoroisobutyl group, a nonafluoro-t-butyl group or a perfluoroisopentyl group, and more preferably a hexafluoroisopropyl group or a heptafluoroisopropyl group.

Specific examples of the group represented by Formula (F4) may include —C(CF₃)₂OH, —C(C₂F₅)OH, —C(CF₃)(CH₃)OH and —CH(CF₃)OH, and preferably —C(CF₃)₂OH is particularly preferred.

The partial structure containing a fluorine atom may be bonded directly to the main chain or may be bonded to the main chain via a group selected from the group consisting of an alkylene group, a phenylene group, an ether bond, a thioether bond, a carbonyl group, an ester bond, an amide bond, a urethane bond and a ureylene bond, or a group formed by combining two or more thereof.

Suitable examples of the partial structure containing a fluorine atom are shown below.

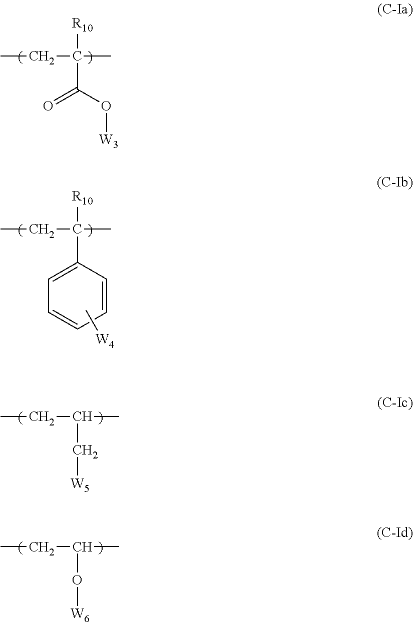

In Formulas (C-1a) to (C-1d), R₁₀ and R₁₁ each independently represent a hydrogen atom, a fluorine atom or an alkyl group. The alkyl group is preferably a straight or branched alkyl group having 1 to 4 carbon atoms, and may have a substituent, and examples of the alkyl group having a substituent may include particularly a fluorinated alkyl group.

W₃ to W₆ each independently represent an organic group having at least one fluorine atom. Specific examples thereof may include atomic groups of Formulas (P2) to (F4).

Further, in addition to them, the hydrophobic resin may have the same unit as described below as a repeating unit having a fluorine atom.

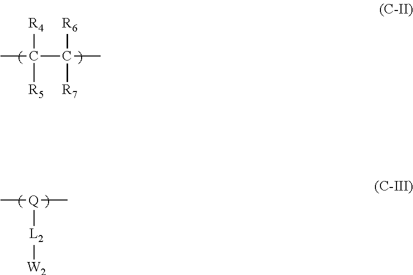

In Formulas (C-II) and (C-III), R₄ to R₇ each independently represent a hydrogen atom, a fluorine atom or alkyl group. The alkyl group is preferably a straight or branched alkyl group having 1 to 4 carbon atoms, and may have a substituent, and examples of the alkyl group having a substituent may include a fluorinated alkyl group.

However, at least one of R₄ to R₁ represents a fluorine atom. R₄ and R₅, or R₆ and R₇ may form a ring.

W₂ represents an organic group containing at least one fluorine atom. Specific examples thereof may include atomic groups of (F2) to (F4).

L₂ represents a single bond or a divalent linking group. Examples of the divalent linking group may include a substituted or unsubstituted arylene group, a substituted or unsubstituted alkylene group, a substituted or unsubstituted cycloalkylene group, —O—, — SO₂—, —CO—, —N(R)— (in which, R represents a hydrogen atom or an alkyl group), —NHSO₂— and a divalent linking group formed by combining two or more thereof.

Q represents an alicyclic structure. The alicyclic structure may have a substituent and may be monocyclic or polycyclic, and the polycyclic structure may be bridged. The monocyclic structure is preferably a cycloalkyl group having 3 to 8 carbon atoms, and examples thereof may include a cyclopentyl group, a cyclohexyl group, a cyclobutyl group, and a cyclooctyl group. The polycyclic structure may include a group having a bicyclo, tricyclo, tetracyclo structure or the like having 5 or more carbon atoms, and is preferably a cycloalkyl group having 6 to 20 carbon atoms, and examples thereof may include an adamantyl group, a norbornyl group, a dicyclopentyl group, a tricyclodecanyl group, and a tetracyclododecyl group. Further, at least one carbon atom in the cycloalkyl group may be substituted by a heteroatom such as an oxygen atom. Q may be particularly preferably a norbornyl group, a tricyclodecanyl group, or a tetracyclododecyl group.

The hydrophobic resin may contain a silicon atom.

It is preferred that the resin has an alkylsilyl structure (preferably a trialkylsilyl group) or a cyclic siloxane structure as a partial structure having a silicon atom.

Specific examples of the alkylsilyl structure or the cyclic siloxane structure may include groups represented by the following Formula (CS-1) to (CS-3).

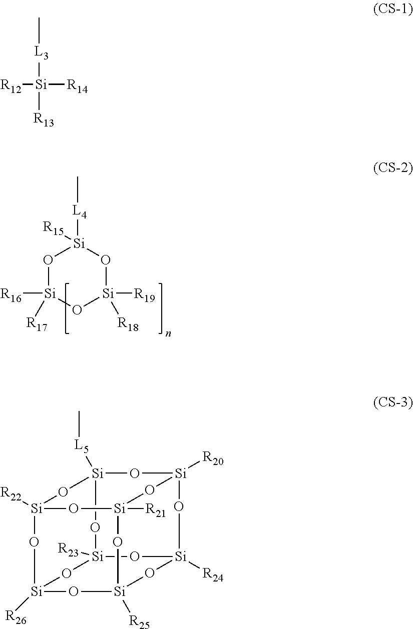

In formula (CS-1) to (CS-3),

R₁₂ to R₂₆ each independently represent straight or branched alkyl group (preferably having 1 to 20 carbon atoms) or a cycloalkyl group (preferably having 3 to 20 carbon atoms).

L₃ to L₅ represent a single bond or a divalent linking group. Examples of the divalent linking group may include one group or a combination of two or more groups selected from the group consisting of an alkylene group, a phenylene group, an ether bond, a thioether bond, a carbonyl group, an ester bond, an amide bond, a urethane bond and a ureylene bond.

n represents an integer of 1 to 5. n is preferably an integer of 2 to 4.

The repeating unit having at least one of a fluorine atom and a silicon atom is preferably a (meth)acrylate-based repeating unit.

Specific examples of the repeating unit having at least one of a fluorine atom and a silicon atom are shown below, but the present invention is not limited thereto. Meanwhile, in specific examples, X₁ represents a hydrogen atom, —CH₃, —F or —CF₃, and X₂ represents —F or —CF₃.

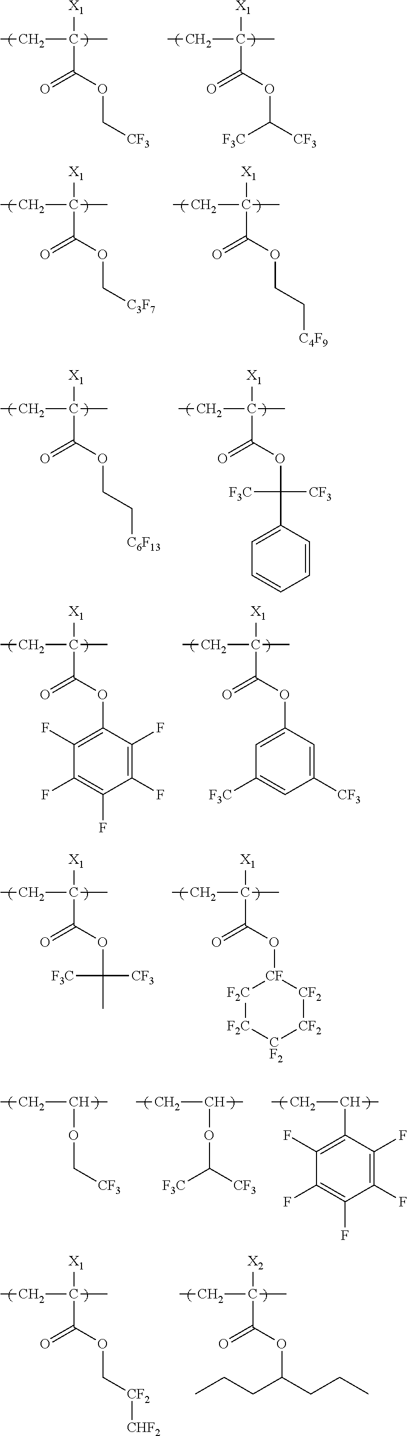

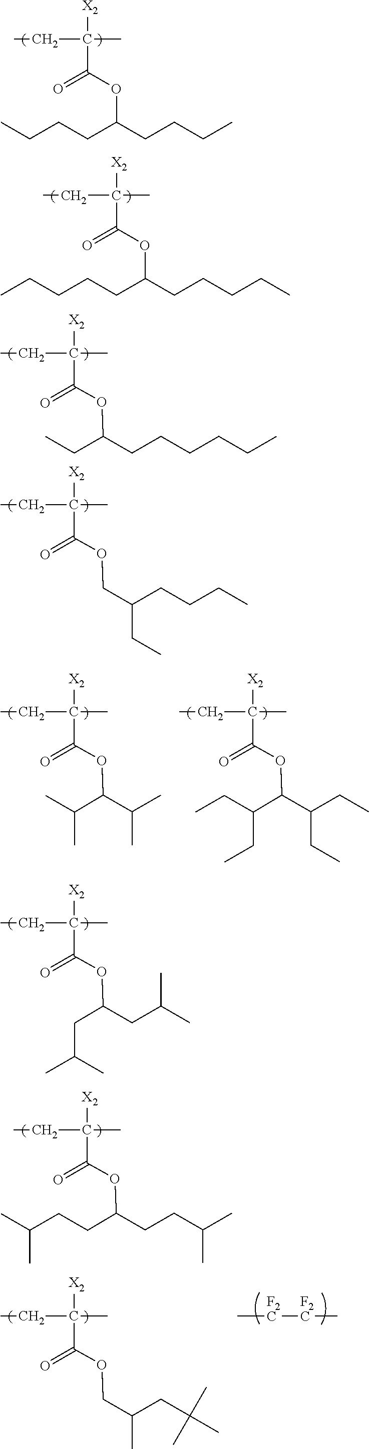

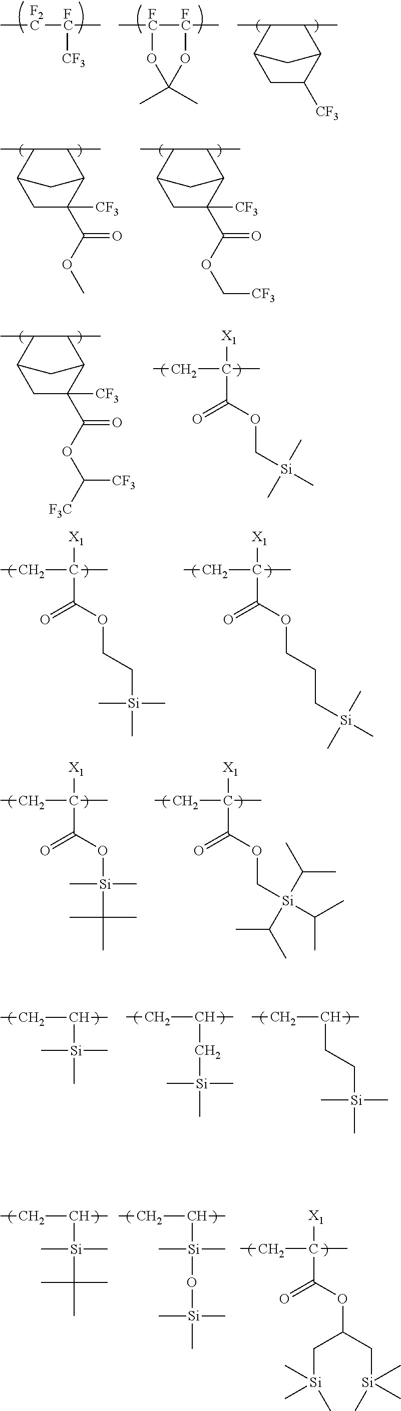

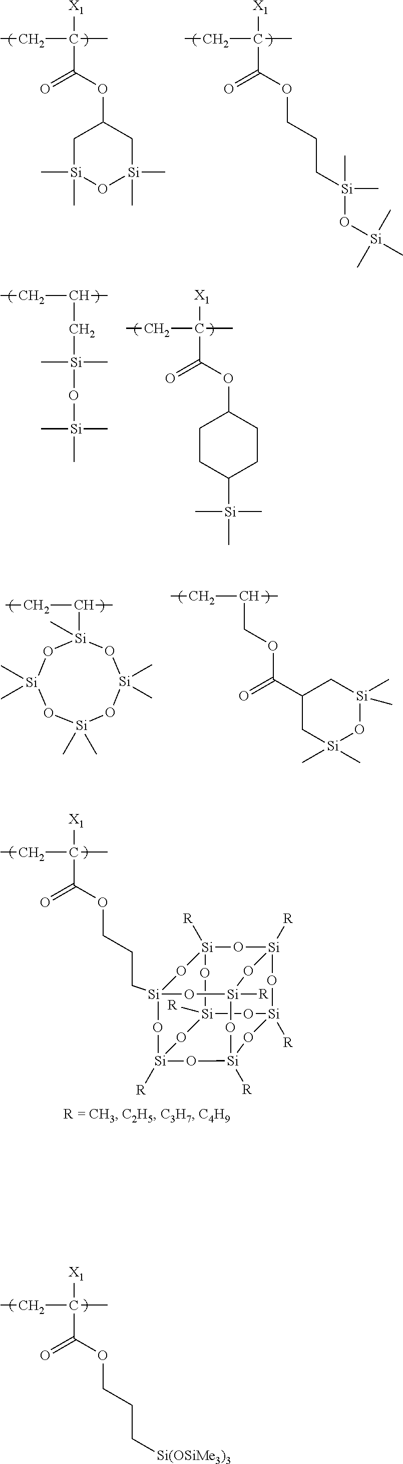

It is preferred that the hydrophobic resin has a repeating unit (b) having at least one group selected from the group consisting of the following (x) to (z).
(x) An alkali-soluble group
(y) A group capable of decomposing by the action of an alkali developer to increase the solubility in the alkali developer (hereinafter, also referred to as a polarity conversion group)
(z) A group capable of decomposing by the action of an acid to increase the solubility in an alkali developer

The repeating unit (b) may be categorized as follows.
A repeating unit (b′) which has at least one of a fluorine atom and a silicon atom, and at least one group selected from the group consisting of (x) to (z) on one side chain
A repeating unit (b*) which has at least one group selected from the group consisting of (x) to (z), but does not have a fluorine atom and a silicon atom
A repeating unit (b″) which has at least one group selected from the group consisting of (x) to (z) on one side chain, and at least one of a fluorine atom and a silicon atom on a side chain different from the side chain within the same repeating unit

It is more preferred that the hydrophobic resin has a repeating unit (b) as the repeating unit (b). That is, it is more preferred that the repeating unit (b) which has at least one group selected from the group consisting of (x) to (z) has at least one of a fluorine atom and a silicon atom.

Meanwhile, in the case where the hydrophobic resin has a repeating unit (b*), the resin is preferably a copolymer with a repeating unit having at least one of a fluorine atom and a silicon atom (a different repeating unit from the repeating units (b′) and (b″)). Further, in the repeating unit (b″), the side chain having at least one group selected from the group consisting of (x) to (z), and the side chain having at least one of a fluorine atom and a silicon atom are preferably bonded to the same carbon atom in the main chain, that is, in a positional relationship as in the following Formula (K1).

In the formula, BI represents a partial structure having at least one group selected from the group consisting of (x) to (z), and B2 represents a partial structure having at least one of a fluorine atom and a silicon atom.

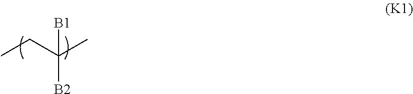

The group selected from the group consisting of (x) to (z) is preferably (x) an alkali-soluble group or (y) a polarity conversion group, and more preferably a (y) polarity conversion group.

Examples of the alkali-soluble group (x) may include a phenolic hydroxyl group, a carboxylic acid group, a fluorinated alcohol group, a sulfonic acid group, a sulfonamide group, a sulfonylimide group, a (alkylsulfonyl)(alkylcarbonyl)methylene group, a (alkylsulfonyl)(alkylcarbonyl)imide group, a bis(alkylcarbonyl)methylene group, a bis(alkylcarbonyl)imide group, a bis(alkylsulfonyl)methylene group, a bis(alkylsulfonyl)imide group, a tris(alkylcarbonyl)methylene group and a tris(alkylsulfonyl)methylene group.

Preferred examples of the alkali-soluble group may include a fluorinated alcohol group (preferably hexafluoroisopropanol), a sulfonimide group, and a bis(carbonyl)methylene group.

A repeating unit (bx) having the alkali-soluble group (x) may be a repeating unit, in which the alkali-soluble group is directly bonded to the main chain of the resin, such as a repeating unit by an acrylic acid or a methacrylic acid, or a repeating unit in which the alkali-soluble group is bonded to the main chain of the resin through a linking group. Further, the alkali-soluble group may be introduced into the terminal of the polymer chain by using a polymerization initiator having an alkali-soluble group or a chain transfer agent at the time of polymerization. All of these cases are preferred.

When the repeating unit (bx) is a repeating unit having at least one of a fluorine atom and a silicon atom (that is, corresponding to the repeating units (b′) and (b″)), examples of the partial structure having a fluorine atom in the repeating unit (bx) may be the same as those exemplified in the repeating unit having at least one of a fluorine atom and a silicon atom, and may preferably include the groups represented by Formula (F2) to (F4). Further, in this case, the partial structure having a silicon atom in the repeating unit (bx) may be the same as those exemplified in the repeating unit having at least one of a fluorine atom and a silicon atom, and may preferably include the groups represented by Formulas (CS-1) to (CS-3).

The content of the repeating unit (bx) having the alkali-soluble group (x) preferably is 1 mol % to 50 mol %, more preferably 3 mol % to 35 mol %, and still more preferably 5 mol % to 20 mol % based on the whole repeating units in the hydrophobic resin.

Specific examples of the repeating unit (bx) having the alkali-soluble group (x) are shown below, but the present invention is not limited thereto. Meanwhile, in specific examples, X₁ represents a hydrogen atom, —CH₃, —F or —CF₃.

In the formulas, Rx represents H, CH₃, CH₂OH or CF₃.

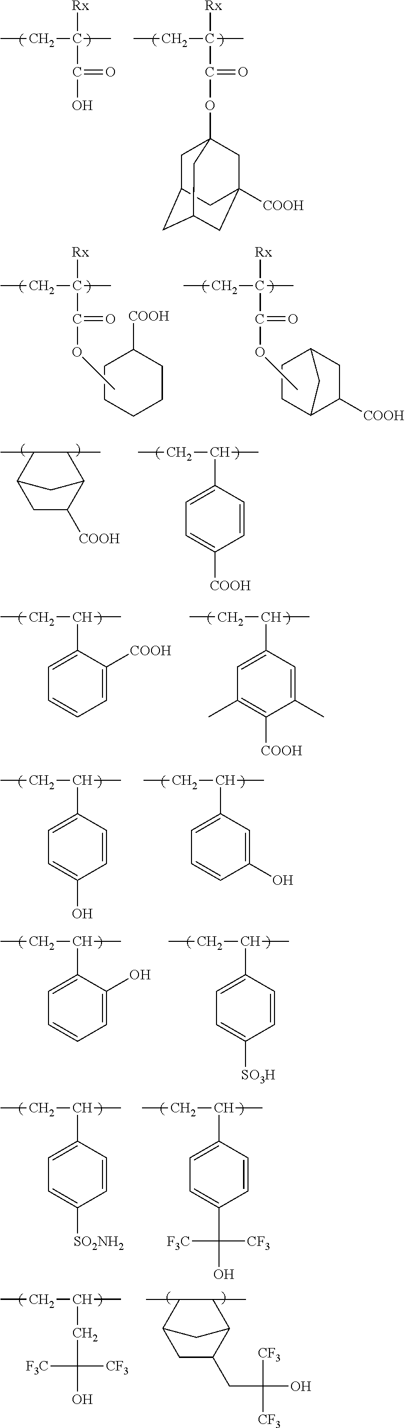

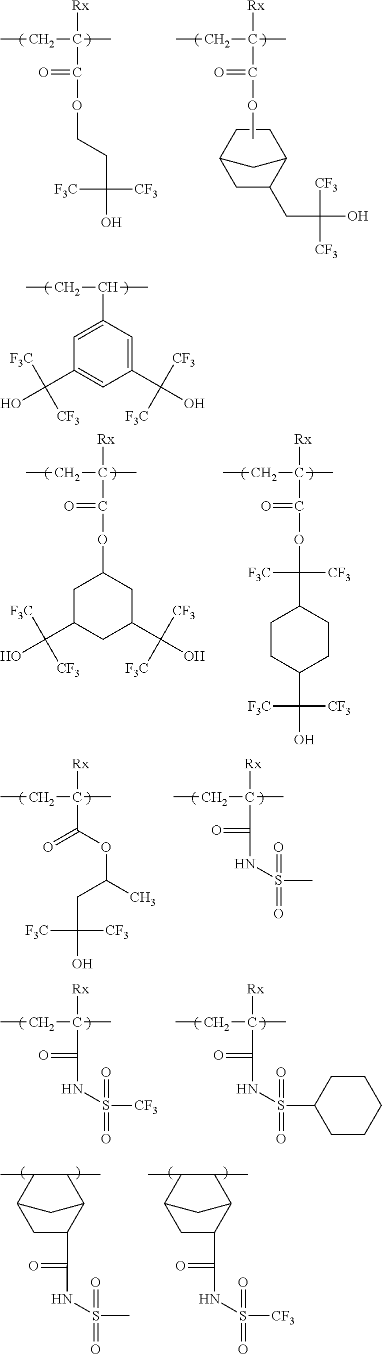

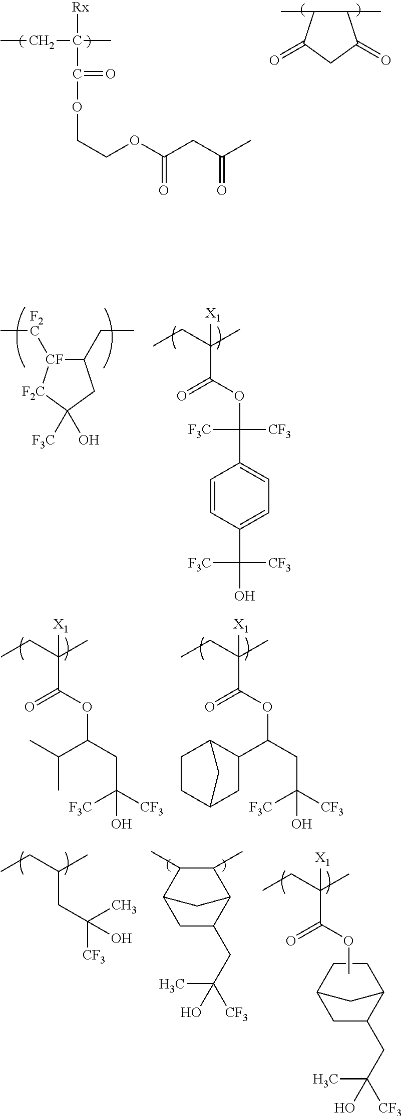

Examples of the polarity conversion group (y) may include a lactone group, a carboxylate ester group (—COO—), an acid anhydride group (—C(O)OC(O)—), an acid imide group (—NHCONH—), a carboxylate thioester group (—COS—), a carbonate ester group (—OC(O)O—), a sulfate ester group (—OSO₂O—) and a sulfonate ester group (—SO₂O—), and preferably a lactone group.

For example, it is preferred that the polarity conversion group (y) may be introduced at the side chain of the resin by being contained in a repeating unit by acrylate ester or methacrylate ester, or may be introduced into the terminal of a polymer chain by using a polymerization initiator or a chain transfer agent having the polarity conversion group (y) at the time of polymerization.

Specific examples of a repeating unit (by) having the polarity conversion group (y) may include repeating units having a lactone structure represented by Formulas (KA-1-1) to (KA-1-18) as described below.

Further, the repeating unit (by) having the polarity conversion group (y) is preferably a repeating unit having at least one of a fluorine atom and a silicon atom (that is, corresponding to the repeating units (b′) and (b″)). The resin having the repeating unit (by) has hydrophobicity, which is preferred particularly from the viewpoint of reduction in development defects.

Example of the repeating unit (by) may include a repeating unit represented by Formula (K0).

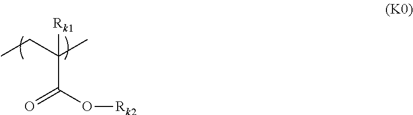

In the formula, Rₖ₁ represents a hydrogen atom, a halogen atom, a hydroxyl group, an alkyl group, a cycloalkyl group, an aryl group or a group containing a polarity conversion group.

Rₖ₂ represents an alkyl group, a cycloalkyl group, an aryl group or a group containing a polarity conversion group.

However, at least one of Rₖ₁ and Rₖ₂ represents a group containing a polarity conversion group.

A polarity conversion group refers to a group capable of decomposing by the action of an alkali developer to increase the solubility in the alkali developer, as described above. The polarity conversion group is preferably a group represented by X in the partial structure represented by Formula (KA-1) or (KB-1).

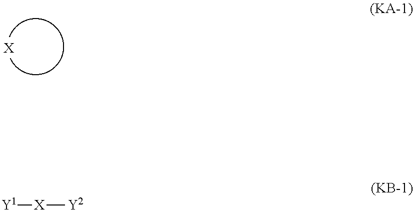

In Formula (KA-1) or (KB-1), X represents a carboxylate ester group: —COO—, an acid anhydride group: —C(O)OC(O)—, an acid imide group: —NHCONH—, a carboxylate thioester group: —COS—, a carbonate ester group: —OC(O)O—, a sulfate ester group: —OSO₂O—, a sulfonate ester group: —SO₂O—.

Y¹ and Y² each may be the same or different, and represents an electron-withdrawing group.

Meanwhile, the repeating unit (by) has a preferred group capable of increasing the solubility in an alkali developer by having a group having the partial structure represented by Formula (KA-1) or (KB-1), but as in the case of the partial structure represented by Formula (KA-1) or the partial structure represented by (KB-1) where Y¹ and Y² are monovalent, when the partial structure does not have a bonding hand, the group having the partial structure is a group having a monovalent or higher valent group formed by removing at least one arbitrary hydrogen atom in the partial structure.

The partial structure represented by Formula (KA-1) or (KB-1) is linked to the main chain of the hydrophobic resin at an arbitrary position through a substituent.

The partial structure represented by Formula (KA-1) is a structure that forms a ring structure together with a group as X.

In Formula (KA-1), X is preferably a carboxylate ester group (that is, a case of forming a lactone ring structure as KA-1), an acid anhydride group or a carbonate group.

A carboxylate ester group is more preferred.

The ring structure represented by Formula (KA-1) may have a substituent, and may have, for example, nka substituents Zₖₛ₁'s.

When a plurality of Zₖₐ₁s, Zₖₐ₁'s is present, Zₖₐ₁'s each independently represent a halogen atom, an alkyl group, a cycloalkyl group, ether group, a hydroxyl group, an amide group, an aryl group, an lactone ring group or an electron-withdrawing group.

Zₖₐ₁'s may be linked to each other to form a ring. Examples of the ring formed by Zₖₐ₁'s being linked to each other may include a cycloalkyl ring and a heterocylic ring (a cyclic ether ring, a lactone ring and the like).

nka represents an integer of 0 to 10. nka is preferably an integer of 0 to 8, more preferably an integer of 0 to 5, still more preferably an integer of 1 to 4, and most preferably an integer of 1 to 3.

The electron-withdrawing group as Zₖₐ₁ is the same as the electron-withdrawing group as Y¹ or Y² as described below. Meanwhile, the electron-withdrawing group may be substituted with another electron-withdrawing group.

Zₖₐ₁ is preferably an alkyl group, a cycloalkyl group, an ether group, a hydroxyl group or an electron-withdrawing group, and more preferably an alkyl group, a cycloalkyl group or an electron-withdrawing group. Meanwhile, the ether group is preferably an ether group substituted with an alkyl group, a cycloalkyl group and the like, that is, an alkyl ether group. The electron-withdrawing group has the same meaning as above.

The halogen atom as Zₖₐ₁ may be a fluorine atom, a chlorine atom, a bromine atom or an iodine atom, and preferably a fluorine atom.

The alkyl group as Zₖₐ₁ may have a substituent, and may be straight or branched. The straight alkyl group preferably has 1 to 30 carbon atoms, and more preferably has 1 to 20 carbon atoms, and examples thereof may include a methyl group, an ethyl group, a n-propyl group, a n-butyl group, a sec-butyl group, a t-butyl group, a n-pentyl group, a n-hexyl group, a n-heptyl group, a n-octyl group, a n-nonyl group, and a n-decanyl group. The branched alkyl group preferably has 3 to 30 carbon atoms, and more preferably 3 to 20 carbon atoms, and examples thereof may include an i-propyl group, an i-butyl group, a t-butyl group, an i-pentyl group, a t-pentyl group, an i-hexyl group, a t-hexyl group, an i-heptyl group, a t-heptyl group, an i-octyl group, a t-octyl group, an i-nonyl group, and a t-decanoyl group. The alkyl group is preferably an alkyl group having 1 to 4 carbon atoms such as a methyl group, an ethyl group, a n-propyl group, an i-propyl group, a n-butyl group, an i-butyl group, or a t-butyl group is preferred.

The cycloalkyl group as Zₖₐ₁ may have a substituent, and may be monocyclic or polycyclic. In the case of a polycyclic group, the cycloalkyl group may be bridged. That is, in this case, the cycloalkyl group may have a bridged structure. The monocyclic cycloalkyl group is preferably a cycloalkyl group having 3 to 8 carbon atoms, and examples thereof may include a cyclopropyl group, a cyclopentyl group, a cyclohexyl group, a cyclobutyl group, and a cyclooctyl group. Examples of the polycyclic cycloalkyl group may include a group having a bicyclo, tricyclo, or tetracyclo structure and having 5 or more carbon atoms. A cycloalkyl group having 6 to 20 carbon atoms is preferred, and examples thereof may include an adamantyl group, a norbornyl group, an isoboronyl group, a camphanyl group, a dicyclopentyl group, an α-pinel group, a tricyclodecanyl group, a tetracyclododecyl group and an androstanyl group. The cycloalkyl group may preferably include the following structures. Meanwhile, at least one carbon atom in the cycloalkyl group may be substituted with a heteroatom such as an oxygen atom.

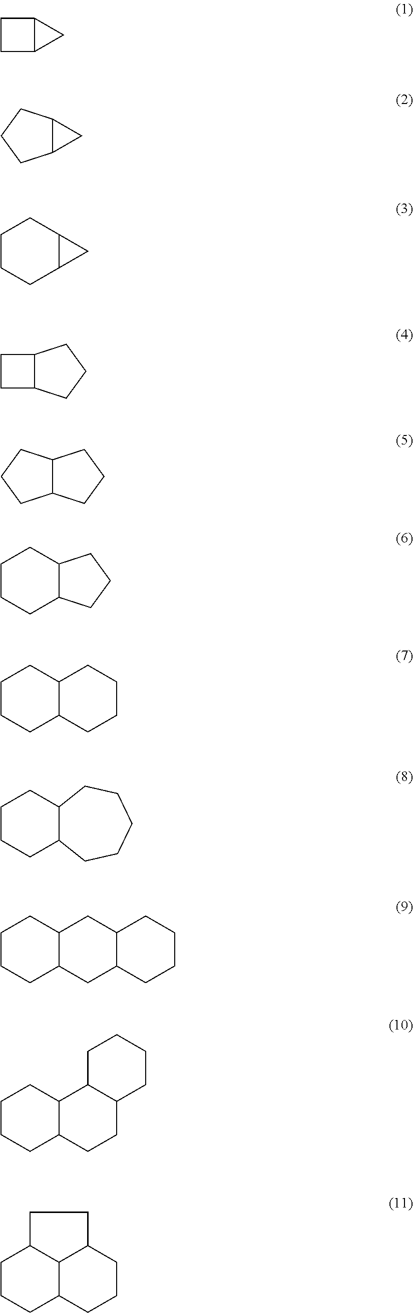

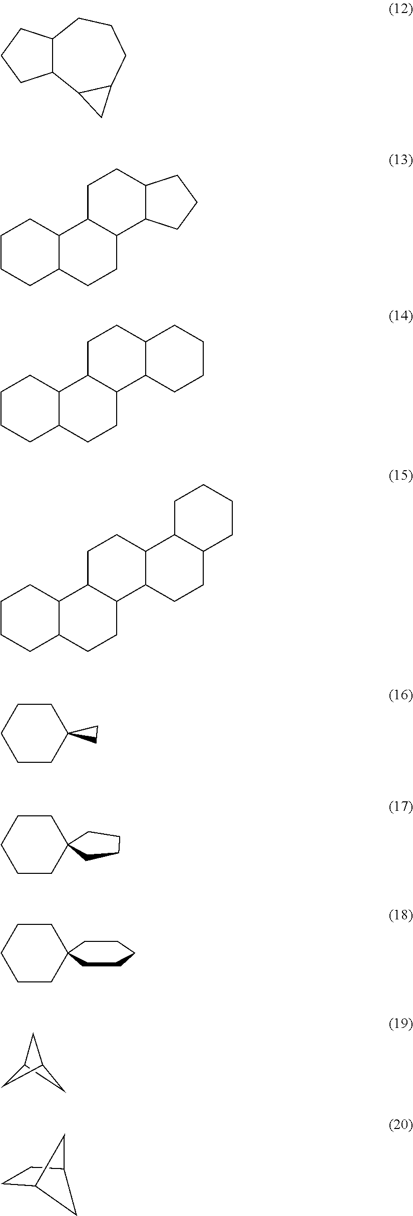

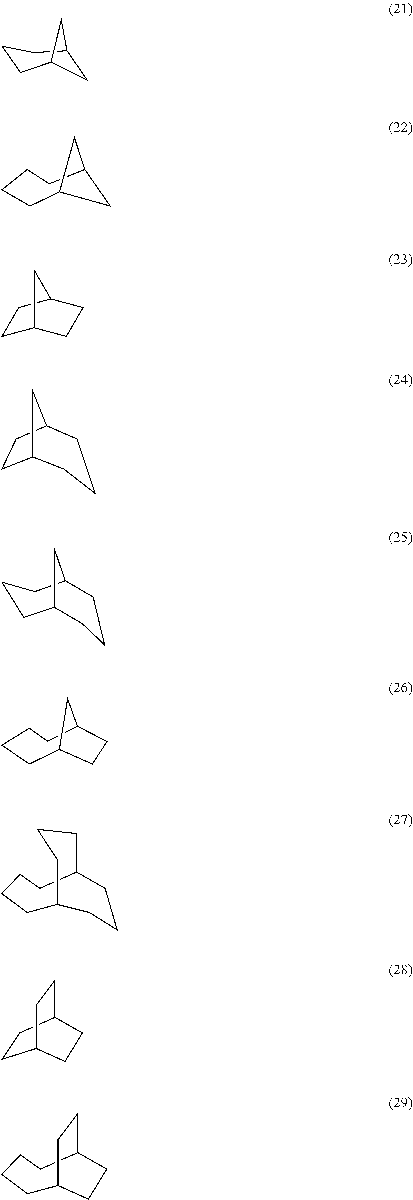

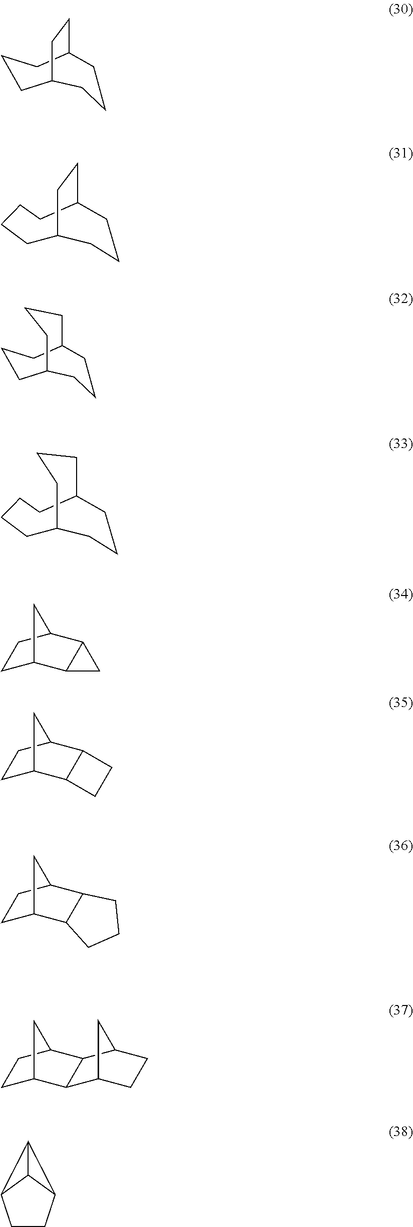

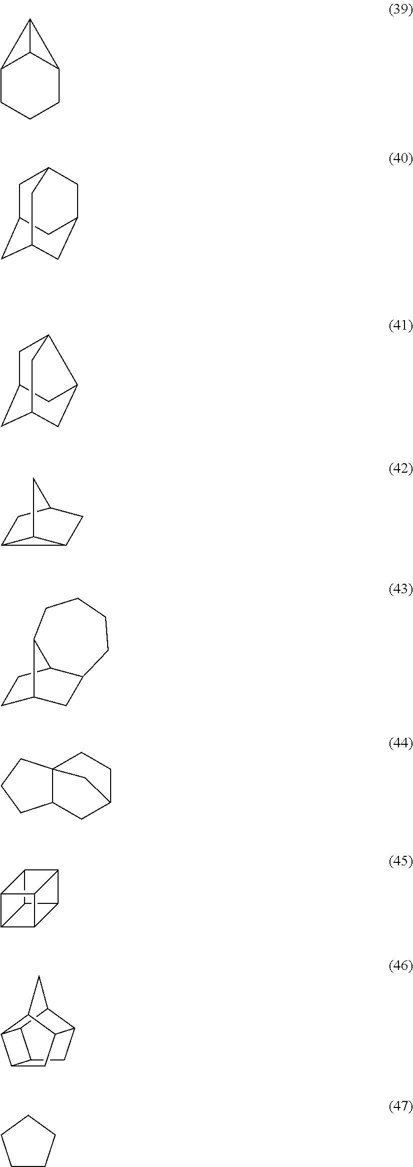

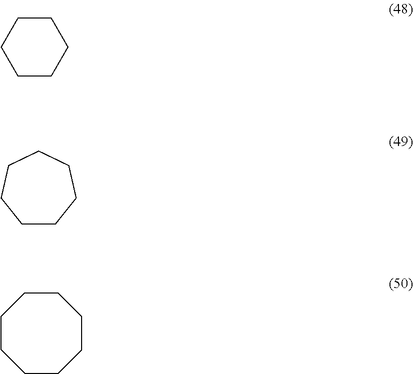 [SMILES: C1CCCCC1 ; C1CCCCCC1 ; C1CCCCCCC1]

Preferred examples of the alicylic moiety may include an adamantyl group, a noradamantyl group, a decalin group, a tricyclodecanyl group, a tetracyclododecanyl group, a norbomyl group, a cedrol group, a cyclohexyl group, a cycloheptyl group, a cyclooctyl group, a cyclodecanyl group and a cyclododecanyl group. The alicyclic moiety is more preferably an adamantyl group, a decalin group, a norbornyl group, a cedrol group, a cyclohexyl group, a cycloheptyl group, a cyclooctyl group, a cyclodecanyl group, a cyclododecanyl group or a tricyclodecanyl group.

Examples of the substituent of the alicyclic structure may include an alkyl group, a halogen atom, a hydroxyl group, an alkoxy group, a carboxyl group and an alkoxycarbonyl group. The alkyl group is preferably a lower alkyl group such as a methyl group, an ethyl group, a propyl group, an isopropyl group and a butyl group, and more preferably a methyl group, an ethyl group, a propyl group or an isopropyl group. Examples of the alkoxy group may include preferably an alkoxy group having 1 to 4 carbon atoms such as a methoxy group, an ethoxy group, a propoxy group and a butoxy group. Examples of the substituent which may be possessed by the alkyl group or the alkoxy group may include a hydroxyl group, a halogen atom and an alkoxy group (preferably having 1 to 4 carbon atoms).

The group may further have a substituent, and examples of the further substituent may include a hydroxyl group, a halogen atom (fluorine, chlorine, bromine and iodine), a nitro group, a cyano group, the aforementioned alkyl group, an alkoxy group such as a methoxy group, an ethoxy group, a hydroxyethoxy group, a propoxy group, a hydroxypropoxy group, a n-butoxy group, an isobutoxy group, a sec-butoxy group and a t-butoxy group, an alkoxycarbonyl group such as a methoxycarbonyl group and an ethoxycarbonyl group, an aralkyl group such as a benzyl group, a phenethyl group and a cumy group, an aralkyloxy group, an acyl group such as a formyl group, an acetyl group, a butyryl group, a benzoyl group, a cinnamyl group and a valeryl group, an acyloxy group such as a butyryloxy group, an alkenyl group such as a vinyl group, a prophenyl group and an allyl group, an alkenyloxy group such as a vinyloxy group, a prophenyloxy group, an allyloxy group and a butenyloxy group, an aryl group such as a phenyl group and a naphthyl group, an aryloxy group such as a phenoxy group, an aryloxycarbonyl group such as a benzoyloxy group and the like.

It is preferred that X in Formula (KA-1) is a carboxylate ester group, and the partial structure represented by Formula (KA-1) is a lactone ring, and the lactone ring is preferably a 5- to 7-membered lactone ring.

Meanwhile, as in (KA-1-1) to (KA-1-18) below, it is preferred that another ring structure is condensed to a 5- to 7-membered ring lactone ring that is the partial structure represented by Formula (KA-1) in the form of forming a bicyclo or spiro structure.

Examples of the peripheral ring structure to which the ring structure represented by Formula (KA-1) may be bonded may include those in (KA-1-) to (KA-1-18) below or structures based on these structures.

The structure containing the lactone ring structure represented by Formula (KA-1) is more preferably a structure represented by any one of the following (KA-1-1) to (KA-1-18). Meanwhile, the lactone structure may be directly bonded to the main chain. Preferred structures are (KA-1-1), (KA-1-4), (KA-1-5), (KA-1-6), (KA-1-13), (KA-1-14) and (KA-1-17).

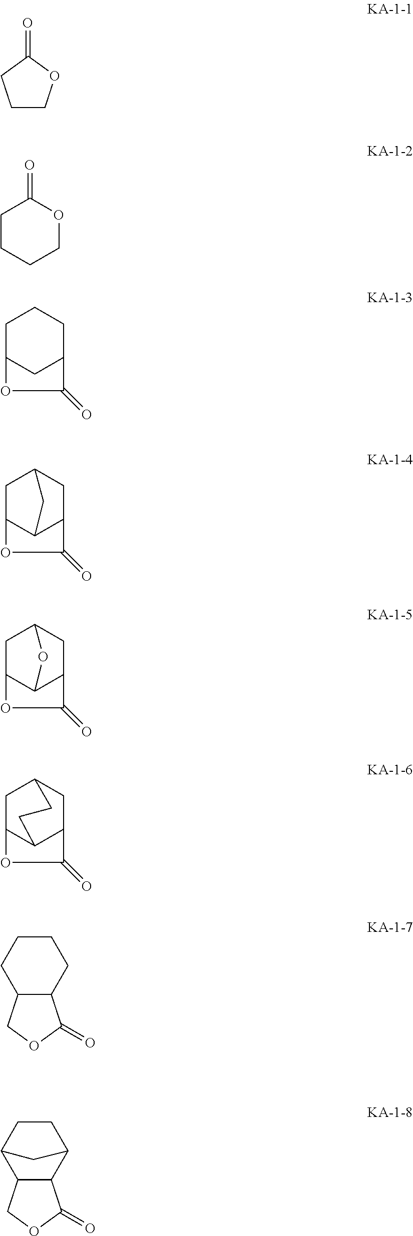

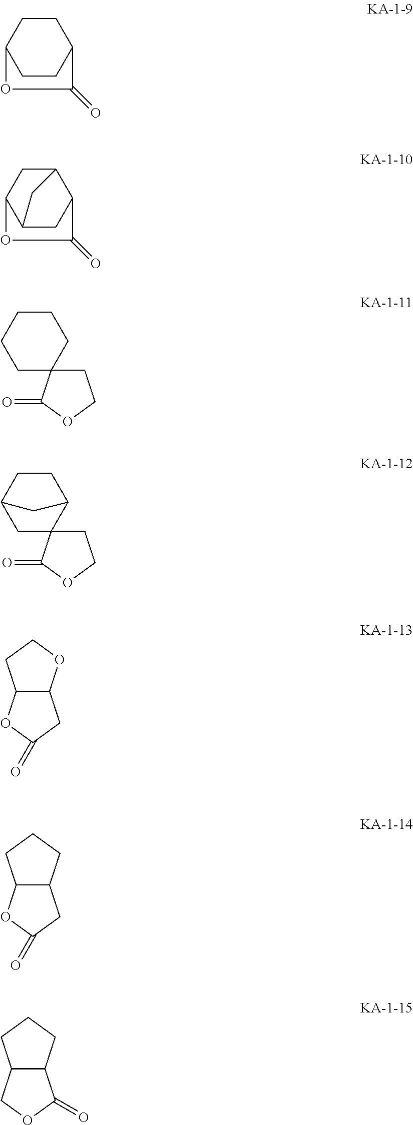

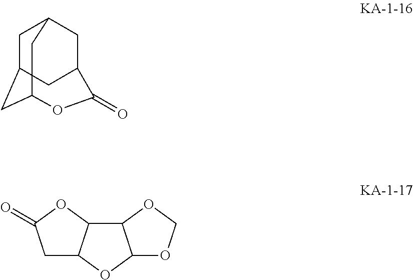 [SMILES: O=C1OC2CC3CC(C2)CC1C3 ; O=C1CC2OC3OCOC3C2O1]

The structure containing a lactone ring structure may or may not have a substituent. Preferred examples of the substituent may be the same as the substituent Zₖₐ₁ which may be possessed by the ring structure represented by Formula (KA-1).

In Formula (KB-1), X may be preferably a carboxylate ester group (—COO—).

In Formula (KB-1), Y¹ and Y² each independently represent an electron-withdrawing group.

The electron-withdrawing group is a partial structure represented by the following Formula (EW). In Formula (EW), * represents a bonding hand directly bonded to (KA-1), or a bonding hand directly bonded to X in (KB-1).

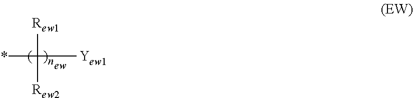

In Formula (EW),

R_{ew1} and R_{ew2} each independently represent an arbitrary substituent, and for example, represent a hydrogen atom, an alkyl group, a cycloalkyl group or an aryl group.

n_{ew} is a repeating number of the linking group represented by —C(R_{ew1})(R_{ew2})—, and represents an integer of 0 or 1. In the case where n_{ew} is 0, this indicates that the bond is a single bond, and Y_{ew1} is directly bonded.

Y_{ew1} is a halogen atom, a cyano group, a nitrile group, a nitro group, a halo(cyclo)alkyl group or haloaryl group represented by —C(R_{f1})(R_{f2})—R_{f3}, an oxy group, a carbonyl group, a sulfonyl group, a sulfinyl group or a combination thereof. The electron-withdrawing group may be, for example, a structure shown below. The term “halo(cyclo)alkyl group” indicates an alkyl group or a cycloalkyl group that is at least partially halogenated, and term “haloaryl group” indicates an aryl group that is at least partially halogenated. In the following structural formulas, R_{ew3} and R_{ew4} each independently represent an arbitrary structure. The partial structure represented by Formula (EW) has an electron-withdrawing property regardless of what structure R_{ew3} or R_{ew4} may take, and R_{ew3} and R_{ew4} may be linked to, for example, the main chain of the resin, but is preferably an alkyl group, a cycloalkyl group, or a fluorinated alkyl group.

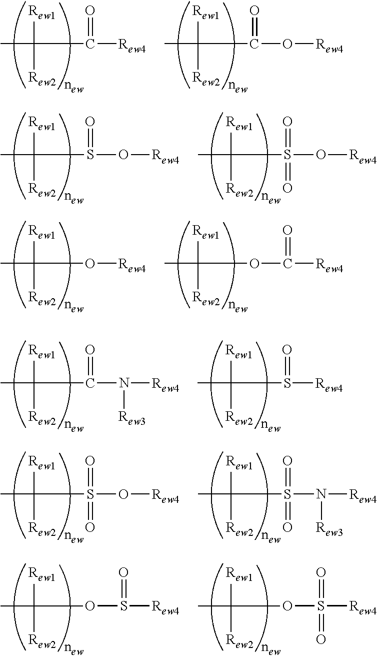

When Y_{ew1} is a divalent or higher valent group, the remaining bonding hands form a bond to an arbitrary atom or substituent. At least one group of Y_{ew1}, R_{ew1} and R_{ew2} may be linked to the main chain of the hydrophobic resin through a further substituent.

Y_{ew1} is preferably a halogen atom or a halo(cyclo)alkyl group or haloaryl group represented by —C(R_{f1})(R_{f2})—R_{f3}.

At least two of R_{ew1}, R_{ew2} and Y_{ew1} may be linked to each other to form a ring.

Herein, R_{f1} represents a halogen atom, a perhaloalkyl group, a perhalocycloalkyl group or a perhaloaryl group, more preferably a fluorine atom, a perfluoroalkyl group or a perfluorocycloalkyl group, and still more preferably a fluorine atom or a trifluoromethyl group.

R_{f2} and R_{f3} each independently represent a hydrogen atom, a halogen atom or an organic group, and R_{f2} and R_{f3} may be linked to each other to form a ring. Examples of the organic group may include an alkyl group, a cycloalkyl group and an alkoxy group. R_{f2} represents the same group as R_{f1}, or is more preferably linked with R_{f3} to form a ring.

R_{f1} to R_{f3} may be linked to each other to form a ring, and examples of the ring formed may include a (halo)cycloalkyl ring, and (halo)aryl ring.

Examples of the (halo)alkyl group in R_{f1} to R_{f3} may include the alkyl group in Zₖₐ₁ as described above, and a halogenated structure thereof.

Examples of the (per)halocycloalkyl group and the (per)haloaryl group in R_{f1} to R_{f3}, or in the ring formed by R_{f2} and R_{f3} being linked to each other may include a structure formed by halogenations of the cycloalkyl group in Zₖₐ₁ as described above, and more preferably a fluorocycloalkyl group represented by —C₍ₙ₎F₍₂ₙ₋₂₎H and a perfluoroaryl group represented by —C₍ₙ₎F₍ₙ₋₁₎. Here, the carbon number n is not particularly limited, but is preferably 5 to 13, and more preferably 6.

The ring which may be formed by at least two of R_{ew1}, R_{ew2} and Y_{ew1} being linked to each other is preferably a cycloalkyl group or a heterocyclic group, and the heterocyclic group is preferably a lactone ring group. Examples of the lactone ring may include structures represented by Formulas (KA-1-1) to (KA-1-17).

Meanwhile, the repeating unit (by) may have a plurality of partial structures represented by Formula (KA-1), a plurality of partial structures represented by Formula (KB-1), or both of a partial structure represented by Formula (KA-1) and a partial structure represented by Formula (KB-1).

Meanwhile, the partial structure of Formula (KA-1) may also serve partially or entirely as the electron-withdrawing group of Y¹ or Y² in Formula (KB-1). For example, in the case where X in Formula (KA-1) is a carboxylate ester group, the carboxylate ester group may function as an electron-withdrawing group of Y¹ or Y² in Formula (KB-1).

When the repeating unit (by) corresponds to the repeating unit (b*) or the repeating unit (b″), and has a partial structure represented by Formula (KA-1), it is more preferred that the polarity conversion group in the partial structure represented by Formula (KA-1) is a partial structure represented by —COO— in the structure represented by Formula (KA-1).

The repeating unit (by) may be a repeating unit having a partial structure represented by Formula (KY-0).

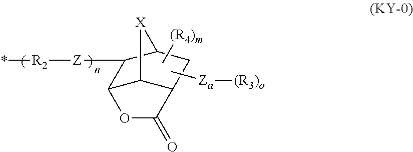

In Formula (KY-0),

R₂ represents a chained or cyclic alkylene group, and when a plurality of R₂'s is present, R₂'s may be the same or different.

R₃ represents a straight, branched or cyclic hydrocarbon group in which a part or all of hydrogen atoms on the constituent carbons are substituted with fluorine atoms.

R₄ represents a halogen atom, a cyano group, a hydroxy group, an amide group, an alkyl group, a cycloalkyl group, an alkoxy group, a phenyl group, an acyl group, an alkoxycarbonyl group or a groupo represented by R—C(═O)— or R—C(═O)O— (wherein R represents an alkyl group or a cycloalkyl group). When a plurality of R₄'s is present, R₄'s may be the same or different, and two or more R₄'s may be bound with each other to form a ring.

X represents an alkylene group, an oxygen atom or a sulfur atom.

Z and Za represent a single bond, an ether bond, an ester bond, an amide bond, a urethane bond or a urea bond, and when a plurality of Z's and Za's are present, Z's and Za's may be the same or different.

* represents a bonding hand to the main or side chain of the resin.

o is the number of substituents, and represents an integer of 1 to 7.

m is the number of substituents, and represents an integer of 0 to 7.

n is a repeating number, and represents an integer of 0 to 5.

Preferably, the structure of —R₂—Z— is preferably a structure represented by —(CH₂)₁—COO— (wherein 1 represents an integer of an 1 to 5).

Preferred range of the carbon number and specific examples of the chained or cyclic alkylene group as R₂ are the same as those described for the chained alkylene group and the cyclic an alkylene group in Z₂ of Formula (bb).

For the straight, branched or cylic hydrocarbon group as R₃, the straight hydrocarbon group preferably has 1 to 30 carbon atoms, and more preferably 1 to 20 carbon atoms, the branched hydrocarbon group preferably has 3 to 30 carbon atoms, and more preferably 3 to 20 carbon atoms, and the cyclic hydrocarbon group has 6 to 20 carbon atoms. Specific examples of R₃ may include specific examples of the alkyl group and the cycloalkyl group as Zₖₐ₁ as described above.

Preferred carbon atoms and specific examples in the alkyl group and the cycloalkyl group as R₄ and R are the same as those described above for the alkyl group and the cycloalkyl group as Zₖₐ₁.

The acyl group as R₄ preferably has 1 to 6 carbon atoms, and examples thereof may include a formyl group, an acetyl group, a propionyl group, a butyryl group, an isobutyryl group, a valeryl group, a pivaloyl group and the like

The alkyl moiety in the alkoxy group and the alkoxycarbonyl group as R₄ may include a straight, branched or cyclic alkyl moiety, and preferred carbon atoms and specific examples of the alkyl moiety are the same as those described above for the alkyl group and the cycloalkyl group as Zₖₐ₁.

The alkylene group as X may be a chained or cyclic alkylene group, and preferred carbon atoms and specific examples thereof are the same as those described above for the the chained alkylene group and the cyclic alkylene group as R₂.

Further, specific structures of the repeating unit (by) may include repeating units having the following partial structures.

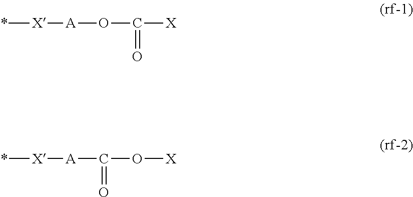

In Formulas (rf-1) and (rf-2),

X′ represents an electron-withdrawing substituent, and is preferably a carbonyloxy group, an oxycarbonyl group, an alkylene group substituted with a fluorine atom, a cycloalkylene group substituted with a fluorine atom.

A represents a single bond or a divalent linking group represented by —C(Rx)Ry)-.

Here, Rx and Ry each independently represent a hydrogen atom, a fluorine atom, an alkyl group (which preferably has 1 to 6 carbon atoms, and may be substituted with a fluorine atom and the like) or a cycloalkyl group (which preferably has 5 to 12 carbo atoms, and may be substituted with a fluorine atom and the like). Rx and Ry are preferably a hydrogen atom, an alkyl group or an alkyl group substituted with a fluorine atom.

X represents an electron-withdrawing group, and specific examples thereof may include the electron-withdrawing groups as Y¹ and Y² as described above, and preferably a fluoroalkyl group, a fluorocycloalkyl group, an aryl group substituted with a fluorine or a fluoroalkyl group, an aralkyl group substituted with a fluorine or a fluoroalkyl group, a cyano group or a nitro group.

* represents a bonding hand to the main chain or side chain of the resin. That is, it represents a bonding hand bonding to the main chain through a single bond or a linking group.

Meanwhile, when X′ is a carbonyloxy group or an oxycarbonyl group, A is not a single bond.

The polarity conversion group is decomposed by the action of an alkali developer to effect polarity conversion, whereby the receding contact angle with water of the resist film after alkali development may be decreased. Decrease in the receding contact angle with water of the film after alkali development is preferred from the viewpoint of suppressing the development defects.

The receding contact angle with water of the resist film after alkali development is preferably 50° or less, more preferably 40° or less, still more preferably 35° or less, and most preferably 30° or less, at a temperature of 23±3C and a humidity of 45+5%.

The receding contact angle is a contact angle measured when a contact line recedes on the liquid droplet-substrate interface, and this is generally known to be useful in simulating the mobility of a liquid droplet in the dynamic state. In a simple manner, the receding contact angle may be defined as a contact angle at the time of the liquid droplet interface receding when a liquid droplet ejected from a needle tip is landed on a substrate and then the liquid droplet is again suctioned into the needle. In general, the receding contact angle may be measured by a contact angle measuring method called an expansion/contraction method.

The hydrolysis rate of the hydrophobic resin for an alkali developer is preferably 0.001 nm/sec or more, more preferably 0.01 nm/sec or more, still more preferably 0.1 nm/sec or more, and most preferably 1 nm/sec or more.

Here, the hydrolysis rate of the hydrophobic resin(1-IR) for an alkali developer is the rate at which the thickness of a resin film formed of only the hydrophobic resin decreases with respect to TMAH (an aqueous tetramethylammonium hydroxide solution) (2.38% by mass) at 23° C.

Further, the repeating unit (by) is more preferably a repeating having at least two or more polarity conversion groups.

In the case where the repeating unit (by) has at least two polarity conversion groups, the repeating unit preferably has a group containing a partial structure having two polarity conversion groups represented by the following Formula (KY-1). Incidentally, when the structure represented by Formula (KY-1) does not have a bonding hand, this is a group containing a monovalent or greater valent group formed by removing at least one arbitrary hydrogen atom from the structure.

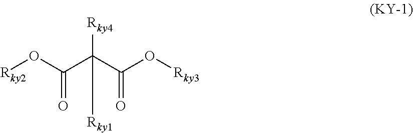

In Formula (KY-1).

R_{ky1} and R_{ky4} each independently represent a hydrogen atom, a halogen atom, an alkyl group, a cycloalkyl group, a carbonyl group, a carbonyloxy group, an oxycarbonyl group, an ether group, a hydroxyl group, a cyano group, an amide group or an aryl group. Alternatively. R_{ky1} and R_{ky4} may be bonded to the same atom to form a double bond. For example, R_{ky1} and R_{ky1} may be bonded to the same oxygen atom to form a part (═O) of a carbonyl group.

R_{ky2} and R_{ky3} each independently represent an electron-withdrawing group, or while R_{ky1} and R_{ky2} are linked with each other to form a lactone ring. R_{ky3} is an electron-withdrawing group. The formed lactone ring is preferably a structure of (KA-1-1) to (KA-1-18).

Examples of the electron-withdrawing group may be the same as those for Y¹ and Y² in Formula (KB-1), and preferably a halogen atom or a halo(cyclo)alkyl or haloaryl group represented by —C(R_{f1})(R_{f2})—R_{f3}. Preferably, R_{ky3} is a halogen atom or a halo(cyclo)alkyl or haloaryl group represented by —C(R_{f1})(R_{f2})—R_{f3}, and R_{ky2} is linked with R_{ky1} to form a lactone ring or is an electron-withdrawing group containing no halogen atom.

R_{ky1}, R_{ky2} and R_{ky4} may be linked to each other to form a monocyclic or polycyclic structure.

Specific examples of R_{ky1} and R_{ky4} include the same groups as those for Zₖₐ₁ in Formula (KA-1).

The lactone ring formed by R_{ky1} and R_{ky2} being linked to each other is preferably a structure of (KA-1-1) to (KA-1-17). Examples of the electron-withdrawing group may be the same as those for Y¹ and Y² in Formula (KB-1).

The structure represented by Formula (KY-1) is more preferably a structure represented by the following Formula (KY-2). Meanwhile, the structure represented by Formula (KY-2) is a group having a monovalent or higher valent group formed by removing at least one arbitrary hydrogen atom from the structure.

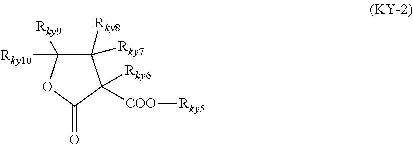

In Formula (KY-2),

R_{ky6} to R_{ky10} each independently represent a hydrogen atom, a halogen atom, an alkyl group, a cycloalkyl group, a carbonyl group, a carbonyloxy group, an oxycarbonyl group, an ether group, a hydroxyl group, a cyano group, an amide group or an aryl group.

Two or more of R_{ky6} to R_{ky10} may be linked to each other to form a monocyclic or polycyclic structure.

R_{ky5} represents an electron-withdrawing group. The electron-withdrawing group may be the same as those for Y¹ and Y², and is preferably a halogen atom or a halo(cyclo)alkyl group or a haloaryl group represented by —C(R_{f1})(R_{f2})—R_{f3}.

Specific examples of R_{ky5} to R_{ky10} may include the same groups as those for Zₖₐ₄ in Formula (KA-1).

The structure represented by Formula (KY-2) is more preferably a partial structure represented by the following Formula (KY-3).

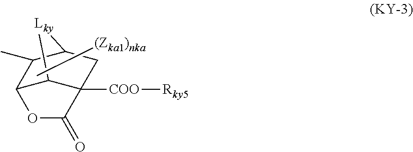

In (KY-3), Zₖₐ₁ and nka have the same meaning as those in Formula (KA-1), respectively. R_{ky5} has the same meaning as that in (KY-2).

L_{ky} represents an alkylene group, an oxygen atom or a sulfur atom. Examples of the alkylene group of L_{ky} may include a methylene group, an ethylene group and like. L_{ky} is preferably an oxygen atom or a methylene group, and is more preferably a methylene group.

The repeating unit (b) is not limited as long as it is a repeating unit obtained by polymerization such as addition polymerization, condensation polymerization and addition condensation, but this repeating unit is preferably a repeating unit obtained by addition polymerization of a carbon-carbon double bond. Examples thereof include an acrylate-based repeating unit (including a system having a substituent at the α- or β-position), a styrene-based repeating unit (including a system having a substituent at the α- or β-position), a vinyl ether-based repeating unit, a norbornene-based repeating unit, and a maleic acid derivative (such as maleic anhydride, its derivative, and maleimide) repeating unit, and preferably an acrylate-based repeating unit, a styrene-based repeating unit, a vinyl ether-based repeating unit and a norbornene-based repeating unit, more preferably an acrylate-based repeating unit, a vinyl ether-based repeating unit and a norbornene-based repeating unit, and most preferably an acrylate-based repeating unit.

In the case where the repeating unit (by) is a repeating unit having at least one of a fluorine atom or a silicon atom (that is, a repeating unit corresponding to the repeating unit (b′) or (b″)), examples of the fluorine atom-containing partial structure in the repeating unit (by) may be the same as those exemplified in the repeating unit having at least any one of a fluorine atom and a silicon atom, and preferably the groups represented by Formulas (F2) to (F4). Further, examples of the silicon atom-containing partial structure in the repeating unit (by) may be the same as those exemplified in the repeating unit having at least one of a fluorine atom and a silicon atom, and preferably the groups represented by Formulas (CS-1) to (CS-3).

In the hydrophobic resin, the content of the repeating unit (by) is preferably 10 mol % to 100 mol %, more preferably 20 mol % to 99 mol %, still more preferably 30 mol % to 97 mol %, and most preferably 40 mol % to 95 mol % based on the whole repeating units in the hydrophobic resin.

Specific examples of the repeating unit (by) having a group capable of increasing the solubility in an alkali developer are shown below, but the present invention is not limited thereto.

In the specific examples as shown below, Rₐ represents a hydrogen atom, a fluorine atom, a methyl group or a trifluoromethyl group.

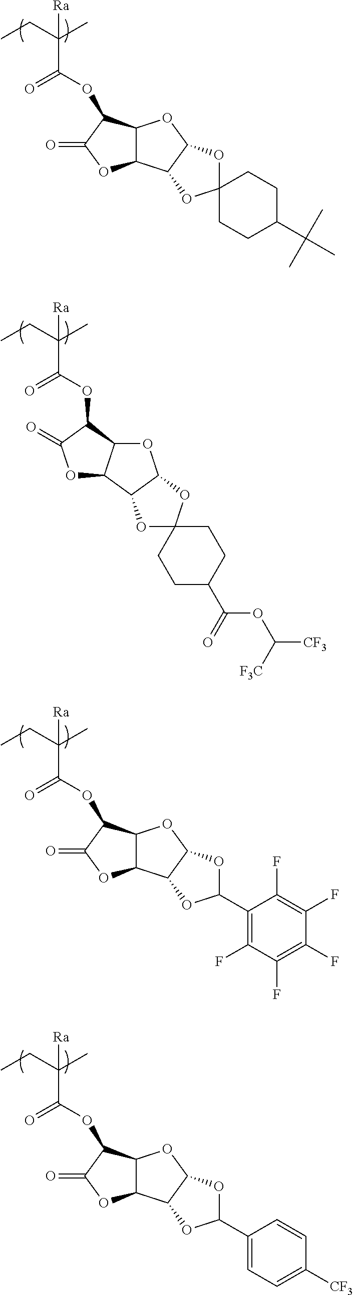

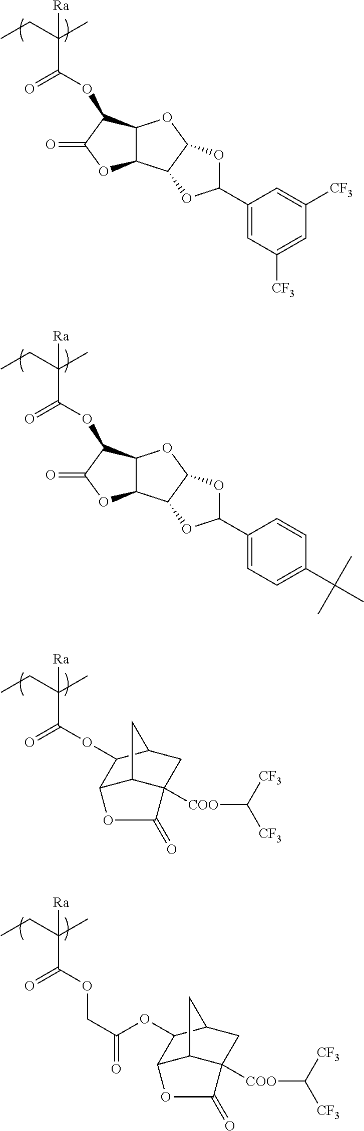

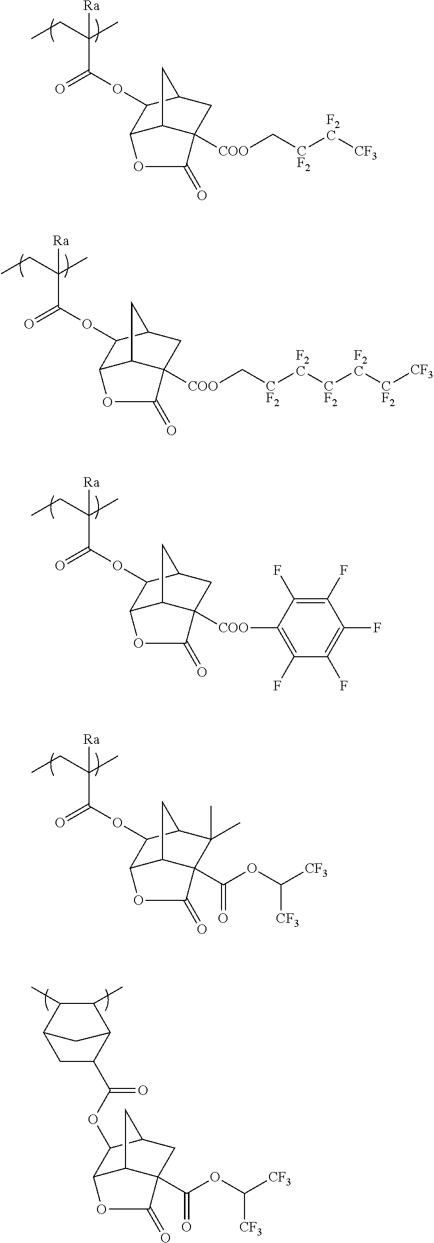

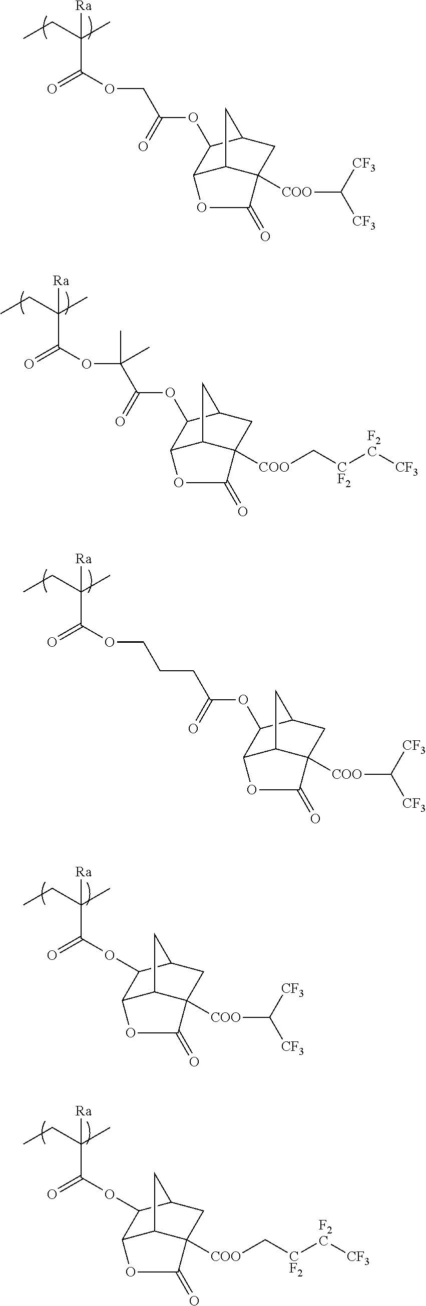

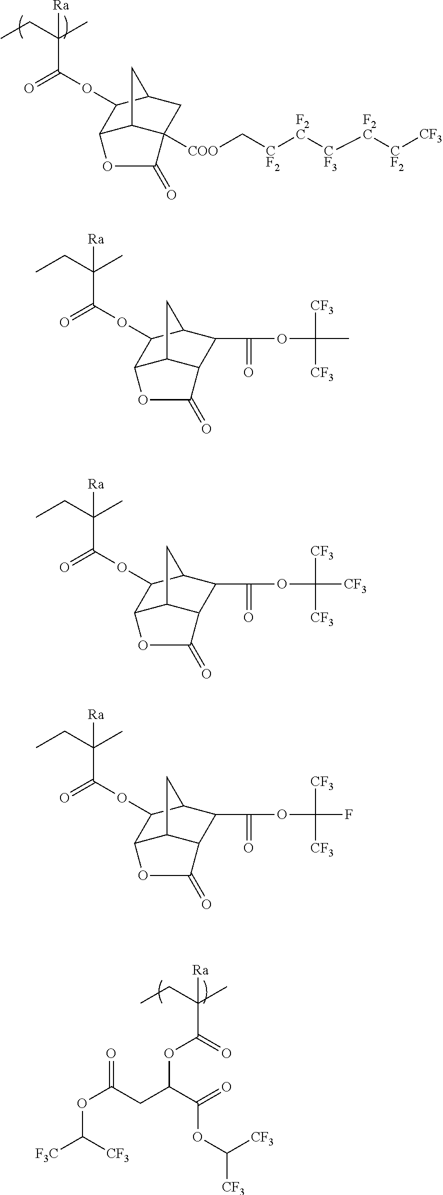

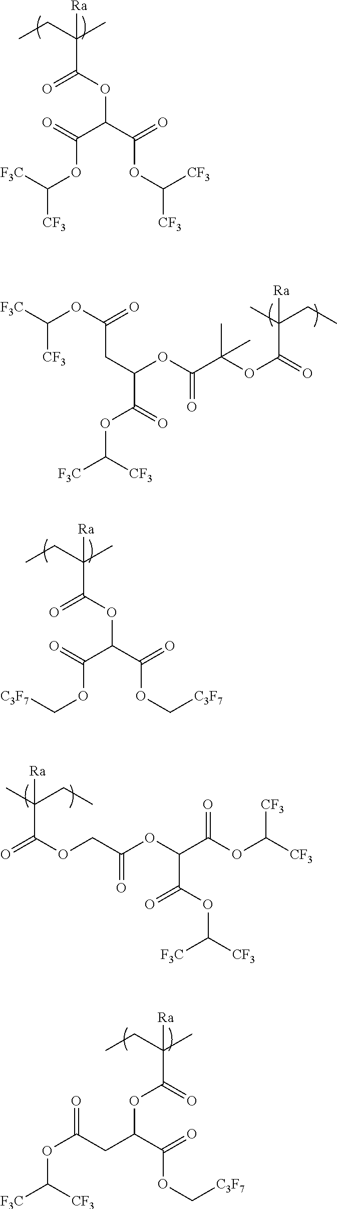

The synthesis of the monomers corresponding to the aforementioned repeating units (by) containing the polarity conversion group (y) may be carried out with reference to, for example, International Publication No. 2010/067905 or the methods described in International Publication No. 2010/067905.

In the hydrophobic resin, the repeating unit (bz) containing a group (z) capable of decomposing by the action of an acid may be the same as the repeating units containing an acid-decomposable group exemplified in the resin (B).

In the case where the repeating unit (bz) is a repeating unit having at least one of a fluorine atom or a silicon atom (that is, in the case of corresponding to the aforementioned repeating unit (b′) or repeating unit (b″)), the partial structure containing a fluorine atom contained in the repeating unit (bz) may be the same as set forth above in the repeating unit having at least one of a fluorine atom or a silicon atom, and preferred examples thereof may include the groups represented by Formulas (F2) to (F4). Further, in this case, the partial structure having a silicon atom contained in the repeating unit (bz) may be the same as set forth above in the repeating unit having at least one of a fluorine atom or a silicon atom, and preferred examples thereof may include the groups represented by Formulas (CS-1) to (CS-3).

In the hydrophobic resin, the content of the repeating unit (bz) having the group (z) capable of decomposing by the action of an acid is preferably 1 mol % to 80 mol %, more preferably 10 mol % to 80 mol/o, and still more preferably 20 mol % to 60 mol % based on the whole repeating units in the hydrophobic resin.

The repeating unit (b) having at least one group selected from the group consisting (x) to (z) has been described, but the content of the repeating unit (b) in the hydrophobic resin is preferably 1 mol % to 98 mol %, more preferably 3 mol % to 98 mol %, still more preferably 5 mol % to 97 mol %, and most preferably 10 mol % to 95 mol % based on the whole repeating units in the hydrophobic resin.

The content of the repeating unit (b′) is preferably 1 mol % to 100 mol %, more preferably 3 mol % to 99 mol %, still more preferably 5 mol % to 97 mol %, and most preferably 10 mol % to 95 mol % based on the whole repeating units in the hydrophobic resin.

The content of the repeating unit (b*) is preferably 1 mol % to 90 mol %, more preferably 3 mol % to 80 mol %, still more preferably 5 mol % to 70 mol %, and most preferably 10 mol % to 60 mol % based on the whole repeating units in the hydrophobic resin. The content of the repeating unit having at least one of a fluorine atom and a silicon atom used in combination with the repeating unit (b*) is preferably 10 mol % to 99 mol %, more preferably 20 mol % to 97 mol %, still more preferably 30 mol % to 95 mol %, and most preferably 40 mol % to 90 mol % based on the whole repeating units in the hydrophobic resin.

The content of the repeating unit (b″) is preferably 1 mol % to 100 mol %, more preferably 3 mol % to 99 mol %, still more preferably 5 mol % to 97 mol %, and most preferably 10 mol % to 95 mol % based on the whole repeating units in the hydrophobic resin.

The hydrophobic resin may further have a repeating unit represented by the following Formula (CIII).

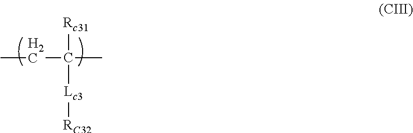

In Formula (CIII),

R_{c31} represents a hydrogen atom, an alkyl group (which may be substituted with a fluorine atom or the like), a cyano group or a —CH₂—O—Rac₂ group. In the formula, Rac₂ represents a hydrogen atom, an alkyl group or an acyl group. R_{c31} is preferably a hydrogen atom, a methyl group, a hydroxymethyl group or a trifluoromethyl group, and particularly preferably a hydrogen atom or a methyl group.

R_{c32} represents a group having an alkyl group, a cycloalkyl group, an alkenyl group, a cycloalkenyl group or an aryl group. These groups may be substituted with a group containing a fluorine atom or a silicon atom.

L_{c3} represents a single bond or a divalent linking group.

In Formula (CIII), the alkyl group of R₃₂ is preferably a straight or branched alkyl group having 3 to 20 carbon atoms.

The cycloalkyl group is preferably a cycloalkyl group having 3 to 20 carbon atoms.

The alkenyl group is preferably an alkenyl group having 3 to 20 carbon atoms.

The cycloalkenyl group is preferably a cycloalkenyl group having 3 to 20 carbon atoms.

The aryl group is preferably a phenyl group or a naphthyl group having 6 to 20 carbon atoms and these groups may have a substituent.

R_{c32} is preferably an unsubstituted alkyl group or an alkyl group substituted with a fluorine atom.

The divalent linking group of L_{c3} is preferably an alkylene group (preferably having to 1 to 5 carbon atoms), an oxy group, a phenylene group, or an ester bond (a group represented by —COO—).

It is also preferred that the hydrophobic resin further has a repeating unit represented by the following Formula (BII-AB).

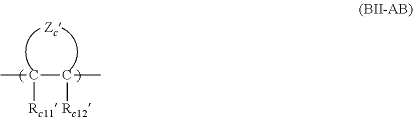

In Formula (BII-AB),

R_{c11}′ and R_{c12}′ each independently represent a hydrogen atom, a cyano group, a halogen atom or alkyl group.

Zc′ includes two carbon atoms (C—C) to which Zc′ is bonded and represents an atomic group for forming an alicyclic structure.

When each group in the repeating unit represented by Formula (CIII) or (BII-AB) is substituted with a group containing a fluorine atom or a silicon atom, the repeating unit also corresponds to the aforementioned repeating unit having at least one of a fluorine atom and a silicon atom.

Hereinafter, specific examples of the repeating units represented by Formulas (CIII) or (BII-AB) will be described below, but the present invention is not limited thereto. In the formulas, Rₐ represents H, CH₃, CH₂OH, CF₃ or CN. Meanwhile, the repeating unit in which Rₐ is CF₃ also corresponds to the repeating unit having at least one of a fluorine atom and a silicon atom.

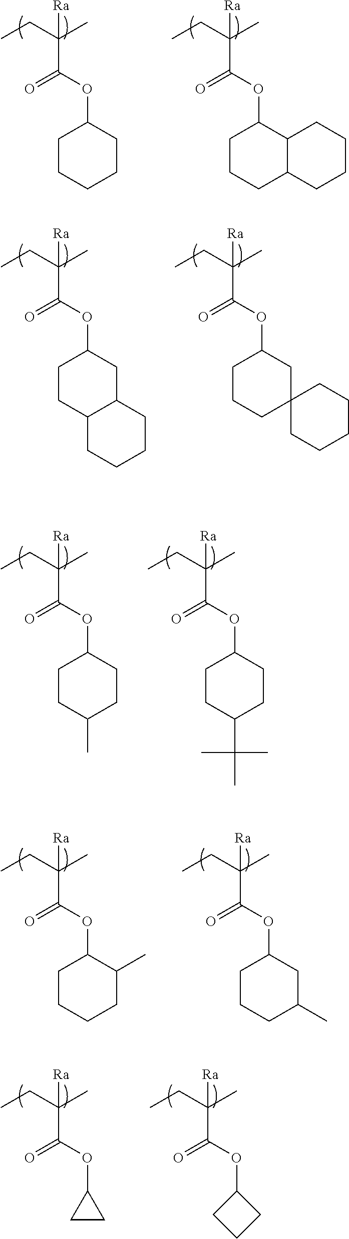

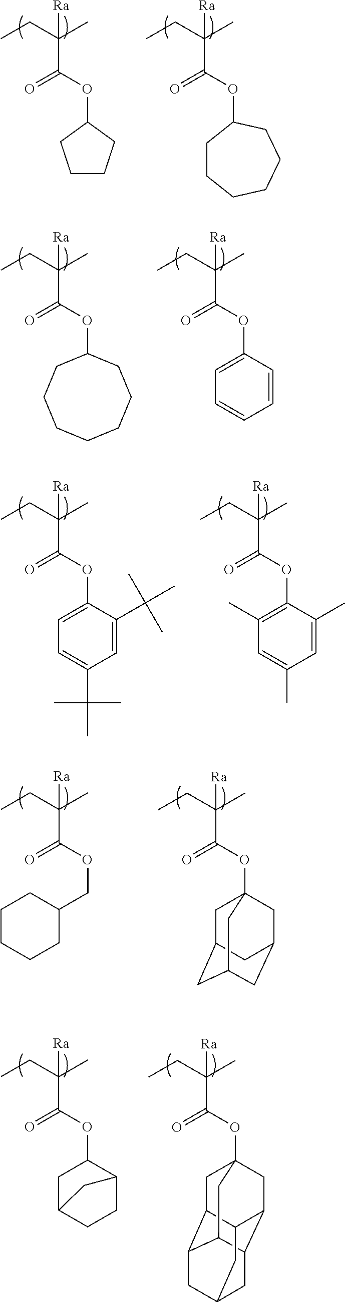

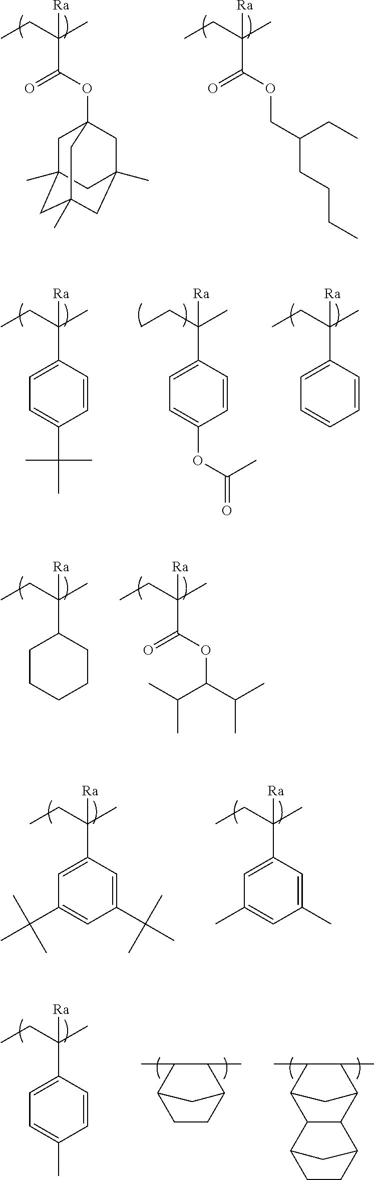

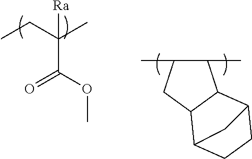

In the hydrophobic resin, like in the aforementioned resin (C), it is natural that the content of impurities such as metal and the like is small, and the content of residual monomers or oligomer components is preferably 0% by mass to 10% by mass, more preferably 0% by mass to 5% by mass, and still more preferably 0% by mass to 1% by mass. Accordingly, it is possible to obtain a resist composition free from extraneous substances in liquid and change in sensitivity or the like over time. Further, from the viewpoint of resolution, resist shape, side wall of resist pattern, roughness and the like, the molecular weight distribution (Mw/Mn, also referred to as polydispersity) is in a range of preferably 1 to 3, more preferably 1 to 2, still more preferably 1 to 1.8, and most preferably of 1 to 1.5.

As for the hydrophobic resin, various commercially available products may be used, and the hydrophobic resin may be synthesized by a conventional method (for example, radical polymerization). Examples of a general synthesis method may include a batch polymerization method of dissolving monomer species and an initiator in a solvent and heating the solution, thereby performing the polymerization, a dropping polymerization method of adding dropwise a solution containing monomer species and an initiator to a heated solvent over 1 to 10 hours, and the like, and a dropping polymerization method is preferred.

The reaction solvent, polymerization initiator, reaction conditions (temperature, concentration and the like) and purification method after reaction are the same as those described above in the resin (C).

Hereinafter, specific examples of the hydrophobic resin (HR) will be described. In addition, the molar ratio (in each resin as specific examples, the positional relationship of each repeating unit corresponds to the positional relationship of numbers in a composition ratio in Table 1), the weight average molecular weight and the polydispersity of the repeating unit in each resin are noted in the following Table 1.

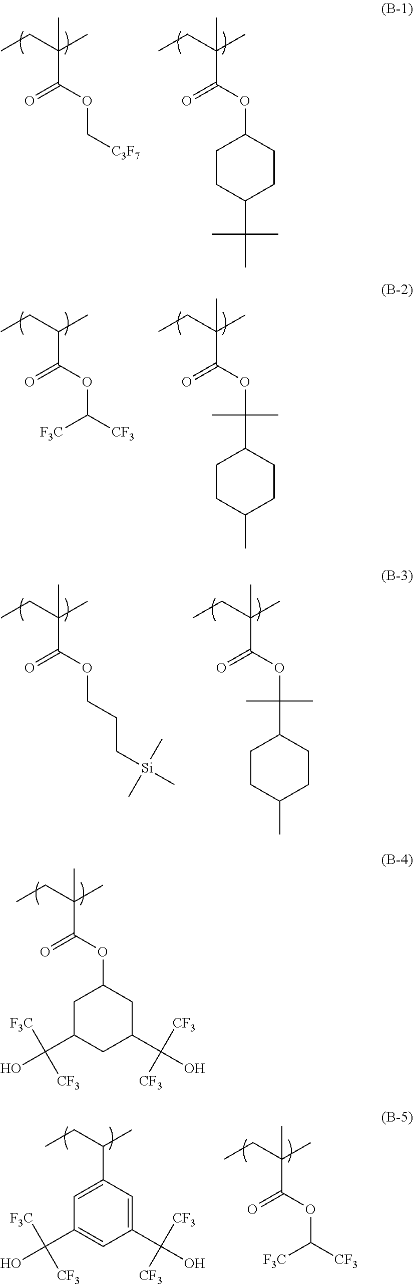

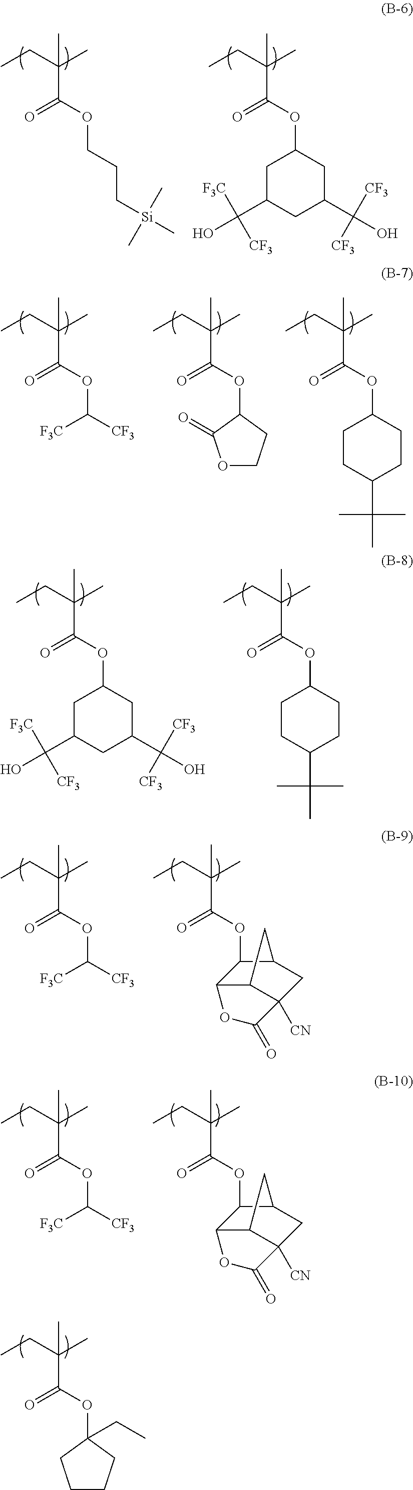

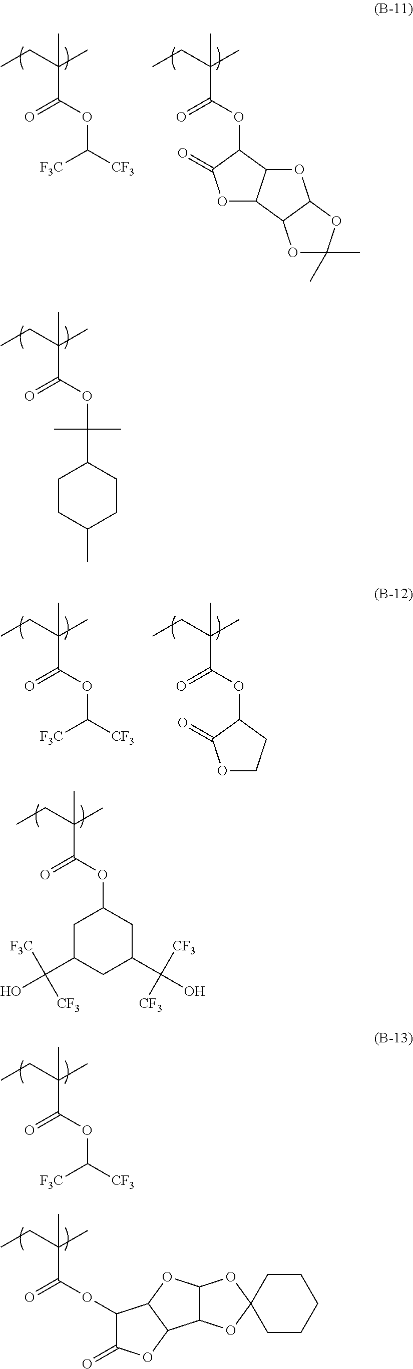

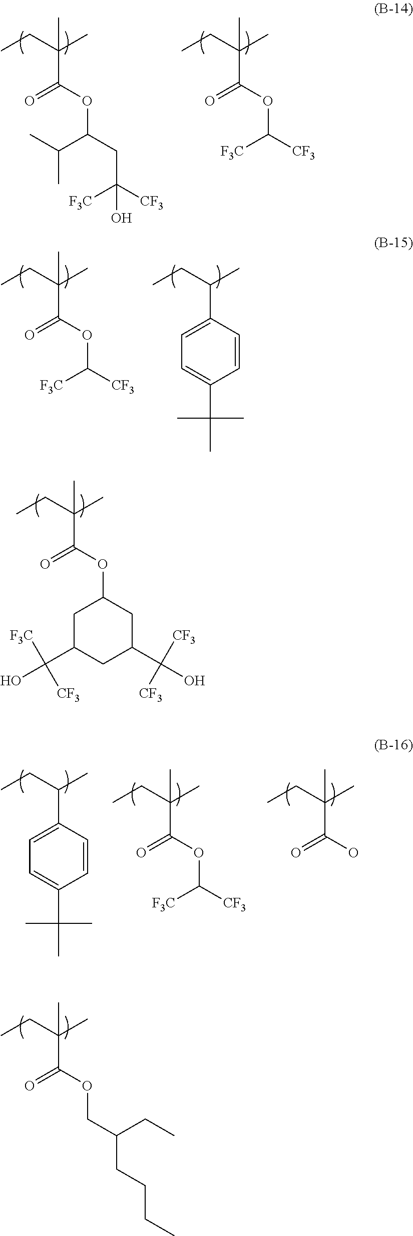

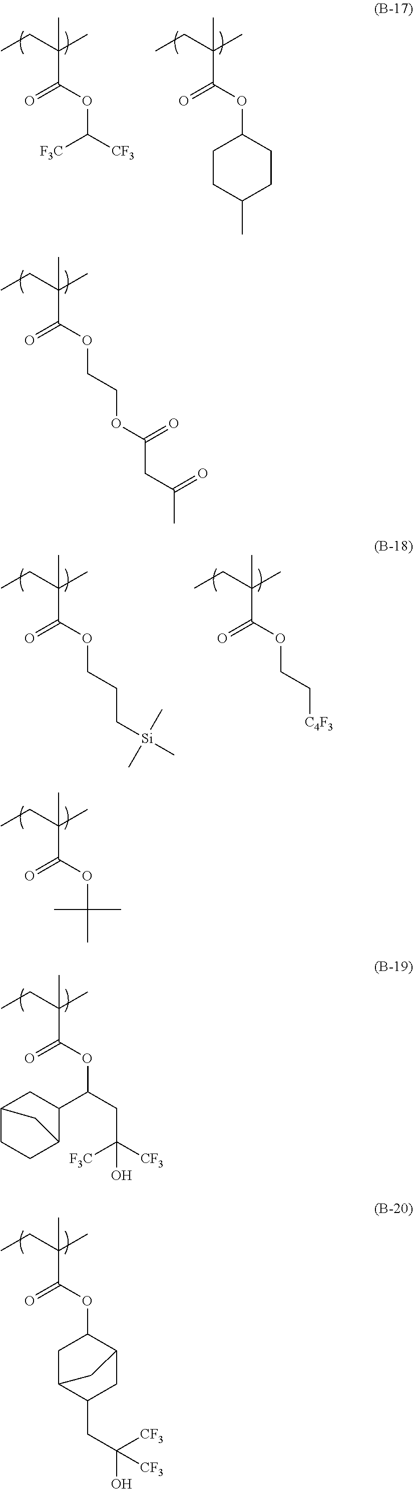

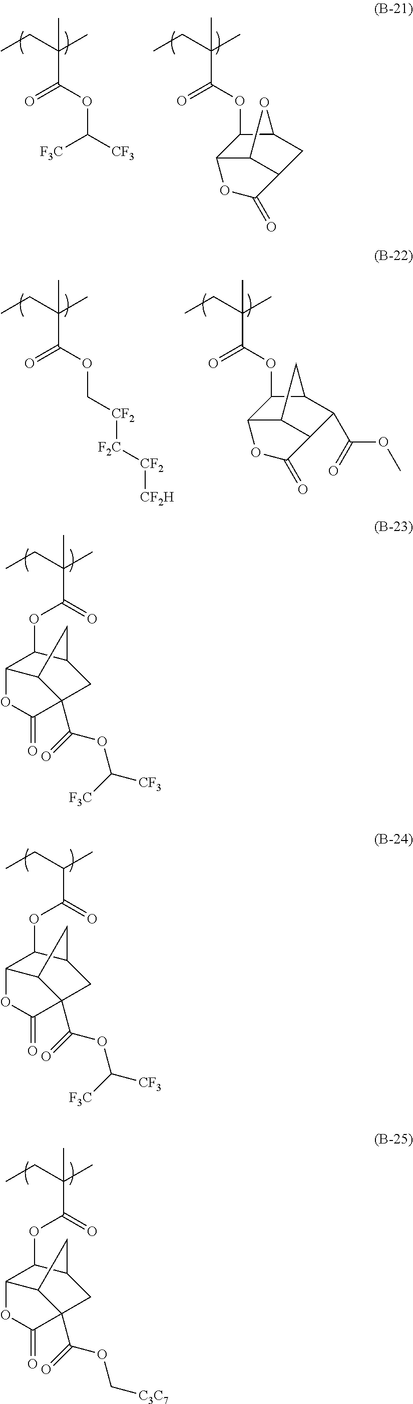

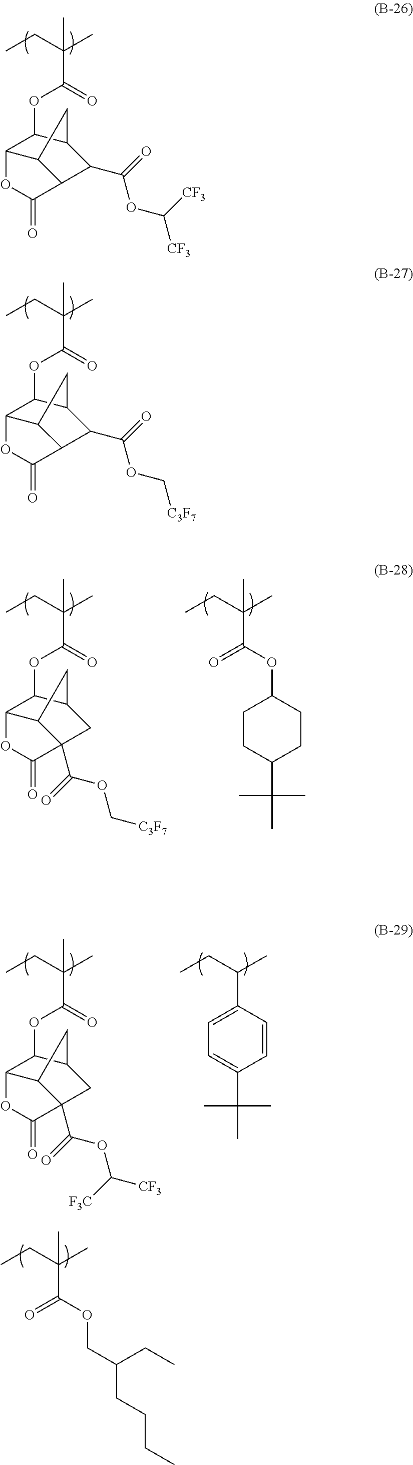

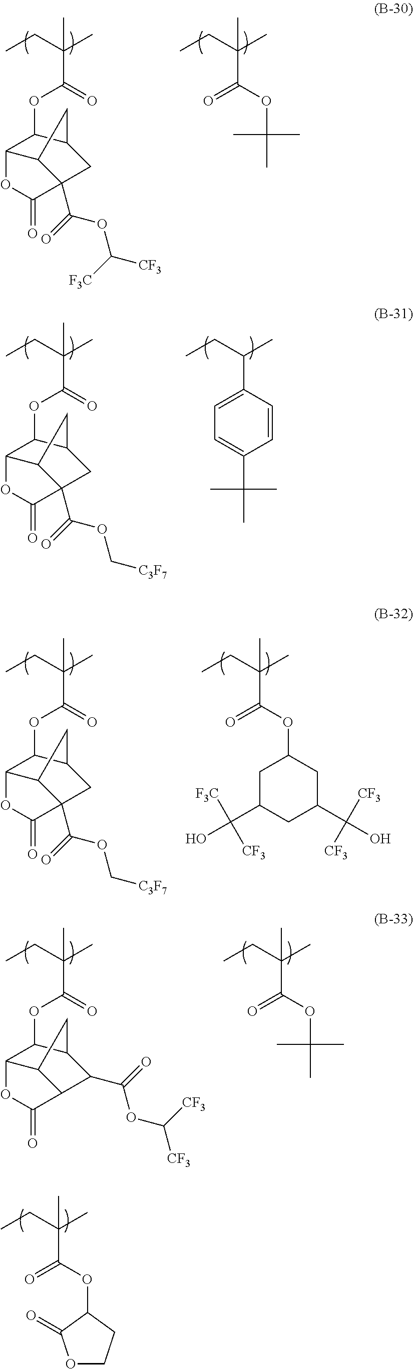

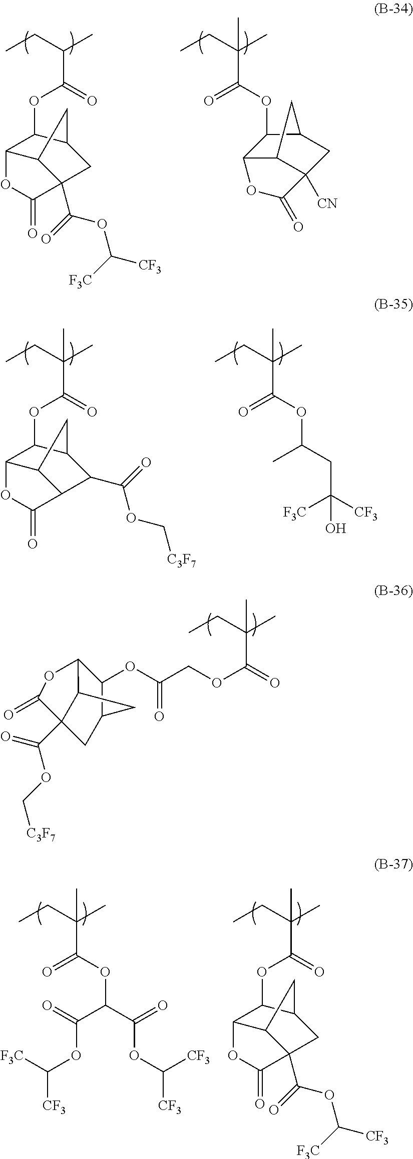

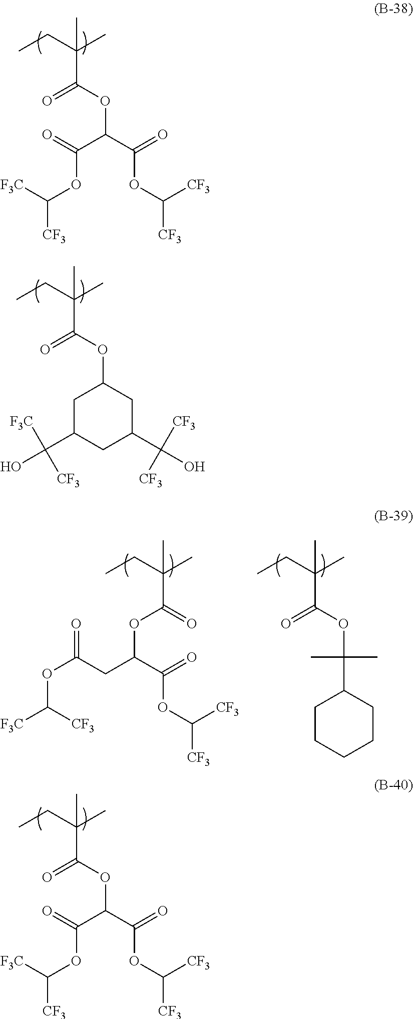

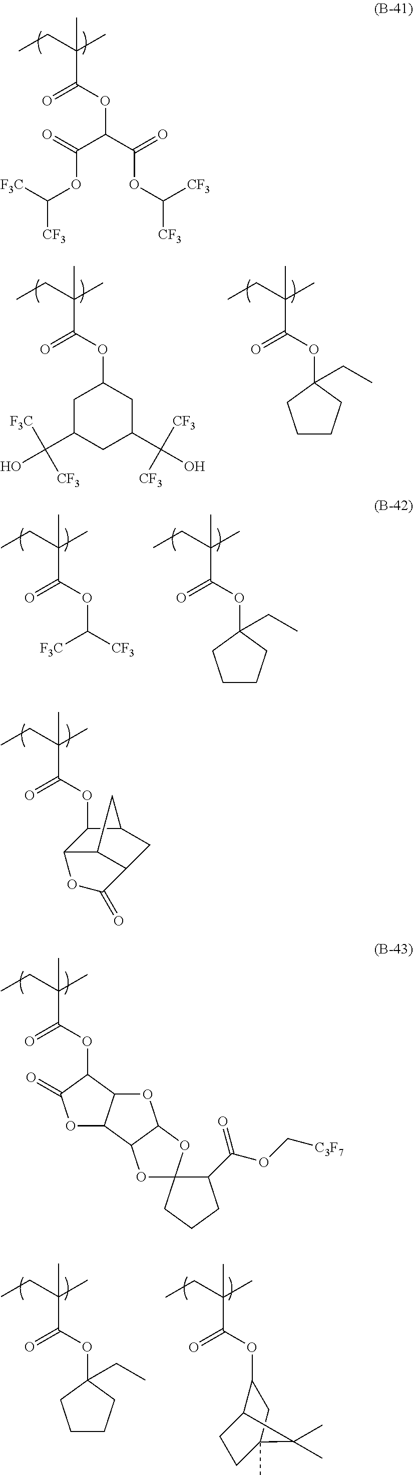

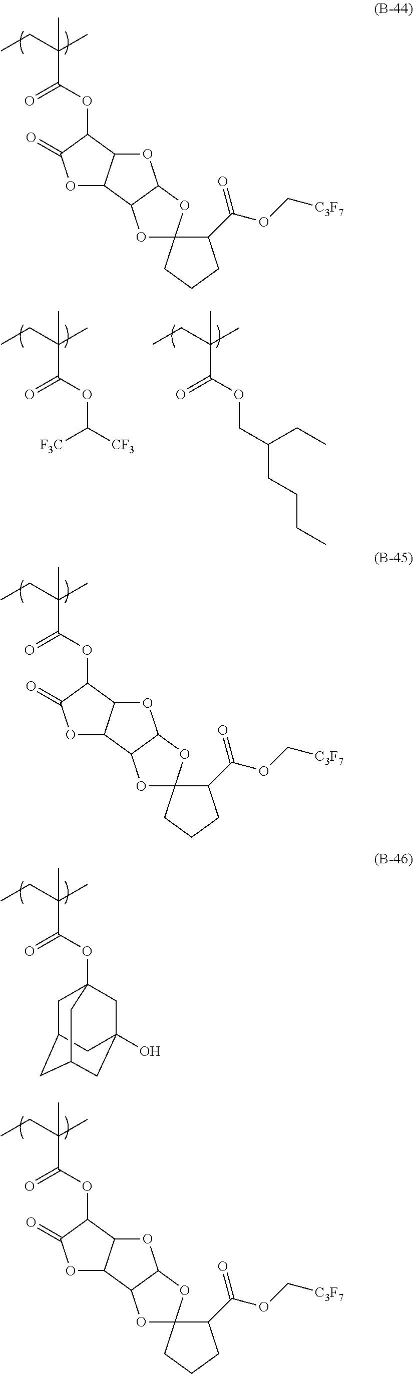

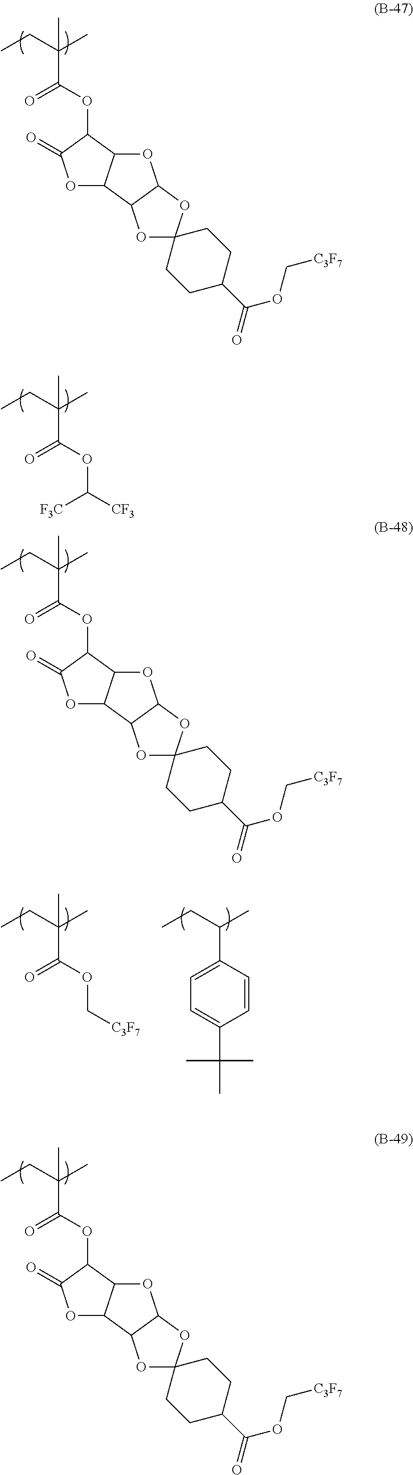

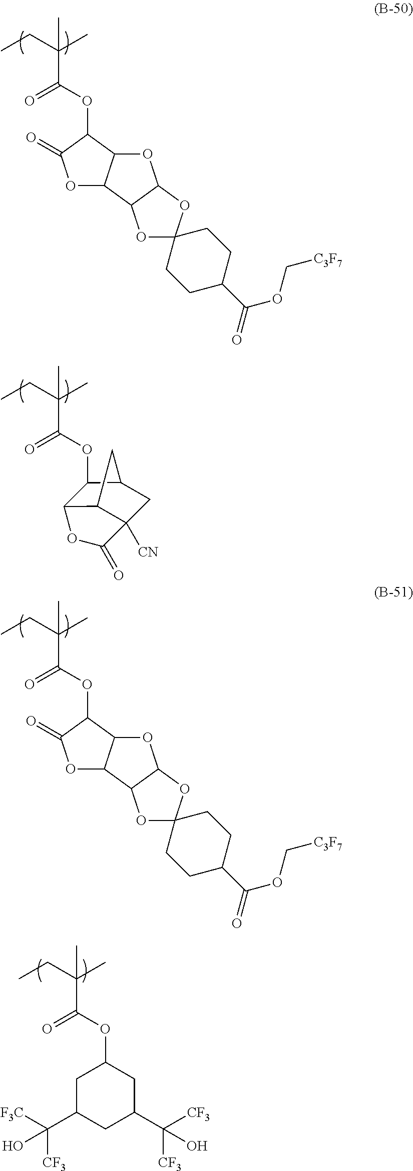

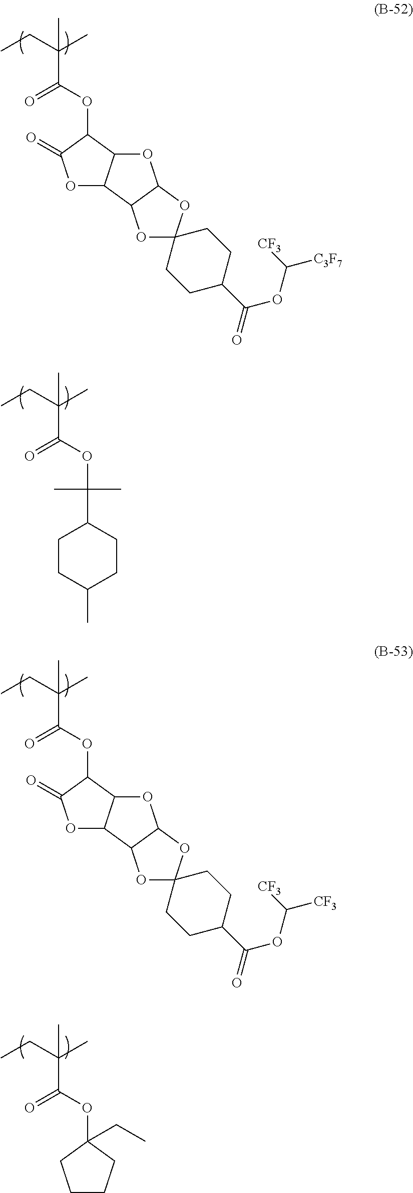

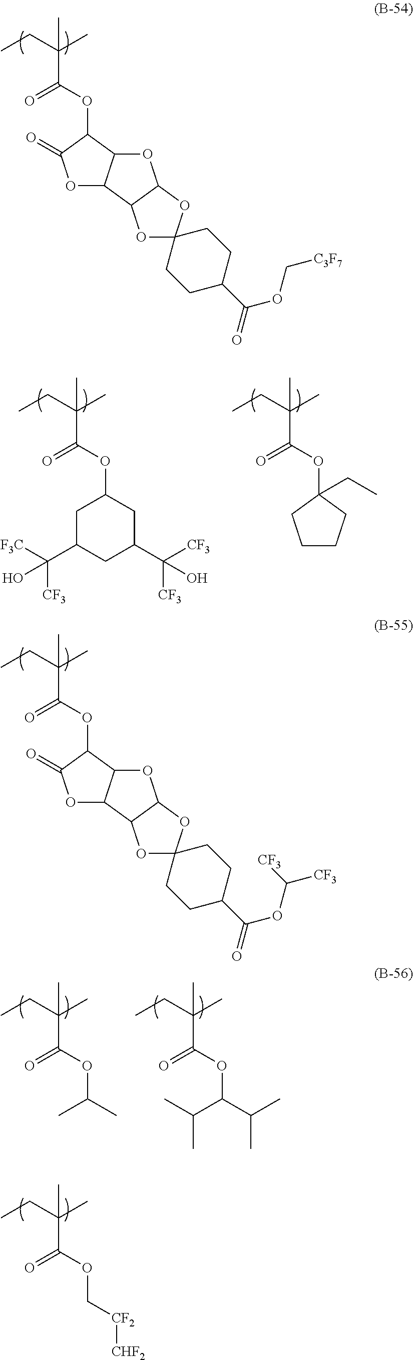

TABLE 1

| | Composition | | |
|---|---|---|---|
| Polyer | ratio (mol %) | Mw | Mw/Mn |
| B-1 | 50/50 | 6000 | 1.5 |
| B-2 | 30/70 | 6500 | 1.4 |
| B-3 | 45/55 | 8000 | 1.4 |
| B-4 | 100 | 15000 | 1.7 |
| B-5 | 60/40 | 6000 | 1.4 |
| B-6 | 40/60 | 8000 | 1.4 |
| B-7 | 30/40/30 | 8000 | 1.4 |
| B-8 | 60/40 | 8000 | 1.3 |
| B-9 | 50/50 | 6000 | 1.4 |
| B-10 | 40/40/20 | 7000 | 1.4 |
| B-11 | 40/30/30 | 9000 | 1.6 |
| B-12 | 30/30/40 | 6000 | 1.4 |
| B-13 | 60/40 | 9500 | 1.4 |
| B-14 | 60/40 | 8000 | 1.4 |
| B-15 | 35/35/30 | 7000 | 1.4 |
| B-16 | 50/40/5/5 | 6800 | 1.3 |
| B-17 | 20/30/50 | 8000 | 1.4 |
| B-18 | 25/25/50 | 6000 | 1.4 |
| B-19 | 100 | 9500 | 1.5 |
| B-20 | 100 | 7000 | 1.5 |
| B-21 | 50/50 | 6000 | 1.6 |
| B-22 | 40/60 | 9600 | 1.3 |
| B-23 | 100 | 20000 | 1.7 |
| B-24 | 100 | 25000 | 1.4 |
| B-25 | 100 | 15000 | 1.7 |
| B-26 | 100 | 12000 | 1.8 |
| B-27 | 100 | 18000 | 1.3 |
| B-28 | 70/30 | 15000 | 2.0 |
| B-29 | 80/15/5 | 18000 | 1.8 |
| B-30 | 60/40 | 25000 | 1.8 |
| B-31 | 90/10 | 19000 | 1.6 |
| B-32 | 60/40 | 20000 | 1.8 |
| B-33 | 50/30/20 | 11000 | 1.6 |
| B-34 | 60/40 | 12000 | 1.8 |
| B-35 | 60/40 | 15000 | 1.6 |
| B-36 | 100 | 22000 | 1.8 |
| B-37 | 20/80 | 35000 | 1.6 |
| B-38 | 30/70 | 12000 | 1.7 |
| B-39 | 30/70 | 9000 | 1.5 |
| B-40 | 100 | 9000 | 1.5 |
| B-41 | 40/15/15 | 12000 | 1.9 |
| B-42 | 30/30/40 | 13000 | 2.0 |
| B-43 | 40/40/20 | 23000 | 2.1 |
| B-44 | 65/30/5 | 25000 | 1.6 |
| B-45 | 100 | 15000 | 1.7 |
| B-46 | 20/80 | 9000 | 1.7 |
| B-47 | 70/30 | 18000 | 1.5 |
| B-48 | 60/20/20 | 18000 | 1.8 |
| B-49 | 100 | 12000 | 1.4 |
| B-50 | 60/40 | 20000 | 1.6 |
| B-51 | 70/30 | 33000 | 2.0 |
| B-52 | 60/40 | 19000 | 1.8 |
| B-53 | 50/50 | 15000 | 1.5 |
| B-54 | 40/20/40 | 35000 | 1.9 |
| B-55 | 100 | 16000 | 1.4 |
| B-56 | 30/65/5 | 28000 | 1.7 |

The actinic ray-sensitive or radiation-sensitive resin composition according to the present invention contains the hydrophobic resin containing at least one of a fluorine atom and a silicon atom. Accordingly, the hydrophobic resin is localized in the top layer of the film formed by the actinic ray-sensitive or radiation-sensitive resin composition. Thus, when the immersion medium is water, the receding contact angle of the film surface with respect to water after baking and before exposure is increased so that the immersion-liquid follow-up properties may be enhanced.

The receding contact angle of the coated film composed of the actinic ray-sensitive or radiation-sensitive resin composition of the present invention after the following of the film but prior to the exposure thereof is preferably in the range of 60° to 90°, more preferably 65° or greater, further more preferably 70° or greater and particularly preferably 75° or greater at the exposure temperature, generally room temperature 23±3° C. in a humidity of 45+5%.

Although the hydrophobic resin is designed to be localized at the interface as described above, unlike a surfactant, the hydrophobic resin does not necessarily have a hydrophilic group in the molecule thereof, and may not contribute to the homogeneous mixing of polar/non-polar materials.

In the operation of immersion exposure, it is needed for the immersion liquid to move on a wafer while following the movement of an exposure head involving high-speed scanning on the wafer and thus forming an exposure pattern. Therefore, the contact angle of the immersion liquid with respect to the resist film in dynamic condition is important, and it is required for the resist to be capable of following the high-speed scanning of the exposure head without leaving droplets.

The hydrophobic resin, due to its hydrophobicity, is likely to cause development residue (scum) and BLOB defect after alkali development to deteriorate. When the hydrophobic resin has three polymer chains via at least one branched portion, as compared with a straight resin, the alkali dissolution rate is increased to thereby improve the development residue (scum) and BLOB defect performances.

When the hydrophobic resin has a fluorine atom, the content of the fluorine atom is preferably 5% by mass to 80% by mass, and more preferably 10% by mass to 80% by mass based on the weight average molecular weight of the hydrophobic resin. The content of the repeating unit containing the fluorine atom is preferably 10 mol % to 100 mol %, and more preferably 30 mol % to 100 mol % based on the whole repeating units in the hydrophobic resin.

When the hydrophobic resin has a silicon atom, the content of the silicon atom is preferably 2% by mass to 50% by mass, and more preferably 2% by mass to 30% by mass based on the weight average molecular weight of the hydrophobic resin. The content of the repeating unit containing the silicon atom is preferably 10 mol % to 90 mol %, and more preferably 20 mol % to 80 mol % based on the whole repeating units in the hydrophobic resin.

The weight average molecular weight of the hydrophobic resin preferably is 1,000 to 100,000, more preferably 2,000 to 50,000, and still more preferably 3,000 to 35,000. Here, the weight average molecular weight of a resin refers to the polystyrene-equivalent molecular weight measured by GPC (carrier: tetrahydrofuran (THF)).

The content of the hydrophobic resin in the actinic ray-sensitive or radiation-sensitive resin composition may be appropriately adjusted so that the receding contact angle of the resist film may be within the above described range. Based on the total solid content of the actinic ray-sensitive or radiation-sensitive resin composition, the content of the resin is preferably 0.01% by mass to 20% by mass, more preferably 0.1% by mass to 15% by mass, still more preferably 0.1% by mass to 10% by mass, and particularly preferably 0.2% by mass to 8% by mass.

The hydrophobic resin may be used either alone or in combination of two or more kinds thereof.

[6] Resin (D) containing substantially no fluorine atom and silicon atom, which is different from the resin (B)

The actinic ray-sensitive or radiation-sensitive resin composition according to the present invention may contain a resin (D) containing substantially no fluorine atom and silicon atom, which is different from the resin (B) (hereinafter, simply referred to as a “resin (D)”) in an amount of 0.1% by mass or more and less than 10% by mass based on the total solid of the actinic ray-sensitive or radiation-sensitive resin composition.

Here, the resin (D) contains substantially no fluorine atom and silicon atom, but specifically, the content of the repeating unit having a fluorine atom or a silicon atom is preferably 5 mol % or less, more preferably 3 mol % or less, and still more preferably 1 mol % or less based on the whole repeating units in the resin (D), and is ideally 0 mol %, that is, contains no fluorine atom and silicon atom.

From the viewpoints of improving the uniformity of a local pattern dimension and EL, and achieving reduction in watermark defect by localizing the resin (D) in the top layer portion of the resist film, the content of the resin (D) of the present invention is preferably 0.1% by mass or more and less than 10% by mass, more preferably 0.2% by mass to 8% by mass, still more preferably 0.3% by mass to 6% by mass, and particularly preferably 0.5% by mass to 5% by mass based on the total solid of the actinic ray-sensitive or radiation-sensitive resin composition.

Further, the mass content ratio of the CH₃ partial structure possessed by the side chain moiety in the resin (D) is 12.0% or more, and preferably 18.0% or more in the resin (D). Accordingly, a low surface free energy may be achieved and the localization of the resin (D) in the top layer portion of the resist film may be achieved. As a result, the uniformity of a local pattern dimension (uniformity of the hole diameter in formation of a fine hole pattern) and EL are excellent, and the reduction in watermark defect in the immersion exposure may be achieved.

Further, the upper limit of the mass content ratio of the CH₃ partial structure possessed by the side chain moiety in the resin (D) is preferably 50% or less, and more preferably 40% or less.

Here, a methyl group (for example, an α-methyl group of the repeating unit having a methacrylic acid structure) directly bonded to the main chain of the resin (1) slightly contributes to the surface uneven distribution of the resin (D) due to the effects of the main chain and thus is not included in the CH₃ partial structure in the present invention and is not counted. More specifically, when the resin (D) includes a repeating unit derived from a monomer having a polymerizable moiety having a carbon-carbon double bond, such as, for example, a repeating unit represented by the following Formula (M) and when R₁₁ to R₁₄ are a CH₃ “as it is”, the CH₃ is not included (not counted) in the CH₃ partial structure in the present invention possessed by the side chain moiety.

Meanwhile, the CH₃ partial structure present through any atom from the C—C main chain is counted as a CH₃ partial structure in the present invention. For example, when R₁, is an ethyl group (CH₂CH₃), R₁₁ is counted to have “one” CH₃ partial structure in the present invention.

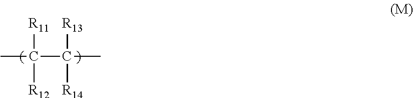

In Formula (M).

R₁₁ to R₁₄ each independently represent a side chain moiety.

Examples of R₁₁ to R₁₄ in the side chain moiety may include a hydrogen atom, a monovalent organic group and the like.

Examples of the monovalent organic group for R₁₁ to R₁₄ may include an alkyl group, a cycloalkyl group, an aryl group, an alkyloxycarbonyl group, a cycloalkyloxycarbonyl group, an aryloxycarbonyl group, an alkylaminocarbonyl group, a cycloalkylaminocarbonyl group, an arylaminocarbonyl group and the like.

The monovalent organic group may further have a substituent, and examples of the substituent may be the same as specific examples and preferred examples described below as a substituent which may be possessed by the aromatic group Ar₂₁ in Formula (11).

In the present invention, the CH₃ partial structure (hereinafter, simply referred to as a “side chain CH₃ partial structure”) possessed by the side chain moiety in the resin (D) includes a CH₃ partial structure possessed by an ethyl group, a propyl group and the like.

Hereinafter, the mass content ratio (hereinafter, simply referred to as a “mass content ratio of the side chain CH₃ partial structure in the resin (D)”) of the CH₃ partial structure possessed by the side chain moiety in the resin (D), occupied in the resin (D), will be described.

Here, the mass content ratio of the side chain CH₃ partial structure in the resin (D) will be described by exemplifying the case in which the resin (D) is composed of the repeating units D1, D2, . . . , Dx, . . . , and Dn, and each of the molar ratios of the repeating units D1, D2, . . . , Dx, . . . , and Dn in the resin (D) is ω1, ω2, . . . , ωx, . . . , and on.

(1) First, the mass content ratio (MCx) of the side chain CH₃ partial structure of the repeating unit Dx may be calculated by an equation of “100×15.03×(the number of CH₃ partial structures in the side chain moiety in the repeating unit Dx)/molecular weight (Mx) of the repeating unit Dx”.

Here, the number of CH₃ partial structures in the side chain moiety in the repeating unit Dx does not include the number of methyl groups directly bonded to the main chain thereof.

(2) Next, the mass content ratio of the side chain CH₃ partial structure in the resin (D) may be calculated by the following equation by using the mass content ratio of the side chain CH₃ partial structure calculated for each repeating unit.

Mass content ratio of the side chain CH₃ partial structure in the resin (D):

DMC=Σ[(ω1×MC1)+(ω2×MC2)+ . . . +(ωx×MCx)+ . . . +(ωn×MCn)]

Specific examples of the mass content ratio of the CH₃ partial structure in the side chain moiety in the repeating unit Dx are described below, but the present invention is not limited thereto.

| | | | Mass |
|---|---|---|---|
| | | Number of | content of |
| Structure of | Mw of | CH₃ partial | side chain |
| repeating | repeating | structure | CH₃ partial |
| unit | unit | in side chain | structure |
| 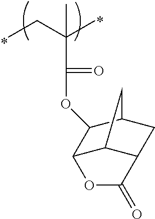 | 222.24 | 0 | 0.0% |
| 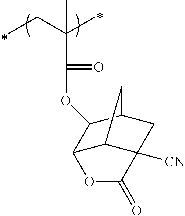 | 247.25 | 0 | 0.0% |
| 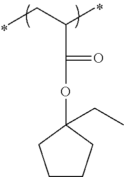 | 168.23 | 1 | 8.9% |
| 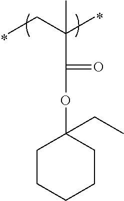 | 196.29 | 1 | 7.7% |
| 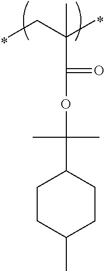 | 224.34 | 3 | 20.1% |
| 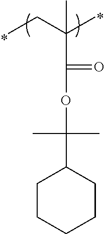 | 210.31 | 2 | 14.3% |
| 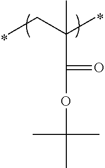 | 142.2 | 3 | 31.7% |
| 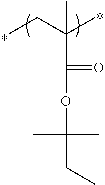 | 156.22 | 3 | 28.9% |
| 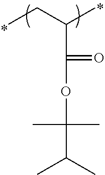 | 156.22 | 4 | 38.5% |
| 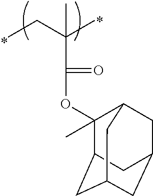 | 234.33 | 1 | 6.4% |
| 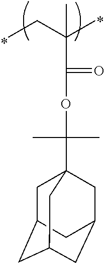 | 262.39 | 2 | 11.5% |
| 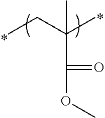 | 100.12 | 1 | 15.0% |
| 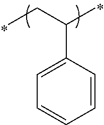 | 104.15 | 0 | 0.0% |
| 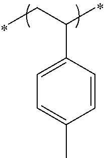 | 118.18 | 1 | 12.7% |
| 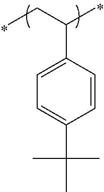 | 160.26 | 3 | 28.1% |
| 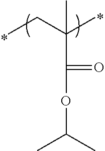 | 128.17 | 2 | 23.5% |
| 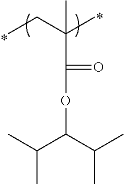 | 184.28 | 4 | 32.6% |
| 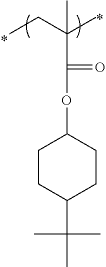 | 224.34 | 3 | 20.1% |
| 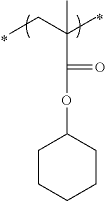 | 168.23 | 0 | 0.0% |
| 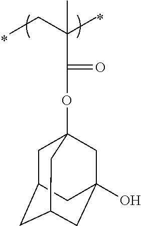 | 236.31 | 0 | 0.0% |

Specific examples of the mass content ratio of the side chain CH₃ partial structure in the resin (D) are noted in Table 3 below, but the present invention is not limited thereto.

| | | Mass content of |
|---|---|---|
| | | side chain CH₃ |
| Structure of | Composition | partial structure |
| resin (D) | ratio (mol %) | in resin (D) |
| 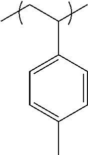 | 100 | 12.7 |
| 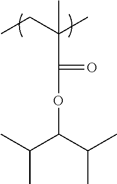 | 100 | 32.6 |
| 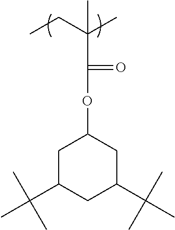 | 100 | 32.2 |
| 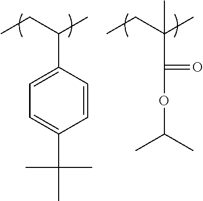 | 30/70 | 25.9 |
| 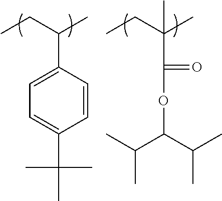 | 10/90 | 32.5 |
| 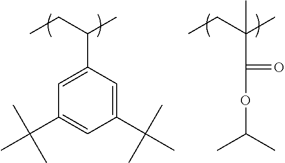 | 15/85 | 26.2 |
| 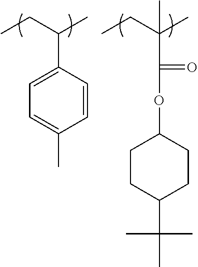 | 15/85 | 19.0 |
| 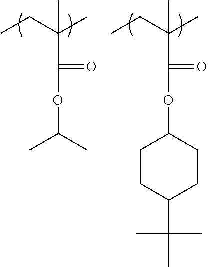 | 50/50 | 21.8 |
| 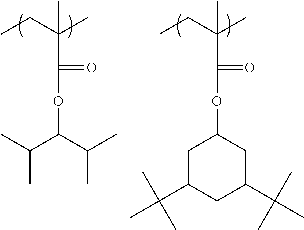 | 60/40 | 32.4 |
| 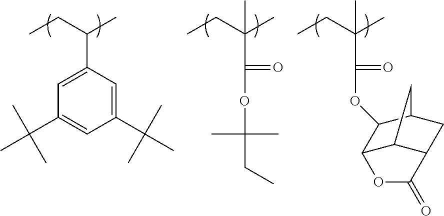 | 40/50/10 | 31.1 |
| 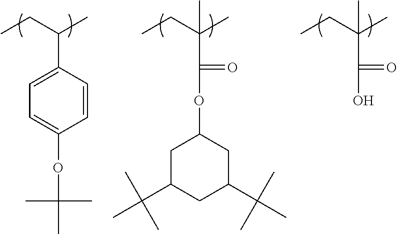 | 10/85/5 | 29.9 |
| 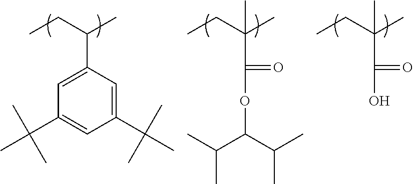 | 40/55/5 | 38.8 |
| 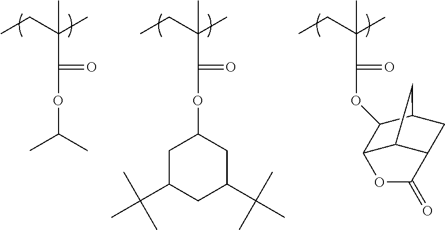 | 50/45/5 | 26.2 |
| 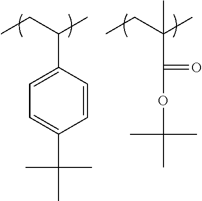 | 20/80 | 28.1 |
| 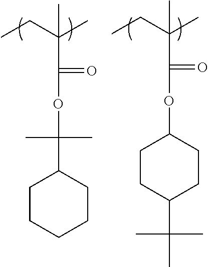 | 50/50 | 20.1 |
| 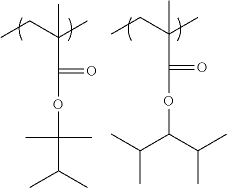 | 40/60 | 33.7 |

The resin (D) preferably has at least one of the repeating units represented by the following Formula (V) or (VI), and is more preferably composed only of at least one of the repeating units represented by the following Formula (V) or (VI).

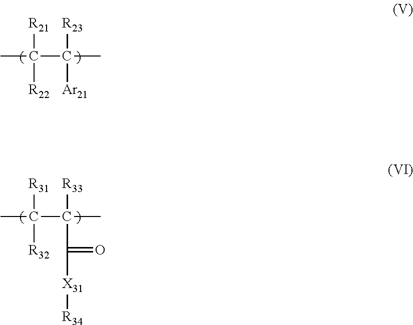

In Formula (V),

R₂₁ to R₂₃ each independently represent a hydrogen atom or an alkyl group.

Ar₂₁ represents an aromatic group. R₂₂ and Ar₂₁ may form a ring, and in this case, R₂₂ represents an alkylene group.

In Formula (VI),

R₃₁ to R₃₃ each independently represent a hydrogen atom or alkyl group.

X₃₁ represents —O— or —NR₃₅—. R₃₅ represents a hydrogen atom or an alkyl group.

R₃₄ represents an alkyl group or a cycloalkyl group.

In Formula (V), the alkyl group of R₂₁ to R₂₃ is preferably an alkyl group having 1 to 4 carbon atoms (a methyl group, an ethyl group, a propyl group or a butyl group), more preferably a methyl group or an ethyl group, and particularly preferably a methyl group.

When R₂₂ and Ar₂₁ form a ring, examples of the alkylene group may include a methylene group, an ethylene group and the like.

In Formula (V), R₂₁ to R₂₃ are particularly preferably a hydrogen atom or a methyl group.

The aromatic group of Ar₂₁ in Formula (V) may have a substituent, and examples thereof may include an aryl group having 6 to 14 carbon atoms, such as a phenyl group and a naphthyl group, or an aromatic group containing a heterocyclic ring such as thiophene, furan, pyrrole, benzothiophene, benzofuran, benzopyrrole, triazine, imidazole, benzoimidazole, triazole, thiadiazole and thiazole. An aryl group which may have a substituent having 6 to 14 carbon atoms such as a phenyl group or a naphthyl group is preferred.

Examples of the substituent which may be possessed by the aromatic group Ar₂₁ may include an alkyl group, an alkoxy group, an aryl group and the like, but from the viewpoint of increasing the mass content ratio of the CH₃ partial structure contained in the side chain moiety in the resin (D), and lowering the surface free energy, the substituent is preferably an alkyl group or an alkoxy group, more preferably an alkyl group having 1 to 4 carbon atoms or an alkoxy group, and particularly preferably a methyl group, an isopropyl group, a t-butyl group or a t-butoxy group.

Further, the aromatic group for Ar₂₁ may have two or more substituents.

In Formula (VI), the alkyl group of R₃₁ to R₃₃ and R₃₅ is preferably an alkyl group having 1 to 4 carbon atoms (a methyl group, an ethyl group, a propyl group and a butyl group), more preferably a methyl group and an ethyl group, and particularly preferably a methyl group.

It is particularly preferred that R₃₁ to R₃₃ in Formula (III) each independently a hydrogen atom or a methyl group.

In Formula (VI), X₃₁ is preferably —O—, or —NH— (that is, when R₃₅ in —NR₃₅— is a hydrogen atom) and particularly preferably —O—.

In Formula (VI), the alkyl group for R₃₄ may be either chained or branched, and examples thereof may include a chained alkyl group (for example, a methyl group, an ethyl group, a n-propyl group, a n-butyl group, a n-hexyl group, a n-octyl group, a n-dodecyl group and the like) and a branched alkyl group (for example, an isopropyl group, an isobutyl group, a tert-butyl group, a methylbutyl group, a dimethylpentyl group and the like), but from the viewpoint of increasing the mass content ratio of the CH₃ partial structure contained in the side chain moiety in the resin (D) to lower the surface free energy, the alkyl group is preferably a branched alkyl group, more preferably a branched alkyl group having 3 to 10 carbon atoms, and particularly preferably a branched alkyl group having 3 to 8 carbon atoms.

In Formula (III), the cycloalkyl group for R₃₄ may have a substituent, and examples thereof may include a monocyclic cycloalkyl group such as a cyclobutyl group, a cyclopentyl group and a cyclohexyl group, and a polycyclic cycloalkyl group such as a norbornyl group, a tetracyclodecanyl group and an adamantyl group, but the cycloalkyl group is preferably a monocyclic cycloalkyl group, more preferably a monocycle cycloalkyl group having 5 to 6 carbon atoms, and particularly preferably a cyclohexyl group.

Examples of the substituent which may be possessed by R₃₄ may include an alkyl group, an alkoxy group, an aryl group and the like, but from the viewpoint of increasing the mass content ratio of the CH₃ partial structure contained in the side chain moiety in the resin (D) to lower the surface free energy, the substituent is preferably an alkyl group or an alkoxy group, more preferably an alkyl group having 1 to 4 carbon atoms or an alkoxy group, and particularly preferably a methyl group, an isopropyl group, a t-butyl group and a t-butoxy group.

Further, the cycloalkyl group for R₃₄ may have two or more substituents.

It is preferred that R₃₄ is not a group capable of decomposing and leaving by the action of an acid, that is, the repeating unit represented by Formula (VI) is not a repeating unit having an acid-decomposable group.

In Formula (VI), R₃₄ is most preferably a cyclohexyl group substituted with a branched alkyl group having 3 to 8 carbon atoms, an alkyl group having 1 to 4 carbon atoms or an alkoxy group.

Specific examples of the repeating unit represented by Formula (V) or (VI) are shown below, but the present invention is not limited thereto.

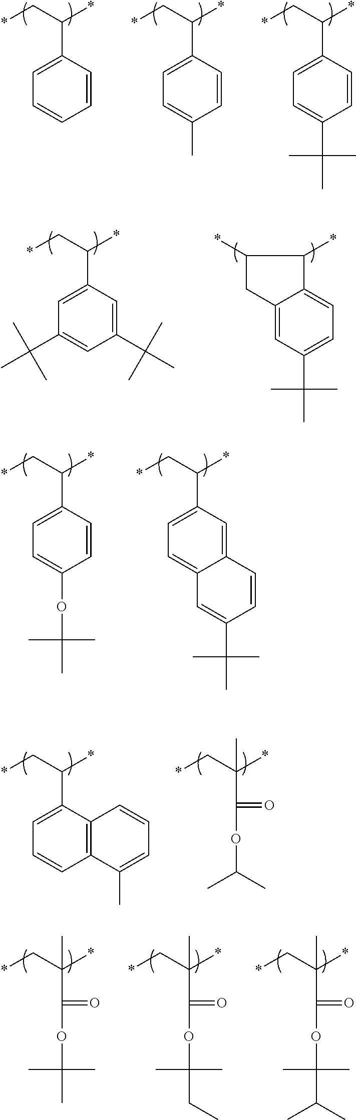

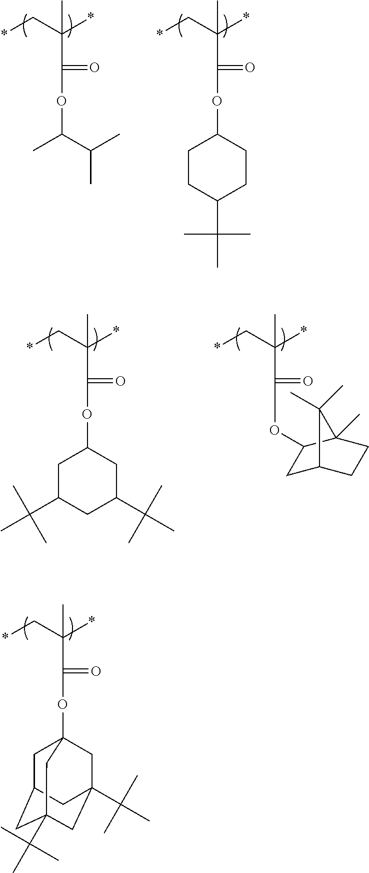

When the resin (D) has the repeating unit represented by Formula (V) or (VI), the content of the repeating unit represented by Formula (V) or (VI) is preferably in a range of 50 mol % to 100 mol %, more preferably in a range of 65 mol % to 100 mol %, and particularly preferably in a range of 80 mol % to 100 mol % based on the whole repeating units in the resin (D), from the viewpoint of lowering the surface free energy, thereby achieving the effects of the present invention.

The resin (D) may further have, as appropriate, a repeating unit having an acid-decomposable group, a repeating unit having a lactone structure, a repeating unit having a hydroxyl group or a cyano group, a repeating unit having an acid group (an alkali-soluble group), or a repeating unit having an alicyclic hydrocarbon structure having no polar group and not exhibiting acid decomposability, as described above for the resin (B).

Specific examples and preferred examples of each repeating unit that the resin (D) may have are the same as the specific examples and preferred examples of each repeating unit described above for the resin (B).

However, from the viewpoint of achieving the effects of the present invention, it is more preferred that the resin (D) does not have a repeating unit having an acid-decomposable group, an alkali-soluble repeating unit and a repeating unit having a lactone structure.

The weight average molecular weight of the resin (D) according to the present invention is not particularly limited, but the weight average molecular weight is preferably in a range of 3.000 to 100,000, more preferably in a range of 6,000 to 70,000, and particularly preferably in a range of 10,000 to 40,000. In particular, by adjusting the weight average molecular weight in a range of 10,000 to 40,000, a Local CDU and an exposure latitude are excellent in forming a fine hole pattern, and defect reduction performance is excellent in the immersion exposure. Here, the weight average molecular weight of the resin represents the molecular weight in terms of polystyrene measured by GPC (carrier: THF or N-methyl-2-pyrrolidone (NMP)).

In addition, the polydispersity (Mw/Mn) is preferably 1.00 to 5.00, more preferably 1.03 to 3.50 and still more preferably 1.05 to 2.50. The smaller the molecular weight distribution is, the better the resolution and resist pattern shape are.

The resin (D) according to present invention may be used either alone or in combination of two or more thereof.

As for the resin (D), various commercially available products may be used, and the resin (D) may be synthesized by a conventional method (for example, radical polymerization). Examples of a general synthesis method may include a batch polymerization method of dissolving monomer species and an initiator in a solvent and heating the solution, thereby performing the polymerization, a dropping polymerization method of adding dropwise a solution containing monomer species and an initiator to a heated solvent over 1 to 10 hours, and the like, and a dropping polymerization method is preferred.

The reaction solvent, polymerization initiator, reaction conditions (temperature, concentration and the like) and purification method after reaction are the same as those described in the resin (C), but in the synthesis of the resin (D), the reaction concentration is preferably 10% by mass to 50% by mass.

Specific examples of the resin (D) are shown below, but the present invention is not limited thereto.

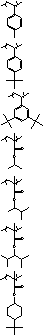

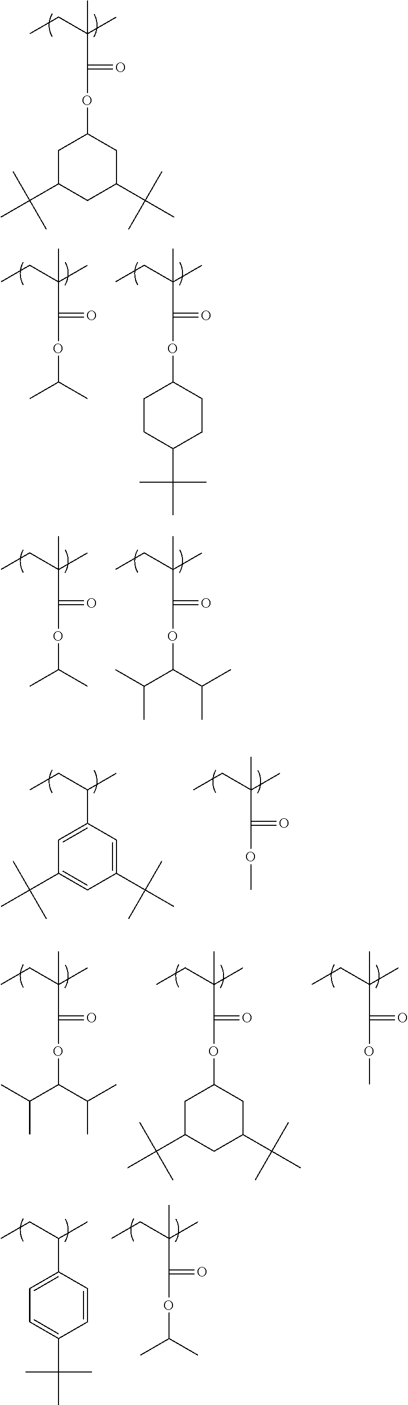

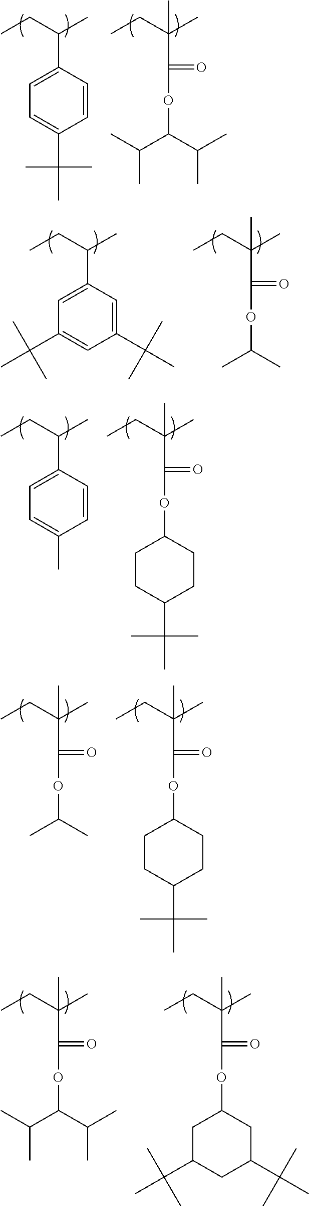

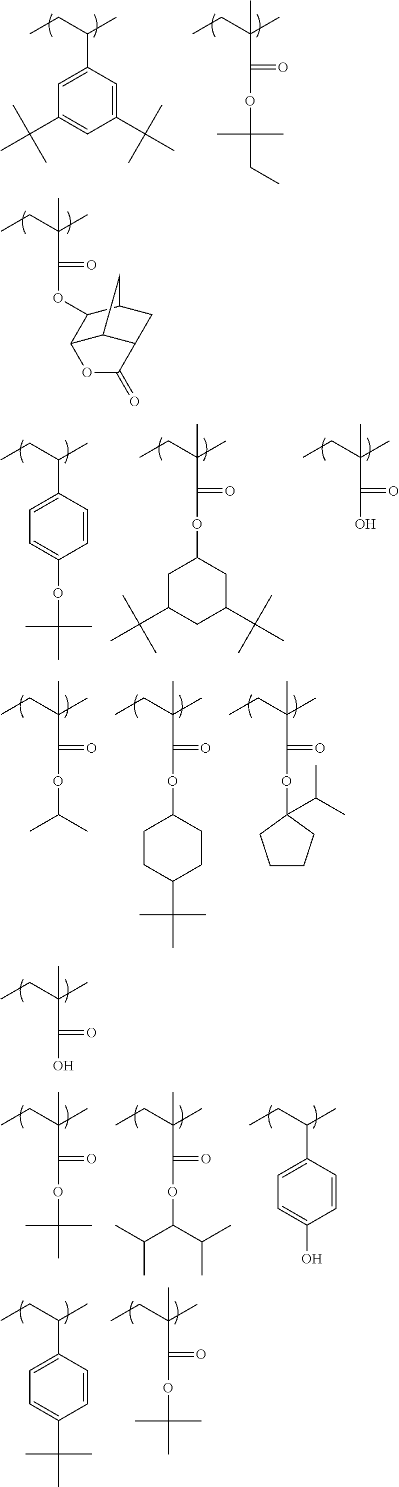

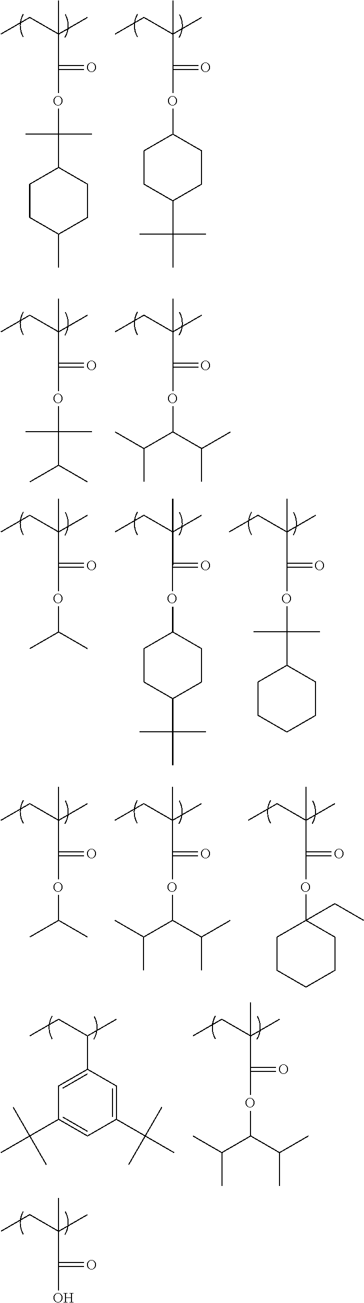

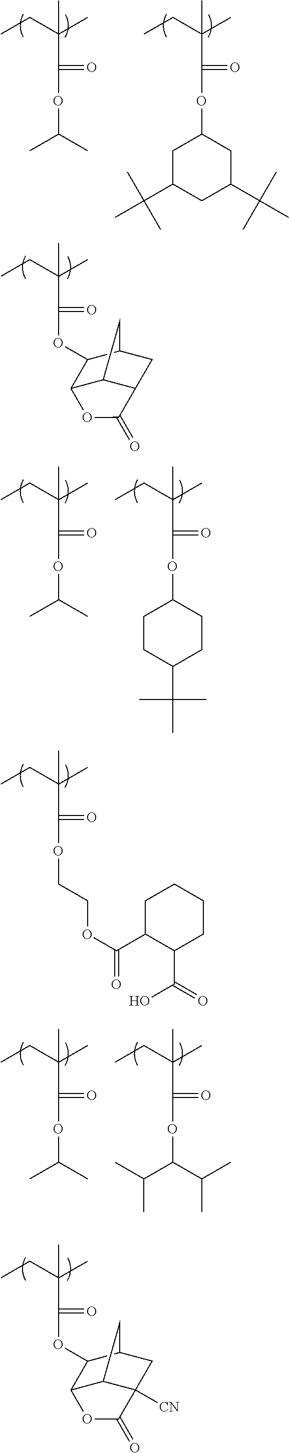

[7] Surfactant

The composition of the present invention may or may not further contain a surfactant. The surfactant is preferably fluorine-based and/or silicon-based surfactants.

Examples of the surfactant corresponding to them may include Megafac F176 and Megafac R08 (manufactured by DIC Corporation), PF656 and PF6320 (manufactured by OMNOVA Inc.), Troy Sol S-366 (manufactured by Troy Chemical Co., Ltd.), Fluorad FC430 (manufactured by Sumitomo 3M Limited), a polysiloxane polymer KP-341 (manufactured by Shin-Etsu Chemical Co., Ltd.) and the like.

Further, it is possible to use other surfactants other than flurorine-based and/or silicon-based surfactants. More specific examples thereof may include polyoxyethylene alkyl ethers, polyoxyethylene alkylaryl ethers and the like.

Besides, known surfactants may be appropriately used. Examples of available surfactants may include surfactants described after [0273] of U.S. Patent Application Laid-Open No. 2008/0248425A1.

The surfactants may be used either alone or in combination of two or more thereof.

The actinic ray-sensitive or radiation-sensitive resin composition of the present invention may or may not further contain a surfactant, but in the case of containing a surfactant, the amount of the surfactant used is preferably 0% by mass to 2% by mass, more preferably 0.0001% by mass to 2% by mass, and particularly preferably 0.0005 mol % to 1 mol % based on the total solid of the composition. Meanwhile, by setting the amount of the surfactant added to 10 ppm or less, the surface localization of the hydrophobic resin is increased, and accordingly, the surface of the resist film may be made more hydrophobic, thereby improving the water follow-up property at the time of immersion exposure.

[8] Solvent

The actinic ray-sensitive or radiation-sensitive resin composition according to the present invention usually further contains a solvent.

Examples of the solvent may include an organic solvent such as alkylene glycol monoalkyl ether carboxylate, alkylene glycol monoalkyl ether, alkyl lactate ester, alkyl alkoxypropionate, cyclic lactone (preferably having 4 to 10 carbon atoms), a monoketone compound (preferably having 4 to 10 carbon atoms) which may contain a ring, alkylene carbonate, alkyl alkoxyacetate and alkyl pyruvate.

Examples of alkylene glycol monoalkyl ether carboxylate may include preferably propylene glycol monomethyl ether acetate (POMEA; another name: 1-methoxy-2acetoxylpropane), propylene glycol monoethyl ether acetate, propylene glycol monopropyl ether acetate, propylene glycol monobutyl ether acetate, propylene glycol monomethyl ether propionate, propylene glycol monoethyl ether propionate, ethylene glycol monomethyl ether acetate and ethylene glycol monoethyl ether acetate.

Examples of alkylene glycol monoalkyl ether may include preferably propylene glycol monomethyl ether (PGME; another name: 1-methoxy-2-propanol), propylene glycol monoethyl ether, propylene glycol monopropyl ether, propylene glycol monobutyl ether, ethylene glycol monomethyl ether and ethylene glycol monoethyl ether.

Examples of alkyl lactate ester may include preferably methyl lactate, ethyl lactate, propyl lactate and butyl lactate.

Examples of alkyl alkoxypropionate may include preferably ethyl 3-ethoxypropionate, methyl 3-methoxypropionate, methyl 3-ethoxypropionate and ethyl 3-methoxypropionate.

Examples of cyclic lactone may include preferably β-propiolactone, β-butyrolactone, γ-butyrolactone, α-methyl-γ-butyrolactone, β-methyl-γ-butyrolactone, γ-valerolactone, γ-caprolactone, γ-octanoic lactone and α-hydroxy-γ-butyrolactone.

Examples of the monoketone compound which may contain a ring may include preferably 2-butanone, 3-methylbutanone, pinacolone, 2-pentanone, 3-pentanone, 3-methyl-2-pentanone, 4-methyl-2-pentanone, 2-methyl-3-pentanone, 4,4-dimethyl-2-pentanone, 2,4-dimethyl-3-pentanone, 2,2,4,4-tetramethyl-3-pentanone, 2-hexanone, 3-hexanone, 5-methyl-3-hexanone, 2-heptanone, 3-heptanone, 4-heptanone, 2-methyl-3-heptanone, 5-methyl-3-heptanone, 2,6-dimethyl-4-heptanone, 2-octanone, 3-octanone, 2-nonanone, 3-nonanone, 5-nonanone, 2-decanone, 3-decanone, 4-decanone, 5-hexen-2-one, 3-penten-2-one, cyclopentanone, 2-methylcyclopentanone, 3-methylcyclopentanone, 2,2-dimethylcyclopentanone, 2,4,4-trimethylcyclopentanone, cyclohexanone, 3-methylcyclohexanone, 4-methylcyclohexanone, 4-ethylcyclohexanone, 2,2-dimethylcyclohexanone, 2,6-dimethylcyclohexanone, 2,2,6-trimethylcyclohexanone, cycloheptanone, 2-methylcycloheptanone and 3-methylcycloheptanone.

Examples of alkylene carbonate may include preferably propylene carbonate, vinylene carbonate, ethylene carbonate and butylene carbonate.

Examples of alkyl alkoxyacetate may include preferably 2-methoxyethyl acetate, 2-ethoxyethyl acetate, 2-(2-ethoxyethoxyl)ethyl acetate, 3-methoxy-3methylbutyl acetate and 1-methoxy-2-propyl acetate.

Examples of alkyl pyruvate may include preferably methyl pyruvate, ethyl pyruvate and propyl pyruvate.

Examples of a solvent which may be used preferably may include a solvent whose boiling point is 130° C. or higher at room temperature and atmospheric pressure. Specific examples thereof may include cyclopentanone, γ-butyrolactone, cyclohexanone, ethyl lactate, ethylene glycol monoethyl ether acetate, PGMEA, ethyl 3-ethoxypropionate, ethyl pyruvate, 2-ethoxyethyl pyruvate 2-(2-ethoxyethoxyl)ethyl acetate and propylene carbonate.

In the present invention, the solvent may be used either alone or in combination of two or more thereof.

In the present invention, a mixed solvent prepared by mixing a solvent containing a hydroxyl group in the structure and a solvent containing no hydroxyl group may be used as the organic solvent.

The solvent containing a hydroxyl group and the solvent containing no hydroxyl group may be appropriately selected from the compounds exemplified above. The solvent containing a hydroxyl group is preferably alkylene glycol monoalkyl ether, alkyl lactate or the like, and more preferably propylene glycol monomethyl ether, ethyl lactate or the like. The solvent containing no hydroxyl group is preferably alkylene glycol monoalkyl ether acetate, alkyl alkoxypropionate, a monoketone compound which may contain a ring, a cyclic lactone, alkyl acetate or the like, and among them, particularly preferably propylene glycol monomethyl ether acetate, ethyl ethoxypropionate, 2-heptanone, γ-butyrolactone, cyclohexanone or butyl acetate, and most preferably propylene glycol monomethyl ether acetate, ethylethoxypropionate or 2-heptanone.

The mixing ratio (by mass) of the solvent containing a hydroxyl group to the solvent containing no hydroxyl group is 1/99 to 99/1, preferably 10/90 to 90/10, and more preferably 20/80 to 60/40. A mixed solvent containing the solvent containing no hydroxyl group in an amount of 50% by mass or more is particularly preferred in view of coating uniformity.

The solvent is preferably a mixed solvent of two or more kinds of solvents containing propylene glycol monomethyl ether acetate.

[9] Dissolution Inhibiting Compound Having a Molecular Weight of 3,000 or Less and Capable of Decomposing by the Action of an Acid to Increase the Solubility in an Alkali Developer

A dissolution inhibiting compound having a molecular weight of 3,000 or less and capable of decomposing by the action of an acid to increase the solubility in an alkali developer (hereinafter, also referred to as a “dissolution inhibiting compound”) is preferably an alicyclic or aliphatic compound containing an acid-decomposable group, such as acid-decomposable group-containing cholic acid derivative described in Proceeding of SPIE, 2724, 355 (1996), so as not to reduce the transparency to light at 220 nm or less. Examples of the acid-decomposable group and alicyclic structure are the same as those described above with respect to the resin as the component (B).

Meanwhile, in the case where the actinic ray-sensitive or radiation-sensitive resin composition of the present invention is exposed to KrF excimer laser or irradiated with electron beam, the dissolution inhibiting compound preferably contains a structure where the phenolic hydroxyl group of a phenol compound is substituted by an acid-decomposable group. The phenol compound is preferably a compound containing 1 to 9 phenol skeletons, and more preferably 2 to 6 phenol skeletons.

The amount of the dissolution inhibiting compound added is preferably 0.5% by mass to 50% by mass, more preferably 0.5% by mass to 40% by mass based on the solid of the actinic ray-sensitive or radiation-sensitive resin composition.

Specific examples of the dissolution inhibiting compound are shown below, but the present invention is not limited thereto.

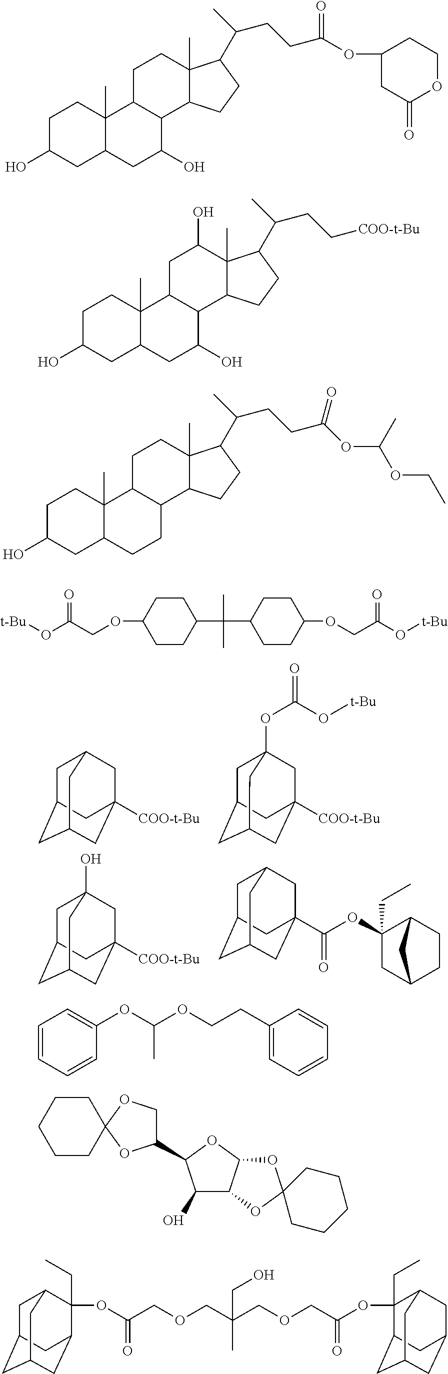

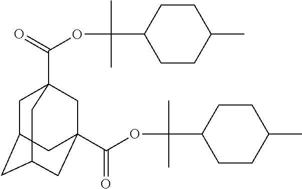 [SMILES: CC1CCC(C(C)(C)OC(=O)C23CC4CC(C2)CC(C(=O)OC(C)(C)C2CCC(C)CC2)(C4)C3)CC1]

[10] Other Components

The composition of the present invention may contain a carboxylate onium salt, a dye, a plasticizer, a photosensitizer, a light absorber and the like as appropriate, in addition to the aforementioned components.

[11] Pattern Forming Method

The pattern forming method includes exposing a resist film and developing the exposed film.

The resist film is formed with the actinic ray-sensitive or radiation-sensitive resin composition of the present invention as described above, and more specifically, is preferably formed on a substrate. In the pattern forming method of the present invention, the process of forming a film by the actinic ray-sensitive or radiation-sensitive resin composition on a substrate, the process of exposing the film, and the process of performing development may be performed by a generally known method.

From the viewpoint of improving the resolution, the actinic ray-sensitive or radiation-sensitive resin composition of the present invention is preferably used in a film thickness of 30 nm to 250 nm, and more preferably in a film thickness of 30 nm to 200 nm. Such a film thickness may be achieved by setting a solid concentration in the composition to an adequate range to have an appropriate viscosity, thereby improving coatability and film-formation property.

The solid concentration of the actinic ray-sensitive or radiation-sensitive resin composition on the present invention is usually 1.0% by mass to 10% by mass, preferably 1% by mass to 8.0% by mass, and more preferably 1.0% by mass to 6.0% by mass.

The actinic ray-sensitive or radiation-sensitive resin composition of the present invention is used by dissolving the aforementioned components in a solvent, filtering the solution through a filter, and then applying the filtered solution on a predetermined support. The filter is preferably a polytetrafluoroethylene-, polyethylene- or nylon-made filter having a pore size of 0.1 μm or less, more preferably 0.05 μm or less, and still more preferably 0.03 μm or less. Meanwhile, the filter may be used by connecting a plurality of kinds of filters in series or in parallel. In addition, the composition may be filtered several times. Further, a deaeration treatment or the like may be applied to the composition before or after filtration.

The composition is coated on a substrate for use in manufacturing integrated circuit devices (e.g., silicon/silicon dioxide coating) by a spiner, a coater and the like. Thereafter, a photosensitive resist film may be formed by drying the composition.

The film is irradiated with an actinic ray or radiation through a predetermined mask, subjected to preferably bake (heating), developed and rinsed. Accordingly, a good pattern can be obtained. Meanwhile, in irradiation with an electron beam, drawing not by passing through a mask (direct drawing) is common.

It is also preferred that the method includes a pre-baking process (PB) after film formation but before the exposure process.

Further, it is also preferred that the method includes a post-exposure baking process (PEB) after the exposure process but before the development process.

As for the heating temperature, both PB and PEB are performed preferably at 70° C. to 120° C., and more preferably at 80° C. to 110° C.

The heating time is preferably 30 to 300 seconds, more preferably 30 to 180 seconds, and still more preferably 30 to 90 seconds.

The heating may be performed using a means equipped with a typical exposure/developing machine or may be performed using a hot plate or the like.

By means of baking, the reaction in the exposed portion is accelerated, and thus the sensitivity or pattern profile is improved. It is also preferred that a heating process (post baking) is included after the rinsing process. The developer and rinse liquid remaining between patterns and in the inside of the pattern are removed by the baking.

The actinic ray or radiation is not particularly limited, but, examples thereof include a KrF excimer laser, an ArF excimer laser, an EUV light, an electron beam and the like, and preferably an ArF excimer laser, an EUV light and an electron beam.

The developer used in developing the resist film formed using the actinic ray-sensitive or radiation-sensitive resin composition of the present invention is not particularly limited, but, for example, an alkali developer or a developer containing an organic solvent (hereinafter, also referred to as an organic-based developer) may be used.

As the alkali developer, it is possible to use, for example, an alkaline aqueous solution of inorganic alkalis such as sodium hydroxide, potassium hydroxide, sodium carbonate, sodium silicate, sodium metasilicate, aqueous ammonia and the like, primary amines such as ethylamine and n-propylamine, secondary amines such as diethylamine and di-n-butylamine, tertiary amines such as triethylamine and methyldiethylamine, alcohol amines such as dimethylethanolamine and triethanolamine, quaternary ammonium salts such as tetramethylammonium hydroxide and tetraethylammonium hydroxide, cyclic amines such as pyrrole and piperidine, and the like. Further, alcohols and a surfactant may be added to the alkaline aqueous solution each in an appropriate amount and the mixture may be used. The alkali concentration of the alkali developer is usually 0.1% by mass to 20% by mass. The pH of the alkali developer is usually 10.0 to 15.0.

As the organic-based developer, it is possible to use a polar solvent such as a ketone-based solvent, an ester-based solvent, an alcohol-based solvent, an amide-based solvent and an ether-based solvent, and a hydrocarbon-based solvent.

Examples of the ketone-based solvent may include 1-octanone, 2-octanone, 1-nonanone, 2-nonanone, acetone, 2-heptanone(methyl amyl ketone), 4-heptanone, 1-hexanone, 2-hexanone, diisobutyl ketone, cyclohexanone, methylcyclohexanone, phenylacetone, methyl ethyl ketone, methyl isobutyl ketone, acetyl acetone, acetonyl acetone, ionone, diacetonyl alcohol, acetyl carbinol, acetophenone, methyl naphthyl ketone, isophorone, propylene carbonate and the like.

Examples of the ester-based solvent may include methyl acetate, butyl acetate, ethyl acetate, isopropyl acetate, pentyl acetate, isopentyl acetate, amyl acetate, cyclohexyl acetate, isobutyl isobutyrate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate and the like.

Examples of the alcohol-based solvent may include an alcohol such as methyl alcohol, ethyl alcohol, n-propyl alcohol, isopropyl alcohol, n-butyl alcohol, sec-butyl alcohol, tert-butyl alcohol, isobutyl alcohol, n-hexyl alcohol, n-heptyl alcohol, n-octyl alcohol, n-decanol and the like, a glycol-based solvent such as ethylene glycol, diethylene glycol and triethylene glycol, a glycol ether-based solvent such as ethylene glycol monomethyl ether, propylene glycol monomethyl ether, ethylene glycol monoethyl ether, propylene glycol monoethyl ether, diethylene glycol monomethyl ether, triethylene glycol monoethyl ether and methoxymethyl butanol, and the like.

Examples of the ether-based solvent may include, in addition to the glycol ether-based solvents, dioxane, tetrahydrofuran, and the like.

Examples of the amide-based solvent may include N-methyl-2-pyrrolidone, N,N-dimethylacetamide, N,N-dimethylformamide, hexamethylphosphoric triamide, 1,3-dimethyl-2-imidazolidinone and the like.

Examples of the hydrocarbon-based solvent may include an aromatic hydrocarbon-based solvent such as toluene and xylene, and an aliphatic hydrocarbon-based solvent such as pentane, hexane, octane and decane.

A plurality of the aforementioned solvents may be mixed, or the solvents may be used by being mixing with a solvent other than those described above or with water. However, in order to sufficiently exhibit the effects of the present invention, it is preferred that the water content ratio of the entire developer is less than 10% by mass, and it is more preferred that the developer contains substantially no moisture.

That is, the amount of the organic solvent used in the organic-based developer is preferably 90% by mass to 100% by mass, and preferably 95% by mass to 100%/o by mass, based on the total amount of the developer.

In particular, the organic-based developer is preferably a developer containing at least one of organic solvents selected from the group consisting of a ketone-based solvent, an ester-based solvent, an alcohol-based solvent, an amide-based solvent and an ether-based solvent.

To the organic-based developer, a surfactant may be added in an appropriate amount, if necessary.

The surfactant is not particularly limited but, for example, ionic or nonionic fluorine-based and/or silicon-based surfactant and the like may be used. Examples of the fluorine and/or silicon-based surfactants may include surfactants described in Japanese Patent Application Laid-Open Nos. 862-36663, S61-226746, S61-226745, S62-170950, S63-34540, H7-230165, H8-62834, H9-54432 and H9-5988, and U.S. Pat. Nos. 5,405,720, 5,360,692, 5,529,881, 5,296,330, 5,436,098, 5,576,143, 5,294,511 and 5,824,451, and a nonionic surfactant is preferred. The nonionic surfactant is not particularly limited, but a fluorine-based surfactant or a silicon-based surfactant is more preferably used. The amount of the surfactant used is usually 0.001% by mass to 5% by mass, preferably 0.005% by mass to 2% by mass, and more preferably 0.01% by mass to 0.5% by mass based on the total amount of the developer.

As for the rinse liquid, pure water is used, and an appropriate amount of a surfactant may be added thereto.

As for the developing method, it is possible to apply, for example, a method of dipping a substrate in a bath filled with a developer for a predetermined time (a dipping method), a method of raising a developer on a substrate surface sufficiently by the effect of a surface tension and keeping the substrate still for a predetermined time, thereby performing development (a puddle method), a method of spraying a developer on a substrate surface (a spray method), a method of continuously ejecting a developer on a substrate spinning at a constant speed while scanning a developer ejecting nozzle at a constant rate (a dynamic dispense method) and the like.

Further, after the development treatment or rinse treatment, a treatment of removing the developer or rinse liquid adhering on the pattern by a supercritical fluid may be performed.

Meanwhile, before forming a photosensitive film (resist film), an antireflection film may be formed on a substrate in advance.

Examples of the antireflection film may include an inorganic film type such as titanium, titanium dioxide, titanium nitride, chromium oxide, carbon or amorphous silicon, and an organic film type composed of a light absorbent and a polymer material. Further, as an organic antireflection film, a commercially available organic antireflection film such as DUV 30 series or DUV-40 series manufactured by Brewer Science, Inc., and AR-2, AR-3 and AR-5 manufactured by Shipley Company may be used.

Exposure (liquid immersion exposure) may be carried out after filling the interstice between a film and a lens with a liquid (liquid immersion medium) of refractive index higher than that of air upon irradiation with an actinic rays or radiation. Accordingly the resolution may be enhanced. The available liquid immersion medium is preferably water. Water is preferred from the viewpoint of a refractive index with a low temperature coefficient, easy availability and easy handling.

Further, from the viewpoint of enhancing the refractive index, a medium having a refractive index of 1.5 or higher may be used. The medium may be an aqueous solution or an organic solvent.

When water is used as an immersion liquid, an additive intended for enhancement of refractive index and the like may be added in slight proportion. Examples of the additive are described in detail in Chapter 12 of “Process and Material of Liquid Immersion Lithography” published by CMC Publishing Co., Ltd. Meanwhile, the presence of a substance which is opaque in 193-nm light or the presence of an impurity whose refractive index is greatly different from that of water causes a distortion of optical image projected on the film. Accordingly, it is preferred that the water use is distilled water. Further, pure water purified by an ion exchange filter or the like may be used. The electrical resistance of pure water is preferably 18.3 MQcm or higher, and the TOC (organic matter concentration) thereof is preferably 20 ppb or below. And, a deaeration treatment may be preferably carried out.

In order to avoid any contact of the resist film with the immersion liquid, a film that is sparingly soluble in the immersion liquid (hereinafter, also referred to as a “top coat”) may be formed between the resist film and the immersion liquid. The functions required for the top coat are coating suitability onto the resist film, transparency in radiation especially having a wavelength of 193 nm and sparing solubility in the immersion liquid. It is preferred to use, as the top coat, one that does not mix with the resist film and is uniformly coating onto the resist film.

From the viewpoint of transparency at 193 nm, the top coat is preferably comprised of a polymer containing no aromatic moiety. Examples of the polymer may include a hydrocarbon polymer, an acrylate ester polymer, polymethacrylic acid, polyacrylic acid, polyvinyl ether, a silicon-containing polymer and a fluorine-containing polymer. The aforementioned hydrophobic resin is appropriate as the top coat. Since an optical lens is contaminated by leaching of impurities from the top coat into the immersion liquid, it is preferred to reduce the amount of residual monomer components of polymer contained in the top coat.

When the topcoat is peeled off, a developer may be used, or another peeling agent may be used. As the peeling agent, a solvent that rarely penetrates the film is preferred. From the viewpoint that the peeling process may be performed simultaneously with the developing treatment process of the film, it is preferred that the topcoat may be peeled off by an alkali developer. From the viewpoint of peeling off the topcoat with an alkali developer, the topcoat is preferably acidic, but from the viewpoint of a non-intermixture property with respect to the film, the topcoat may be neutral or alkaline.

It is preferred that there is no difference or a small difference in the refractive index between the topcoat and the liquid for liquid immersion. In this case, the resolution may be improved. When the exposure light source is an ArF excimer laser (wavelength: 193 nm), it is preferred that water is used as the liquid for liquid immersion, and thus the topcoat for ArF liquid immersion exposure preferably has a refractive index close to the refractive index (1.44) of water.

Further, from the viewpoint of transparency and refractive index, the topcoat is preferably a thin film. It is preferred that the topcoat is not mixed with the film and the liquid for liquid immersion. From this viewpoint, when the liquid for liquid immersion is water, it is preferred that the solvent used for the topcoat is sparingly soluble in the solvent used for the the actinic ray-sensitive or radiation-sensitive resin composition of the present invention and is a water-insoluble medium. Further, when the liquid for liquid immersion is an organic solvent, the topcoat may be water-soluble or water-insoluble.

Further, the present invention also relates to a method for manufacturing an electronic device, including the aforementioned pattern forming method of the present invention, and an electronic device manufactured by this manufacturing method.

The electronic device of the present invention is suitably mounted on electric electronic devices (such as home appliances, OA media-related devices, optical devices and communication devices).

### EXAMPLE

Hereinafter, the present invention will be described with reference to the examples, but the present invention is not limited thereto.

### Synthesis Example 1

### Synthesis of Compound A-35

The compound A-35 was synthesized in accordance with the following scheme.

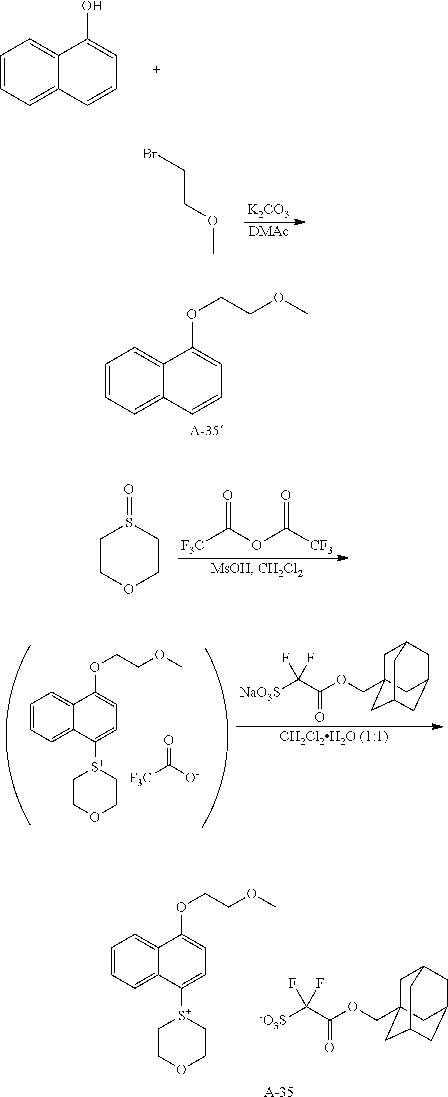

<<Synthesis of A-35′>>

10 g (69.4 mmol) of 2-naphthol, 14.63 g (76.3 mmol) of 1-bromo-2-methoxyethane, 19.2 g (138.4 mmol) of potassium carbonate and 50 g of dimethylacetamide (DMAc) were placed in a three-necked flask, and stirred for 12 hours while heating at 90° C. Thereafter, 100 ml of water and 100 ml of ethyl acetate were added therein to separate an organic phase, followed by washing with 100 ml of 0.5 M aqueous hydrochloric acid solution, 50 g of a saturated sodium bicarbonate solution and 50 g of a saturated aqueous sodium chloride solution successively. Thereafter, the organic phase was concentrated to obtain 13.3 g (65.9 mmol) of the desired compound A-35′.

¹H-NMR, 400 MHz, 6 (CDCl₃) ppm: 3.51 (3H, s), 3.89 (2H, t), 4.30 (2H, t), 6.81 (1H, d), 7.56 (1H, t), 7.41-7.54 (3H, m), 7.76-7.81 (1H, m), 8.28-8.31 (1H, m)

<<Synthesis of A-35>>

2 g (9.8 mmol) of A-35′ was placed in a three-necked flask and dissolved in 20 g of dichloromethane. Then, 4.2 g (19.6 mmol) of trifluoroacetic anhydride and 1.15 g (11.8 mmol) of methanesulfonic acid were added thereto, and cooled to an internal temperature of 4° C. in an ice bath. Subsequently, 1.3 g (10.8 mmol) of 1,4-thioxan-4-oxide was dissolved in 5 g of dichloromethane, and the solution was added dropwise to the reaction solution by using a dropping funnel. The internal temperature was adjusted to 10° C. or below during the dropwise addition. Further, stirring was carried out at the internal temperature of 4° C. for 1 hour, 20 g of water was added, and 3.7 g (9.8 mmol) of sodium (adamantan-1-ylmethoxycarbonyl)-difluoromethanesulfonate was added, followed by stirring at room temperature for 1 hour. An organic matter was separated, washed with 20 g of water, concentrated and then crystallized to obtain 5.7 g (9.1 mmol) of the desired compound A-35.

¹H-NMR, 400 MHz, δ (CDCL₃) ppm: 1.52 (6H, brs), 1.56-1.69 (6H, m), 1.91 (3H, s), 3.49 (31H, s), 3.77-3.93 (8H, m), 4.22 (2H, ddd), 4.37 (2H, brt), 4.44 (2H, td), 7.15 (1H, d), 7.58 (1H, t), 7.74 (1H, t), 8.30-8.40 (3H, m) Other compounds (A) listed in Table 2 below were synthesized by the same synthesis method as in the the compound A-35.

### Synthesis Example 2

### Synthesis of Resin (3)

11.5 g of cyclohexanone was placed in a three-necked flask and heated at 85° C. under nitrogen flow. To this, a solution in which 1.98 g, 3.05 g, 0.95 g, 2.19 g and 2.76 g of the following compounds (monomers) successively from the left, and a polymerization initiator V-601 (manufactured by Wako Pure Chemical Industries, Ltd., 0.453 g) were dissolved to 21.0 g of cyclohexanone, was added dropwise over 6 hours. After the completion of dropwise addition, the solution was further allowed to react at 85° C. for 2 hours. The reaction solution was allowed to cool, and then added dropwise to a mixed solution of 420 g of hexane/180 g of ethyl acetate over 20 minutes, and a precipitated powder was taken by filtration and dried to obtain 9.1 g of the following resin (3), which was an acid-decomposable resin. The composition ratio of the polymer was 20/25/10/30/15 as calculated by NMR. The weight average molecular weight of the obtained resin (3) was 10,400 in terms of the standard polystyrene, and the polydispersity (Mw/Mn) was 1.56.

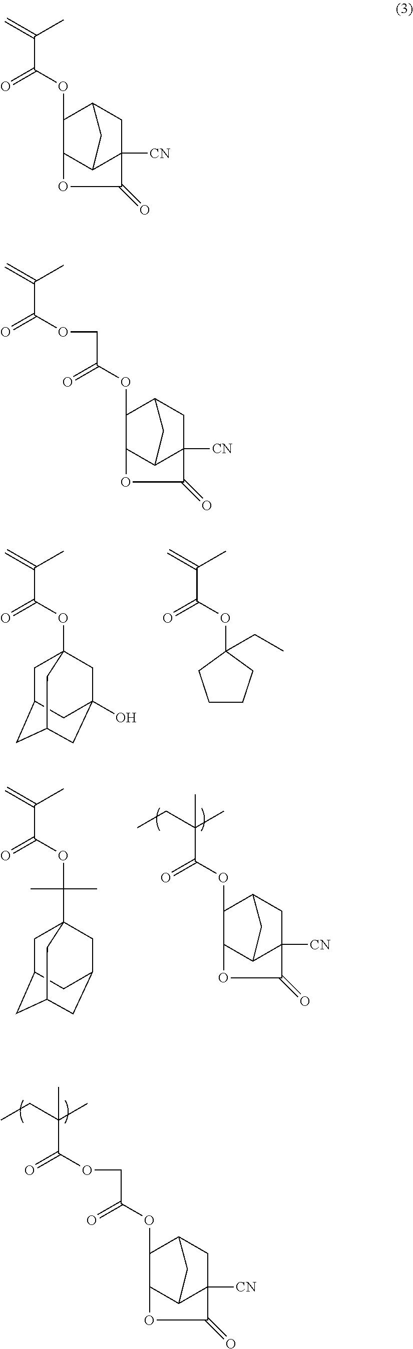

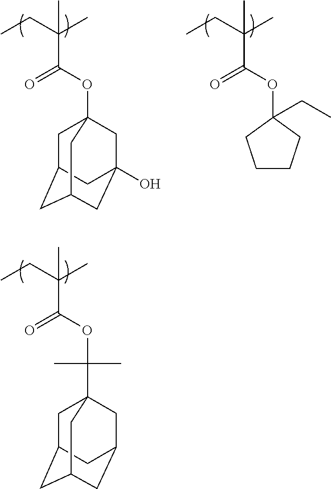

The resins (1), (2), (4) to (6) as described below as an acid-decomposable resin were synthesized in the same manner as in Synthesis Example 2.

### Examples 1 to 25 and Comparative Examples 1 to 6

<Preparation of Resist>

The components as indicated in Table 2 below were dissolved in a solvent to prepare a solution having a solid concentration of 4% by mass, respectively, and each was filtered through a polyethylene filter having a pore size of 0.05 μm to prepare an actinic ray-sensitive or radiation-sensitive resin composition (positive type resist composition). The actinic ray-sensitive or radiation-sensitive resin compositions were evaluated by the following methods, and the results are given in Table 2.

With respect to each of the components in Table 2, the ratio indicated when multiple types are used represents a mass ratio.

In Table 2, when the actinic ray-sensitive or radiation-sensitive resin composition did not contain any hydrophobic resin (HR) and when after the formation of a film, a top coat protective film containing a hydrophobic resin (HR) was formed on an upper layer of the film, “TC” is noted as the form of usage of the hydrophobic resin.

<Evaluation of Resist Using an Alkali Developer>

<Evaluation of Resist>

(Exposure Condition 1: ArF Liquid Immersion Exposure)

An organic antireflection film ARC29SR (manufactured by Nissan Chemical Industries, Ltd.) was coated onto a 12-inch silicon wafer and baked at 205° C. for 60 seconds, to form an antireflection film with a thickness of 98 nm. The prepared actinic ray-sensitive or radiation-sensitive resin composition was coated thereonto and baked at 130° C. for 60 seconds to form a resist film with a thickness of 120 nm. When a top coat was used, 3% by mass of a solution obtained by dissolving a top coat resin in decane/octanol (mass ratio 9/1) was coated onto the resist film and baked at 85° C. for 60 seconds to form a 50 nm-thick top coat layer. The resultant wafer was exposed through a 6% half-tone mask of 48 nm line width 1:1 line and space pattern to light by means of an ArF excimer laser liquid immersion scanner (manufactured by ASML Co., Ltd., XT-1700i, NA 1.20, C-Quad, outer sigma 0.981, inner sigma 0.895, XY deflection). Ultrapure water was used as the immersion liquid. Thereafter, the exposed wafer was baked at 100° C. for 60 seconds, developed by puddling with an aqueous solution of tetramethylammonium hydroxide (2.38% by mass) for 30 seconds, rinsed by puddling with pure water and spin dried to obtain a resist pattern.

(Exposure Condition 2: ArF Dry Exposure)

An organic antireflection film ARC29A (manufactured by Nissan Chemical Industries, Ltd.) was coated onto a 12-inch silicon wafer and baked at 205° C. for 60 seconds to form an antireflection film with a thickness of 75 nm. The prepared positive resist composition was coated thereonto and baked at 130° C. for 60 seconds to form a resist film with a thickness of 120 nm. The resultant wafer was exposed through a 6% half-tone mask of 75 nm line width 1:1 line and space pattern to light by means of an ArF excimer laser scanner (manufactured by ASML, PAS5500/1100, NA0.75, dipole, σo/σi=0.89/0.65). Thereafter, the exposed wafer was baked at 100° C. for 60 seconds, developed with an aqueous solution of tetramethylammonium hydroxide (2.38% by mass) for 30 seconds, rinsed with pure water and spin dried to obtain a resist pattern.

(Evaluation of Exposure Latitude)

In exposure condition 1, the optimum exposure amount was defined as the exposure amount in which a 1:1 line-and-space mask pattern of 48 nm line width was reproduced. The exposure amount range in which the pattern size allowed 48 nm±10% when the exposure amount was varied was measured. The exposure latitude is the quotient of the value of the exposure amount range divided by the optimum exposure amount, the quotient expressed by a percentage. In exposure condition 2, the optimum exposure amount was defined as the exposure amount in which a 1:1 line-and-space mask pattern of 75 nm line width was reproduced. The exposure amount range in which the pattern size allowed 75 nm k 10% when the exposure amount was varied was measured. The exposure latitude is the quotient of the value of the exposure amount range divided by the optimum exposure amount, the quotient expressed by a percentage. A larger value indicates less change in performance by exposure amount changes and better exposure latitude.

(Evaluation of LWR)

The obtained line pattern of line/space=1/1 (75 nm line width in ArF dry exposure, 48 nm line width in ArF liquid immersion exposure) was observed by means of a scanning electron microscope (S9380 manufactured by Hitachi, Ltd.). In an edge 2 μm region along the longitudinal direction of the line pattern, the line width was measured at 50 points. With respect to the distribution of measurements, the standard deviation was determined, and 3a was computed therefrom. A smaller value indicates more favorable performance.

(Evaluation of Pattern Collapse)

In exposure condition 1, the optimum exposure amount was defined as the exposure amount in which a 1:1 line-and-space mask pattern of 48 nm line width was reproduced. And in exposure condition 2, the optimum exposure amount was defined as the exposure amount in which a 1:1 line-and-space mask pattern of 75 nm line width was reproduced. Then, when thinning the line width of the line pattern formed by further increasing the exposure amount from the optimum exposure amount, the pattern collapse was defined with a threshold minimum line width resolved while the pattern is not collapsed. A smaller value indicates that finer pattern is resolved without being collapsed, and thus, the pattern collapse hardly occurs and the resolution is high.

(Aging Stability of Resist)

The aging stability of resist was judged on the basis of a period guaranteeing no change of resist performance. The aging stability was evaluated by the following (1) aging stability test for contact angle and (2) aging stability test for line width.

[Aging Stability Test for Line Width: Exposure Condition (1)]

The line widths of the films prepared by using resist liquids respectively aged at 40° C., 50° C. and 60° C. for 30 days were compared with that of the film prepared by using a resist liquid aged at 0° C. for 30 days (reference resist film), and the stability was evaluated by any line width differences therebetween.

Specifically, first, with respect to the film prepared by using the resist liquid aged at 0° C. for 30 days, the exposure amount (E) that reproduced a mask pattern of 45 nm line width (line/space: 1/1) was determined. Subsequently, E₁ exposure was performed on each of three types of resist films aged at raised temperatures for 30 days. The line widths of the obtained patterns were measured by means of a scanning electron microscope (S-9260 manufactured by Hitachi. Ltd.), and pattern line width variations from the line width (45 nm) obtained from the reference resist were calculated.

On the basis of the obtained 3-point data, plotting was performed on a semi logarithmic graph wherein the X-axis indicated the reciprocal of aging temperature (Celsius converted to Kelvin) while the Y-axis indicated the reciprocal of line width variation per day (namely, quotient of the determined line width variation divided by 30), and a collinear approximation was applied. On the the obtained line, the Y-coordinate value at the X-coordinate corresponding to the aging temperature 25° C. was read. The read Y-coordinate value was denoted as the 1 nm-line-width guaranteed days in room temperature condition (25° C.).

[Aging Stability Test for Line Width: Exposure Condition (2)]

The line widths of the films prepared by using resist liquids respectively aged at 40° C., 50° C. and 60° C. for 30 days were compared with that of the film prepared by using the resist aged at 0° C. for 30 days (reference resist), and the stability was evaluated by any line width differences therebetween.

Specifically, first, with respect to the film prepared by using the resist liquid aged at 0° C. for 30 days, the exposure amount (E₁) that reproduced a mask pattern of 75 nm line width (line/space: 1/1) was determined. Subsequently, exposure was performed on each of three types of resist films aged at raised temperatures for 30 days. The line widths of the obtained patterns were measured by means of a scanning electron microscope (S-9260 manufactured by Hitachi, Ltd.), and pattern line width variations from the line width (75 nm) obtained from the reference resist were calculated.

On the basis of the obtained 3-point data, plotting was performed on a semi logarithmic graph wherein the X-axis indicated the reciprocal of aging temperature (Celsius converted to Kelvin) while the Y-axis indicated the reciprocal of line width variation per day (namely, quotient of the determined line width variation divided by 30), and a collinear approximation was applied. On the the obtained line, the Y-coordinate value at the X-coordinate corresponding to the aging temperature 25° C. was read. The the read Y-coordinate value was denoted as the 1 nm-line-width guaranteed days in room temperature condition (25° C.).

[Aging Stability of Contact Angle: Exposure Conditions (1) and (2)]

The 1°-contact-angle guaranteed days (reciprocal of dynamic receding contact angle variation per day) in room temperature condition (25° C.) was determined by evaluating and plotting the contact angle variation over time in the same manner as described above with respect to [Aging stability of line width: Exposure Conditions (1) and (2)]. Meanwhile, in the measurement of contact angle, the dynamic receding contact angle before exposure with respect to pure water was measured by means of a fully automated contact angle meter (Drop aster 700 manufactured by Kyowa Interface Science Co., Ltd.).

TABLE 2

| | Compound (A) | Resin (B) | Basic compound and | | Hydrophobic resin (HR) | Solvent | Surfactant |
|---|---|---|---|---|---|---|---|
| | (g) | (50 g) | Compound (C) (g) | | (35 mmg) | (molar ratio) | (10 mg) |
| Ex. 1 | A-7 (2.1) | Resin (1) | PBI | (0.23) | B-29 | A1/A2 = 80/20 | W-1 |
| Ex. 2 | A-18 (2.3) | Resin (2) | DIA | (0.30) | TC (B-8) | A1/A2 = 70/30 | W-1 |
| Ex. 3 | A-23 (1.8) | Resin (3) | DIA | (0.41) | B-29 | A1 | W-1 |
| Ex. 4 | A-24 (1.7) | Resin (1) | D-13 | (0.30) | B-30 | A1 | W-1 |
| Ex. 5 | A-25 (2.3) | Resin (2) | D-52 | (0.33) | B-10 | A1/B1 = 90/10 | W-2 |
| Ex. 6 | A-27 (2.4) | Resin (3) | TEA | (0.41) | B-29 | A1/A2 = 70/30 | W-4 |
| Ex. 7 | A-34 (2.3) | Resin (4) | PBI | (0.29) | B-10 | A1/B1 = 90/10 | W-1 |
| Ex. 8 | A-35 (1.7) | Resin (6) | PBI | (0.30) | B-47 | A1 | W-1 |
| Ex. 9 | A-36 (2.2) | Resin (1) | DBA | (0.31) | B-29 | A1 | W-1 |
| Ex. 10 | A-37 (2.1) | Resin (3) | D-52 | (0.42) | B-2 | A1/A2 = 80/20 | W-1 |
| Ex. 11 | A-38 (1.8) | Resin (5) | D-13 | (0.28) | B-16 | A1 | W-1 |
| Ex. 12 | A-39 (1.7) | Resin (4) | PBI | (0.33) | B-10 | A1/A3 = 95/5 | W-2 |
| Ex. 13 | A-40 (2.1) | Resin (1) | DIA | (0.30) | B-47 | A1/A2 = 60/40 | W-1 |
| Ex. 14 | A-41 (2.2) | Resin (3) | D-52 | (0.31) | B-2 | A1/A2 = 90/10 | W-1 |
| Ex. 15 | A-42 (2.3) | Resin (1)/Resin (4) (6 g/4 g) | D-13 | (0.29) | B-29 | A1/B2 = 90/10 | W-1 |
| Ex. 16 | A-43 (2.3) | Resin (1) | D-13/DIA | (0.2/0.15) | TC (B-8) | A1/A2/A3 = 90/5/5 | W-1 |
| Ex. 17 | A-44 (2.3) | Resin (2) | DIA | (0.29) | B-29 | A1 | W-2 |
| Ex. 18 | A-49 (2.3) | Resin (3) | PBI | (0.31) | B-47 | A1 | W-1 |
| Ex. 19 | A-46 (2.3) | Resin (4) | D-52 | (0.30) | B-29 | A1/A2/B1 = 95/4/1 | W-1 |
| Ex. 20 | A-53 (2.3) | Resin (5) | D-13 | (0.29) | B-30 | A1/A2 = 70/30 | W-1 |
| Ex. 21 | A-44 (1.2)/z95 (1.1) | Resin (6) | D-13/DIA | (0.2/0.15) | B-10 | A1 | W-1 |
| Ex. 22 | A-35 (1.2)/z93 (1.1) | Resin (2) | D-52 | (0.32) | B-2 | A1/A2 = 70/30 | W-1 |
| Ex. 23 | A-37 (1.2)/z76 (1.1) | Resin (5) | D-13 | (0.28) | B-39 | A1/B2 = 80/20 | W-2 |
| Ex. 24 | A-11 (1.7)/z95 (0.2) | Resin (4) | D-52/DBA | (0.15/0.1) | B-26 | A1/A2/B1 = 95/3/2 | W-1 |
| Ex. 25 | A-9 (1.0)/A-43 (1.0) | Resin (6) | D-13/DIA | (0.2/0.15) | B-10 | A1 | W-1 |
| C. Ex. 1 | RA-1 (1.7) | Resin (1) | PBI | (0.28) | B-10 | A1 | W-1 |
| C. Ex. 2 | RA-2 (1.8) | Resin (1) | PEA | (0.3) | B-29 | A1/A2 = 90/10 | W-1 |
| C. Ex. 3 | RA-3 (1.9) | Resin (1) | D-13 | (0.28) | B-10 | A1 | W-1 |
| C. Ex. 4 | RA-4 (2.1) | Resin (1) | PBI | (0.30) | B-2 | A1 | W-1 |
| C. Ex. 5 | RA-5 (2.0) | Resin (2) | DIA | (0.40) | B-10 | A1/B2 = 90/10 | W-1 |
| C. Ex. 6 | RA-6 (1.9) | Resin (2) | DIA | (0.40) | B-10 | A1/B2 = 90/10 | W-1 |

| | | Evaluation item 1 | Evaluation item 2 | Evaluation item 3 | Evaluation item 4 Resist aging stability | |
|---|---|---|---|---|---|---|
| | Exposure | Exposure latitude | LWR | Collapse | Number of aging stable days | Number of aging stable days |
| | condition | (%) | (mm) | (nm) | of contact angle (day) | of line width (day) |
| Ex. 1 | 1 | 19.2 | 4.3 | 30 | 420 | 390 |
| Ex. 2 | 1 | 18.1 | 4.6 | 30.1 | 550 | 520 |
| Ex. 3 | 2 | 17.9 | 5.7 | 29 | 410 | 390 |
| Ex. 4 | 2 | 17.5 | 5.8 | 29.8 | 400 | 390 |
| Ex. 5 | 1 | 18.6 | 4.3 | 29.5 | 510 | 500 |
| Ex. 6 | 1 | 19.5 | 4.5 | 30.1 | 610 | 560 |
| Ex. 7 | 1 | 20.1 | 4.4 | 30.6 | 510 | 500 |
| Ex. 8 | 1 | 19.8 | 4.4 | 31.1 | 480 | 450 |
| Ex. 9 | 1 | 19.4 | 4.2 | 30.4 | 470 | 440 |
| Ex. 10 | 1 | 19.2 | 4.3 | 29.8 | 480 | 500 |
| Ex. 11 | 1 | 19.9 | 4.1 | 31.1 | 500 | 510 |
| Ex. 12 | 1 | 18.6 | 4.8 | 33.2 | 450 | 390 |
| Ex. 13 | 1 | 20.1 | 4.7 | 29.9 | 530 | 510 |
| Ex. 14 | 1 | 19.6 | 4.5 | 30 | 500 | 480 |
| Ex. 15 | 1 | 19.8 | 4.8 | 30.5 | 550 | 490 |
| Ex. 16 | 1 | 19.6 | 4.3 | 31.2 | 530 | 510 |
| Ex. 17 | 1 | 20.1 | 4.1 | 29.8 | 550 | 510 |
| Ex. 18 | 1 | 19.2 | 4.2 | 30.9 | 500 | 490 |
| Ex. 19 | 1 | 18.5 | 4.6 | 33.1 | 600 | 500 |
| Ex. 20 | 1 | 19.6 | 4.6 | 31.3 | 700 | 620 |
| Ex. 21 | 1 | 19.9 | 4.3 | 29.8 | 610 | 600 |
| Ex. 22 | 1 | 19.5 | 4.1 | 30.1 | 550 | 560 |
| Ex. 23 | 1 | 20.2 | 4.2 | 31 | 540 | 500 |
| Ex. 24 | 1 | 19.5 | 4.1 | 30.5 | 420 | 450 |
| Ex. 25 | 1 | 19.8 | 4.2 | 30.2 | 420 | 390 |
| C. Ex. 1 | 1 | 17.5 | 5.3 | 36.3 | 200 | 190 |
| C. Ex. 2 | 1 | 17.6 | 4.9 | 36.6 | 220 | 200 |
| C. Ex. 3 | 2 | 12.1 | 6.5 | 43.2 | 230 | 220 |
| C. Ex. 4 | 1 | 17.1 | 4.9 | 35.6 | 70 | 60 |
| C. Ex. 5 | 1 | 16.5 | 5.1 | 34.6 | 100 | 80 |
| C. Ex. 6 | 1 | 16.9 | 5 | 35.2 | 310 | 300 |

The abbreviations in the table are used as follows in the specific examples.

<Compound (A)>

The compound (A) used in Examples and the fluorine content (MnF) of the compound (A), which represented by (the sum of mass of the total fluorine atoms contained in the compound)/(the sum of mass of the total atoms contained in the compound), are shown below.

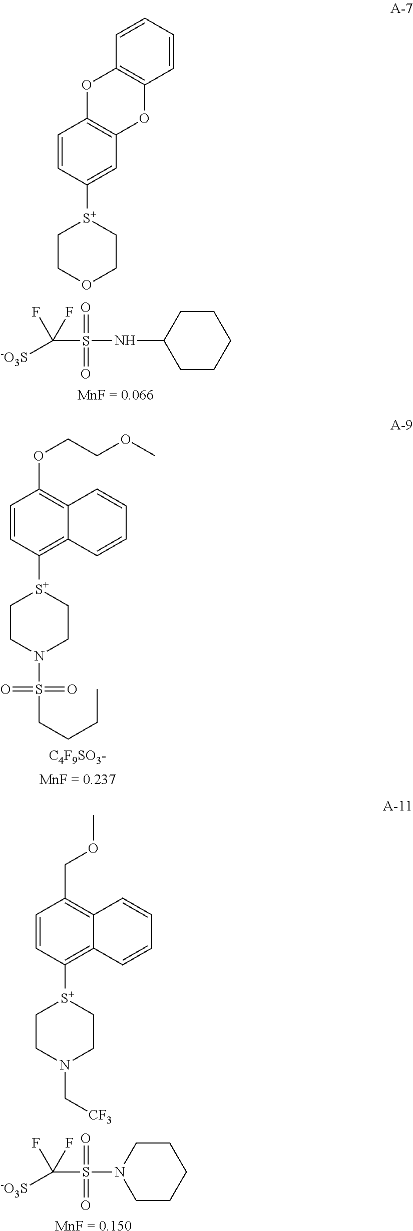

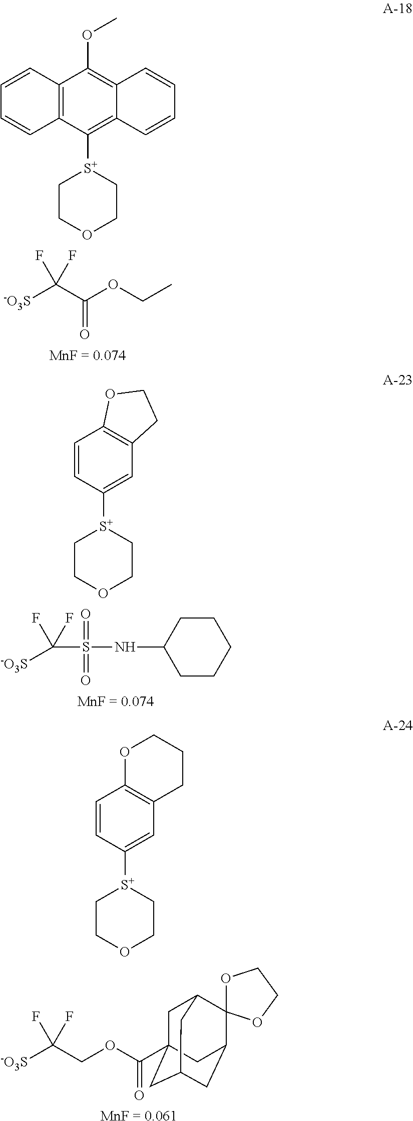

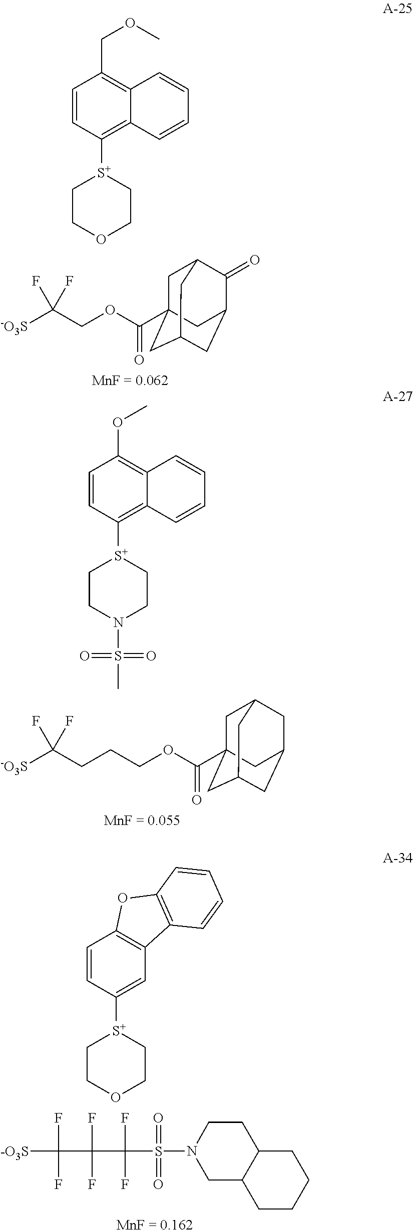

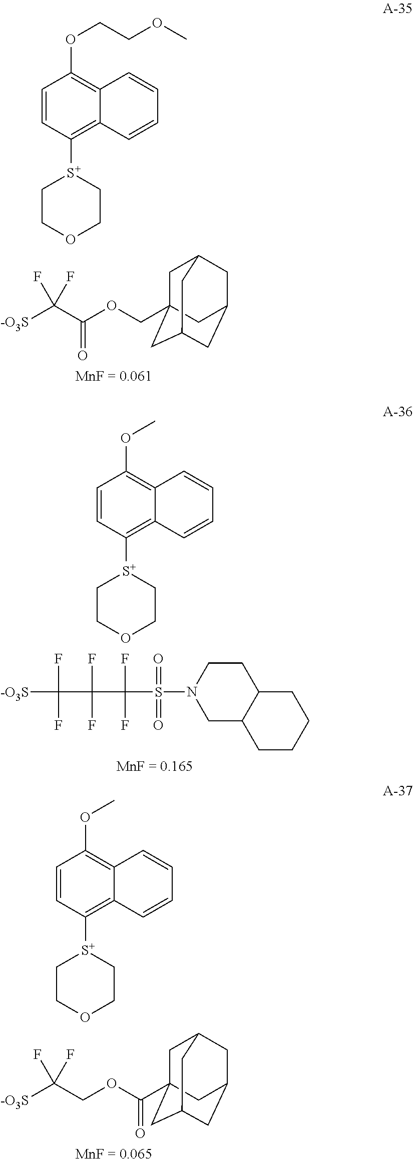

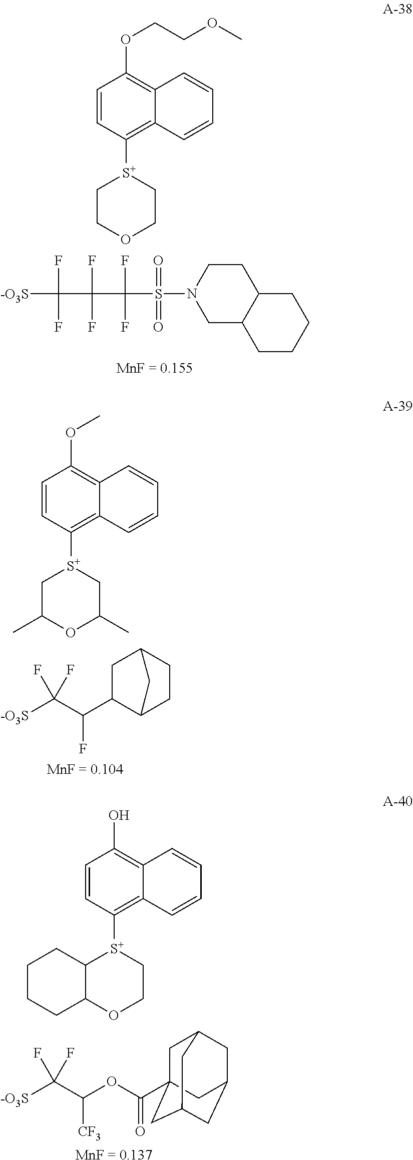

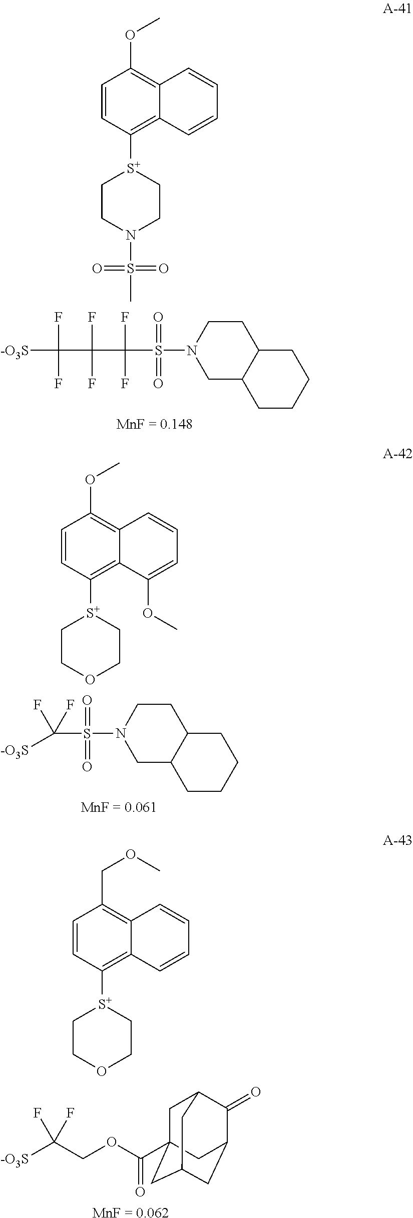

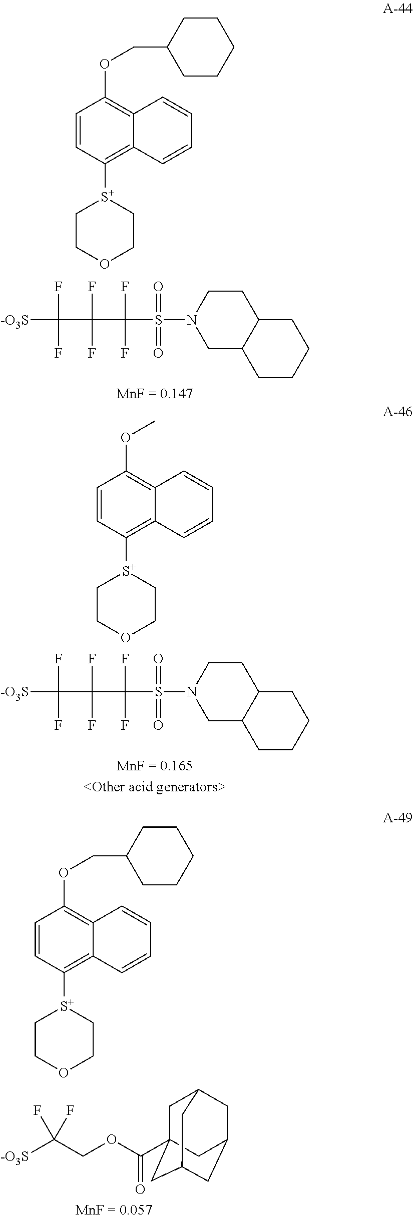

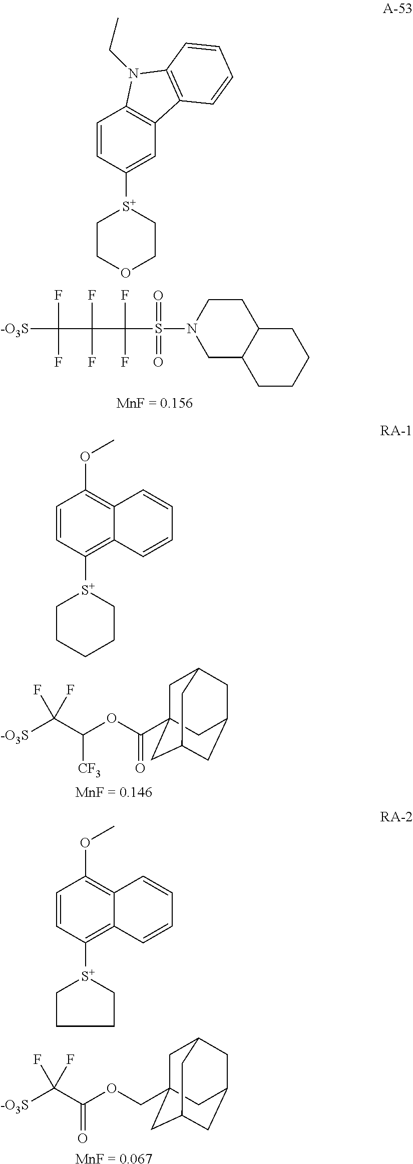

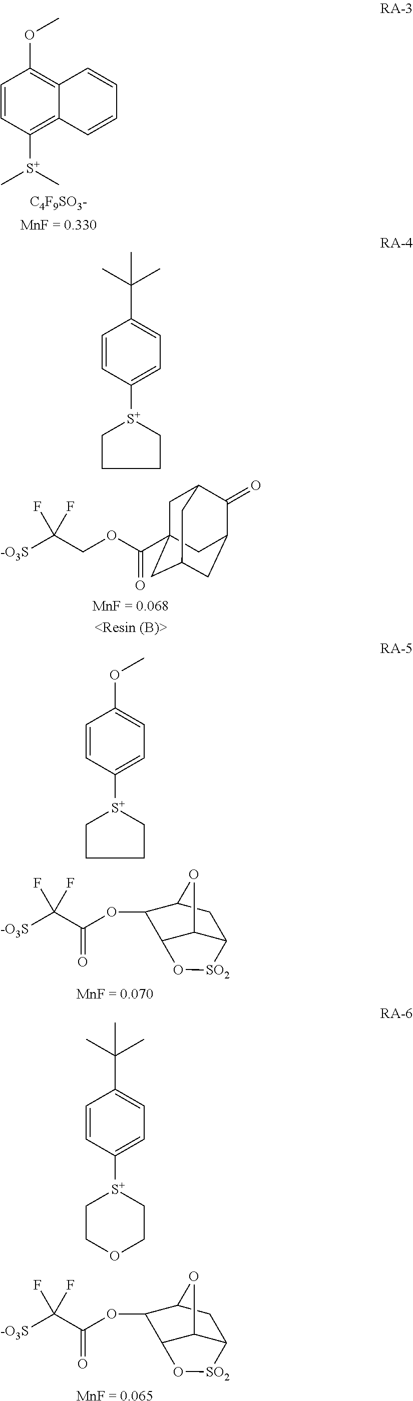

The composition ratios of the repeaing units for each of the following resins are in molar ratio.

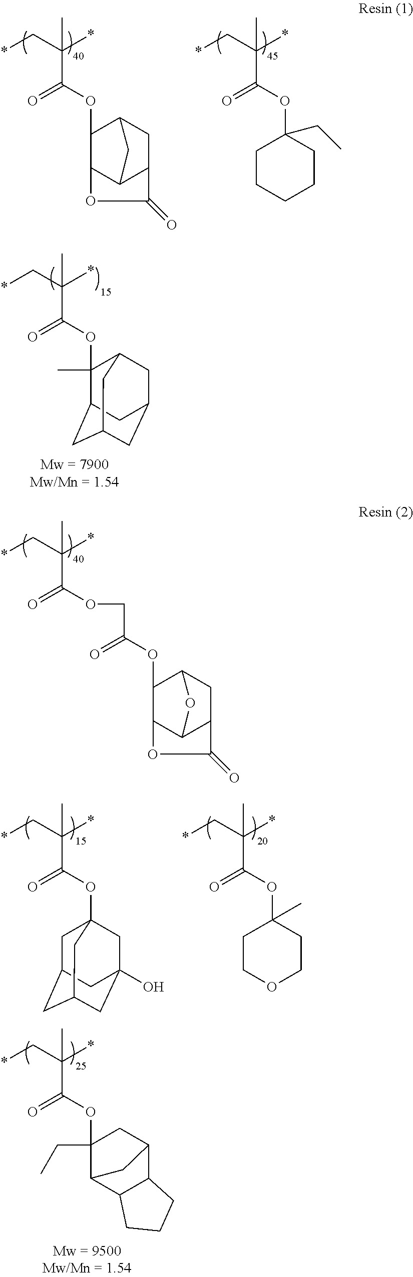

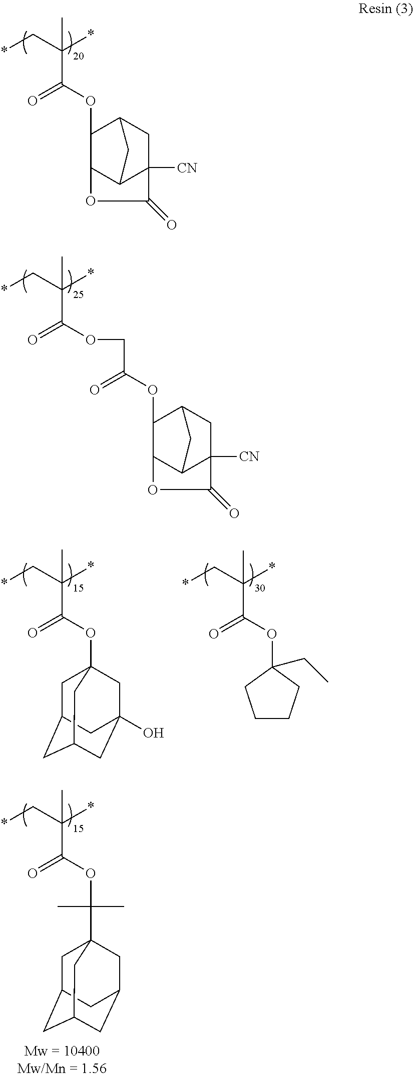

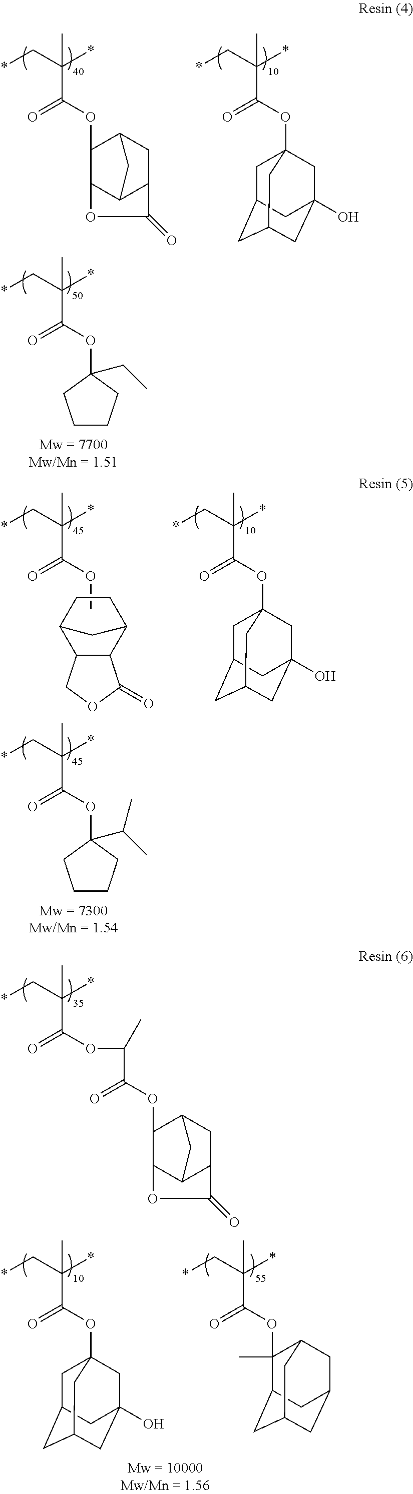

<Basic Compound>

DIA: 2,6-diisopropylaniline

TEA: triethanolamine

DBA: N,N-dibutylaniline

PBI: 2-phenylbenzimidazole

PEA: N-phenyldiethanolamine

[Low Molecular Weight Compound (C) Capable of Leaving by the Action of an Acid (Compound (C))]

Low molecular weight compound (C)

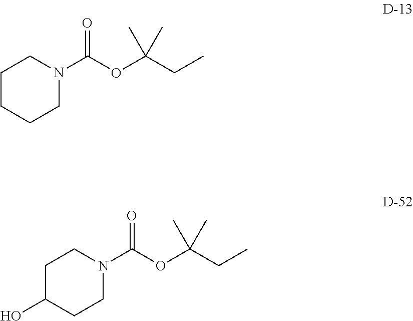

<Hydrophobic Resin (HR)>

The hydrophobic resin (HR) was appropriately selected from the resins (B−1) to (B-56) exemplified above.

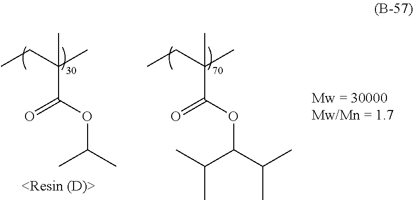

<Surfactant>

W-1: Megafac F176 (manufactured by DIC Corporation) (fluorine-based)

W-2: Megafac R08 (manufactured by DIC Corporation) (fluorine- and silicon-based)

W-3: PF6320 (manufactured by OMNOVA Solutions Inc.) (fluorine-based)

W-4: Troy sol S-366 (manufactured by Troy Chemical Co., Ltd.)

[Solvent]

A1: propylene glycol monomethylether acetate (PGMEA)

A2: cyclohexanone

A3: γ-butyrolactone

B1: propylene glycol monomethylether (PGME)

B2: ethyl lactate

As apparent from the results shown in Table 2, it is understood that Comparative Examples 1 to 6 in which an acid generator not satisfying Formula (1) is used have small exposure latitude and large LWR so that both of pattern collapse performance and aging stability are deteriorated.

Meanwhile, Examples 1 to 25 in which the compound (A) satisfying Formula (1) is used as an acid generator have large exposure latitude and small LWR so that both of pattern collapse performance and aging stability are excellent.

Especially, in the liquid immersion exposure, it is understood that Examples 1, 2 and 5 to 25 in which a pattern formation is performed have larger exposure latitude and smaller LWR so that the aging stability at a contact angle is more excellent.

The composition of the present invention may be suitably used for lithography processes in manufacturing electronic devices such as various semiconductor devices and recording media.

<Evaluation of Resist Using an Organic Solvent-Based Developer>

### Synthesis Example 3

### Synthesis of Resin (7))

102.3 parts by mass of cyclohexanone was heated at 80° C. under nitrogen flow. While stirring the liquid, a mixed solution of 22.2 parts by mass of a monomer represented by the following structural formula M-1, 22.8 parts by mass of a monomer represented by the following structural formula M-2, 6.6 parts by mass of a monomer represented by the following structural formula M-3, 189.9 parts by mass of cyclohexanone and 2.40 parts by mass of 2,2′-dimethyl azobisisobutyrate [V-601, manufactured by Wako Pure Chemical Industries, Ltd.] was added dropwise thereto over 5 hours. After the completion of dropwise addition, the solution was further stirred at 80° C. for 2 hours. The reaction solution was allowed to cool, then subjected to reprecipitation with a large amount of hexane/ethyl acetate (mass ratio 9:1), and filtered to obtain a solid, and the obtained solid was vacuum dried to obtain 41.1 parts by mass of the resin (7) of the present invention.

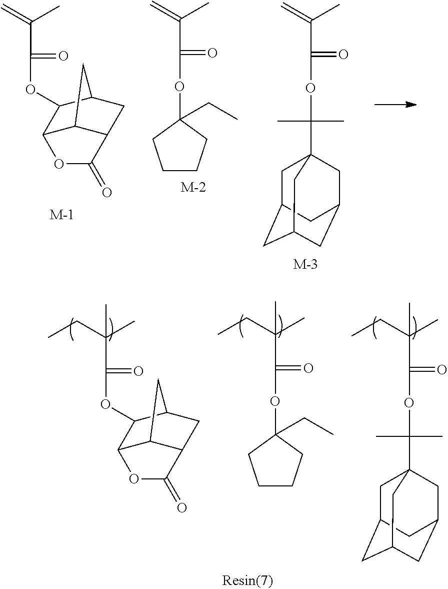

The weight average molecular weight (Mw: in terms of polystyrene) obtained from the GPC (carrier: tetrahydrofuran (THF)) of the obtained resin (7) is Mw=:9,500 with the polydispersity Mw/Mn=1.60. The composition ratio measured by ¹³C-NMR is 40/50/10.

The resins (8) to (15) were synthesized in the same manner as in Synthesis Example 3. The synthesized polymer structures are shown below.

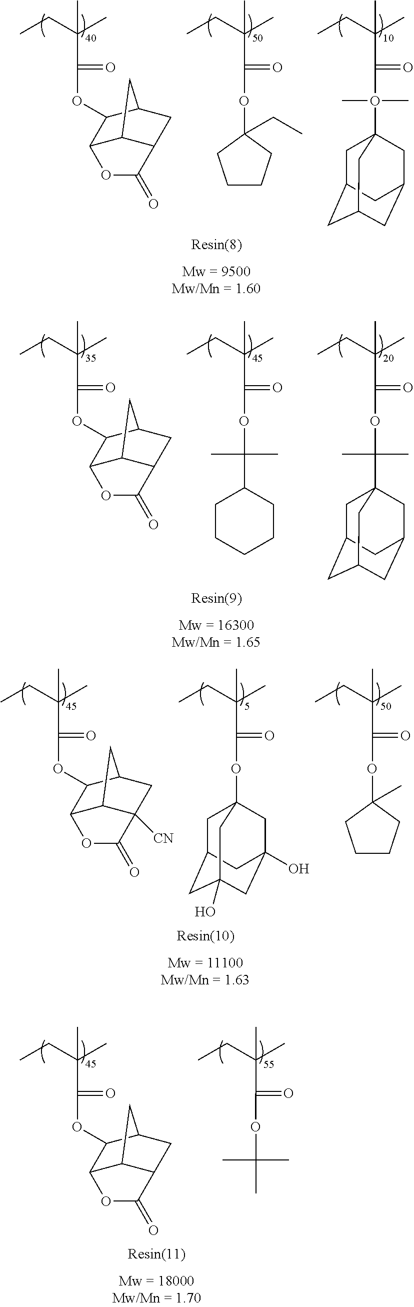

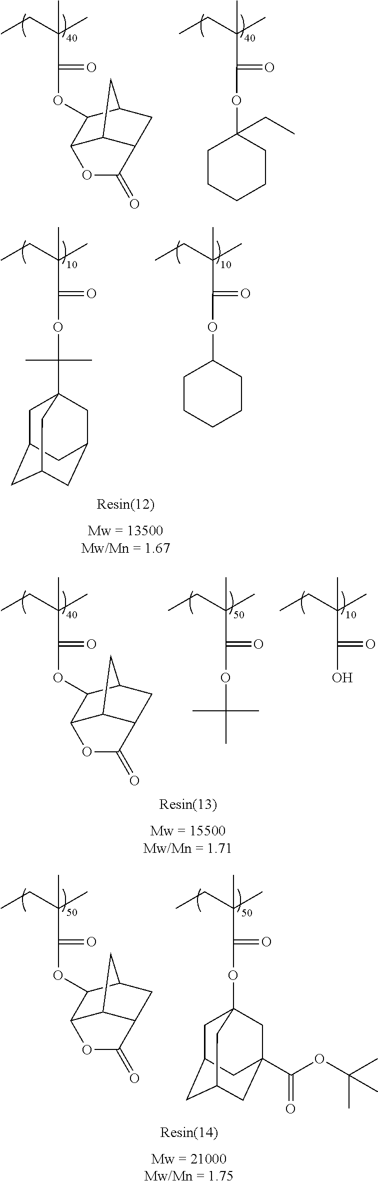

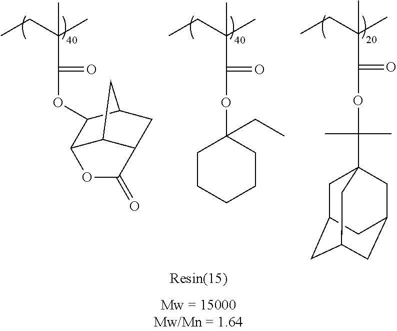

### Examples 26 to 50 and Comparative Examples 7 to 12

<Preparation of Resist>

The components shown in the following Table 3 were dissolved in a solvent to adjust a solution having an solid concentration of 3.8 by mass, and each was filtered through a polyethylene filter having a pore size of 0.03 μm to prepare an actinic ray-sensitive or radiation-sensitive resin composition (resist composition).

<Evaluation of Resist>

(ArF Liquid Immersion Exposure)

An organic antireflection film ARC29SR (manufactured by Nissan Chemical Industries, Ltd.) was coated on a silicon wafer and baked at 205° C. for 60 seconds to form an antireflection film having a thickness of 95 nm. The actinic ray-sensitive or radiation-sensitive resin composition was coated thereon and baked (PB: prebake) at 100° C. over 60 seconds to form a resist film having a thickness of 100 nm.

The obtained wafer was exposed by using an ArF excimer laser liquid immersion scanner (manufactured by ASML Co., Ltd.; XT700i, NA 1.20, C-Quad, outer sigma 0.900, inner sigma 0.812, XY deflection) through a 6% half-tone mask of 48 nm line width 1:1 line and space pattern. As the liquid for liquid immersion, ultrapure water was used. Thereafter, heating (PEB: post exposure bake) was performed at 105° C. for 60 seconds. Subsequently, the wafer was developed by performing puddling using the organic solvent-based developer (butyl acetate) for 30 seconds, and then rinsed by performing puddling using the rinse liquid [methyl isobutyl carbinol (MIBC)] for 30 seconds. Subsequently, a 48 nm line width 1:1 line and space pattern was obtained by spinning the wafer at a rotational speed of 4,000 rpm for 30 seconds.

The exposure latitude, LWR, collapse and aging stability were evaluated in the same manner as the above evaluation method. The evaluation results are shown in Table 3.

TABLE 3

| | Compound (A) | Resin (B) | Basic compound and | | Hydrophobic resin (HR) | Solvent | Surfactant |
|---|---|---|---|---|---|---|---|
| | (g) | (10 g) | Compound (C) (g) | | (35 mmg) | (molar ratio) | (10 mg) |
| Ex. 26 | A-7 (2.1) | Resin (7) | PEA | (0.30) | B-56 | A1/A2 = 80/20 | W-1 |
| Ex. 27 | A-18 (2.3) | Resin (8) | PBI | (0.32) | B-15 | A1/A2 = 70/30 | W-1 |
| Ex. 28 | A-23 (1.8) | Resin (9) | D-52 | (0.31) | B-16 | A1 | W-1 |
| Ex. 29 | A-24 (1.7) | Resin (10) | D-13 | (0.30) | B-42 | A1 | W-2 |
| Ex. 30 | A-25 (2.3) | Resin (11) | PBI | (0.30) | B-57 | A1/B1 = 90/10 | W-1 |
| Ex. 31 | A-27 (2.4) | Resin (12) | D-52 | (0.35) | B-8 | A1/A2 = 70/30 | W-1 |
| Ex. 32 | A-34 (2.3) | Resin (13) | D-13 | (0.28) | B-1 | A1/B1 = 90/10 | W-3 |
| Ex. 33 | A-35 (1.7) | Resin (7) | PBI | (0.30) | B-16 | A1/B1 = 80/20 | W-1 |
| Ex. 34 | A-36 (2.2) | Resin (8) | PBI | (0.31) | B-42 | A1/A2 = 80/20 | W-1 |
| Ex. 35 | A-37 (2.1) | Resin (9) | D-52 | (0.42) | B-56 | A1 | W-2 |
| Ex. 36 | A-38 (1.8) | Resin (10) | D-13 | (0.28) | B-16 | A1 | W-1 |
| Ex. 37 | A-39 (1.7) | Resin (11) | PBI | (0.33) | B-57 | A1/A3 = 95/5 | W-1 |
| Ex. 38 | A-40 (2.1) | Resin (14) | DIA | (0.30) | B-1 | A1 | W-1 |
| Ex. 39 | A-41 (2.2) | Resin (15) | D-52 | (0.31) | B-56 | A1 | W-4 |
| Ex. 40 | A-42 (2.3) | Resin (7) | D-13 | (0.29) | B-15 | A1/B2 = 90/10 | W-1 |
| Ex. 41 | A-43 (2.3) | Resin (8) | D-13/DIA | (0.2/0.15) | B-16 | A1/A2/A3 = 90/5/5 | W-1 |
| Ex. 42 | A-44 (2.3) | Resin (10) | PBI | (0.29) | B-56 | A1/A2 = 90/10 | W-2 |
| Ex. 43 | A-49 (2.3) | Resin (10) | PBI | (0.31) | B-57 | A1/A2 = 90/10 | W-1 |
| Ex. 44 | A-46 (2.3) | Resin (7) | D-52 | (0.30) | B-16 | A1/A2/B1 = 95/4/1 | W-1 |
| Ex. 45 | A-53 (2.3) | Resin (8) | D-13 | (0.29) | B-56 | A1 | W-1 |
| Ex. 46 | A-44 (1.2)/z95 (1.1) | Resin (9) | PBI | (0.35) | B-56 | A1/A2 = 90/10 | W-1 |
| Ex. 47 | A-35 (1.2)/z93 (1.1) | Resin (10) | PBI | (0.32) | B-56 | A1/A2 = 90/10 | W-1 |
| Ex. 48 | A-37 (1.2)/z76 (1.1) | Resin (7) | PBI | (0.28) | B-57 | A1/B2 = 80/20 | W-1 |
| Ex. 49 | A-11 (1.7)/z95 (0.2) | Resin (8) | D-52/PBI | (0.15/0.1) | B-1 | A1/A2/B1 = 95/3/2 | W-1 |
| Ex. 50 | A-9 (1.6)/z96 (0.3) | Resin (9) | DIA/PBI | (0.15/0.15) | B-16 | A1/A2 = 80/20 | W-1 |
| C. Ex. 7 | RA-1 (1.7) | Resin (7) | PBI | (0.28) | B-8 | A1/A2 = 90/10 | W-1 |
| C. Ex. 8 | RA-2 (1.8) | Resin (7) | PEA | (0.3) | B-15 | A1 | W-1 |
| C. Ex. 9 | RA-3 (1.9) | Resin (7) | D-13 | (0.28) | B-56 | A1 | W-1 |
| C. Ex. 10 | RA-4 (2.1) | Resin (7) | PBI | (0.30) | B-15 | A1/A2 = 90/10 | W-1 |
| C. Ex. 11 | RA-5 (2.0) | Resin (7) | DIA | (0.31) | B-57 | A1/B2 = 90/10 | W-1 |
| C. Ex. 12 | RA-6 (1.9) | Resin (7) | PEA | (0.40) | B-15 | A1/B2 = 80/20 | W-1 |

| | Evaluation item 1 | Evaluation item 2 | Evaluation item 3 | Evaluation item 4 Resist aging stability | |
|---|---|---|---|---|---|
| | Exposure latitude | LWR | Collapse | Number of temporally stable | Number of temporally stable |
| | (%) | (mm) | (nm) | days of contact angle (day) | days of line width (day) |
| Ex. 26 | 19.3 | 4.8 | 31.2 | 430 | 400 |
| Ex. 27 | 18.1 | 4.7 | 30.5 | 520 | 500 |
| Ex. 28 | 19.6 | 4.6 | 30.2 | 450 | 420 |
| Ex. 29 | 18.9 | 4.5 | 30.8 | 450 | 400 |
| Ex. 30 | 19.2 | 4.7 | 29.8 | 490 | 480 |
| Ex. 31 | 19.8 | 4.5 | 30.8 | 550 | 520 |
| Ex. 32 | 19.9 | 4.7 | 30 | 490 | 450 |
| Ex. 33 | 19.8 | 4.5 | 31.2 | 510 | 460 |
| Ex. 34 | 18.1 | 4.6 | 31.5 | 440 | 420 |
| Ex. 35 | 18.5 | 4.8 | 32.1 | 490 | 430 |
| Ex. 36 | 18.3 | 4.3 | 30.6 | 510 | 490 |
| Ex. 37 | 19.2 | 4.6 | 30.8 | 600 | 510 |
| Ex. 38 | 20 | 4.4 | 31.1 | 560 | 510 |
| Ex. 39 | 18.2 | 4.8 | 32.3 | 450 | 450 |
| Ex. 40 | 19.1 | 4.9 | 31.4 | 550 | 510 |
| Ex. 41 | 19.3 | 4.5 | 29.8 | 510 | 500 |
| Ex. 42 | 19.9 | 4.4 | 28.9 | 550 | 510 |
| Ex. 43 | 18.6 | 4.5 | 29.8 | 510 | 500 |
| Ex. 44 | 19.1 | 4.5 | 30.8 | 620 | 520 |
| Ex. 45 | 18.8 | 4.9 | 30.9 | 690 | 630 |
| Ex. 46 | 20.4 | 4.3 | 29.5 | 620 | 610 |
| Ex. 47 | 19.2 | 4.4 | 30.9 | 570 | 550 |
| Ex. 48 | 20.2 | 4.6 | 29.1 | 550 | 520 |
| Ex. 49 | 19.3 | 4.3 | 28.8 | 410 | 400 |
| Ex. 50 | 18.9 | 4.4 | 29.7 | 390 | 380 |
| C. Ex. 7 | 17.5 | 5.1 | 36.3 | 210 | 200 |
| C. Ex. 8 | 17.9 | 5.2 | 37.2 | 210 | 190 |
| C. Ex. 9 | 12.6 | 5.7 | 38.1 | 150 | 120 |
| C. Ex. 10 | 17.2 | 5.1 | 39.8 | 60 | 50 |
| C. Ex. 11 | 17.1 | 5.1 | 40.8 | 90 | 70 |
| C. Ex. 12 | 17.2 | 5.3 | 39.5 | 330 | 320 |

As apparent from the results shown in Table 3, it is understood that Comparative Examples 1 to 6 in which an actinic-sensitive or radiation-sensitive composition not containing the compound represented by Formula (1) is used have small exposure latitude and large LWR so that both of pattern collapse performance and aging stability are deteriorated.

Meanwhile, Examples 1 to 25 in which the compound represented by Formula (1) is used as an acid generator have large exposure latitude and small LWR so that both of pattern collapse performance and aging stability are excellent.

The composition of the present invention may be suitably used for lithography processes in manufacturing electronic devices such as various semiconductor devices and recording media.

### INDUSTRIAL APPLICABILITY

According to the present invention, it is possible to provide an actinic ray-sensitive or radiation-sensitive resin composition satisfying reduction in pattern collapse, enhancement of pattern roughness characteristics such as exposure latitude and LWR, and excellent aging stability at the same time, a resist film and a pattern forming method using the same, a method for manufacturing an electronic device and an electronic device.

This application is based on Japanese patent application No. 2012-191849 filed on Aug. 31, 2012, the entire content of which is hereby incorporated by reference, the same as if set forth at length.

1. An actinic ray-sensitive or radiation-sensitive resin composition comprising a compound represented by Formula (1): 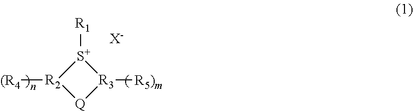
wherein R₁ represents a polycyclic aromatic group or a polycyclic heterocyclic aromatic group,
R₂ represents a (n+2)-valent saturated hydrocarbon group,
R₃ represents a (m+2)-valent saturated hydrocarbon group,
R₄ and R₅ each independently represent a substituent,
Q represents a linking group containing a heteroatom,
m and n each independently represent an integer of 0 to 12, when n is 2 or more, R₄'s may be the same or different, R₄'s may be linked to each other to form a non-aromatic ring together with R₂, when m is 2 or more, R₅'s may be the same or different, and R₅'s may be linked to each other to form a non-aromatic ring together with R₃, and
X′ represents a non-nucleophilic anion.

2. The actinic ray-sensitive or radiation-sensitive resin composition according to claim 1,
wherein in Formula (1), Q is any one linking group selected from the group (G) consisting of the following linking groups: 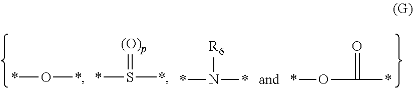
wherein R₆ represents a hydrogen atom or a substituent,
p represents an integer of 0 to 2, and
* represents a bonding hand linking to R₂ or R₃ in Formula (1).

3. The actinic ray-sensitive or radiation-sensitive resin composition according to claim 1,
wherein in Formula (1), X⁻ is a non-nucleophilic anion represented by Formula (2): 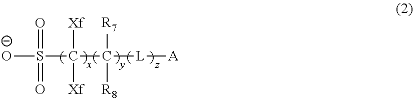
wherein in Formula (2), Xf's each independently represent a fluorine atom or an alkyl group substituted with at least one fluorine atom,
R₇ and R₈ each independently represent a hydrogen atom, a florine atom, an alkyl group or an alkyl group substituted with at least one fluorine atom, when a plurality of R₇'s is present, R₇'s may be the same or different, and when a plurality of R₈'s is present, R₈'s may be the same or different,
L represents a divalent linking group, and when a plurality of L's is present, L's may be the same as or different,
A represents an organic group containing a cyclic structure,
x represents an integer of 1 to 20,
y represents an integer of 0 to 10, and
z represents an integer of 0 to 10.

4. The actinic ray-sensitive or radiation-sensitive resin composition according to claim 1,
wherein a fluorine content of the compound represented by Formula (1) is 0.25 or less, as calculated by (sum of mass of total fluorine atoms contained in the compound represented by Formula (1))/(sum of mass of total atoms contained in the compound represented by Formula (1)).

5. The actinic ray-sensitive or radiation-sensitive resin composition according to claim 1,
wherein in Formula (1), R₁ represents a naphthyl group.

6. The actinic ray-sensitive or radiation-sensitive resin composition according to claim 5,
wherein the compound represented by Formula (1) is a compound represented by Formula (1a): 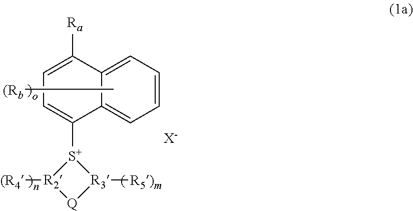
wherein in Formula (1a), Ra represents a hydrogen atom or a substituent,
Rb represents a substituent,
R₂′ and R₃′ each independently represent an alkylene group, and R₄′ and R₅′ each independently represent a substituent,
Q represents a linking group containing a heteroatom,
o represents an integer of 0 to 6, when o is 2 or more, Rb's may be the same or different,
n and m each independently represent an integer of 0 to 12, when n is 2 or more, R₄′'s may be the same or different, and R₄′'s may be linked to each other to form a non-aromatic ring together with R₂′, and when m is 2 or more, R₅′'s may be the same or different, and R₅′'s may be linked to each other to form a non-aromatic ring together with R₃′, and
X′ represents a non-nucleophilic anion.

7. The actinic ray-sensitive or radiation-sensitive resin composition according to claim 1,
wherein in Formula (1a), Ra represents a group represented by Formula (1a′):
*-A-R₆)ₛ (1a′)
wherein in Formula (1a′), A represents a divalent or trivalent heteroatom,
R₆ represents a hydrogen atom or a substituent,
s represents 1 when A is a divalent heteroatom, and s represents 2 when A is a trivalent heteroatom, and when s is 2, two R₆'s may be the same or different, and
* represents a bonding hand connecting to the benzene ring in Formula (1a).

8. The actinic ray-sensitive or radiation-sensitive resin composition according to claim 1, further comprising a resin which is decomposed by the action of an acid to change solubility in a developer.

9. The actinic ray-sensitive or radiation-sensitive resin composition according to claim 1, further comprising a low molecular weight compound having a nitrogen atom and a group capable of leaving by the action of an acid, or a basic compound.

10. A resist film formed by the actinic ray-sensitive or radiation-sensitive resin composition according to claim 1.

11. A pattern forming method comprising:
exposing the resist film according to claim 10; and
developing the exposed resist film.

12. The pattern forming method according to claim 11,
wherein the exposing is liquid immersion exposure.

13. A method for manufacturing an electronic device, comprising the pattern forming method according to claim 11.

14. An electronic device manufactured by the method for manufacturing an electronic device according to claim 13.

15. A compound represented by Formula (4): 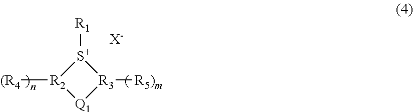
wherein in Formula (4), R₁ represents a polycyclic aromatic group ro a polycyclic heterocyclic aromatic group,
R₂ and R₃ each independently represent a (m+2)-valent saturated hydrocarbon group,
R₄ and R₅ each independently represent a substituent,
n and m each independently represent an integer of 0 to 12, when n is 2 or more, R₄'s may be the same or different, R₄'s may be linked to each other to form a non-aromatic ring together with R₂, and when m is 2 or more, R₅'s may be the same or different, and R₅'s may be linked to each other to form a non-aromatic ring together with R₃,
X′ represents a non-nucleophilic anion, and
Q₁ represents any one linking group selected from the group consisting of the linking groups shown below: 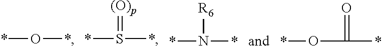
wherein R₆ represents a hydrogen atom or a substituent,
p represents an integer of 0 to 2, and
* represents a bonding hand linking to R₂ or R₃ in Formula (4).

16. The actinic ray-sensitive or radiation-sensitive resin composition according to claim 2,
wherein in Formula (1), X′ is a non-nucleophilic anion represented by Formula (2): 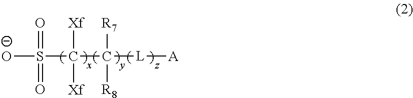
wherein in Formula (2), Xf's each independently represent a fluorine atom or an alkyl group substituted with at least one fluorine atom,
R₇ and R₈ each independently represent a hydrogen atom, a florine atom, an alkyl group or an alkyl group substituted with at least one fluorine atom, when a plurality of R₇'s is present, R₇'s may be the same or different, and when a plurality of R₈'s is present, R₈'s may be the same or different,
L represents a divalent linking group, and when a plurality of L's is present, L's may be the same as or different,
A represents an organic group containing a cyclic structure,
x represents an integer of 1 to 20,
y represents an integer of 0 to 10, and
z represents an integer of 0 to 10.
